(19) United States
(12) Patent Application Publication
(10) Pub. No.: US 2025/0372258 A1
(43) Pub. Date: Dec. 4, 2025

(54) COMPUTATIONAL FRAMEWORK FOR ENHANCING A SIGNAL-TO-NOISE RATIO (SNR) IN PROCESSING NOISY READ SIGNALS

(71) Applicant: The Broad Institute, Inc., Cambridge, MA (US)

(72) Inventors: Ruolin LIU, Cambridge, MA (US); Viktor ADALSTEINSSON, Cambridge, MA (US)

(73) Assignee: The Broad Institute, Inc., Cambridge, MA (US)

(21) Appl. No.: 19/231,368

(22) Filed: Jun. 6, 2025

Related U.S. Application Data
(63) Continuation-in-part of application No. PCT/US2023/083199, filed on Dec. 8, 2023.
(60) Provisional application No. 63/431,572, filed on Dec. 9, 2022.

(51) Int. Cl. G16H 50/20

(52) U.S. Cl. CPC G16H 50/20

(57) ABSTRACT

The present disclosure relates to a computational framework for detecting localized disruptions in noisy, low-coverage signals. Signature classes may be assigned to detected localized disruptions based on one or more features. A trained model may be applied to classified disruptions in determining associations with target medical conditions.

### CROSS-REFERENCE TO RELATED APPLICATIONS

This application is a bypass continuation-in-part of International Patent Application No. PCT/US2023/083199 filed Dec. 8, 2023, which claims priority to and the benefit of U.S. Provisional Patent Application No. 63/431,572 filed Dec. 9, 2022, each of which is incorporated herein by reference in its entirety.

### SEQUENCE LISTING

This application contains a Sequence Listing which has been submitted electronically in XML format and is hereby incorporated by reference in its entirety. The Sequence Listing XML file, created on Aug. 21, 2025, is named 114203-1775_SL.xml and is 124,240 bytes in size.

### BACKGROUND

High-throughput sequencing devices generate large datasets that present significant challenges in terms of data processing, pattern recognition, and classification. These challenges are particularly salient in noisy datasets generated from low-purity sources.

### SUMMARY

This disclosure provides a highly sensitive and computationally efficient approach for detecting homologous recombination deficiency (HRD) from read signals generated using samples with low-tumor purity. The techniques discussed herein enable detection of complex mutational patterns within noisy, low-purity sequencing data to effectively distinguish signal from background variation.

In one aspect, various embodiments of the disclosure are directed to a computing system comprising one or more processors and a non-transitory computer-readable storage medium storing instructions executable by the one or more processors. The computing system may comprise a detector module configured to detect, in a signal series, localized structural disruptions based on reads in the signal series. The detector module may be configured to execute one or more signal processing heuristics to exclude localized disruption candidates based at least in part on relative positioning of the localized disruption candidates in the signal series. The system may comprise a classifier configured to assign signature classes to the detected localized disruptions based on one or more features of the localized disruptions to obtain classified localized disruptions. The system may comprise a modeler configured to apply a trained model to the classified localized disruptions to obtain scores indicative of a degree to which the classified localized disruptions are associated with a repair pathway. The system may comprise an output module and/or an output device configured to provide, based on the scores, one or more outputs related to at least one of a target condition corresponding to the repair pathway and/or a treatment for the target condition.

In various embodiments, at least one of the signal processing heuristics or the classifier increases a signal-to-noise ratio (SNR) in analysis of the signal series. In various embodiments, the signal processing heuristics exclude candidate localized disruptions based at least in part on a distance between adjacent candidate localized disruptions. In various embodiments, the signal processing heuristics exclude candidate localized disruptions based at least in part on whether the candidate localized disruptions are situated in low-complexity regions of the signal series. In various embodiments, the localized disruptions are microhomology deletions. In various embodiments, all scores on which the one or more outputs are based correspond to microhomology deletions. In various embodiments, the repair pathway corresponds to a homologous recombination repair pathway. In various embodiments, the target condition is homologous recombination deficiency and/or neoplasia. In various embodiments, the treatment comprises at least one of a PARP inhibitor and/or platinum chemotherapy. In various embodiments, the trained model comprises a multinomial mixture model. In various embodiments, the system comprises a training module configured to train the model. In various embodiments, the training module is configured to optimize the model based at least in part on an expectation-maximization optimization algorithm.

In another aspect, various embodiments of the disclosure are directed to a method. The method may comprise detecting, in a signal series, localized structural disruptions based on reads in the signal series. Detecting the localized disruptions may comprise applying one or more signal processing heuristics to exclude localized disruption candidates based at least in part on relative positioning of the localized disruption candidates in the signal series. The method may comprise assigning signature classes to the detected localized disruptions based on one or more features of the localized disruptions to obtain classified localized disruptions. The method may comprise applying a trained model to the classified localized disruptions to obtain scores indicative of a degree to which the classified localized disruptions are associated with a repair pathway. The method may comprise providing, based on the scores, one or more outputs related to at least one of a target condition corresponding to the repair pathway or a treatment for the target condition.

In various embodiments, at least one of the signal processing heuristics and/or the classifier increases a signal-to-noise ratio (SNR) in analysis of the signal series. In various embodiments, the signal processing heuristics exclude candidate localized disruptions based at least in part on a distance between adjacent candidate localized disruptions. In various embodiments the signal processing heuristics exclude candidate localized disruptions based at least in part on whether the candidate localized disruptions are situated in low-complexity regions of the signal series. In various embodiments, the localized disruptions are microhomology deletions. In various embodiments all scores on which the one or more outputs are based correspond to microhomology deletions. In various embodiments the repair pathway corresponds to a homologous recombination repair pathway. In various embodiments, the target condition is homologous recombination deficiency and/or neoplasia. In various embodiments, the treatment comprises at least one of a PARP inhibitor or platinum chemotherapy. In various embodiments, the trained model comprises a multinomial mixture model. In various embodiments the method comprises training the model. In various embodiments, training the model comprises optimizing the model based at least in part on an expectation-maximization optimization algorithm.

### BRIEF DESCRIPTION OF THE DRAWINGS

FIG. 1 is an illustration of a computational environment that is operable to employ the techniques disclosed herein, according to various example embodiments.

FIG. 2 depicts an example procedure in which example processes are performed, according to various example embodiments.

FIG. 3 illustrates an example system including various components of an example device that can be implemented as any type of computing device as described and/or utilized with reference to FIGS. 1 and 2 to implement examples of the techniques described herein, according to various example embodiments.

FIGS. 4A-4E provide schematics, models, and plots showing an example DirectHRD workflow, according to various example embodiments.

FIGS. 5A-5B provide a table and plot showing HRD detection from liquid biopsies, according to various example embodiments.

FIGS. 6A-6F provide illustrations and plots depicting an optimized mhDel caller in DirectHRD, according to various example embodiments.

FIGS. 7A-7D provide plots showing DirectHRD performance on tumor biopsies (n=27), according to various example embodiments.

FIGS. 8A-8E provide diagrams and plots showing a metastatic breast cancer cohort, according to various example embodiments.

FIGS. 9A-9C provide plots showing an early stage triple negative breast cancer (TNBC) cohort, according to various example embodiments.

FIGS. 10A-B illustrates HRD detection from tumor biopsies and in-silico dilutions, according to various example embodiments.

FIGS. 11A-C depict HRD detection from liquid biopsies, according to various example embodiments.

FIGS. 12A-D provides additional information related to DirectHRD model development.

FIGS. 13A-C show an optimized mhDel caller in DirectHRD.

FIGS. 14A-D depicts additional results for the tumor biopsy samples in stage II-III breast cancer (TNBC) cohort.

FIGS. 15A-E depicts additional results for the prostate (FIG. 15A-B) and ovarian (FIG. 15C-E) cancers cfDNA cohorts.

FIG. 16 is a graphical representation of an exemplary DirectHRD workflow.

### DETAILED DESCRIPTION

Traditional approaches to analyzing sequenced reads often rely on rule-based or threshold-driven techniques that lack adaptability and sensitivity in low-quality or sparse signal environments. These limitations are especially pronounced in applications involving low-input samples, such as those derived from minimally invasive collection methods or degraded sources.

The disclosure provides embodiments of computational systems and methods that can efficiently process the signals obtained based on outputs from sequencers, identify structural disruptions (used interchangeably with localized sequence disruptions, disruption events, and/or localized disruptions) localized to regions in the signal series, classify the structural disruptions, and model classified disruptions to identify conditions using enhanced computational models. The systems are capable of operating on diverse sequencing platforms and data formats, and support scalable, automated analysis pipelines suitable for integration into broader data processing infrastructures.

The disclosure provides example computer-implemented frameworks for the classification of sequence-derived structural disruptions using modeling and signature-based pattern recognition. The disclosed system improves the accuracy and sensitivity of localized regions in signal series classification in low-purity data sets, and enables the generation of quantitative scores that reflect the likelihood of specific mutational processes.

Certain embodiments of the disclosure provide techniques for processing raw sequencing data to identify and classify discrete sequence disruptions (e.g., microhomology deletions) using a structured feature representation and a classification model. The system employs a multinomial mixture model to assign posterior probabilities to each event, enabling the generation of a quantitative HRD score. This score is generated through a series of technical steps including read alignment, indel detection, feature extraction, and inference. The method is specifically adapted to operate on low-input, high-noise data and produces a technically meaningful output that supports downstream clinical decision-making.

Example embodiments, referred to in places as “DirectHRD”, address a specific technical challenge of detecting HRD in low tumor-purity samples (e.g., 1-10% tumor DNA). Previous methods failed at this technical task, whereas DirectHRD can achieve greater sensitivity (e.g., 10× greater). The disclosed embodiments thus provide a practical application embedded in a technological ecosystem, and represent a significant improvement to how computers process genomic signals, extracting meaningful signal from noisy genomic data. Example embodiments enable HRD testing from liquid biopsies rather than requiring invasive tumor biopsies, making precision medicine accessible to patients who cannot undergo tissue biopsies. Implementations directly influence selection of treatment protocols (e.g., whether a treatment such as PARP inhibitors or platinum chemotherapy is warranted), and thus enables treatment decisions that were previously impossible with liquid biopsies. Direct HRD provides a significant technological leap in precision oncology.

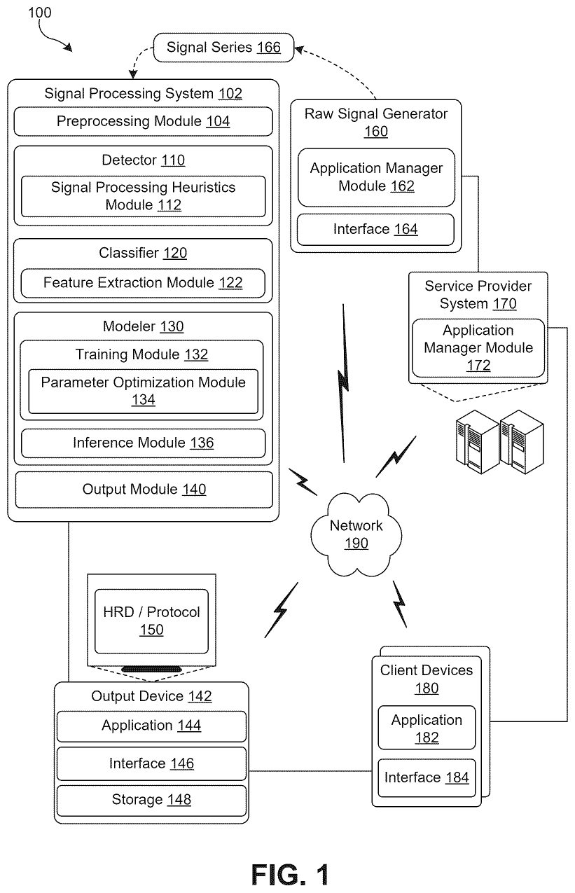

FIG. 1 is an illustration of an environment 100 in an example implementation that is operable to employ dynamic HRD classification as described herein. The illustrated environment 100, which may be implemented using a combination of hardware and software, includes a signal processing system 102, an output device 142, a raw signal generator 160 (e.g., comprising one or more genomic sequencing devices), a service provider system 170, one or more client devices 180 (which may be referred to herein in the singular form “device”), that are communicatively coupled, one to another, directly (through wired or wireless coupling) and/or via a network 190 (e.g., a local area network, wide area network, the internet, cellular network, etc.). Although the signal processing system 102 (which can implement a signal processing pipeline), the output device 142, and the raw signal generator 160 are illustrated as separate from the service provider system 170 and the client device 180, this functionality may be incorporated as part of the service provider system 170 and/or the client device 180, or further divided among other entities or system components, and so forth. By way of example, an entirety of or portions of the functionality of the signal processing system 102 may be incorporated as part of the service provider system 170 and/or the client device 180. Additionally, or alternatively, an entirety or portions of the client device 180 may be incorporated as part of the service provider system 170.

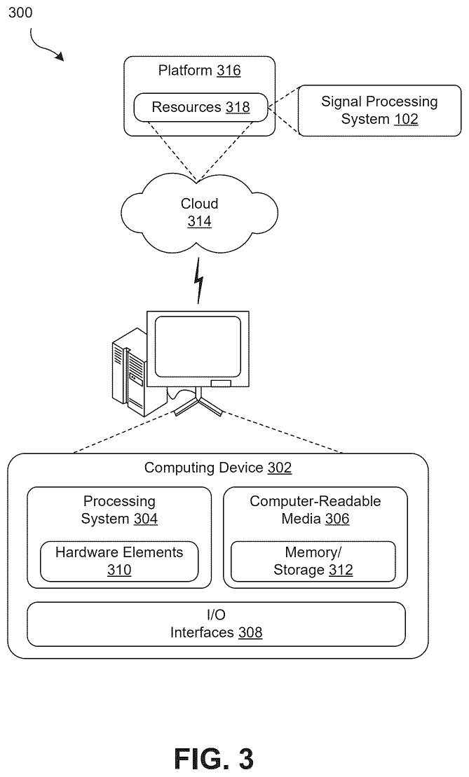

Computing devices that are usable to implement the service provider system 170, the client device 180, the raw signal generator 160, and the signal processing system 102 may be configured in a variety of ways. A computing device, for instance, may be configured as a desktop computer, a laptop computer, a mobile device (e.g., assuming a handheld configuration such as a tablet or mobile phone), and so forth. Thus, the computing device may range from full resource devices with substantial memory and processor resources (e.g., personal computers, game consoles) to a low-resource device with limited memory and/or processing resources (e.g., mobile devices). Additionally, a computing device may be representative of a plurality of different devices, such as multiple servers utilized to perform operations “over the cloud,” as further described in relation to FIG. 3.

The service provider system 170 is illustrated as including an application manager module 172 that is representative of functionality to provide access to signal processing system 102 to a user of a client device 180 via the network 190. The application manager module 172, for instance, may expose content or functionality of the signal processing system 102 or the raw signal generator 160 that is accessible via the network 190 by an application 182 of the client device 180. The application 182 may be configured as a network-enabled application, a browser, a native application, and so on, that exchanges data with the service provider system 170 via the network 190. The data can be employed by the application 182 to enable the user of the client device 180 to communicate with the service provider system 170, such as to receive application updates and features when the service provider system 170 provides functionality to manage the application 182. In various embodiments, service provider system 170 may be associated with a medical information system (MIS)/health information system (HIS), a healthcare provider (e.g., a hospital or clinic), and/or a sample testing laboratory.

In the context of the described techniques, the application 182 includes functionality to analyze data generated by at least one sequencing event. In the illustrated example, the application 182 includes an interface 184 that is implemented at least partially in hardware of the client device 180 for facilitating communication between the client device 180 and the signal processing system 102. By way of example, the interface 184 includes functionality to receive inputs to the signal processing system 102 from the client device 180 (e.g., from a user of the client device 180) and output information, data, and so forth from the signal processing system 102 to the client device 180 and/or the output device 142, as will be further elaborated herein.

The sequencing event includes determining an order of nucleotides (e.g., adenine, thymine or uracil, cytosine, and guanine) in one or more samples of nucleic acids, such as derived from one or more biological samples. Identifiers for nucleotides may be referred to herein as signals, and the ordering of signals of is referred to herein as a “sequence” or “series.” The nucleotides are also referred to as “bases.” The sequencing event will be described herein with respect to deoxyribonucleic acid (DNA) sequencing, and particularly with respect to cell-free DNA (cfDNA), such as generated by using the techniques described herein. Such techniques produce targeted cfDNA sequencing data (e.g., signal series 166) that is analyzed by the signal processing system 102 to determine an HRD status of the corresponding sample. For instance, the corresponding sample is classified as HRD positive (e.g., a tumor shows signs of an HRD) or HRD negative (e.g., a tumor does not show signs of a HRD) by the signal processing system 102. The HRD status may be output as an HRD classification or treatment protocol 150. In at least one implementation, the signal series 166 comprises a text-based file format, such as FASTQ files that store both nucleotide sequence information and quality scores for the bases in a sequencing read. In variations, the signal series 166 may comprise targeted cfDNA sequencing data in another type of file format.

In at least one implementation, the signal processing system 102 receives the signal series 166 and performs preprocessing such as read alignment and deduplication functions via a preprocessing module 104. In accordance with the techniques described herein, the signal processing system 102 includes a detector 110. The detector 110 (used interchangeably with “detection module”) may analyze raw signals and/or preprocessed signals to identify structural variations (also referred to as disruptions) in localized regions of the signal series 166. Example disruptions may include mutations such as indels.

The detector 110 includes a signal processing heuristics module 112 (used interchangeably with “heuristics module”) that is selective about which disruptions are passed along in the signal processing pipeline. The heuristics module 112 may, for example, require certain characteristics or features for each detected disruption, and if one or more criteria are not met based on a heuristic (also referred to as a signal filtering or signal conditioning), filter out or exclude the disruption. In various embodiments, the following heuristics (with particular criteria or conditions) may be applied:
(1) Fragment support heuristic—for example, disruptions not supported by a minimal number of unique sequencing fragments, such as two or more, are excluded. This helps ensure the mutation is not a sequencing artifact, and thereby enhances reliability and reduces false positives.
(2) Distance from rear ends heuristic—for example, disruptions located within a number of base pairs (bp), such as 8 bp, from the ends of a sequencing read are excluded. That is, disruptions are required to be at least the certain number of base pairs from the rear ends. This helps reduce errors due to poor-quality base calls near read termini.
(3) Proximity to germline variants heuristic—for example, disruptions within a number of base pairs (e.g., 8 bp) from a germline SNP or other disruption may be excluded. Germline variants may be defined as having ≥25% variant allele frequency (VAF) with ≥10 supporting reads. This helps prevent misclassification of inherited variants as somatic mutations.
(4) Proximity to other disruptions heuristic—for example, disruptions within a number of base pairs (e.g., 3 bp) from a SNP, or within a number of base pairs (e.g., 10 bp) from another disruption, may be excluded. This helps reduce the chances of miscalling in regions with complex or overlapping mutations.
(5) Regional complexity heuristic—for example, disruptions in low-complexity regions may be excluded, and only disruptions in high-complexity genomic regions (e.g., GIAB V3.0 “easy” regions) are retained. This helps avoid repetitive or ambiguous regions that are difficult to sequence accurately.
(6) Minimum read depth heuristic—disruptions may be required to have a minimum unique read depth (e.g., 10) at the disruption site. This helps ensure sufficient coverage for confident variant calling.

In various embodiments, other disruptions may be retained for classification purposes (e.g., classification as may be performed by classifier 120), but excluded for scoring purposes (e.g., scoring based on modeler 130), both further discussed below.

The signal processing system 102 includes a classifier 120 (used interchangeably with “classification module”) that receives disruptions detected by detector 110. Classifier 120 may employ multiple classification techniques based on the type of disruptions and other processes in the computational pipeline. Classifier 120 may include a feature extraction module 122 that generates features corresponding to disruptions. For example, for each disruption, the feature extraction module 122 may determine: disruption type (e.g., type of mutations, such as insertion or deletion); scope or length of the disruption (e.g., the number of base pairs affected, such as the number of base pairs inserted or deleted), and context (e.g., information on the surroundings of the disruption, such as whether the disruption is in a repeat region (e.g., homopolymer), or whether there is a microhomology (short identical sequences flanking the deletion)); and/or repeat or homology length (e.g., number of repeated or homologous bases).

In various embodiments, classifier 120 may classify into a format (e.g., each indel may be assigned to one of 83 predefined classes of COSMIC ID83 based on size category (e.g., 1 bp, 2-4 bp, ≥5 bps) and context (e.g., C-rich repeat, T-rich repeat, random (non-repetitive, or microhomology)). Two example class codes are 1:Del:C:2 (corresponding to 1 bp deletion in a C-rich repeat with 2 bp repeat) and 5:Del:M:3 (corresponding to ≥5 bp deletion with 3 bp microhomology). As further discussed below, in various embodiments of Direct HRD, only certain classes (e.g., mhDels ≥5 bp with ≥1 bp homology) are used in the HRD scoring model, as each class contributes differently to the final HRD score based on its association with HRD-positive or HRD-negative signatures.

In various embodiments, DirectHRD may exclusively target small deletions that occur near microhomology regions: short, identical sequences flanking the deletion site. These deletions are considered genomic scars of homologous recombination deficiency (HRD) because they are likely the result of microhomology-mediated end joining (MMEJ), an error-prone DNA repair pathway activated when HR is impaired. Classifier 120 may assign a classification to each disruption. In example embodiments, categories may be assigned to disruptions using a structured classification system based on a format (e.g., COSMIC ID83 format). This system allows each indel to be categorized according to its size, sequence context, and structural features. Advantageously, unlike other HRD classifiers that rely on large-scale genomic alterations (e.g., LOH, TAI, LST), embodiments of DirectHRD focus on small-scale, sequence-level features. This makes it 10× more sensitive, for example, than traditional methods in low tumor fraction samples, such as cfDNA from liquid biopsies.

Modeler 130 is configured to train models (e.g., via training module 132, which includes parameter optimization module 134) and/or use models (e.g., via inference module 136). In various embodiments, modeling is performed using a machine learning approach to classify and score small disruptions based on their likelihood of being associated with a condition (e.g., likelihood of being associated with homologous recombination deficiency (HRD)). In various embodiments, the model applies, comprises, or is a multinomial mixture model (MMM).

Parameter optimization module 134 may be configured to, for example, optimize the model (during training and/or updating of the model) by applying an expectation-maximization optimization algorithm. To initialize parameters, the model begins with initial estimates of the mixing proportion (π), which reflects the contribution of the HRD-positive signature to the sample, and the class probabilities for HRD-positive and HRD-negative signatures. The model assumes that the observed indel profile in a sample is a mixture of two known distributions: pᵢ, the probability of indel type i in the HRD-positive signature; and dᵢ, the probability of indel type i in the HRD-negative signature.

In an E-Step (Expectation), for each indel type, parameter optimization module 134 may generate the posterior probability that it originated from the HRD-positive signature using the current parameter estimates. In an M-Step (Maximization), the mixing proportion may be updated using the weighted average of the posterior probabilities, and optionally the signature class probabilities (pᵢ and di) may be refined using a decay-weighted update rule (e.g., using a decay parameter alpha to prevent overfitting). Parameter optimization module 134 may iterate between the E-step and M-step until the change in π is below a small threshold (e.g., 10⁻⁵), indicating convergence. If convergence is not reached, the process loops back to the E-step and continues iterating. This iterative optimization helps ensure that the model accurately reflects the contribution of HRD-associated mutational patterns in the sample. Advantageously, this optimization approach is effective with noisy, low-purity samples. In various embodiments, this approach provides unsupervised training that does not require labeled training data for each sample. Also, various embodiments provide confidence scores for each indel, with an adaptable model that can be updated with new data to refine the model.

In some embodiments, modeler 130 may perform signature assignment. For example, once classified, each mhDel may be assigned a posterior probability of being HRD-positive or HRD-negative. In example embodiments, two reference distributions are used: HRD-positive mutational signatures (e.g., COSMIC ID6) and HRD-negative mutational signatures (e.g., from PCAWG HR-proficient tumors).

Modeling in DirectHRD, as disclosed herein, provides a confidence-weighted score rather than a binary yes/no, is sensitive to low tumor fraction by being able to work with samples containing as little as ˜1% tumor DNA, is signature-based, using biologically meaningful patterns (mhDels) linked to HRD, is quantitative, enabling nuanced interpretation and longitudinal tracking, and has non-invasive compatibility by being effective on cfDNA from liquid biopsies.

Output module 140 generates one or more outputs, and/or causes one or more outputs to be stored, transmitted, or presented via audiovisual devices, based on the results of signal processing steps. In various embodiments, output module 140 may store in one or more computer-readable memory units for later access (e.g., at signal processing system 102, output device 142, service provider system 170, and/or client device 180), transmit (using secure wired and/or wireless communication protocols) to another system or device for storage, review, and/or further processing, print or cause to be printed using a printer, generate sound outputs or cause generation of sound using an audible speaker, and/or generate a visual representation, or cause a visual representation to be generated, for presentation using a display device (e.g., a touchscreen, computer monitor, etc.).

In some embodiments, output module 140 may provide one or more outputs to output device 142, which includes an application 144 for exchanging information with users and other devices and systems, interface 146 for interfacing with users and/or networks, storage 148 for saving outputs for later retrieval, and display device 150 for visual presentation of outputs (e.g., likelihood of being positive for HRD or another condition, and/or suitable treatment protocols for the condition).

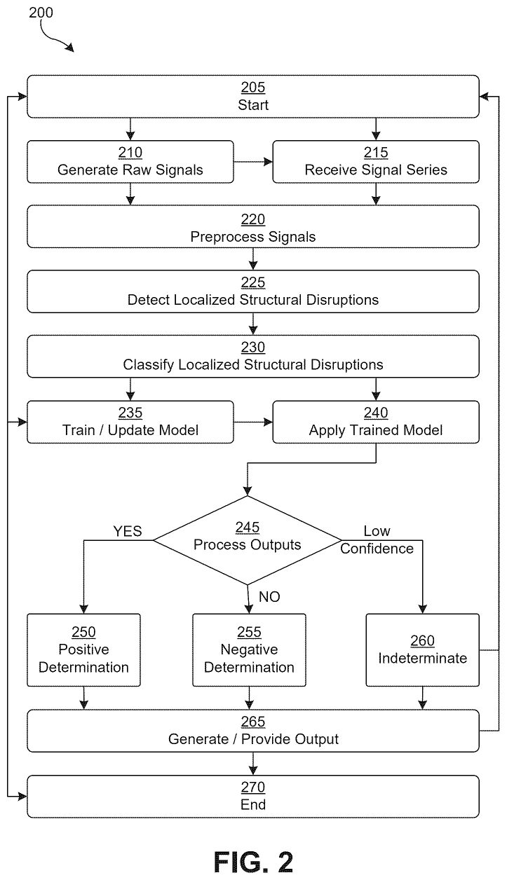

FIG. 2 illustrates a flow diagram representing a computerized platform for detecting, for example, homologous recombination deficiency (HRD) using a signal processing and classification system, such as that implemented in the DirectHRD platform. Method 200 begins at block 205, where the system (e.g., signal processing system 102) initiates analysis. At step 210, raw signals are generated (e.g., by raw signal generator) from biological inputs, such as using whole genome sequencing (WGS) reads derived from tumor tissue or circulating cell-free DNA (cfDNA). These raw signals represent discrete sequence disruption events, including insertions and deletions (indels), which are then received by the system (e.g., system 102) as a structured signal series at step 215. If the signal series is already available, method 200 may proceed to block 215 from block 205 (skipping block 210).

At step 220, the signal series is preprocessed (e.g., by preprocessing module 104) to condition and prepare the signals for detection of localized structural disruptions. Following preprocessing, the system proceeds to step 225, where it detects localized structural disruptions (e.g., using detector 110). These disruptions may include deletions exhibiting microhomology or other sequence-level anomalies that are indicative of, for example, DNA repair deficiencies associated with HRD. Detection may include application of signal heuristics (e.g., by signal processing heuristics module 112) to, for example, exclude out low-confidence localized disruptions, exclude variants located near sequencing read ends or known germline polymorphisms, and retain only disruption events that occur in high-complexity genomic regions.

In block 230, each localized structural disruption that was detected is classified (e.g., by classifier 120) into a category based on its structural and contextual features. For example, in example DirectHRD embodiments, disruption events may be categorized using the COSMIC ID83 format, which considers deletion size, microhomology length, and sequence context. If the system is operating in a training mode, block 235 may update the parameters of a machine learning model (e.g., by training module 132), such as a multinomial mixture model, using, for example, reference datasets comprising known HRD-positive and HRD-negative samples. Method 200 may, in certain embodiments, begin at block 205, proceed to block 235 for training or updating of the model, and return to starting block 205 or skipping ahead to ending block 270. In inference mode, the trained model is applied (e.g., by inference module 136) at block 240 to assign posterior probabilities to each classified event, estimating the likelihood that the event is associated with, for example, an HRD-related mutational signature.

At block 245, the model outputs are processed to generate a cumulative HRD score. This score may be obtained by summing the posterior probabilities of qualifying disruption events, such as deletions of at least five base pairs in length with at least one base pair of microhomology. Based on the generated score, the system proceeds to a decision-making stage. If the score exceeds a predefined threshold, the sample is classified as HRD-positive at block 250. If the score falls below the threshold, the sample is classified as HRD-negative at block 255.

If the system determines that the confidence in the result is insufficient—such as in cases of low tumor fraction or ambiguous signal quality—the result is marked as indeterminate at block 260. At block 265, the system generates and provides an output based on the HRD score, classification result, and optionally, supporting metadata for clinical interpretation or downstream decision-making. The process concludes at block 270. Method 200 may restart (going from block 260 and/or block 265 back to block 205). This method enables robust, automated HRD detection from low-purity samples and supports non-invasive diagnostics, longitudinal monitoring, and personalized treatment planning.

FIG. 3 illustrates an example system generally at 300 that includes an example computing device 302 that is representative of one or more computing systems and/or devices that may implement the various techniques described herein. This is illustrated through inclusion of the sequencing data processor 102. The computing device 302 may be, for example, a server of a service provider, a device associated with a client (e.g., a client device), an on-chip system, and/or any other suitable computing device or computing system.

The example computing device 302 as illustrated includes a processing system 304, one or more computer-readable media 306, and one or more I/O interfaces 308 that are communicatively coupled, one to another. Although not shown, the computing device 302 may further include a system bus or other data and command transfer system that couples the various components, one to another. A system bus can include any one or combination of different bus structures, such as a memory bus or memory controller, a peripheral bus, a universal serial bus, and/or a processor or local bus that utilizes any of a variety of bus architectures. A variety of other examples are also contemplated, such as control and data lines.

The processing system 304 is representative of functionality to perform one or more operations using hardware. Accordingly, the processing system 304 is illustrated as including hardware elements 310 that may be configured as processors, functional blocks, and so forth. This may include implementation in hardware as an application specific integrated circuit or other logic device formed using one or more semiconductors. The hardware elements 310 are not limited by the materials from which they are formed or the processing mechanisms employed therein. For example, processors may be comprised of semiconductor(s) and/or transistors (e.g., electronic integrated circuits (ICs)). In such a context, processor-executable instructions may be electronically executable instructions.

The computer-readable storage media 306 is illustrated as including memory/storage 312. The memory/storage 312 represents memory/storage capacity associated with one or more computer-readable media. The memory/storage 312 may include volatile media (such as random-access memory (RAM)) and/or nonvolatile media (such as read only memory (ROM), Flash memory, optical disks, magnetic disks, and so forth). The memory/storage 312 may include fixed media (e.g., RAM, ROM, a fixed hard drive, and so on) as well as removable media (e.g., flash memory, a removable hard drive, an optical disc, and so forth). The computer-readable media 306 may be configured in a variety of other ways as further described below.

Input/output interface(s) 308 are representative of functionality to allow a user to enter commands and information to computing device 302, and also allow information to be presented to the user and/or other components or devices using various input/output devices. Examples of input devices include a keyboard, a cursor control device (e.g., a mouse), a microphone, a scanner, touch functionality (e.g., capacitive or other sensors that are configured to detect physical touch), a camera (e.g., which may employ visible or non-visible wavelengths such as infrared frequencies to recognize movement as gestures that do not involve touch), and so forth. Examples of output devices include a display device (e.g., a monitor or projector), speakers, a printer, a network card, tactile-response device, and so forth. Thus, the computing device 302 may be configured in a variety of ways as further described herein to support user interaction.

Various techniques may be described herein in the general context of software, hardware elements, or program modules. Generally, such modules include routines, programs, objects, elements, components, data structures, and so forth that perform particular tasks or implement particular abstract data types. The terms “module,” “functionality,” and “component” as used herein generally represent software, firmware, hardware, or a combination thereof. The features of the techniques described herein are platform-independent, meaning that the techniques may be implemented on a variety of commercial computing platforms having a variety of processors.

For instance, the terms “module,” “functionality,” “engine,” and “component” may include a hardware and/or software system that operates to perform one or more functions. For example, a module, functionality, or component may include a computer processor, a controller, or another logic-based device that performs operations based on instructions stored on a tangible and non-transitory computer-readable storage medium, such as a computer memory. Alternatively, a module, functionality, or component may include a hard-wired device that performs operations based on hard-wired logic of the device. Various modules, systems, and components shown in the attached figures may represent the hardware that operates based on software or hardwired instructions, the software that directs hardware to perform the operations, or a combination thereof.

An implementation of the described modules and techniques may be stored on or transmitted across some form of computer-readable media. The computer-readable media may include a variety of media that may be accessed by the computing device 302. By way of example, and not limitation, computer-readable media may include “computer-readable storage media” and “computer-readable signal media.”

“Computer-readable storage media” may refer to media and/or devices that enable persistent and/or non-transitory storage of information in contrast to mere signal transmission, carrier waves, or signals per se. Thus, computer-readable storage media refers to non-signal bearing media. The computer-readable storage media includes hardware such as volatile and non-volatile, removable and non-removable media, and/or storage devices implemented in a method or technology suitable for storage of information such as computer readable instructions, data structures, program modules, logic elements/circuits, or other data. Examples of computer-readable storage media may include, but are not limited to, RAM, ROM, EEPROM, flash memory or other memory technology, CD-ROM, digital versatile disks (DVD) or other optical storage, hard disks, magnetic cassettes, magnetic tape, magnetic disk storage or other magnetic storage devices, or other storage device, tangible media, article of manufacture suitable to store the desired information and which may be accessed by a computer.

“Computer-readable signal media” may refer to a signal-bearing medium that is configured to transmit instructions to the hardware of the computing device 302, such as via a network. Signal media typically may embody computer readable instructions, data structures, program modules, or other data in a modulated data signal, such as carrier waves, data signals, or other transport mechanism. Signal media also include any information delivery media. The term “modulated data signal” means a signal that has one or more of its characteristics set or changed in such a manner as to encode information in the signal. By way of example, and not limitation, communication media include wired media such as a wired network or direct-wired connection, and wireless media such as acoustic, RF, infrared, and other wireless media.

As previously described, hardware elements 310 and computer-readable media 306 are representative of modules, programmable device logic and/or fixed device logic implemented in a hardware form that may be employed in some embodiments to implement at least some aspects of the techniques described herein, such as to perform one or more instructions. Hardware may include components of an integrated circuit or on-chip system, an application-specific integrated circuit (ASIC), a field-programmable gate array (FPGA), a complex programmable logic device (CPLD), and other implementations in silicon or other hardware. In this context, hardware may operate as a processing device that performs program tasks defined by instructions and/or logic embodied by the hardware as well as a hardware utilized to store instructions for execution, e.g., the computer-readable storage media described previously.

Combinations of the foregoing may also be employed to implement various techniques described herein. Accordingly, software, hardware, or executable modules may be implemented as one or more instructions and/or logic embodied on some form of computer-readable storage media and/or by one or more hardware elements 310. The computing device 302 may be configured to implement particular instructions and/or functions corresponding to the software and/or hardware modules. Accordingly, implementation of a module that is executable by the computing device 302 as software may be achieved at least partially in hardware, e.g., through use of computer-readable storage media and/or hardware elements 310 of the processing system 304. The instructions and/or functions may be executable/operable by one or more articles of manufacture (for example, one or more computing devices 302 and/or processing systems 304) to implement techniques, modules, and examples described herein.

The techniques described herein may be supported by various configurations of the computing device 302 and are not limited to the specific examples of the techniques described herein. This functionality may also be implemented all or in part through use of a distributed system, such as over a “cloud” 314 via a platform 316 as described below.

The cloud 314 includes and/or is representative of a platform 316 for resources 318, which are depicted including signal processing system 102. The platform 316 abstracts underlying functionality of hardware (e.g., servers) and software resources of the cloud 314. The resources 318 may include applications and/or data that can be utilized while computer processing is executed on servers that are remote from the computing device 302. Resources 318 can also include services provided over the Internet and/or through a subscriber network, such as a cellular or Wi-Fi network.

The platform 316 may abstract resources and functions to connect the computing device 302 with other computing devices. The platform 316 may also serve to abstract scaling of resources to provide a corresponding level of scale to encountered demand for the resources 318 that are implemented via the platform 316. Accordingly, in an interconnected device embodiment, implementation of functionality described herein may be distributed throughout the system 300. For example, the functionality may be implemented in part on the computing device 302 as well as via the platform 316 that abstracts the functionality of the cloud 314.

In addition to the embodiments expressly described herein, it is to be understood that all of the features disclosed in this disclosure may be combined in any combination (e.g., permutation, combination). Each element disclosed in the disclosure may be replaced by an alternative feature serving the same, equivalent, or similar purpose. Thus, unless expressly stated otherwise, each feature disclosed is only an example of a generic series of equivalent or similar features.

From the description herein, one skilled in the art can easily ascertain the essential characteristics of various embodiments of the present disclosure, and without departing from the spirit and scope thereof, and can make various changes and modifications of the disclosed embodiments to adapt it to various usages and conditions. Thus, other embodiments are also within the claims.

Equivalents and scope: In the articles such as “a,” “an,” and “the” may mean one or more than one unless indicated to the contrary or otherwise evident from the context. Embodiments or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claims that is dependent on the same base claim. Where elements are presented as lists, e.g., in Markush group format, each subgroup of the elements is also disclosed, and any element(s) can be removed from the group. It should it be understood that, in general, where the disclosure, or aspects of the disclosure, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the embodiments. Because such embodiments are deemed to be known to one of ordinary skill in the art, they may be excluded even if the exclusion is not set forth explicitly herein. Any particular embodiment of the disclosure can be excluded from any embodiment, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended embodiments. Those of ordinary skill in the art will appreciate that various changes and modifications to this description may be made without departing from the spirit or scope of the present disclosure, as defined in the following embodiments.

### EXAMPLE EMBODIMENTS

Example embodiments relate to detecting homologous recombination deficiency (HRD). Prior methods are inadequate for analyzing biological samples with low tumor content, such as circulating cell-free DNA (cfDNA). These methods often rely on aggregate genomic features or statistical thresholds that lack sensitivity and specificity in low-purity contexts. As a result, they fail to provide reliable outputs in clinically relevant scenarios where invasive tissue biopsies are not feasible. A technical problem addressed herein is the development of a computer-implemented system and method for accurately identifying HRD-associated mutational patterns by processing and classifying discrete sequence disruptions using a trained model. The approach operates effectively on sparse, noisy input data and produces quantitative outputs that reflect the likelihood of HRD, enabling reliable downstream clinical interpretation.

Homologous recombination (HR) deficiency (HRD) is a clinically significant phenotype reflecting cells' inability to repair DNA double-stranded breaks (DSB). Cancer patients with HRD stand to benefit from synthetic lethal therapies, such as PARP inhibitors (PARPi) and platinum chemotherapies, but the detection of HRD remains challenging. HRD tests assay either or both of (a) loss-of-function mutations in HR pathway genes, which may cause HRD, and/or (b) genomic scars resulting from HRD. Genomic scars have been identified in up to twice the number of ovarian cancer patients as mutations in HR repair (HRR) pathway genes, while large phase III trials, such as PAOLA-1 have demonstrated that patients with HRD scars benefit from PARPi irrespective of whether they have BRCA1/2 mutations. However, genomic scars can typically only be assayed in samples with high amounts of tumor DNA such as tissue biopsies, and these are not available for all patients and not at all time points in care. Being able to detect HRD scars from low purity samples, such as liquid biopsies, would broaden access to HRD testing.

Current HRD scar detection methods (e.g. MyChoice, CHORD, HRDetect, ShallowHRD) were all developed for tumor biopsies. In such samples with high fractions of tumor DNA, it is readily feasible to identify genomic scars of HRD, such as loss of heterozygosity, telomeric allelic imbalance, large-scale state transitions, and more. Yet, it becomes difficult to resolve these features apart from technical and biological noise when the fraction of tumor DNA in a sample is lower. For liquid biopsies, which often contain much lower fractions of tumor DNA, prevailing HRD detection methods assay mutations in HR pathway genes. Indeed, targeted gene sequencing panels identify somatic point mutations at variant allele frequencies of 0.1% or lower from cell-free DNA (cfDNA). Yet, detecting other mechanisms of gene inactivation, such as copy loss and epigenetic silencing, is difficult at low tumor fractions and/or requires distinct assay methodologies. Studies have sought to detect HRD scars from cfDNA, but typically only work when there is >10% tumor DNA; whereas many patients, such as those with early-stage cancer, have less tumor DNA in blood. Accordingly, there is an urgent need to increase the detection limits of HRD scar methods allowing analysis of patient samples (e.g., liquid biopsy samples).

Summary of aspects of Example Embodiments: This section provides compositions and methods for detecting Homologous Recombination Deficiency (HRD) in biological samples (e.g., liquid biopsies, tissue samples).

In an aspect, the present disclosure provides a computer-implemented method for detecting homologous recombination deficiency (HRD) in a biological sample of a subject that has or is suspected of having a neoplasia. The method involves:
a) generating a data set including a sequence of a polynucleotide or fragment thereof present in a biological sample of a subject;
b) software driven alignment of the sequence data to a reference genome;
c) analyzing the mapping results using indel-calling software to detect mutations that are insertions/deletions (indels);
d) assigning a deletion detected in step c) to a mutational signature class; and
e) assigning a score to each deletion, wherein the score reflects the distribution of deletions.

In another aspect, the present disclosure provides a computer-implemented method for detecting homologous recombination deficiency (HRD) in a biological sample of a subject that has or is suspected of having a neoplasia. The method involves:
a) generating a data set including a sequence of a polynucleotide or fragment thereof present in a biological sample of a subject;
b) software driven alignment of the sequence data to a reference genome;
c) analyzing the mapping results using indel-calling software to detect mutations that are insertions/deletions (indels) that are characterized by a deletion size of 1 bp or greater and proximal to a homology region of 1 bp or greater;
d) assigning a probability for each indel detected in step c) to be classified in a mutational signature class associated with HRD; and
e) determining a score based on the sum of the probabilities assigned to each indel in step d).

In another aspect, the present disclosure provides a method of identifying a therapy for a subject having or suspected of having a neoplasia associated with homologous recombination deficiency. The method involves:
a) generating a data set including a sequence of a polynucleotide or fragment thereof present in a biological sample of a subject;
b) software driven alignment of the sequence data to a reference genome, deduplication and indel realignment;
c) analyzing the mapping results using indel-calling software to detect mutations that are insertions/deletions (indels);
d) assigning a deletion to a mutational signature class; and
e) assigning a score to each deletion, where the score reflects the distribution of deletions, where a positive score indicates that the subject should be treated with PARP inhibitors (PARPi) or platinum chemotherapies.

In another aspect, the present disclosure provides a method of treating a selected subject. The method involves administering to the subject PARP inhibitors (PARPi) or platinum chemotherapy, where the patient is selected by the method of any one of the above aspects, or embodiments thereof.

In any of the above aspects, or embodiments thereof, the deletion is a microhomology deletion (mhDel).

In any of the above aspects, or embodiments thereof, each indel detected in step c) is characterized by a deletion size of 5 bp or greater.

In any of the above aspects, or embodiments thereof, the mutational signature class is an indel signature class. In any of the above aspects, or embodiments thereof, the indel signature class is an ID83 format COSMIC signature class. In any of the above aspects, or embodiments thereof, the mutational signature class associated with HRD is ID6 or ID8.

In any of the above aspects, or embodiments thereof, step d) further involves assigning a probability for each indel detected in step c) to be classified in a mutational signature class not associated with HRD. In any of the above aspects, or embodiments thereof, the mutational signature class not associated with HRD is a HRD negative mutational signature class of Table 9.

In any of the above aspects, or embodiments thereof, the polynucleotide is DNA or RNA.

In any of the above aspects, or embodiments thereof, the data set includes whole genome sequence data, targeted sequencing data, whole-exome sequencing data, whole-genome bisulfite sequencing data, methylation sequencing data, and/or RNA sequencing data. In any of the above aspects, or embodiments thereof, the data set includes whole genome sequence data of circulating tumor DNA and/or germline DNA.

In any of the above aspects, or embodiments thereof, the indel-calling software uses a minimum of two unique fragments to support each indel. In any of the above aspects, or embodiments thereof, the indel-calling software excludes indels that are less than 8 bp from the ends of a read. In any of the above aspects, or embodiments thereof, the indel-calling software excludes indels within 3 bp of a single nucleotide polymorphism (SNP) or within 10 bp of another indel. In any of the above aspects, or embodiments thereof, the indel-calling software excludes indels within 5 bp of germline SNPs or indels, wherein germline SNPs or indels are characterized by at least 25% variant allele frequency. In any of the above aspects, or embodiments thereof, the indel-calling software uses a unique read depth of at least 10 to detect indels.

In any of the above aspects, or embodiments thereof, the distribution of deletions includes somatic mhDels not present in germline DNA in the sample.

In any of the above aspects, or embodiments thereof, the score assigned reflects how closely the whole genome sequence of the subject corresponds to the distribution of indels found in HRD-positive vs HRD-negative tumors.

In any of the above aspects, or embodiments thereof, the whole genome sequence of the subject or a fragment thereof is compared to a reference sequence. In any of the above aspects, or embodiments thereof, the reference sequence is a germline DNA sequence, the whole genome sequence of an HRD-negative tumor, or a fragment thereof.

In any of the above aspects, or embodiments thereof, the method further involves: step f) determining that the subject has HRD when the score is greater than or equal to a predetermined score. In any of the above aspects, or embodiments thereof, the predetermined score is 1.

In any of the above aspects, or embodiments thereof, step c) further involves deduplication and indel realignment.

In any of the above aspects, or embodiments thereof, the mutational signature classes are:

| Mutation | 1 bp deletions | | | |
|---|---|---|---|---|
| class | | Base | Repeat | |
| number | Type | Pair | Size | Example |
| 1 | Del | T:A | 1 | ACCCC\|T\|CGCGGC (delete 1 T from a stretch of 1 Ts) |
| 2 | Del | C:G | 1 | ACCAA\|C\|TGCGGC |
| 3 | Del | T:A | 2 | ACCCC\|T\|TGCGGC (delete 1 T from a stretch of 2 Ts) |
| 4 | Del | C:G | 2 | ACCAA\|C\|CGCGGC |
| 5 | Del | T:A | 3 | ACCCC\|T\|TTGCGGC |
| 6 | Del | C:G | 3 | ACCAA\|C\|CCGCGGC |
| 7 | Del | T:A | 4 | ACCCC\|T\|TTTGCGGC |
| 8 | Del | C:G | 4 | ACCAA\|C\|CCCGCGGC |
| 9 | Del | T:A | 5 | ACCCC\|T\|TTTTGCGGC |
| 10 | Del | C:G | 5 | ACCAA\|C\|CCCCGCGGC |
| 11 | Del | T:A | 6+ | ACCCC\|T\|TTTTTGCGGC |
| 12 | Del | C:G | 6+ | ACCAA\|C\|CCCCCTGCGGC |
| | > = 2 bp deletions at micro-homologies | | | |
| | | Deletion | Homology | |
| | Type | size | Size | Example |
| 73 | Del | 2 bp | 1bp | ACCAA\|TC\|TAGCGGC or ACAAC\|TC\|AAGCGGC |
| 74 | Del | 3 bp | 1bp | ACCCA\|TTC\|TAGCGGC or ACCCC\|TTC\|AAGCGGC |
| 75 | Del | 3 bp | 2bp | ACCCA\|TTC\|TTAGCGGC or ACCCTC\|TTC\|AAGCGGC |
| 76 | Del | 4 bp | 1bg | ACCCA\|TATC[TTAGCGGC or ACCCAC\|TATC\|AAGCGGC |
| 77 | Del | 4 bp | 2bp | ACCCA\|TATC\|TAAGCGGC or ACCCGTC\|TATC\|AAGCGGC |
| 78 | Del | 4 bp | 3bp | ACCCA\|TATC\|TATAGCGGC or ACCCATC\|TATC\|AAGCGGC |
| 79 | Del | 5+ bp | 1bp | ACCCA\|TAGTC\|TTAGCGGC or ACCCAC\|TAGTC\|AAGCGGC |
| 80 | Del | 5+ bp | 2bp | ACCCA\|TAGTC[TAAGCGGC or ACCCCTC\|TAGTC\|AAGCGGC |
| 81 | Del | 5+ bp | Bbp | ACCCA\|TAGTC\|TAGAGCGGC or ACCCGTC\|TAGTC\|AAGCGGC |
| 82 | Del | 5+ bp | | ACCCA\|TAGTC\|TAGTAGCGGC of ACCCAGTC\|TAGTC\|AAGCGGC |
| 83 | Del | 5+ bp | 5+66 | ACCCA\|TAGCCTC\|TAGCCTAGCGGC or |
| | | | | ACCCAGCCTC\|TAGCCTC\|AAGCGGC |

In any of the above aspects, or embodiments thereof, the score is the sum of scores of deletions of ≥5 bp and ≥1 bp homology, wherein the deletions are:

| | | | | |
|---|---|---|---|---|
| 79 | Del | 5+ bp | 1 | bp |
| 80 | Del | 5+ bp | 2 | bp |
| 81 | Del | 5+ bp | 3 | bp |
| 82 | Del | 5+ bp | 4 | bp |
| 83 | Del | 5+ bp | 5+ | bp. |

In any of the above aspects, or embodiments thereof, step (e) is carried out using a multinomial mixture model to assign the score/weight to each deletion.

In any of the above aspects, or embodiments thereof, the score of the deletion has a posterior probability of 0-1 of being related to deletion.

In any of the above aspects, or embodiments thereof, the method assigns each deletion to a non-negative probability of being related to HRD.

In any of the above aspects, or embodiments thereof, the biological sample is a blood sample, serum sample, tumor biopsy, or plasma sample.

In any of the above aspects, or embodiments thereof, the subject has or is suspected of having breast, ovarian, prostate, or pancreatic cancer.

In any of the above aspects, or embodiments thereof, the biological sample has low tumor-purity.

In any of the above aspects, or embodiments thereof, the biological sample is a liquid biopsy sample. In any of the above aspects, or embodiments thereof, the liquid biopsy sample includes about 10% or more tumor DNA. In any of the above aspects, or embodiments thereof, the liquid biopsy sample includes less than 10% tumor DNA. In any of the above aspects, or embodiments thereof, the liquid biopsy sample includes about 1% tumor DNA.

In any of the above aspects, or embodiments thereof, Indels are called using Mutect2, Strelka2, Deep Variants, CODEC, or Samtools.

In any of the above aspects, or embodiments thereof, the mutation occurs in a high-complexity region of the reference genome.

In any of the above aspects, or embodiments thereof, the mutation is a deletion whose length is less than or equal to 50 base pairs.

In any of the above aspects, or embodiments thereof, the whole genome sequencing is carried out using next generation sequencing.

In any of the above aspects, or embodiments thereof, the method further involves detecting a mutation in a gene encoding BRCA1/2, CDK12, RAD51C, PALB2, or ATM.

In any of the above aspects, or embodiments thereof, the deletion includes a deletion size between 2 base pairs and at least 5 base pairs, and a homology size of between 1 base pair and at least 5 base pairs.

Compositions and articles defined by the disclosure were isolated or otherwise manufactured in connection with the examples provided below. Other features and advantages of various embodiments will be apparent from the detailed description, and from the claims, of the disclosure.

Definitions: Unless defined otherwise, all technical and scientific terms used herein have the meaning commonly understood by a person skilled in the art to which this technology belongs. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “agent” is meant a polypeptide, polynucleotide, or fragments thereof, or small molecule chemical compound. In some embodiments, the agent is a PARP inhibitor or platinum chemotherapeutic.

As used herein, the term “algorithm” refers to any formula, model, mathematical equation, algorithmic, analytical, or programmed process, or statistical technique or classification analysis that takes one or more inputs or parameters, whether continuous or categorical, and calculates an output value, index, index value or score. Examples of algorithms include but are not limited to ratios, sums, regression operators such as exponents or coefficients, biomarker value transformations and normalizations (including, without limitation, normalization schemes that are based on clinical parameters such as age, gender, ethnicity, etc.), rules and guidelines, statistical classification models, statistical weights, and neural networks trained on populations or datasets. Also, of use in the context of HRD determination as described herein are models useful for inferring an underlying tumor fraction in circulating cell free DNA (cfDNA).

By “ameliorate” is meant decrease, suppress, attenuate, diminish, arrest, or stabilize the development or progression of a disease.

By “alteration” is meant a change in the structure, expression levels or activity of a gene or polypeptide as detected by standard art known methods such as those described herein. The alteration can be an increase or a decrease. As used herein, an alteration includes a 10% change in expression levels, preferably a 25% change, more preferably a 40% change, and most preferably a 50% or greater change in expression levels. In embodiments, the change is an amino acid or nucleobase sequence alteration.

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

In this disclosure, “comprises,” “comprising,” “containing” and “having” and the like can have the meaning ascribed to them in U.S. Patent law and can mean “includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

By “complementary” is meant capable of pairing to form a double-stranded nucleic acid molecule or portion thereof. In one embodiment, an antisense molecule is in large part complementary to a target sequence. The complementarity need not be perfect, but may include mismatches at 1, 2, 3, or more nucleotides.

By “control” or “reference” is meant a standard of comparison. In one aspect, as used herein, “changed as compared to a control” sample or subject is understood as having a level that is statistically different than a sample from a normal, untreated, or control sample. Control samples include, for example, cells in culture, one or more laboratory test animals, or one or more human subjects. Methods to select and test control samples are within the ability of those in the art. Determination of statistical significance is within the ability of those skilled in the art, e.g., the number of standard deviations from the mean that constitute a positive result. In embodiments, a reference is a subject or a sample from a subject that does not have a cancer, does not have HRD, or a subject prior to a change in a treatment or administration of a drug or treatment. In embodiments, the reference is a matched normal sample, where in some instances the matched normal sample is a sample from a healthy subject and/or a subject that does not have a cancer (e.g., a subject prior to being diagnosed with a cancer or neoplasm). In some instances, the reference is a reference sequence that is a genome of a healthy subject or the sequence of cell free DNA from a healthy subject or panel of healthy subjects.

As used herein, the term “coverage” refers to the number of sequence reads that align to a specific locus in a reference sequence. In embodiments, the reference sequence is a reference genome. For example, with regard to the terminal base of the following reference sequence, because there is only one sample base aligned at this locus (the bold cytosine in Read 2), there is 1× coverage of the reference sequence at this locus. At 5′ end, there is 3× coverage of the reference sequence at the 5′ terminus guanine.

Reference Sequence:
(SEQ ID NO: 1)
5′ GGGAAGGGCGATC 3′
Read 1
(SEQ ID NO: 2)
GGGAAGGGCGAT
Read 2
(SEQ ID NO: 3)
GGGAAGGGCGATC
Read 3
(SEQ ID NO: 4)
GGGAAGGGCG

When a genome is sequenced, there will be a large number of nucleotides sequenced. If an individual genome is sequenced only once, there will be a significant number of sequencing errors. To increase the sequencing accuracy, an individual genome will need to be sequenced a large number of times. The average coverage for a whole genome can be calculated from the length of the original genome (G), the number of reads (N), and the average read length (L) as N×L/G. In another example, a hypothetical genome with 2,000 base pairs reconstructed from 8 reads with an average length of 300 nucleotides will have 2× redundancy. This parameter also enables one to estimate other quantities, such as the percentage of the genome covered by reads (sometimes also called breadth of coverage). At a coverage of 0.1×, only 10% of a reference sequence is covered by sequence reads. In embodiments, a sample polynucleotide is sequenced to a coverage of about, at least about, and/or no more than about 1e-8, 1e-7, 1e-6, 1e-5, 1e-4, 1e-3, 1e-2, 0.05×, 0.1×, 0.2×, 0.3×, 0.4×, 0.5×, 1×, 2×, 3×, 4×, 5×, 6×, 7×, 8×, 9×, 10×, 20×, 30×, 40×, 50×, 60×, 70×, 90×, 100×, or more.

By “corresponds” is meant comprising at least a fragment of a double-stranded gene, such that a strand of the double-stranded inhibitory nucleic acid molecule is capable of binding to a complementary strand of the gene.

By “decreases” is meant a reduction by at least about 5% relative to a reference level. A decrease may be by 5%, 10%, 15%, 20%, 25% or 50%, or even by as much as 75%, 85%, 95% or more and any intervening percentages.

“Detect” refers to identifying the presence, absence or amount of the analyte to be detected. In one embodiment, the analyte is a genome or fragment thereof that is analyzed for the presence of alterations indicative of homologous recombination deficiency. In some embodiments, analysis of the genome detects the presence of a mutation, mutational signature class, microhomology deletion (mhdel) (e.g., 2, 3, 4, 5, 6, 7, 8, 9, 10 bp deletion, or a 100, 200, 250, 500, 750, 1000 bp deletion) or insertion (e.g., 2, 3, 4, 5, 6, 7, 8, 9, 10 bp insertion, or a 100, 200, 250, 500, 750, 1000 bp insertion). In some embodiments, analysis of the genome or a fragment thereof detects a repeated nucleobase. In some embodiments, analysis of the genome or a fragment thereof detects a repeat that is 1, 2, 3, 4, 5, 6, 7, 8, 9, 10, 15, 20, or 25 base pairs in length. In some embodiments, the analyte detected is the presence of homology. The homology detected may be 2, 3, 4, 5, 6, 7, 8, 9, 10, 15, 20, 25, 30, 40, 50 bp in length. In some embodiments, the analyte detected is the percentage of tumor DNA (e.g., 0.1, 0.5, 1, 2, 3, 4, 5, 6, 7, 8, 9, 10%) present in a biological sample.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “disease” is meant any condition or disorder that damages or interferes with the normal function of a cell, tissue, or organ. In embodiments, the disease is a neoplasia (e.g., cancer, such as breast cancer, ovarian cancer, prostate cancer, pancreatic cancer).

The term “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell (Sambrook et al., 1989 Molecular Cloning: A Laboratory Manual, 18.1-18.88). Expression of a transfected gene can occur transiently or stably in a cell. During “transient expression” the transfected gene is not transferred to the daughter cell during cell division. Since its expression is restricted to the transfected cell, expression of the gene is lost over time. In contrast, stable expression of a transfected gene can occur when the gene is co-transfected with another gene that confers a selection advantage to the transfected cell. Such a selection advantage may be a resistance towards a certain toxin that is presented to the cell.

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications, or dosages. A therapeutically effective amount of a therapeutic compound (i.e., an effective dosage) depends on the therapeutic compounds selected. The compositions can be administered from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

By “high-throughput sequencing” is meant a sequencing technique that allows for large amounts of nucleic acids to be sequenced.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this disclosure is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule of the disclosure is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the disclosure that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the disclosure. An isolated polypeptide of the disclosure may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker” is meant any protein or polynucleotide having an alteration in expression level or activity that is associated with a disease or disorder. In some embodiments, the marker is a mutation characteristic of HRD (e.g., insertion, deletion, homologous repeat).

By “neoplasia” is meant a disease or disorder characterized by excess proliferation or reduced apoptosis. In embodiments, a neoplasia is a cancer or tumor. Illustrative neoplasms include breast cancer, esophageal cancer, head-and-neck cancer, pancreatic cancer, skin cancer, colorectal cancer, hepatocellular cancer, bladder cancer, bile duct cancer, luminal and non-luminal bladder cancer, basal bladder cancer, muscle-invasive bladder cancer, and non-muscle-invasive bladder cancer, pancreatic cancer, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, liver cancer, cervical cancer, uterine cancer, testicular cancer, lung carcinoma, small cell lung carcinoma, bladder carcinoma, epithelial carcinoma, glioma, glioblastoma multiforme, astrocytoma, medulloblastoma, craniopharyngioma, ependymoma, pinealoma, hemangioblastoma, acoustic neuroma, oligodendroglioma, schwannoma, meningioma, melanoma, neuroblastoma, and retinoblastoma). In embodiments, the neoplasia may be colon adenocarcinoma (COAD), stomach adenocarcinoma (STAD), stomach cancer, and uterine corpus endometrial carcinoma (UCEC). In one embodiment, the neoplasia is breast, ovarian, prostate, or pancreatic cancer.

As used herein, the term “next-generation sequencing (NGS)” refers to a variety of high-throughput sequencing technologies that parallelize the sequencing process, producing thousands or millions of sequence reads at once. NGS parallelization of sequencing reactions can generate hundreds of megabases to gigabases of nucleotide sequence reads in a single instrument run. Unlike conventional sequencing techniques, such as Sanger sequencing, which typically report the average genotype of an aggregate collection of molecules, NGS technologies typically digitally tabulate the sequence of numerous individual DNA fragments (sequence reads discussed in detail below), such that low frequency variants (e.g., variants present at less than about 10%, 5% or 1% frequency in a heterogeneous population of nucleic acid molecules) can be detected. The term “massively parallel” can also be used to refer to the simultaneous generation of sequence information from many different template molecules by NGS. NGS sequencing platforms include, but are not limited to, the following: Massively Parallel Signature Sequencing (Lynx Therapeutics); 454 pyrosequencing (454 Life Sciences/Roche Diagnostics); solid-phase, reversible dye-terminator sequencing (Solexa/Illumina); SOLiD technology (Applied Biosystems); Ion semiconductor sequencing (ion Torrent); and DNA nanoball sequencing (Complete Genomics). Descriptions of certain NGS platforms can be found in the following: Shendure, et al., “Next-generation DNA sequencing,” Nature, 2008, vol. 26, No. 10, 135-1 145; Mardis, “The impact of next-generation sequencing technology on genetics,” Trends in Genetics, 2007, vol. 24, No. 3, pp. 133-141; Su, et al., “Next-generation sequencing and its applications in molecular diagnostics” Expert Rev Mol Diagn, 2011, 11 (3): 333-43; and Zhang et al., “The impact of next-generation sequencing on genomics,” J Genet Genomics, 201, 38(3): 95-109.

By “model” is meant a machine learning model that may be trained using supervised and/or unsupervised learning techniques. A model as used herein may employ, but is not limited to, probabilistic modeling to define relationships between variables based upon one or more distributions. A non-limiting example of a probabilistic model is a Bayesian model, such as an interpretable Bayesian graphical model.

By “RNA-seq” is meant RNA sequencing for detecting and quantifying messenger RNA molecules (mRNA) in a biological sample, which, for example, may be used to study cellular responses. A related term, “scRNA-seq” is single-cell RNA sequencing, which may be, for example, a droplet-based single-cell RNA-seq or “Drop-seq,” that is a sequencing technology for analyzing RNA expression in at least hundreds of thousands of individual cells in embodiments of the disclosure, but may alternatively use any other high-throughput sequencing platform.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. In embodiments a “reference sequence” is the meant a single genome from a healthy donor or a representative genome that reflects input from a set of genomes In some cases, a “reference sequence” is a sequence of a polynucleotide sample (e.g., a cfDNA sample) collected from a healthy subject or from a panel of healthy subjects. In embodiments, the “reference sequence” is a collection of polynucleotide sequences corresponding to a panel of healthy subjects.

By “signal to noise ratio (SNR)” is meant the level of a desired signal relative to the level of undesired background variation.

By “reduces” is meant a negative alteration of at least 10%, 25%, 50%, 75%, or 100%.

By “subject” is meant a mammal, including, but not limited to, a human or non-human mammal, such as a bovine, equine, canine, ovine, or feline.

As used herein, the terms “treatment,” “treating,” “treat” and the like, refer to obtaining a desired pharmacologic and/or physiologic effect. “Treatment,” as used herein, covers any treatment of a disease or condition in a mammal, particularly in a human, and includes inhibiting the disease (e.g., arresting its development) and/or relieving the disease (e.g., causing regression of the disease). In embodiments, the treatment involves selecting a subject for treatment, which selection characterizes the genome of the subject. Such characterization comprises detecting an alteration in the genome, diagnosing the presence of disease (e.g., cancer), monitoring therapy, or determining a prognosis for the subject. In embodiments, treatment ameliorates at least one symptom of a neoplasia. For example, a treatment can result in a reduction in tumor size, tumor growth, cancer cell number, cancer cell growth, or metastasis or risk of metastasis. “Tumor derived DNA” means DNA that is derived from a cancer cell rather than a healthy control cell. Tumor derived DNA often includes structural changes that are indicative of cancer.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

The term “tumor fraction” means the portion of DNA in a sample derived from or predicted to be derived from neoplastic cells. In embodiments, the DNA is cell free DNA (cfDNA).

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

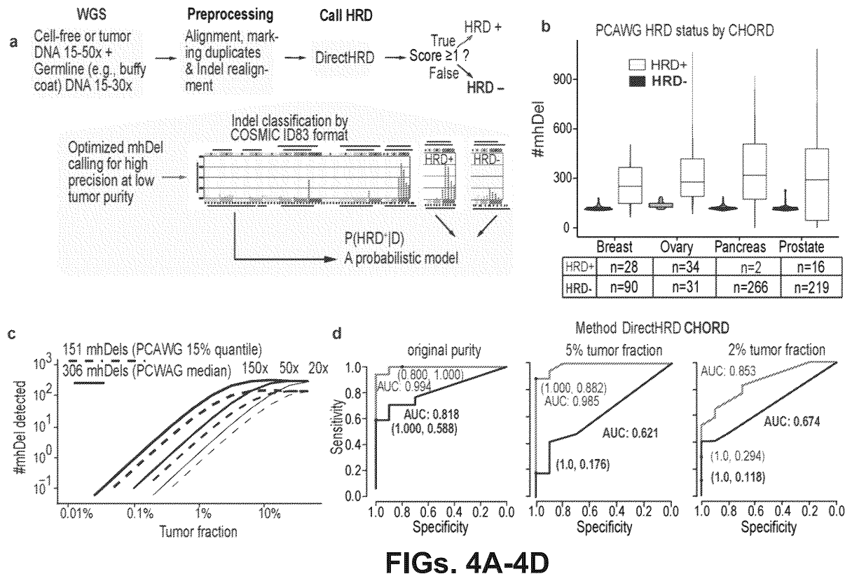

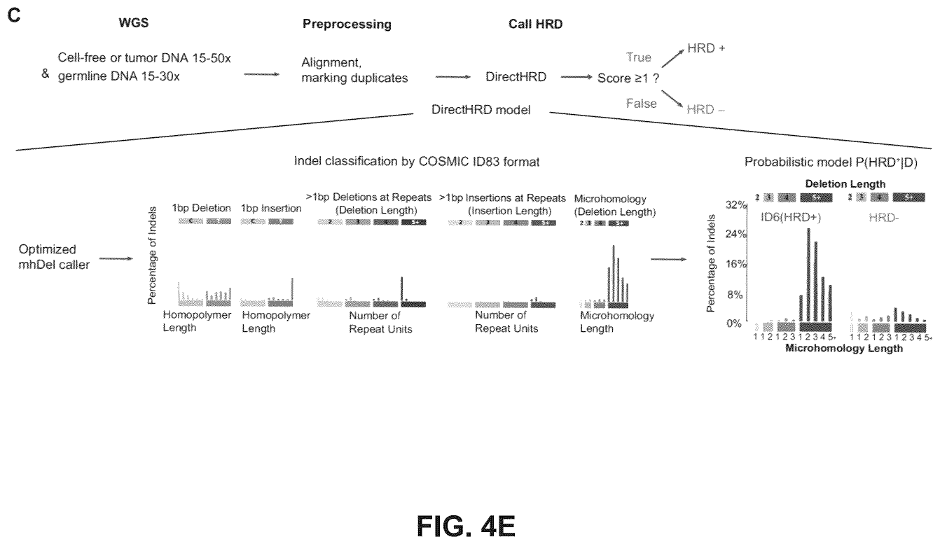

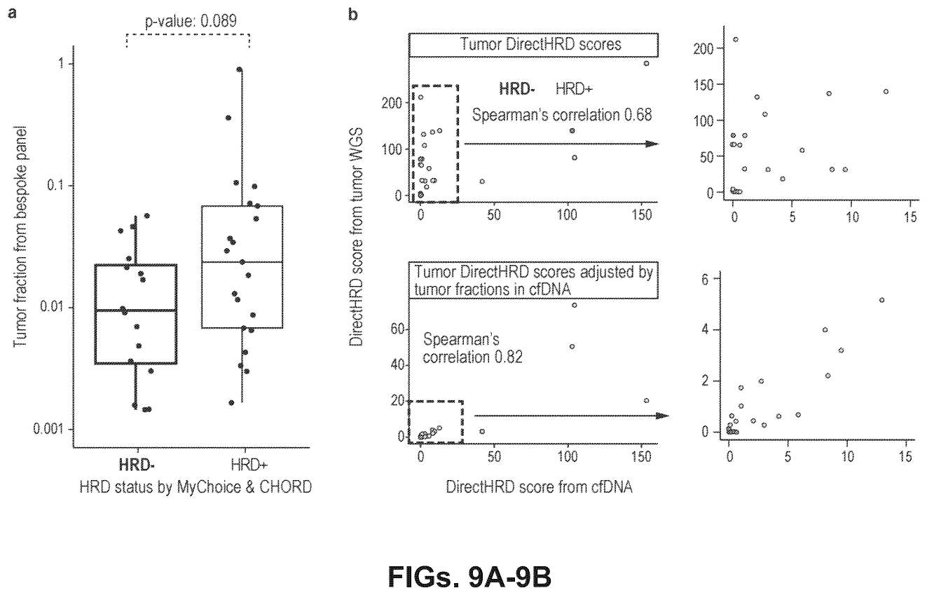

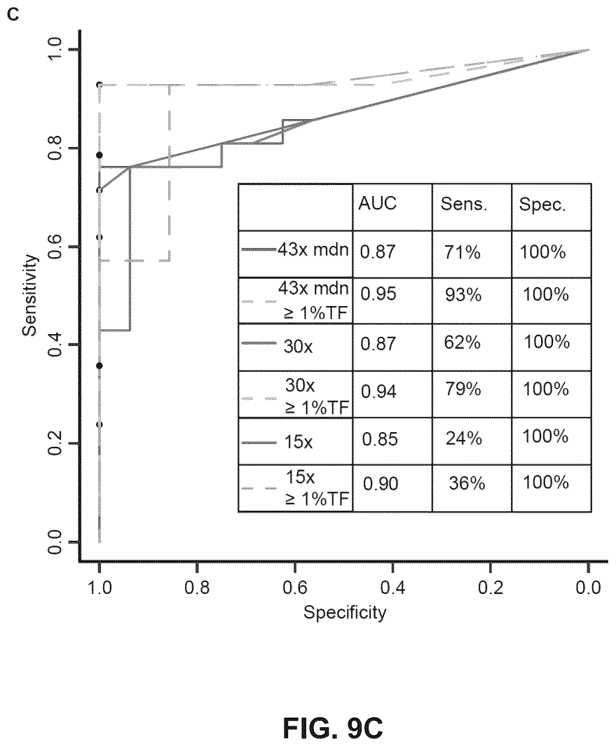

Regarding the figures, FIGS. 4A-4D provide schematics, models, and plots showing the DirectHRD workflow and proof of concept. FIG. 4A shows a schematic of DirectHRD workflow. FIG. 4B provides a plot showing the number of microhomology deletions (mhDels) per sample in Pan-Cancer Analysis of Whole Genomes (PCAWG) data across 4 cancer types, stratified by HRD statuses calculated using the tool CHORD (FIG. 9B shows the number of HRD positive and negative samples used in the plot). FIG. 4C provides a model showing the expected number of mhDels with decreasing tumor fraction. Here, binomial models were used with 3 sequencing depths (20×, 50×, 150×) and 2 starting mhDel burdens corresponding to the 15% quantile (151) and median (306) number of mhDels in the PCAWG tumors classified as HRD positive by CHORD. FIG. 4D provides a series of plots showing receiver operating characteristic (ROC) curves and area under the curve (AUC) of HRD detection from tumor biopsies and simulated dilutions of tumor biopsies, left: undiluted 27 tumors down sampled at 15× coverage (tumor fractions: 14%-74%), middle: 5% tumor fraction, right: 2% tumor fraction at 15×. The black dot and the numbers in the parentheses indicate the performance (specificity, sensitivity) when the default cutoffs for HRD detection were used (DirectHRD score: 1.0, CHORD p-value: 0.5). FIG. 4E shows another schematic of Direct HRD, which employs a specialized mhDel caller to detect mhDels alongside other Indels from WGS of a cfDNA and germline DNA (e.g. buffy-coat DNA). The resulting Indel profile (in ID83 format) is compared to the known Indel signature (COSMIC ID6) using a probabilistic model. A continuous quantitative HRD score is then reported for each sample.

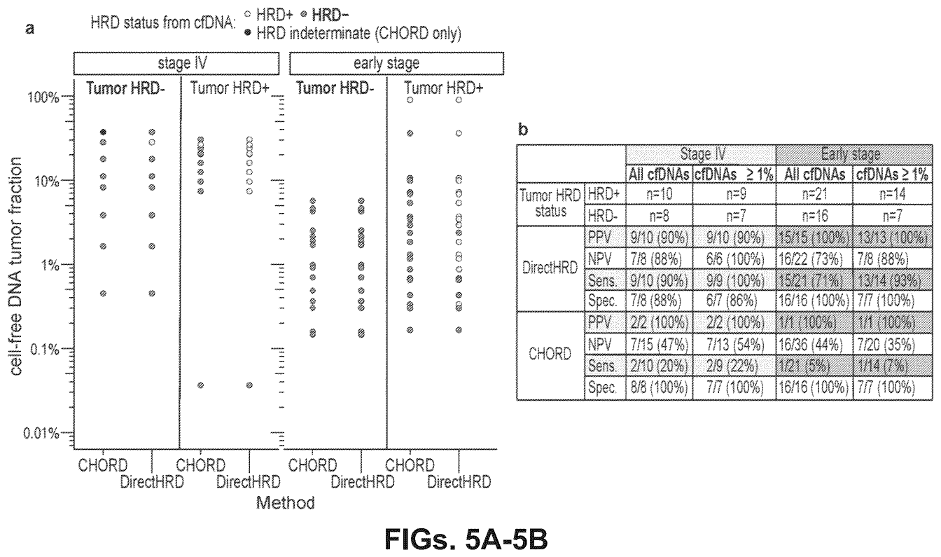

FIGS. 5A-5B provide a table and plot showing HRD detection from liquid biopsies. FIG. 5A shows a comparison of DirectHRD and CHORD on 18 stage IV breast cancer patients' cfDNA samples and 37 early stage TNBC patients' cfDNA samples. The HRD statuses inferred from patients' tumors were used as ground truth and were plotted side by side. HRD statuses inferred from cfDNA were color-coded (light gray: positive, dark gray: negative). CHORD classified 1 cfDNA sample as MSI-high and reported undetermined HRD status (black). FIG. 5B shows tabulated results of Positive Predictive Value (PPV), Negative Predictive Value (NPV), Sensitivity (Sens.) and Specificity (Spec.) of DirectHRD and CHORD.

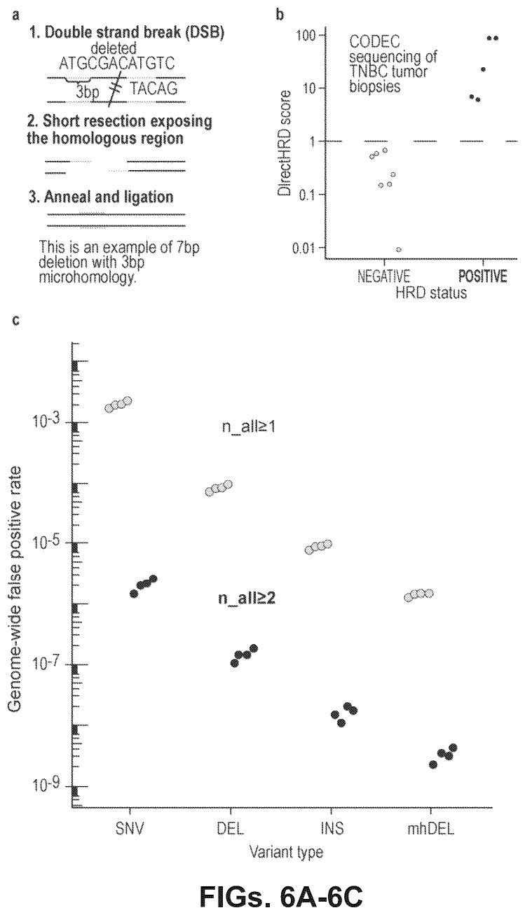

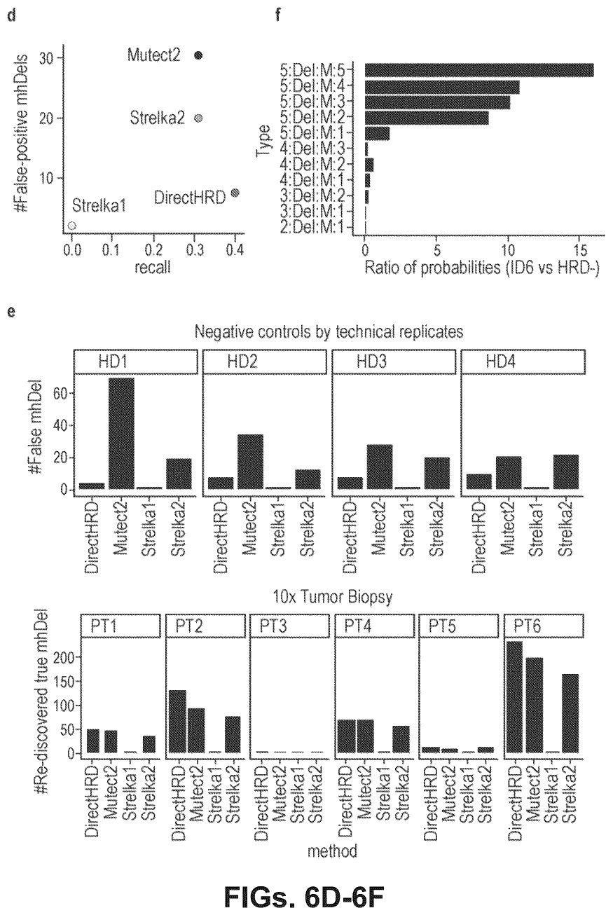

FIGS. 6A-6F provide illustrations and plots showing the optimized mhDel caller in DirectHRD. FIG. 6A shows an example illustration of the creation of an mhDel by repairing a double strand break. FIG. 6B shows DirectHRD scores of 12 TNBC tumor biopsies using shallow CODEC (Bae et al., Nat. Genet., 2023 May; 55(5): 871-879. doi: 10.1038/s41588-023-01376-0). The dashed line represented the HRD score cutoff 1 which correctly classified all samples using Myriad MyChoice HRD testing as ground truth. FIG. 6C shows the error rate of the Single Fragment Caller (SFC), part of DirectHRD, on 4 normal-normal pairs (which were also used in FIGS. 6D, 6E) from Illumina sequencing of different variant types. When requiring at least 2 unique fragments for calling a mutation, the error rate was several orders of magnitude lower than requiring at least 1 unique fragment. FIGS. 6D-6E show the performance of mhDel callers (DirectHRD, Mutect2, Strelka1 and Strelka2) on a set of 6 positive control samples (cancer patients, PTs) and 4 negative control samples (healthy donors, HDs). The recall is defined as the number of rediscovered mhDels in the 10× tumor WGS when the ground-truth set of mhDels were generated from 60× tumor WGS by taking the consensus results of Mutect2 and Strelka2. False positive mhDels were called from two technical replicates of the same HD's buffy coat DNA. FIG. 6D shows the median recall and the median number of false positive mhDels per method. FIG. 6E shows detailed numbers of true positives and false positives for each individual and each method. FIG. 6F shows the 11 mhDel types used in the DirectHRD model. Each mhDel type appears with a different probability in the HRD+ (ID6) signature vs HRD-signatures. The ratios between the two probabilities support HRD positivity when they exceed a value of 1.

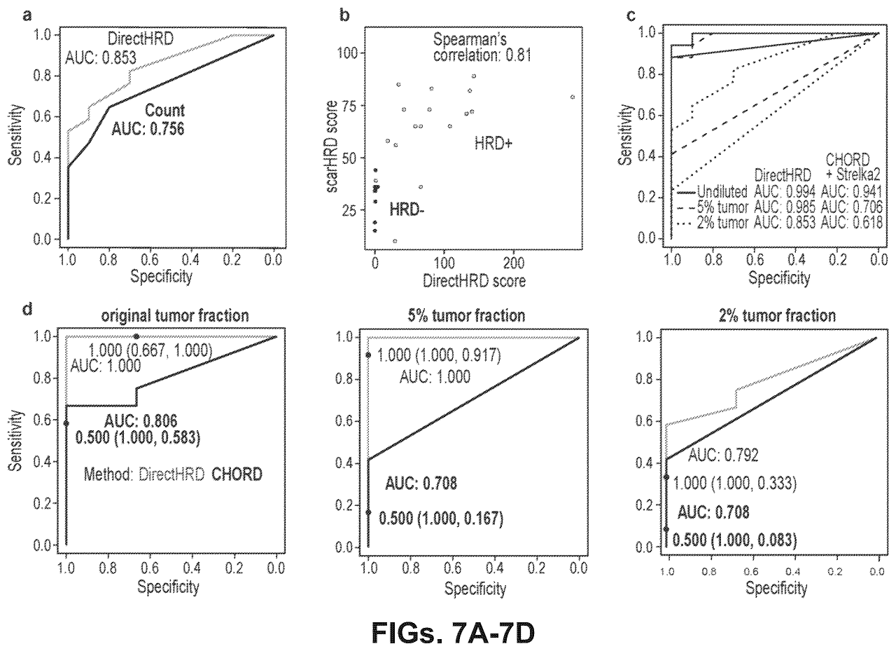

FIGS. 7A-7D provide plots showing DirectHRD performance on tumor biopsies (n=27). FIG. 7A shows a comparison of DirectHRD's classification power using a model to a count model at 2% tumor fraction for the in-silico-diluted tumor biopsy samples. Count model is simply using the number of mhDels with ≥2 microhomology (feature used in CHORD) for classification. FIG. 7B shows a scatter plot of DirectHRD scores vs scarHRD scores, having different darkness levels according to HRD status from the Myriad MyChoice assay. Spearman's correlation is shown. FIG. 7C shows a comparison of DirectHRD vs CHORD+Strelka2 on 15× tumor biopsy samples and the in silico dilutions at 5% and 2% tumor fractions, also at 15×. FIG. 7D shows a replot of performance of DirectHRD vs CHORD using only 15 TNBC tumor samples (3−, 12+) after removing the tumor samples also sequenced by CODEC (n=12). Receiver operating characteristic (ROC) curves, area under the curve (AUC), sensitivity and specificity (black dots) when using default cutoffs for HRD detection (DirectHRD score: 1.0, CHORD p-value: 0.5) are shown for original, 5% and 2% tumor fractions, all at 15× coverage.

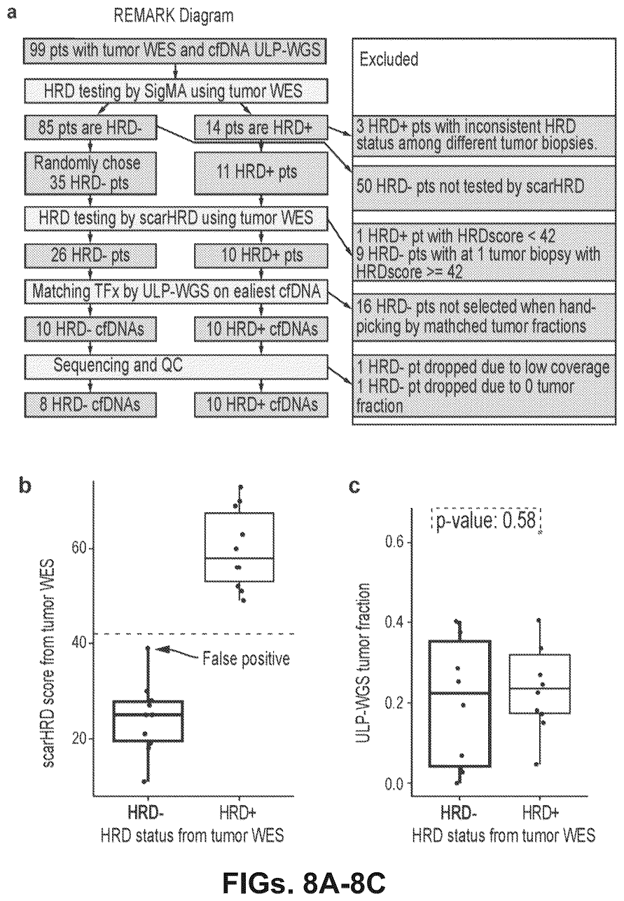

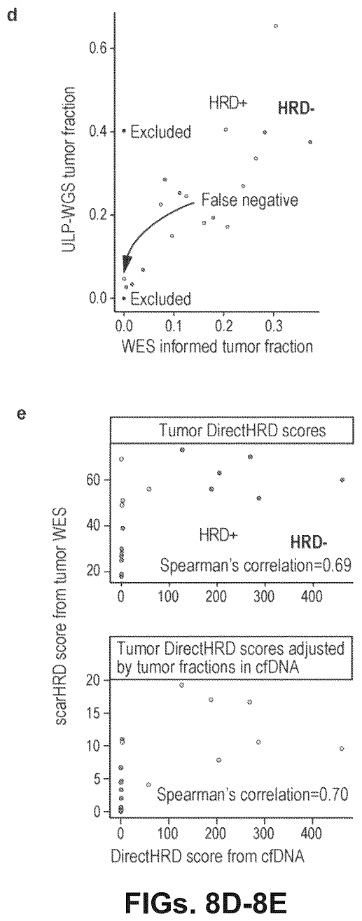

FIGS. 8A-8E provide diagrams and plots showing a metastatic breast cancer cohort. FIG. 8A shows a REMARK diagram. pt: patient; TFx: tumor fractions. FIG. 8B shows a scatter plot overlaid by box plot of 20 scarHRD scores, separated by HRD status inferred from WES of tumor biopsies by consensus results of scarHRD and SigMA. The cutoff value of 42 used here is indicated by a horizontal dashed line. FIGS. 8B, 8D show the false positive and false negative cfDNA samples by DirectHRD (ground truth HRD status by tumor WES, see 12A). FIG. 8C shows a scatter plot overlaid by box plot of tumor fractions by ichorCNA (Adalsteinsson et al., Nat Commun, 2017; 8(1):1324. doi: 10.1038/s41467-017-00965-y) of 20 ULP-WGS libraries that were selected for deeper WGS, separated by HRD status inferred from tumor WES. Mann-Whitney U test p-value showed no statistical difference between the two distributions. FIG. 8D shows tumor fractions estimated from previous ULP-WGS cfDNA samples using ichorCNA vs re-estimation from deeper WGS using tumor WES informed SNVs. 2 out of the 20 samples were excluded for further analysis because no tumor DNA was found in deeper WGS (pt300: ULP-WGS: 0.4, WES informed: 0; pt306: ULP-WGS: 0, WES-informed: 0), and one of the two samples also had low coverage (pt300: 4×). FIG. 8E shows a scatter plot of DirectHRD scores from cfDNA vs scarHRD scores from tumor WES in 18 patients (2 excluded from FIG. 8D). The scores from the tumor samples are shown as both unadjusted (top) and adjusted by tumor fractions in cfDNA (bottom). Spearman's correlations are presented. (FIGS. 8A-8E) Shading corresponds to the HRD status of the patient being positive (thicker line) vs. negative (thinner line) by tumor WES.

FIGS. 9A-9B provide plots showing an early stage TNBC cohort. FIG. 9A shows a scatter plot overlaid by box plot of estimated tumor fractions from personalized targeted panel sequencing of the same cfDNA samples by MAESTRO (Gydush et al., Nat Biomed Eng. 2022 March; 6(3):257-266. doi: 10.1038/s41551-022-00855-9) separated by HRD status from the Myriad MyChoice test (n=29) and CHORD (n=4) applied to the tumor biopsies. Mann-Whitney U test p-value showed no statistical difference between the two distributions. FIG. 9B shows a scatter plot of DirectHRD scores from cfDNA vs tumor WGS. The scores from tumor samples are shown as both unadjusted (top) and adjusted by tumor fraction in cfDNA (bottom). Highlighted regions are shown as zoom-ins on the right. Spearman's correlations are presented. In FIGS. 9A-9B, color coding corresponds to the HRD status of the patient being positive (dark gray) vs. negative (light gray) from MyChoice testing of the tumor. FIG. 9C shows receiver operating characteristic (ROC) curves and tabulated results of area under the curve (AUC), sensitivity (Sens.) and specificity (Spec.) of DirectHRD on the 37 cfDNAs in the stage II-III breast cancer cohort at 3 different sequencing depths: 43× (original median depth, non-downsampled data), 30× and 15×.

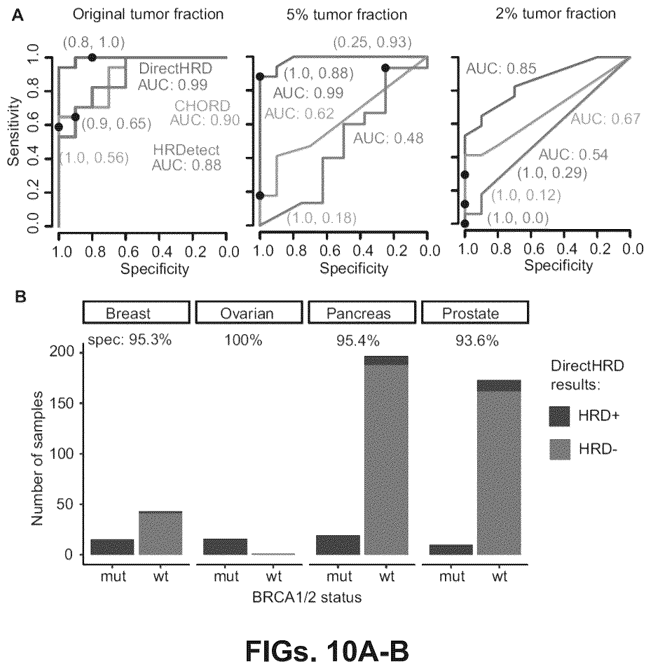

FIGS. 10A-B illustrates HRD detection from tumor biopsies and in-silico dilutions, according to various example embodiments. FIG. 10A shows receiver operating characteristic curves and AUC of HRD detection from tumor biopsies and their in-silico dilutions, left: undiluted 27 tumors downsampled at 15× coverage (tumor fractions: 14%-74%), middle: 5% tumor fraction at 15×, right: 2% tumor fraction at 15×. The dot and the numbers in the parentheses indicate the performance (specificity, sensitivity) when the default cutoffs for HRD detection were used (DirectHRD score: 1.0, HRDetect P-value: 0.7, CHORD P-value: 0.5). FIG. 10B shows DirectHRD results on 474 PCAWG samples used by CHORD for performance evaluation, where the ground-truth HRD status was determined by bi-allelic loss-of-function status of BRCA1/2 genes (mut: mutant, wd: wildtype, spec: specificity).

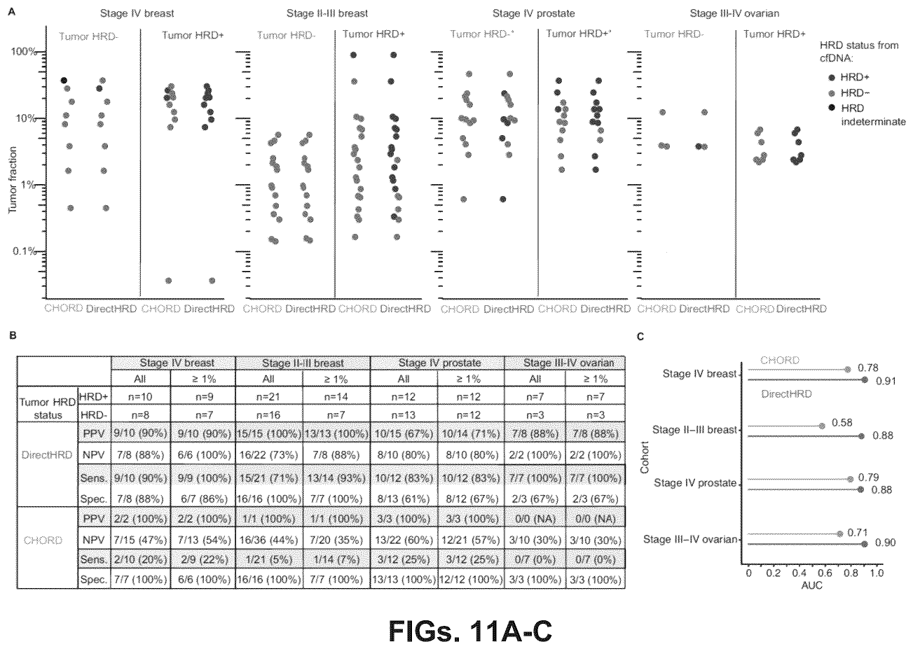

FIGS. 11A-C depicts HRD detection from liquid biopsies, according to various example embodiments. FIG. 11A shows a comparison of DirectHRD and CHORD on (from left to right): 18 cfDNAs from 18 patients with stage IV (metastatic) breast cancer, 37 cfDNAs from 29 patients with stage II and III breast cancer (TNBC), 25 cfDNAs from 20 patients with stage IV (metastatic) prostate cancer, and 10 cfDNAs from 10 patients with stage III and IV ovarian cancer. Excepting prostate cancer, the HRD statuses inferred from patients' tumors by HRD scar methods were used as the ground truth and plotted side by side. For prostate cancer, the HRD statuses were inferred from germline or somatic pathogenic alterations in HR pathway genes from targeted panel sequencing of germline and cfDNA (indicated by *). In stage IV (metastatic) breast cancer, CHORD classified 1 cfDNA sample as microsatellite instable and reported undetermined HRD status. See the ‘Materials and methods’ section for details on HRD status determination and tumor fraction calculation. FIG. 11B shows tabulated results of positive predictive value (PPV), negative predictive value (NPV), sensitivity (Sens.), and specificity (Spec.) of DirectHRD and CHORD across all cfDNA samples and those with tumor fractions >1%. FIG. 11C shows a lollipop plot comparing AUCs of DirectHRD and CHORD across all liquid biopsy cohorts.

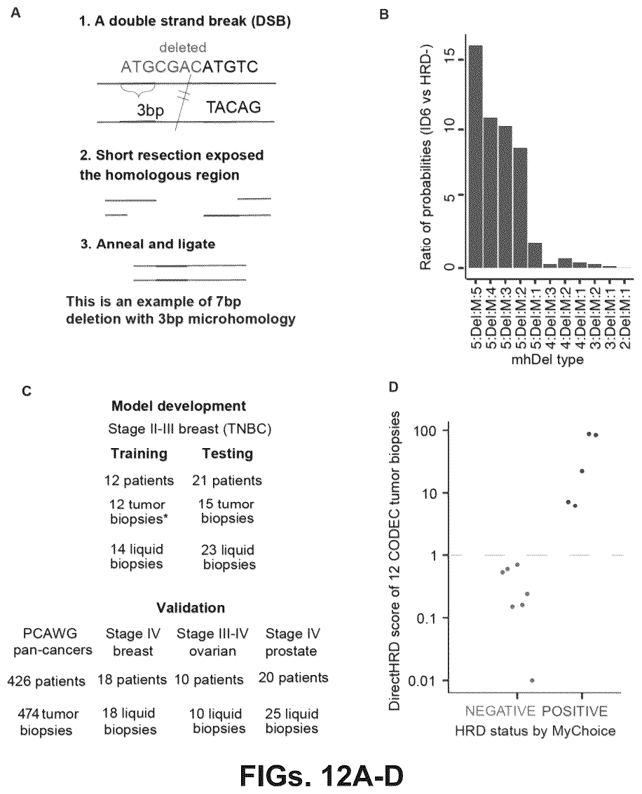

FIGS. 12A-D provides additional information related to DirectHRD model development. FIG. 12A shows an example illustration of the creation of an mhDel by repairing a double strand break. FIG. 12B shows the 11 mhDel types used in the DirectHRD model. Each mhDel type occurs with a different probability in the HRD+ (ID6) signature compared to the HRD-signature. The ratio between the two probabilities supports HRD positivity when it exceeds a value of 1. The definitions of these mhDel types can be found in https://cancer.sanger.ac.uk/signatures/id/. FIG. 12C shows a summary of samples used in this study and their utilization for training, testing and hold-out validation. * indicates samples which were also sequenced with CODEC (2). FIG. 12D shows DirectHRD scores of 12 TNBC tumor biopsies using CODEC. The dashed line represents the HRD score cutoff 1, which correctly classified all samples based on Myriad MyChoice HRD testing as the ground truth.

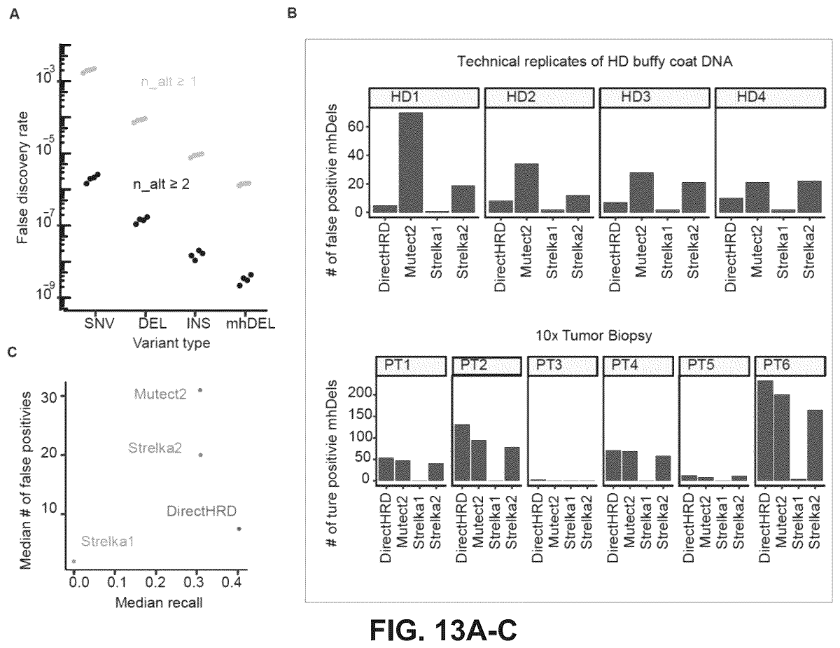

FIGS. 13A-C shows an Optimized mhDel caller in DirectHRD. FIG. 13A shows a genome-wide false discovery rate (FDR) of variants per base pair by variant type using CODEC Single Fragment Caller (SFC), employed by DirectHRD, on 4 pairs of negative controls. Each pair consists of two technical replicates of a healthy donor's DNA, sequenced using Illumina NovaSeq. Requiring at least 2 unique fragments for mutation calling resulted in error rates several orders of magnitude lower compared to requiring only 1 unique fragment. FIGS. 13B, 13C show benchmarking of mhDel callers (DirectHRD, Mutect2, Strelka1 and Strelka2) on 10× downsampled WGS of tumor biopsies from 6 cancer patients (PT) and 30×WGS of buffy coat DNA from 4 health donors (HD). The set of ground-truth variants was determined based on the consensus results of Mutect2 and Strelka2 from 60× tumor WGS. False-positive mhDels were identified from two technical replicates of the same HD's buffy coat DNA. FIG. 13B shows detailed counts of true positives and false positives for each individual and each method. FIG. 13C shows the median recall and the median number of false positives for each method.

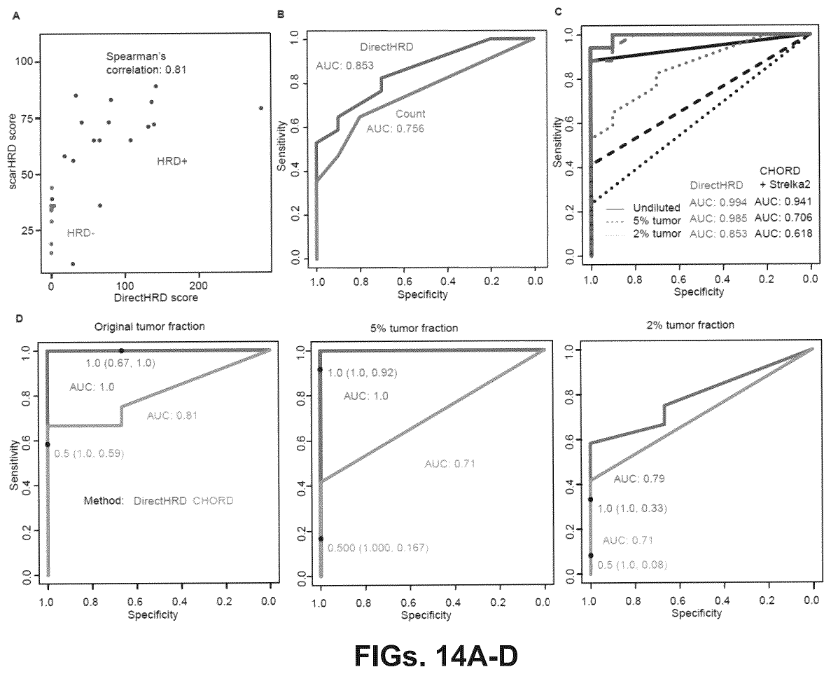

FIGS. 14A-D depicts additional results for the tumor biopsy samples in stage II-III breast cancer (TNBC) cohort. FIG. 14A shows a scatter plot of DirectHRD scores vs. scarHRD scores for the 27 tumor biopsies, colored by HRD status from the Myriad MyChoice assay. Spearman's correlation is displayed. FIG. 14B shows a comparison of DirectHRD's classification power using ROC curves and AUC values for a probabilistic model vs. a count model using in-silico-diluted tumor biopsy samples with a 2% tumor fraction. The count model uses the number of mhDels with ≥2 microhomology (a feature used in CHORD). FIG. 14C shows ROC curves and AUC values for DirectHRD and CHORD+Strelka2 on 15× tumor biopsy samples and their in-silico dilutions at 5% and 2% tumor fractions. FIG. 14D shows re-evaluation of ROC curves and AUC values of DirectHRD vs. CHORD in FIG. 10A, using only 15 TNBC tumor samples (12 HRD+, 3 HRD−) after excluding 12 samples that were also sequenced by CODEC. Sensitivities and specificities (black dots) are shown using default cutoffs for HRD detection (DirectHRD score: 1.0, CHORD p-value: 0.5)

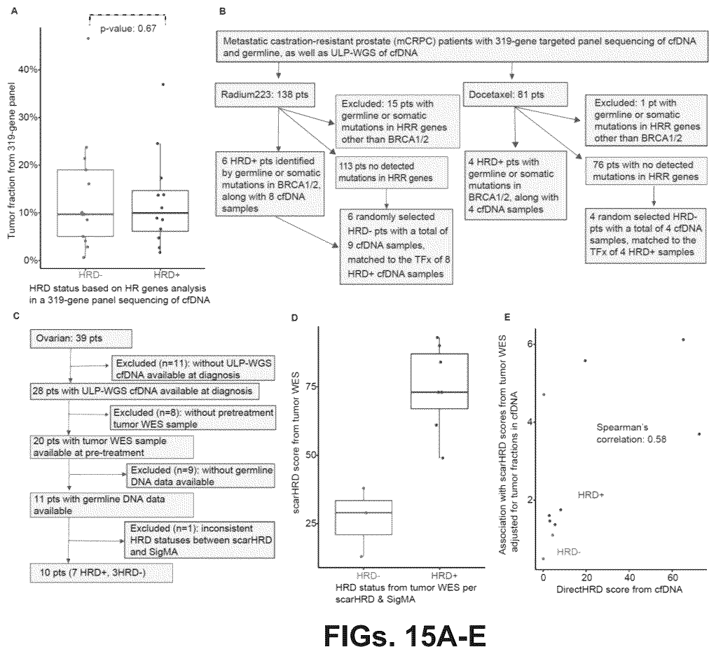

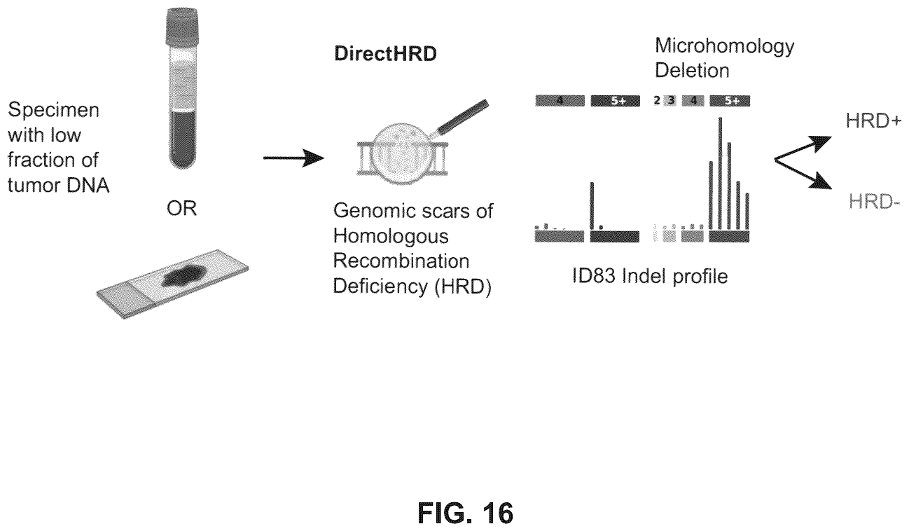

FIGS. 15A-E depicts additional results for the prostate (FIG. 15A-B) and ovarian (FIG. 15C-E) cancers cfDNA cohorts. FIG. 15A shows a scatter plot overlaid by box plot of tumor fractions, separated by HRD status inferred from 319-gene targeted panel. Mann-Whitney U test p-value is pretended and no statistically significant difference between the two distributions was found. FIG. 15B shows a flowchart diagram of sample selection in a prostate cancer cohort (metastatic castration-resistant prostate cancer, mCRPC). Patients who were HRD-positive had pathogenic or likely pathogenic germline or somatic mutations in BRCA1/2 genes (by ClinVar categories 4/5) identified through prostate cancer specific panel sequencing of patients' cfDNA and germline samples. In contrast, no pathogenic or likely pathogenic germline or somatic mutations were found in HRD-patients' HRR genes. pt: patient; TFx: tumor fractions; HRR: Homologous Recombination Repair. FIG. 15C shows a REMARK diagram of ovarian cancer cohort. FIG. 15D shows a scatter plot overlaid by box plot of scarHRD scores of 10 ovarian cancer patients, separated by HRD status inferred from WES of tumor biopsies by consensus results of scarHRD and SigMA. In FIGS. 15A, 15D, center lines, boxes, and whiskers indicate medians, 25% and 75% percentiles, and 5% and 95% percentiles, respectively. FIG. 15E shows an association of DirectHRD scores from cfDNA with adjusted scarHRD scores from tumor WES in 10 ovarian cancer patients. The scores of tumor WES samples were adjusted for tumor fractions in cfDNA. Spearman's correlation is presented and HRD statuses were based on consensus results of scarHRD and SigMA from tumor WES. In FIGS. 15A,15D & 15E, color coding corresponds to the HRD status of the patients being positive (darker gray on right side) vs. negative (lighter gray on left side).

The Example Embodiments section features compositions and methods for detecting homologous recombination deficiency (HRD) in a biological sample of the subject.

The Example Embodiments section is based, at least in part, on the development of a model—(sometimes referred to as DirectHRD herein)—trained to detect the genomic scars of HRD from low purity samples such as liquid biopsies using standard whole genome sequencing data by utilizing Cosmic Indel signatures (Catalog of Somatic Mutations in Cancer (v3.2—June 2022), which is available at https://cancer.sanger.ac.uk/signatures/id/ (for example, ID6). Application of DirectHRD to 37 liquid biopsies from 17 HRD+ and 12 HRD− triple negative breast cancer patients showed sensitive HRD detection down to ˜1% tumor DNA in blood, at best 0.33%, without false detection. At ≥1% tumor DNA, DirectHRD yielded 100% (13/13) PPV and 87.5% (7/8) NPV. In all, DirectHRD enables expanded HRD testing by allowing its direct detection from cancer patients' blood samples.

HRDetect and CHORD perform well in samples having high tumor content, but their performance dropped significantly when used on low tumor-purity samples (e.g., tissue samples with low purity and samples taken from liquid biopsies). The present disclosure provides an improved method for detecting HRD, termed DirectHRD. As disclosed in McVey and Lee (Trends Genet. 2008 November; 24(11): 529-538) microhomology-mediated end-repair (MMER) is an alternative pathway used to repair double stranded breaks (DSB), which creates unique mutation signatures, i.e., small deletions with microhomology, called mhDel. In some embodiments, DirectHRD analyzes only mhDel. The present analysis has found that the longer the length of homology the more likely the mhDel is associated with HRD, rather than with biological and/or sequencing noise. The readily available Indel classification schema (ID83 format) from COSMIC V3.2 was used, which combines the length of homology, as well as the length of deletion of an mhDel (FIGS. 5A-5B). A workflow for DirectHRD is shown in FIGS. 2 and 4A.

Computer Systems: The present disclosure also relates to a computer system involved in carrying out the methods of the disclosure relating to both computations and sequencing.

A computer system (or digital device) may be used to receive, transmit, display and/or store results, analyze the results, and/or produce a report of the results and analysis. A computer system may be understood as a logical apparatus that can read instructions from media (e.g. software) and/or network port (e.g. from the internet), which can optionally be connected to a server having fixed media. A computer system may comprise one or more of a CPU, disk drives, input devices such as keyboard and/or mouse, and a display (e.g. a monitor). Data communication, such as transmission of instructions or reports, can be achieved through a communication medium to a server at a local or a remote location. The communication medium can include any means of transmitting and/or receiving data. For example, the communication medium can be a network connection, a wireless connection, or an internet connection. Such a connection can provide for communication over the World Wide Web. It is envisioned that data relating to the present disclosure can be transmitted over such networks or connections (or any other suitable means for transmitting information, including but not limited to mailing a physical report, such as a print-out) for reception and/or for review by a receiver. The receiver can be but is not limited to an individual, or electronic system (e.g. one or more computers, and/or one or more servers).

In some embodiments, the computer system may comprise one or more processors. Processors may be associated with one or more controllers, calculation units, and/or other units of a computer system, or implanted in firmware as desired. If implemented in software, the routines may be stored in any computer readable memory such as in RAM, ROM, flash memory, a magnetic disk, a laser disk, or other suitable storage medium. Likewise, this software may be delivered to a computing device via any known delivery method including, for example, over a communication channel such as a telephone line, the internet, a wireless connection, etc., or via a transportable medium, such as a computer readable disk, flash drive, etc. The various steps may be implemented as various blocks, operations, tools, modules, and techniques which, in turn, may be implemented in hardware, firmware, software, or any combination of hardware, firmware, and/or software. When implemented in hardware, some or all of the blocks, operations, techniques, etc. may be implemented in, for example, a custom integrated circuit (IC), an application specific integrated circuit (ASIC), a field programmable logic array (FPGA), a programmable logic array (PLA), etc.

A client-server, relational database architecture can be used in embodiments of the disclosure. A client-server architecture is a network architecture in which each computer or processor on the network is either a client or a server. Server computers are typically powerful computers dedicated to managing disk drives (file servers), printers (print servers), or network traffic (network servers). Client computers include PCs (personal computers) or workstations on which users run applications, as well as example output devices as disclosed herein. Client computers rely on server computers for resources, such as files, devices, and even processing power. In some embodiments of the disclosure, the server computer handles all of the database functionality. The client computer can have software that handles all the front-end data management and can also receive data input from users.

A machine readable medium which may comprise computer-executable code may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The subject computer-executable code can be executed on any suitable device which may comprise a processor, including a server, a PC, or a mobile device such as a smartphone or tablet. Any controller or computer optionally includes a monitor, which can be a cathode ray tube (“CRT”) display, a flat panel display (e.g., active matrix liquid crystal display, liquid crystal display, etc.), or others. Computer circuitry is often placed in a box, which includes numerous integrated circuit chips, such as a microprocessor, memory, interface circuits, and others. The box also optionally includes a hard disk drive, a floppy disk drive, a high capacity removable drive such as a writeable CD-ROM, and other common peripheral elements. Inputting devices such as a keyboard, mouse, or touch-sensitive screen, optionally provide for input from a user. The computer can include appropriate software for receiving user instructions, either in the form of user input into a set of parameter fields, e.g., in a GUI, or in the form of preprogrammed instructions, e.g., preprogrammed for a variety of different specific operations.

A computer can transform data into various formats for display. A graphical presentation of the results of a calculation can be displayed on a monitor, display, or other visualizable medium (e.g., a printout). In some embodiments, data or the results of a calculation may be presented in an auditory form.

Types of Samples: This disclosure provides methods to extract and sequence a polynucleotide present in a sample. In one embodiment, the samples are biological samples generally derived from a human subject, preferably a bodily fluid (such as ascites, blood, plasma, pleural fluid, serum, cerebrospinal fluid, phlegm, saliva, stool, urine, semen, prostate fluid, breast milk, or tears, or a tissue sample (e.g. a tissue sample obtained by biopsy). In a further embodiment, the samples are biological samples derived from an animal, preferably a bodily fluid (such as blood, cerebrospinal fluid, phlegm, saliva, or urine) or a tissue sample (e.g. a tissue sample obtained by biopsy). In still another embodiment, the samples are biological samples from in vitro sources (such as cell culture medium). Cell free (cfDNA) attached to a substrate may be first suspended in a liquid medium, such as a buffer or a water, and then subject to sequencing and/or analysis. In yet another embodiment, the sample contains DNA within a cell, which may be extracted, sequenced and subject to the same analysis. In some instances, the sample is a biopsy (e.g., a needle biopsy) or a section.

Classification of Mutations Relative to Reference Sequences: In certain aspects, the disclosure provides methods and kits that involve and/or allow for assessment of the presence or absence of one or more mutations (e.g., deletions, insertions, indels) in a test subject, tissue, cell, or sample, as compared to a corresponding reference sequence. In particular embodiments, a subject, tissue, cell and/or sample is assessed for insertions and/or deletions (e.g., microdeletions associated with HRD).

In particular embodiments, a small deletion or insertion serves as the basis for mutational signature analysis.

Classification of small deletions and insertions for mutational signature analysis ICGC
Pan Cancer Analysis
Working Group on Mutational Signatures.
No. = “Mutation Class No.”; BP = “Base Pair”; RS = “Repeat Size”

| | | | | |
|---|---|---|---|---|
| No. | Type | BP | RS | Example |

1 bp deletion

| | | | | |
|---|---|---|---|---|
| 1 | Del | T:A | 1 | ACCCC\|T\|CGCGGC (delete 1 T from a stretch of 1 Ts) (SEQ ID NO: 5) |
| 2 | Del | C:G | 1 | ACCAA\|C\|TGCGGC (SEQ ID NO: 6) |
| 3 | Del | T:A | 2 | ACCCC\|T\|TGCGGC (delete 1 T from a stretch of 2 Ts) (SEQ ID NO: 7) |
| 4 | Del | C:G | 2 | ACCAA\|C\|CGCGGC (SEQ ID NO: 8) |
| 5 | Del | T:A | 3 | ACCCC\|T\|TTGCGGC (SEQ ID NO: 9) |
| 6 | Del | C:G | 3 | ACCAA\|C\|CCGCGGC (SEQ ID NO: 10) |
| 7 | Del | T:A | 4 | ACCCC\|T\|TTTGCGGC (SEQ ID NO: 11) |
| 8 | Del | C:G | 4 | ACCAA\|C\|CCCGCGGC (SEQ ID NO: 12) |
| 9 | Del | T:A | 5 | ACCCC\|T\|TTTTGCGGC (SEQ ID NO: 13) |
| 10 | Del | C:G | 5 | ACCAA\|C\|CCCCGCGGC (SEQ ID NO: 14) |
| 11 | Del | T:A | 6+ | ACCCC\|T\|TTTTTGCGGC (SEQ ID NO: 15) |
| 12 | Del | C:G | 6+ | ACCAA\|C\|CCCCCGCGGC (SEQ ID NO: 16) |

1 bp insertion

| | | | | |
|---|---|---|---|---|
| 13 | Ins | T:A | 0 | ACCCC\|T\|CGCGGC (insert 1 T with no neighboring Ts) (SEQ ID NO: 17) |
| 14 | Ins | C:G | 0 | ACCAA\|C\|TGCGGC (SEQ ID NO: 18) |
| 15 | Ins | T:A | 1 | ACCCC\|T\|TGCGGC (insert 1 T with 1 neighboring T) (SEQ ID NO: 19) |
| 16 | Ins | C:G | 1 | ACCAA\|C\|CGCGGC (SEQ ID NO: 20) |
| 17 | Ins | T:A | 2 | ACCCC\|T\|TTGCGGC (insert 1 T with 2 neighboring Ts) (SEQ ID NO: 21) |
| 18 | Ins | C:G | 2 | ACCAA\|C\|CCGCGGC (SEQ ID NO: 22) |
| 19 | Ins | T:A | 3 | ACCCC\|T\|TTTGCGGC (SEQ ID NO: 23) |
| 20 | Ins | C:G | 3 | ACCAA\|C\|CCCGCGGC (SEQ ID NO: 24) |
| 21 | Ins | T:A | 4 | ACCCC\|T\|TTTTGCGGC (SEQ ID NO: 25) |
| 22 | Ins | C:G | 4 | ACCAA\|C\|CCCCGCGGC (SEQ ID NO: 26) |
| 23 | Ins | T:A | 5+ | ACCCC\|T\|TTTTTGCGGC (SEQ ID NO: 27) |
| 24 | Ins | C:G | 5+ | ACCAA\|C\|CCCCCGCGGC (SEQ ID NO: 28) |

>2bp deletions

| | | | | |
|---|---|---|---|---|
| 25 | Del | 2 bp | 1 | ACCAA\|TC\|AAGCGGC (delete a single 2-bp sequence with no microhomology) |
| | | | | (SEQ ID NO: 29) |
| 26 | Del | 2 bp | 2 | ACCCC\|TC\|TCGCGGC (delete a single 2-bp sequence from repeat of 2 2-bp |
| | | | | units) (SEQ ID NO: 30) |
| 27 | Del | 2 bp | 3 | ACCCC\|TC\|TCTCGCGGC (SEQ ID NO: 31) |
| 28 | Del | 2 bp | 4 | ACCCC\|TC\|TCTCTCGCGGC (SEQ ID NO: 32) |
| 29 | Del | 2 bp | 5 | ACCCC\|TC\|TCTCTCTCGCGGC (SEQ ID NO: 33) |
| 30 | Del | 2 bp | 6+ | ACCCC\|TC\|TCTCTCTCTCGCGGC (SEQ ID NO: 34) |
| 31 | Del | 3 bp | 1 | ACCAAA\|TTC\|AAAGCGGC (SEQ ID NO: 35) |
| 32 | Del | 3 bp | 2 | ACCAAA\|TTC\|TTCAAAGCGGC (SEQ ID NO: 36) |
| 33 | Del | 3 bp | 3 | ACCAAA\|TTC\|TTCTTCAAAGCGGC (SEQ ID NO: 37) |
| 34 | Del | 3 bp | 4 | ACCAAA\|TTC\|TTCTTCTTCAAAGCGGC (SEQ ID NO: 38) |
| 35 | Del | 3 bp | 5 | ACCAAA\|TTC\|TTCTTCTTCTTCAAAGCGGC (SEQ ID NO: 39) |
| 36 | Del | 3 bp | 6+ | ACCAAA\|TTC\|TTCTTCTTCTTCTTCAAAGCGGC (SEQ ID NO: 40) |
| 37 | Del | 4 bp | 1 | ACCAAAA\|TCTC\|AAAAGCGGC (SEQ ID NO: 41) |
| 38 | Del | 4 bp | 2 | ACCAAAA\|TCTC\|TCTCAAAAGCGGC (SEQ ID NO: 42) |
| 39 | Del | 4 bp | 3 | ACCAAAA\|TCTC\|TCTCTCTCAAAAGCGGC (SEQ ID NO: 43) |
| 40 | Del | 4 bp | 4 | ACCAAAA\|TCTC\|TCTCTCTCTCTCAAAAGCGGC (SEQ ID NO: 44) |
| 41 | Del | 4 bp | 5 | ACCAAAA\|TCTC\|TCTCTCTCTCTCTCTCAAAAGCGGC (SEQ ID NO: 45) |
| 42 | Del | 4 bp | 6+ | ACCAAAA\|TCTC\|TCTCTCTCTCTCTCTCTCTCAAAAGCGGC (SEQ ID |
| | | | | NO: 46) |
| 43 | Del | 5+ bp | 1 | ACCAAAAA\|TCATC\|AAAAAGCGGC (SEQ ID NO: 47) |
| 44 | Del | 5+ bp | 2 | ACCAAAAA\|TCATC\|TCATCAAAAAGCGGC (SEQ ID NO: 48) |
| 45 | Del | 5+ bp | 3 | ACCAAAAA\|TCATC\|TCATCTCATCAAAAAGCGGC (SEQ ID NO: 49) |
| 46 | Del | 5+ bp | 4 | ACCAAAAA\|TCATC\|TCATCTCATCTCATCAAAAAGCGGC (SEQ ID NO: |
| | | | | 50) |
| 47 | Del | 5+ bp | 5 | ACCAAAAA\|TCATC\|TCATCTCATCTCATCTCATCAAAAAGCGGC (SEQ |
| | | | | ID NO: 51) |
| 48 | Del | 5+ bp | 6+ | ACCAAAAA\|TCATC\|TCATCTCATCTCATCTCATCTCATCAAAAAGCGG |
| | | | | C |
| | | | | (SEQ ID NO: 52) |

> = 2 bp insertions

| | | | | |
|---|---|---|---|---|
| 49 | Ins | 2 bp | 0 | ACCAA\|TC\|AAGCGGC (SEQ ID NO: 53) |
| 50 | Ins | 2 bp | 1 | ACCCC\|TC\|TCGCGGC (SEQ ID NO: 54) |
| 51 | Ins | 2 bp | 2 | ACCCC\|TC\|TCTCGCGGC (SEQ ID NO: 55) |
| 52 | Ins | 2 bp | 3 | ACCCC\|TC\|TCTCTCGCGGC (SEQ ID NO: 56) |
| 53 | Ins | 2 bp | 4 | ACCCC\|TC\|TCTCTCTCGCGGC (SEQ ID NO: 57) |
| 54 | Ins | 2 bp | 5+ | ACCCC\|TC\|TCTCTCTCTCGCGGC (SEQ ID NO: 58) |
| 55 | Ins | 3 bp | 1 | ACCAAA\|TTC\|AAAGCGGC (SEQ ID NO: 59) |
| 56 | Ins | 3 bp | 1 | ACCAAA\|TTC\|TTCAAAGCGGC (SEQ ID NO: 60) |
| 57 | Ins | 3 bp | 2 | ACCAAA\|TTC\|TTCTTCAAAGCGGC (SEQ ID NO: 61) |
| 58 | Ins | 3 bp | 3 | ACCAAA\|TTC\|TTCTTCTTCAAAGCGGC (SEQ ID NO: 62) |
| 59 | Ins | 3 bp | 4 | ACCAAA\|TTC\|TTCTTCTTCTTCAAAGCGGC (SEQ ID NO: 63) |
| 60 | Ins | 3 bp | 5+ | ACCAAA\|TTC\|TTCTTCTTCTTCTTCAAAGCGGC (SEQ ID NO: 64) |
| 61 | Ins | 4 bp | 0 | ACCAAAA\|TCTC\|AAAAGCGGC (SEQ ID NO: 65) |
| 62 | Ins | 4 bp | 1 | ACCAAAA\|TCTC\|TCTCAAAAGCGGC (SEQ ID NO: 66) |
| 63 | Ins | 4 bp | 2 | ACCAAAA\|TCTC\|TCTCTCTCAAAAGCGGC (SEQ ID NO: 67) |
| 64 | Ins | 4 bp | 3 | ACCAAAA\|TCTC\|TCTCTCTCTCTCAAAAGCGGC (SEQ ID NO: 68) |
| 65 | Ins | 4 bp | 4 | ACCAAAA\|TCTC\|TCTCTCTCTCTCTCTCAAAAGCGGC (SEQ ID NO: 69) |
| 66 | Ins | 4 bp | 5+ | ACCAAAA\|TCTC\|TCTCTCTCTCTCTCTCTCTCAAAAGCGGC (SEQ ID |
| 67 | Ins | 5 bp | 0 | ACCAAAAA\|TCATC\|AAAAAGCGGC (SEQ ID NO: 71) |
| 68 | Ins | 5 bp | 1 | ACCAAAAA\|TCATC\|TCATCAAAAAGCGGC (SEQ ID NO: 72) |
| 69 | Ins | 5 bp | 2 | ACCAAAAA\|TCATC\|TCATCTCATCAAAAAGCGGC (SEQ ID NO: 73) |
| 70 | Ins | 5 bp | 3 | ACCAAAAA\|TCATC\|TCATCTCATCTCATCAAAAAGCGGC (SEQ ID NO: |
| | | | | 74) |
| 71 | Ins | 5 bp | 4 | ACCAAAAA\|TCATC\|TCATCTCATCTCATCTCATCAAAAAGCGGC (SEQ |
| | | | | ID NO: 75) |
| 72 | Ins | 5 bp | 5+ | ACCAAAAA\|TCATC\|TCATCTCATCTCATCTCATCTCATCAAAAAGCGG |
| | | | | C |
| | | | | (SEQ ID NO: 76) |

> = 2 bp deletions at micro-homologies

| | | | | |
|---|---|---|---|---|
| 73 | Del | 2 bp | 1 bp | ACCAA\|TC\|TAGCGGC (SEQ ID NO: 76) or |
| | | | | ACAAC\|TC\|AAGCGGC (SEQ ID NO: 77) |
| 74 | Del | 3 bp | 1 bp | ACCCA\|TTC\|TAGCGGC (SEQ ID NO: 78) or |
| | | | | ACCCC\|TTC\|AAGCGGC (SEQ ID NO: 79) |
| 75 | Del | 3 bp | 2 bp | ACCCA\|TTC\|TTAGCGGC (SEQ ID NO: 80) or |
| | | | | ACCCTC\|TTC\|AAGCGGC (SEQ ID NO: 81) |
| 76 | Del | 4 bp | 1 bp | ACCCA\|TATC\|TTAGCGGC (SEQ ID NO: 82) or |
| | | | | ACCCAC\|TATC\|AAGCGGC (SEQ ID NO: 83) |
| 77 | Del | 4 bp | 2 bp | ACCCA\|TATC\|TAAGCGGC (SEQ ID NO: 84) or |
| | | | | CCCGTC\|TATCAAGCGGC (SEQ ID NO: 85) |
| 78 | Del | 4 bp | 3 bp | ACCCA\|TATC\|TATAGCGGC (SEQ ID NO: 86) or |
| | | | | ACCCATC\|TATC\|AAGCGGC (SEQ ID NO: 87) |
| 79 | Del | 5+ bp | 1 bp | ACCCA\|TAGTC\|DelAGCGGC (SEQ ID NO: 88) or |
| | | | | ACCCADel\|TAGTC\|AAGCGGC (SEQ ID NO: 89) |
| 80 | Del | 5+ bp | 2 bp | ACCCA\|TAGTC\|DelAGCGGC (SEQ ID NO: 90) or |
| | | | | ACCCCDel\|TAGTC\|AAGCGGC (SEQ ID NO: 91) |
| 81 | Del | 5+ bp | 3 bp | ACCCA\|TAGTC\|TAGAGCGGC (SEQ ID NO: 92) or |
| | | | | ACCCGTC\|TAGTC\|AAGCGGC (SEQ ID NO: 93) |
| 82 | Del | 5+ bp | 4 bp | ACCCA\|TAGTC\|TAGTAGCGGC (SEQ ID NO: 94) or |
| | | | | ACCCAGTC\|TAGTC\|AAGCGGC (SEQ ID NO: 95) |
| 83 | Del | 5+ bp | 5+ | ACCCA\|TAGCCTC\|TAGCCTAGCGGC (SEQ ID NO: 96) or |
| | | | bp | ACCCAGCCTC\|TAGCCTC\|AAGCGGC (SEQ ID NO: 97) |

The reference sequence can correspond to cell free DNA from a healthy subject and/or from a subject prior to having and/or being diagnosed with a neoplasm. A reference sequence can correspond to cell free DNA from a patient-matched normal control. In some embodiments, the reference sequence comprises just a portion of a genome, wherein the portion comprises 10, 11, 12, 13, 14, 15, 16, 17, 18, 19, 20, 21, 22, 23, 24, 25, 30, 40, 50, 60, 70, 80, 90, 100, 200, or more nucleotides.

Sequencing: In various aspects, the methods provided herein involve sequencing of a sample (e.g., circulating tumor DNA). In some embodiments, the sequencing is whole-genome sequencing (WGS) or whole-exome sequencing (WES). In some embodiments, the sequencing is targeted sequencing, whole-genome bisulfite sequencing data, methylation sequencing data, and/or RNA sequencing. The sequencing is performed upon a test sample for purpose of detecting fragment length distributions and somatic copy number alterations in a sample (e.g., in cell free DNA). In certain embodiments, the sequencing can be performed with or without amplification of a sample to be sequenced. In embodiments, a sample is sequenced to a coverage of about, at least about, and/or no more than about 0.01×, 0.05×, 0.1×, 0.2×, 0.3×, 0.4×, 0.5×, 1×, 2×, 3×, 4×, 5×, 6×, 7×, 8×, 9×, 10×, 20×, 30×, 40×, 50×, 60×, 70×, 80×, 90×, 100×, 200×, 300×, 400×, 500×, or more.

Whole genome sequencing (also known as “WGS”, full genome sequencing, complete genome sequencing, or entire genome sequencing) is a process that involves sequencing a complete DNA sequence of an organism's genome. A common strategy used for WGS is shotgun sequencing, in which DNA is broken up randomly into numerous small segments, which are sequenced. Sequence data obtained from one sequencing reaction is termed a “read.” The reads can be assembled together based on sequence overlap. The genome sequence is obtained by assembling the reads into a reconstructed sequence.

Whole exome sequencing (“WES”) is a technique used to sequence all the expressed genes in a cell or subject (known as the exome). It includes first selecting only that portion of a polynucleotide sample that encodes proteins (e.g., cDNA, or a subset of a cfDNA sample), and then sequencing using any DNA sequencing technology well known in the art or as described herein. In a human being, there are about 180,000 exons, which constitute about 1% of the human genome, or approximately 30 million base pairs. In some embodiments, to sequence the exons of a genome, fragments of double-stranded genomic DNA are obtained (e.g., by methods such as sonication, nuclease digestion, or any other appropriate methods). Linkers or adapters are then attached to the DNA fragments, which are then hybridized to a library of polynucleotides designed to capture only the exons. The hybridized DNA fragments are then selectively isolated and subjected to sequencing using any sequencing method known in the art or described herein.

Sequencing may be performed on any high-throughput platform. Methods of sequencing oligonucleotides and nucleic acids are well known in the art (see, e.g., WO93/23564, WO98/28440 and WO98/13523; U.S. Pat. Nos. 5,525,464; 5,202,231; 5,695,940; 4,971,903; 5,902,723; 5,795,782; 5,547,839 and 5,403,708; Sanger et al., Proc. Natl. Acad. Sci. USA 74:5463 (1977); Drmanac et al., Genomics 4:114 (1989); Koster et al., Nature Biotechnology 14:1123 (1996); Hyman, Anal. Biochem. 174:423 (1988); Rosenthal, International Patent Application Publication 761107 (1989); Metzker et al., Nucl. Acids Res. 22:4259 (1994); Jones, Biotechniques 22:938 (1997); Ronaghi et al., Anal. Biochem. 242:84 (1996); Ronaghi et al., Science 281:363 (1998); Nyren et al., Anal. Biochem. 151:504 (1985); Canard and Arzumanov, Gene 11:1 (1994); Dyatkina and Arzumanov, Nucleic Acids Symp Ser 18:117 (1987); Johnson et al., Anal. Biochem. 136:192 (1984); and Elgen and Rigler, Proc. Natl. Acad. Sci. USA 91(13):5740 (1994), all of which are expressly incorporated by reference). In one embodiment, the sequencing of a DNA fragment is carried out using commercially available sequencing technology SBS (sequencing by synthesis) by Illumina. In another embodiment, the sequencing of the DNA fragment is carried out using the chain termination method of DNA sequencing. In yet another embodiment, the sequencing of the DNA fragment is carried out using one of the commercially available next-generation sequencing technologies, including SMRT (single-molecule real-time) sequencing from Pacific Biosciences, Ion Torrent™ sequencing from ThermoFisher Scientific, Pyrosequencing (454) from Roche, and SOLiD® technology from Applied Biosystems. Any appropriate sequencing technology may be chosen for sequencing.

For purpose of this disclosure, the term “amplification” means any method employing a primer and a polymerase capable of replicating a target sequence with reasonable fidelity. Amplification may be carried out by natural or recombinant DNA polymerases such as TaqGold™, T7 DNA polymerase, Klenow fragment of E. coli DNA polymerase, and reverse transcriptase. A preferred amplification method is PCR. Typically, the amplification of a sample results in an exponential increase in copy number of the amplified sequences. Amplification may involve thermocycling or isothermal amplification (such as through the methods RPA or LAMP).

Design and use of oligonucleotides for amplification and/or sequencing is within the knowledge of one of ordinary skill in the art. Oligonucleotides can be modified by any of a number of art-recognized moieties and/or exogenous sequences, e.g., to enhance the processes of amplification, sequencing reactions, and/or detection. Exemplary oligonucleotide modifications that are expressly contemplated for use with the oligonucleotides of the instant disclosure include, e.g., fluorescent and/or radioactive label modifications; labeling one or more oligonucleotides with a universal amplification sequence (optionally of exogenous origin) and/or labeling one or more oligonucleotides of the instant disclosure with a unique identification sequence (e.g., a “bar-code” sequence, optionally of exogenous origin), as well as other modifications known in the art and suitable for use with oligonucleotides.

Patient and/or Treatment Monitoring: In various aspects, the disclosure provides methods for monitoring a patient for a neoplasia. The methods involve sequencing DNA (e.g., circulating tumor DNA) present in a biological sample collected from the subject, analyzing the sequence as described herein, and assigning an HRD score. In some instances, the methods provided herein are used to monitor HRD in polynucleotides (e.g., cfDNA, circulating tumor DNA) in a liquid biopsy of a patient as part of routine monitoring (e.g., as part of a routine physical) for a neoplasia.

The methods described herein include methods for the treatment of a neoplasia (e.g. a cancer or tumor). Generally, the methods include administering a therapeutically effective amount of a treatment as described herein, to a subject who is in need of, or who has been determined to be in need of, such treatment. The methods further involve sequencing polynucleotides present in a biological sample (e.g., cell free DNA, circulating tumor DNA in a blood sample) from the subject and determining whether the subject has HRD according to the methods provided herein.

The methods provided herein can be used for clinical cancer management, such as for characterization of a cancer in a subject for the presence or absence of HRD. In various instances, the methods provided herein are used for early screening and/or in clinical cancer management. Where a subject is found to have HRD, this can be used to inform therapy (e.g., PARP inhibitor, platinum chemotherapy).

In various instances, tumor fraction in a polynucleotide sample taken from a subject is determined. In some cases, tumor fraction is measured as part of a process to monitor a subject for cancer. The polynucleotide sample in various cases is cfDNA or circulating tumor DNA.

The methods of the disclosure advantageously allow for monitoring the efficacy of a neoplasia treatment. In some cases, a treatment is characterized as ineffective (i.e., a tumor is resistant to treatment or has developed resistance to treatment) if tumor fraction increases in a subject being administered the treatment. In embodiments, if a treatment is characterized as ineffective in a subject (i.e., the tumor is resistant to treatment or has developed resistance to treatment), the treatment is changed to an alternative treatment. The increase or decrease in various instances is statistically significant. In some instances, a treatment is characterized as effective if the tumor fraction in cell free DNA is maintained beneath a threshold and is characterized as ineffective if the tumor fraction is not maintained beneath the threshold. In various instances, the threshold is about, at least about, or no more than about 5%, 10%, 15%, 20%, 25%, 30%, 35%, 40%, 45%, or 50%. In some cases, a treatment is characterized as ineffective if the tumor fraction increases significantly. In some instances, a treatment is characterized as ineffective if an increase in tumor fraction of about, or at least about 1%, 2%, 3%, 4%, 5%, 10%, 20%, 30%, 40%, 50%, 1×, 2×, 3×, 4×, 5×, 10×, or 100×, is measured.

Treatments amenable to monitoring using the methods of the invention include, but are not limited to, chemotherapy, radiotherapy, immunotherapy, surgery, or various other methods available to a skilled practitioner or described herein.

Cancer Treatments: Methods of inhibiting and/or treating cancer and tumors in individuals with cancer or a predisposition for developing cancer as identified by methods of the disclosure are also contemplated.

In embodiments, the subject has been diagnosed with a neoplasm (e.g., a cancer) or is at risk of developing a neoplasm (e.g. a cancer or tumor). The subject, in various instances, is a human, dog, cat, horse, or any animal. Illustrative neoplasms include breast cancer, pancreatic cancer, prostate cancer, or ovarian cancer.

The therapeutic agent is for example, a PARP inhibitor, a chemotherapeutic agent (e.g., platinum), radiation, or immunotherapy. Any suitable therapeutic treatment for a particular cancer may be administered. Examples of chemotherapeutic agents include, but are not limited to, aldesleukin, altretamine, amifostine, asparaginase, bleomycin, capecitabine, carboplatin, carmustine, cladribine, cisapride, cisplatin, cyclophosphamide, cytarabine, dacarbazine (DTIC), dactinomycin, docetaxel, doxorubicin, dronabinol, epoetin alpha, etoposide, filgrastim, fludarabine, fluorouracil, gemcitabine, granisetron, hydroxyurea, idarubicin, ifosfamide, interferon alpha, irinotecan, lansoprazole, levamisole, leucovorin, megestrol, mesna, methotrexate, metoclopramide, mitomycin, mitotane, mitoxantrone, omeprazole, ondansetron, paclitaxel (Taxol™), pilocarpine, prochlorperazine, rituximab, tamoxifen, taxol, topotecan hydrochloride, trastuzumab, vinblastine, vincristine and vinorelbine tartrate.

For therapeutic use, administration often begins at the detection or surgical removal of tumors. This is followed by boosting doses until at least symptoms are substantially abated and for a period thereafter.

The pharmaceutical compositions for therapeutic treatment are intended for parenteral, topical, nasal, oral or local administration. Preferably, the pharmaceutical compositions are administered parenterally, e.g., intravenously, subcutaneously, intradermally, or intramuscularly. The compositions may be administered at the site of surgical excision to induce a local immune response to the tumor. The disclosure provides compositions for parenteral administration, which comprise a solution of the peptides and vaccine compositions dissolved or suspended in an acceptable carrier, preferably an aqueous carrier. A variety of aqueous carriers may be used, e.g., water, buffered water, 0.9% saline, 0.3% glycine, hyaluronic acid, and the like. These compositions may be sterilized by conventional, well known sterilization techniques, or may be sterile filtered. The resulting aqueous solutions may be packaged for use as is, or lyophilized, the lyophilized preparation being combined with a sterile solution prior to administration. The compositions may contain pharmaceutically acceptable auxiliary substances as required to approximate physiological conditions, such as pH adjusting and buffering agents, tonicity adjusting agents, wetting agents, and the like, for example, sodium acetate, sodium lactate, sodium chloride, potassium chloride, calcium chloride, sorbitan monolaurate, triethanolamine oleate, etc.

For breast cancer, local therapies such as surgery and radiation are recommended. Breast cancer may also be treated systemically by chemotherapy, hormone therapy (such as, but not limited to, tamoxifen, toremifene, fulvestrant or aromatase inhibitors) or targeted therapy (such as, but not limited to, monoclonal antibodies or other therapeutics that target a HER2 protein, a mTor protein or cyclin-dependent kinases, or kinase inhibitors). If the breast cancer is a BRCA cancer, the cancer may be treated and/or prevented by a mastectomy, sapingo-oophorectomy, or hormonal therapy medicines, such as selective estrogen receptor modulators or aromatase inhibitors. Hormonal therapy medicines include, but are not limited to, tamoxifen, raloxifene, exemestane or anastrozole.

Kits: The disclosure also provides kits containing agents for use in the methods of the present disclosure. Kits of the disclosure may include one or more containers comprising an agent (e.g., a chemotherapeutic agent, PARP inhibitor) of this disclosure and/or may contain agents (e.g., oligonucleotide primers, probes, etc.) for characterizing HRD in a biological sample of the subject. In some embodiments, the kits further include instructions for use in accordance with the methods of this disclosure. In some embodiments, these instructions comprise a description of administration of the agent to treat or diagnose (e.g., a neoplasia) according to any of the methods of this disclosure. In some embodiments, the instructions comprise a description of how to calculate tumor fraction in cfDNA or circulating tumor DNA, for example in an individual, in a tissue sample, or in a cell, and, in some cases, the instructions may describe how such calculations should inform the treatment of a patient.

The instructions generally include information as to dosage, dosing schedule, and route of administration for the intended treatment. The containers may be unit doses, bulk packages (e.g., multi-dose packages) or sub-unit doses. Instructions supplied in the kits of the instant disclosure are typically written instructions on a label or package insert (e.g., a paper sheet included in the kit), but machine-readable instructions (e.g., instructions carried on a magnetic or optical storage disk) are also acceptable.

The label or package insert indicates that the composition is used for treating, e.g., a neoplasia, in a subject. Instructions may be provided for practicing any of the methods described herein.

The kits of this disclosure are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (e.g., the container may be an intravenous solution bag or a vial having a stopper pierceable by a hypodermic injection needle). In certain embodiments, at least one active agent (e.g., a chemotherapeutic agent).

Kits may optionally provide additional components such as buffers and interpretive information. Normally, the kit comprises a container and a label or package insert(s) on or associated with the container.

The practice of the implementations of the Example Embodiments section may use, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### Example 1—Establishment of a Highly Sensitive Method to Detect HRD Scars: DirectHRD

A highly sensitive method was developed to detect homologous recombination deficiency (HRD) scars from low tumor purity samples such as liquid biopsies. In this setting, the tumor signal can easily be obscured by technical and biological noise; thus, the first goal was to identify the features that (a) offer the strongest distinction between HRD positive and HRD negative tumors, and (b) could be reliably detected at low tumor fractions in sequencing data. CHORD and HRDetect both showed that a small deletion with microhomology is the top feature used by their machine learning models. This, coupled with the low error rate for indels in Illumina sequencing (˜5E-06), led to the hypothesis that a probabilistic model for HRD detection based solely upon detecting mhDels may enable detection of HRD scars at low tumor fractions. To test this idea, DirectHRD was developed, which is the first method that solely assays mhDels to detect HRD (FIG. 4).

DirectHRD (FIG. 4A) is an ultrasensitive scar-based classifier to detect HRD from low tumor fraction samples, such as liquid biopsies using whole-genome sequencing (WGS) data. DirectHRD makes use of a specific type of genomic scar¬—a small, microhomology deletion (mhDel, FIG. 6A)—believed to be the direct evidence of microhomology-mediated end joining in the absence of HR—due to its superior classification power and low error rate in next generation sequencing (NGS). However, mhDels represent only a fraction of already rare somatic small deletions. PCAWG data was surveyed for mhDels in 4 cancer types and a median of 306 mhDels (151-537 for 15% and 85% quantile) was found in HRD positive genomes defined by CHORD (FIG. 4B). The number of mhDels that could be recovered at varied sequencing depths and tumor fractions was modeled. It was estimated that about 4-8 (15% quantile and median, respectively) mhDels could be uncovered at 1% tumor fraction using 50× whole-genome sequencing (FIG. 4C). This indicates that one can utilize mhDels for HRD detection from low tumor fraction samples using moderate depths of WGS.

DirectHRD was developed using highly accurate CODEC whole-genome sequencing of 14 tumor biopsy samples in TBCRC030, a prospective study of neoadjuvant paclitaxel vs. cisplatin in stage II-III triple negative breast cancer (TNBC). An HRDscore cutoff of 1 was found in DirectHRD correctly classified tumors when using Myriad MyChoice HRD testing as ground truth (FIG. 6B). However, standard Illumina WGS exhibits exceptionally low error rates for Indels (insertion/deletion polymorphism) in high complexity regions (versus other alterations such as single nucleotide variants, SNVs). Thus, DirectHRD is likely sufficient for mhDel detection (FIG. 6C). Indeed, CODEC single fragment caller (SFC) was adapted to standard Illumina WGS data and found that it detected mhDels with an error rate of 3×10-9 using a cutoff of 2 unique fragments (FIG. 6C). Thereafter, standard Illumina WGS was used for DirectHRD. This approach showed superior performance for mhDel detection as compared to the state-of-the-art Indel detection methods (FIGS. 6D, 6E). After Indel calling, DirectHRD compares the Indel signature in each sample against a known HRD-positive signature (ID6 in COSMIC 3.2) and an HRD-negative signature which was derived from PCAWG HRD-negative breast cancers. An HRD-positive signature was derived from PCAWG breast cancers to use as a backup signature (Methods). DirectHRD used this backup signature in 1/55 cfDNA sample and 1/81 synthetic or down-sampled tumor sample (Table 5). Using an Indel signature such as ID6 allows us to assign posterior probabilities/confidence levels to each deletion and microhomology length combination (FIG. 6F). The probabilistic model had better classification accuracy than using simple count feature (FIG. 7A). DirectHRD requires paired whole-genome sequencing (WGS) of the test specimen (e.g., cfDNA or tumor biopsy) and matched germline DNA. Raw reads are first aligned to human reference genome HG19 using the Burrows-Wheeler Alignment Tool BWA, followed by Picard deduplication and indel realignment by GATK. Then, the single fragment caller (SFC, Bae, J. H., et al. Single duplex DNA sequencing with CODEC detects mutations with high sensitivity. Nat Genet 55, 871-879 (2023). https://doi.org/10.1038/s41588-023-01376-0) was used to call somatic indels, requiring a minimum of two unique fragments supporting each indel. Of note, other current indel callers are not tuned to detect mhDels with the requisite sensitivity and specificity from liquid biopsies. Instead, the simple hard-filtered and count-based SFC caller was used that was originally developed for highly accurate sequencing using CODEC (Bae, J. H., et al. Nat Genet 55, 871-879 (2023)). It was discovered that using a cutoff of 2 unique fragments in standard next generation sequencing (NGS) could give similar error rates as in CODEC sequencing. Overall, the single fragment caller (SFC) showed superior performance for mhDel detection as compared to the state-of-the-art indel detection methods such as Mutect2, Strelka1, and Strelka2.

Next, the indels were categorized into ID83 format using SigProfilerExtractor (Islam et al. bioRxiv 2020.12.13.422570; doi: https://doi.org/10.1101/2020.12.13.422570). DirectHRD is the first to the use an indel signature. HRDetect, for example, uses the fraction of mhDels out of the total deletions. CHORD improved upon HRDetect by separating mhDels into 1 bp homology and >1 bp homology. Compared to HRDetect and CHORD, DirectHRD uses a much richer feature set: each class of mhDels in COSMIC ID83 format is treated as a separate feature. This is important because longer mhDels with longer lengths of homology are more likely to be associated with HRD (FIG. 9A). Thereby, using an indel signature like ID6 allows the harnessing of each length of a deletion and each length of a microhomology in a much finer manner.

Finally, the raw counts of the 11 types of mhDels were used by DirectHRD to produce an HRD score (whereby an HRD score ≥1 is considered HRD positive) using a probabilistic model. Gulhan et al. (Nat Genet. 2019 May; 51(5):912-919. Doi: 10.1038/s41588-019-0390-2. Epub 2019 Apr. 15. PMID: 30988514) had shown that the probability-based measure is more effective for a binary classification of the existence of a particular signature as opposed to detecting all signatures in a sample. A tailored approach was created of detecting the HRD positive signature (ID6) vs the HRD negative signature derived from breast cancers sequenced in the Pancancer Analysis of Whole Genome study. The probabilistic model was confirmed to have better classification accuracy than using simple count features (FIG. 9B).

### Example 2—Detection of HRD from Tumor Biopsies and Simulated Low Purity Samples

To validate DirectHRD, it was applied to 15×WGS of tumor biopsies from 27 triple negative breast cancer patients from the TBCRC030 trial including the 12 patients sequenced by CODEC. All 27 patients had tumor HRD status determined by Myriad MyChoice. When compared to this gold standard commercial assay, DirectHRD outperformed CHORD, a leading WGS-based classifier: AUC 0.99 vs 0.82 (FIG. 4D). Additionally, DirectHRD was compared to an orthogonal method, scarHRD, which employs an HRD score from copy number and large structural variants similar to Myriad MyChoice but derived from next-generation sequencing data. Good correlation (0.81) was found between DirectHRD and scarHRD (FIG. 7B).

Next, to explore the performance of DirectHRD at low tumor purities, in silico dilutions were created of the same 27 tumor biopsies at 5% and 2% tumor purities by diluting tumor WGS reads into matched normal WGS reads. In the diluted samples, DirectHRD maintained high performance at 5% (AUC: 0.99) and declined slightly at 2% (AUC: 0.85) (FIGS. 5B-5B). By comparison, CHORD yielded lower AUCs of 0.62 and 0.67 at 5% and 2% tumor purities, respectively. Of note, it was found that CHORD's performance improved using Strelka2 instead of Strelka1 as published for indel calling, but it still underperformed as compared to DirectHRD at low tumor purity (FIG. 7C). Finally, to account for possible data leakage between the training of DirectHRD's model using CODEC data and testing using standard WGS data, it was confirmed that the performance held up after removing the 12 tumor biopsies that had also been previously sequenced by CODEC (FIG. 4D).

### Example 3—Performance on cfDNA

To test the feasibility of detecting HRD from liquid biopsies, 20 cfDNA samples were sequenced from 10 HRD-positive patients and 10 HRD-negative patients with metastatic breast cancer. The HRD statuses were found by the consensus results of scarHRD4 and SigMA7 on tumor whole-exome sequencing (WES) (FIGS. 8A, 8B). Selected cfDNA samples were matched on cfDNA tumor fractions (FIG. 8C) determined by ichorCNA and further refined the tumor fraction estimates using tumor WES-informed SNVs (Methods). The 20 cfDNA libraries were sequenced to a median 19×WGS coverage (range 4-27×; Table 2, 3). Two samples were excluded because the recalibration showed 0 tumor fraction (FIG. 8D). DirectHRD correctly identified 9/10 HRD positive cases (sensitivity 90%) and 7/8 HRD negative cases (specificity 87.5%), while CHORD identified 2/10 HRD positive samples (sensitivity 20%), both of which had tumor fractions >20% (FIG. 5). This is consistent with CHORD being suited for HRD detection in samples with higher tumor fractions. By comparison, DirectHRD missed 1 HRD+ sample with the lowest tumor fraction by WES-informed re-estimation (0.04%, FIG. 8D). DirectHRD also falsely identified 1 HRD negative sample as positive. Interestingly, this patient's tumor biopsy had the highest scarHRD score (39) among all HRD-negative patients, close to the cutoff 42 (FIG. 8B). We further explored how the HRD scores compared between the cfDNA and tumor biopsy of each patient (FIG. 6E) and found DirectHRD scores from cfDNA samples corrected for tumor fraction were correlated with scarHRD scores from the tissue biopsies (spearman correlation ρ=0.7).

Lastly, the performance of DirectHRD on liquid biopsies from patients with early-stage cancer was explored. Knowledge of the tumor fractions in cfDNA was utilized from prior testing using the MAESTRO MRD assay, and 37 cfDNA samples from 29 patients with TNBC were selected from TBCRC030 with tumor fractions of at least 0.1%, 92% of the cfDNA samples had tumor fractions <10%, and 43% had tumor fractions <1% (FIG. 9A, Table 1). 25 patients had HRD status from Myriad MyChoice, and CHORD was run on the remaining 4 patients using their tumor WGS samples to determine their HRD statuses. WGS was then performed on all 37 cfDNA samples (median 43×, range 15-65.5× coverage) and DirectHRD and CHORD was applied to all cfDNA samples. CHORD identified 1 cfDNA sample with 90% tumor fraction as HRD positive and exhibited no false detection in the HRD-negative patients. DirectHRD correctly detected HRD down to ˜1% tumor fraction in cfDNA samples from HRD-positive patients, range (0.33%-90%), and with no false detection in cfDNA samples from HRD-negative patients (FIG. 5A). In all 37 cfDNA samples, DirectHRD achieved 100% (15/15) PPV and 71% (15/21) sensitivity. At ≥1% tumor DNA, DirectHRD yielded 93% (13/14) sensitivity (FIG. 5B). Similar performance was also seen within the 25 patients with MyChoice results for the first available cfDNA time point from each, mostly (n=23) at diagnosis (Table 7). A high correlation (ρ=0.82, FIG. 7B) was observed between cfDNA and tissue HRD scores both from DirectHRD after correcting for tumor fraction. Since HRD score could be viewed as a quantitative measure of the HRD intensity, it indicates that DirectHRD on cfDNA could reveal the HRD intensity in a patient's tumor biopsy. Lastly, comparable results were seen (Table 7) after removing 14 cfDNA samples from 12 patients whose tumor biopsies had also been previously sequenced with CODEC and used in the original DirectHRD model derivation (FIG. 9B).

In all, DirectHRD detected HRD from low tumor fraction samples such as liquid biopsies with 10× greater sensitivity than the leading methods. This could enable detection of HRD in early and late stages of cancer, including acquisition of HRD, when tissue biopsies are insufficient or infeasible. In the future, tumor fraction estimation will be incorporated into DirectHRD so that the patients with negative results but cfDNA tumor fractions below the limits of detection could be recommended for other follow up testing. Higher sensitivity can be achieved with deeper WGS—the cost of which is plummeting—and targeting sequencing for HR pathway mutations could also be performed from the same cfDNA libraries as DirectHRD. Lastly, DirectHRD assays a well-defined scar of HRD, and it showed high concordance with tissue-based HRD classifiers.

The results described herein were obtained using the following materials and methods.

Indel classification and modeling number of mhDels in liquid biopsies: The PCWAG Indel VCFs (Variant Calling Format) of 4 cancer types, Breast, Ovary, Pancreas, Prostate, was downloaded from ICGC (https://dcc.icgc.org/). The HRD status of each sample was provided by Nguyen et al Nat Commun 11, 5584 (2020) in the supplementary table. Indels were first restricted to high complexity and high mapping quality regions (GIAB V3.0 easy regions, total 2.3B, https://ftp-trace.ncbi.nlm.nih.gov/giab/ftp/release/genome-stratifications/v3.0/GRCh37/LowComplexity/GRCh37_notinAllTandemRepeatsandHomopolymers_slop5.bed.gz) and then used the SigProfilerExtractor to categorize each Indel to one of the 83 classes according to COSMIC 3.2 (https://cancer.sanger.ac.uk/signatures/). There are 11 classes belonging to mhDels, and we summed all of them to get the total mhDel burden per sample. The median was taken and 15% quantile of total mhDel counts in all HRD positive samples across the 4 cancer types. A binomial model was then used where the success probability equaled half of the tumor fractions (assuming the mhDel is heterozygous) and the number of trials equaled the coverages. The probability of recovering an mhDel was calculated based on at least two successes.

Indel calling: DirectHRD's Indel caller is based on the single-fragment caller (SFC) from previous work (Bae et al.). MuTect2 (Benjamin et al bioRxiv; 2019. DOI: 10.1101/861054) and Strelka1&2 were also used in this study for comparison. For the comparison, a low-complexity region filter was applied to callers, which means an indel must occur in the high-complexity regions of the reference genome hg19 (˜2.3 billion bps). The SFC is described further in Bae et al, which is incorporated by reference herein. Some filters were implemented specifically for CODEC and were modified accordingly for standard next generation sequencing (NGS). A mutation was also required to be supported by at least two unique fragments for error correction in the standard NGS data. Additional SFC Indel-filters include the following:
An Indel is at least 8 bp from the ends of a read.
No germline SNP or Indel are adjacent (within 5 bp) to a somatic Indel. In cfDNA, a germline variant is defined as >25% variant allele frequency (VAF; 10 reads at least).
The read, which contains a somatic Indel, does not have another SNP or Indel nearby (for SNP±3 bp, for Indel±10 bp).

Benchmarking Indel calling: The SFC Indel caller used in DirectHRD was benchmarked against established callers such as Mutect2 and Strelka1&2 using a set of 6 positive control samples and 4 negative control samples. For a fair comparison, we applied a high-complexity region filter to all callers, which means an Indel must occur in the high-complexity regions of the reference genome hg19 (GIAB V3.0 easy regions, total 2.3B). We included Strelka1 because it is the default Indel caller by CHORD, but we also tested the next generation caller (Strelka2) for comparison. For the 6 positive control samples, the 60× tumor biopsies were downsampled to 10× to mimic situations of low purities and mutations were looked for that were called in both 10× and 60×. In the negative control data, technical replicates were utilized of germline DNA of 4 healthy donors. Each healthy donor had 2 WGS replicates that were sequenced in separate batches. The first batch had an average coverage of 27× (range 23-30×). We used the higher (case) vs. lower (control) coverage data for each patient for paired “normal-normal” analysis.

DirectHRD model and HRD score: As disclosed in McVey and Lee (Trends Genet. 2008 November; 24(11): 529-538) microhomology-mediated end-repair (MMER) is an alternative pathway to repair DNA double-stranded breaks (DSB), creates unique mutation signatures, i.e., small deletions with microhomology, referred to as mhDel in this study (FIG. 4). Two Cosmic Indel signatures, i.e., ID6 and ID8, are found to be caused by repairing DNA DSBs by non-homologous DNA end-joining mechanisms. However, ID8's etiology is less clear than ID6, and it has been found in a much higher prevalence and in tumor types that are less common to have HRD. Therefore, ID6 was chosen to be used as the HRD positive signature. An HRD negative indel signature was also created using a public WGS dataset, PCAWG: 66 breast cancer samples of 0 HRD probability were first identified according to CHORD's prediction; the Indel VCFs from the ICGC portal were then downloaded and the indel frequency was summarized according to the ID83 format from COSMIC. In ID83 format, mhDel is subclassified by the deletion length and the length of the microhomology. PCAWG HRD positive signature was created from 27 breast cancer WGS of at least 75% HRD probability by CHORD. As expected, the cosine similarity between PCAWG HRD positive signature and ID6 was high (93%) whereas it dropped to 73% when compared to ID8. PCAWG HRD positive signature will be substituted in if ID6 fails to converge in the EM algorithm. In ID83 format, an mhDel is subclassified by the deletion length and the length of the microhomology. There are 11 types of mhDel and the count vector of each mhDel type plus the deletion of 5 or more base pairs that is not repeat or microhomology mediated (5:Del:R:0) is used as the input of DirectHRD, which probabilistically assigns each mhDel to these signatures (ID6, PCAWG HRD negative signature) using a multinomial mixture model (MMM). The score of each mhDel is essentially a posterior probability (thus 0-1) of being related to HRD. The final HRD score of DirectHRD is the sum of scores of deletions of ≥5 bp and ≥1 bp homology. To be more specific, the likelihood of the MMM model can be represented by the following equation:

$$Lik = π {\prod }_{i = 1}^{12} {p}_{i}^{{n}_{i}} + ( 1 - π ) {\prod }_{i = 1}^{12} {d}_{i}^{{n}_{i}} \qquad ( 1 )$$

Here, n_i is the observed count of mhDel type i and π is the mixing proportion and can be considered as the fraction of HRD positive signature. P,D are two multinomial distributions with parameters p_id_i representing the probability of observing each mhDel type from the positive and negative signatures, respectively. To maximize the likelihood function (1) with respect to π,p_i,d_i, an Expectation-maximization (EM) algorithm was used. The initial values of π=0.5 and P,D are, respectively, probability vectors of ID6 and PCAWG HRD negative signatures. The EM algorithm iterates between taking the expectation of the log-likelihood evaluated at the current estimates of the parameters and a maximization step which calculates a new set of parameters. Since more parameters are present than data, and to avoid overfitting the data, a decay parameter α=1/N_mhDel was added when updating P,Q: for example, P\hat{ }(j+1)=αP\hat{ }(j−1)+(1−α) P\hat{ }j, and similarly, for Q. Here, j indexes the iterations and P\hat{ }j means the optimized results in the M step. Intuitively, P,Q allow a sample to have its own signatures, but this decay parameter α slows down the change of P,Q from their initial values and, the less the data, the less they should deviate from the consensus signatures. The iteration is terminated if the change of π between two consecutive iterations is less than 10-5. In practice, the number of iterations was often <30 times. After obtaining P,Q and π, the posterior probabilities of mhDel type i were calculated by: p_i n/(p_i π+q_i (1−π)).

In silico down-sampling and simulated dilution of tumor biopsies: In order to understand DirectHRD performance on lower tumor content samples, in silico dilution series of tumor WGS were first constructed from the early stage TNBC cohort (Mayer et al., Ann Oncol., 2020 November; 31(11):1518-1525. Doi: 10.1016/j.annonc.2020.08.2064, and Parsons et al. Ann Oncol. 2023 October; 34(10):899-906. Doi: 10.1016/j.annonc.2023.08.004). Due to the limit of sequencing depths of matched germline controls (˜15×, Table 1) (which effectively limited the depths when creating low purity in silico tumors), uniform coverage of ˜15× was selected for all in silico tumors at various tumor fractions (undiluted, 5% and 2%). To do so, the tumor and normal were downsampled from each patient separately based on the targeted depth and tumor fraction. For tumors, the downsample fraction was calculated as (15×*targeted_tumor_fraction)/(original_tumor_depth*original_tumor_fraction). For normals, the downsample fraction was calculated as (15−tumor_downsample_fraction*original_tumor_depth)/(original_normal_depth). Here, the original_tumor_fractions were calculated by Absolute (Carter et al., Nat Biotechnol. 2012 May; 30(5):413-21. Doi: 10.1038/nbt.2203). To be consistent, the 15× downsampled undiluted samples instead of the original samples were used consistently throughout this study for calculating HRD scores from tumor biopsies in the early stage TNBC cohort.

Selecting case-control samples and establishing patients' HRD statuses in the metastatic breast cancer cohort: In the metastatic breast cancer cohort, the ground-truth HRD status was established from patients' tumor WES. Two orthogonal methods were run and the consensus results of the two methods was taken. scarHRD sums a HRD score from large-scale variations including Lost of Heterozygosity (LoH), Large State Transition (LST) and Telomeric Allelic Imbalance (TSI). Whereas, SigMA uses Signature-3 from COSMIC, i.e., the HRD signature, which is derived from point mutations. To run SigMA, we first ran Mutect2 on the tumor-normal WES pairs to get the list of somatic SNVs, which SigMA used as input to its multivariate analysis (MVA) model. Two levels of confidence are output by MVA: MVA-pass and MVA-pass-strict. The later represents a more stringent cutoff for HRD positivity. We first found 14 out of the 99 patients have at least 1 tumor sample which were HRD-positive by SigMA (MVA-pass-strict). However, 3 out of the 14 had inconsistent results from different tumor samples from the same patient. The remaining 11 patients were further tested by scarHRD. One patient did not meet the cutoff of HRD positivity (42) and was thus excluded. Since there were a lot of HRD negative patients by SigMA (n=85) (expected from an ER+ breast cancer cohort), we randomly selected 35 out of them and ran scarHRD on their tumor samples. 9 patients had at least one HRD-positive tumor sample according to scarHRD (>42) and were thus excluded. At last, we manually selected 10 out of the remaining 26 negative patients whose tumor fractions of the first available liquid biopsies matched those from the 10 selected HRD positive patients (FIG. 9A). The tumor fractions of the cfDNAs were estimated from ultra-low-pass whole-genome sequencing (ULP-WGS) using ichorCNA. All WES and ULP-WGS samples were sequenced as part of a previous study.

Recalibration of cell-free DNA tumor fractions in the metastatic breast cancer cohort: In the metastatic breast cancer cohort, we selected samples with both tumor WES and ichorCNA tumor fraction estimation from cfDNA ULP-WGS. We first used the ULP-WGS tumor fractions to select cfDNA libraries for deeper WGS. We then recalibrated the tumor fractions estimated in the deeper WGS libraries. To do this, we first derived a list of somatic SNVs (we called fingerprints) from each patient's tumor-normal WES data, a.k.a. the Mutect call from the previous section. We then used MIREDAS to filter reads and calculate mutation allele frequencies on the fingerprints and multiplied the mean VAF by 2 to derive the recalibrated tumor fractions.

Comparing HRD scores between tumor and liquid biopsies: When comparing the HRD scores from cfDNA samples and tumor biopsies of the same patients, the raw HRD scores were used in the tumor biopsies and adjusted the scores by the tumor fractions in the cfDNA samples. The adjusted scores represented the expected HRD intensities from cfDNA and were calculated by simply multiplying the raw scores from the tumor biopsies by the cfDNA tumor fractions in the same sample. In the metastatic breast cancer cohort, the raw tumor HRD scores were calculated from WES by scarHRD. In the early stage TNBC cohort, the raw tumor HRD scores were calculated from WGS by DirectHRD and scarHRD.

Statistics and reproducibility: The sample size was determined by the availability of tissue biopsies in each cohort, the number of HRD-positive patients, and the cfDNA tumor fractions. No statistical method was used to predetermine the sample size. The experiments were not randomized. The investigators were not blinded to allocation during experiments and outcome assessment. All statistical analyses in this work can be reproduced by codes on our GitHub repository https://github.com/broadinstitute/DirectHRD.

Code availability: The SFC/mhDel caller is available at https://doi.org/10.5281/zenodo.7630357 and https://github.com/broadinstitute/CODECsuite. DirectHRD and code required to reproduce the analyses is available at https://github.com/broadinstitute/DirectHRD.

Calling Indels in low purity samples: In various embodiments, the standard Illumina WGS exhibits low error rates for Indels (insertion/deletion polymorphism), with even greater accuracy for mhDels compared to other alterations, such as single nucleotide variants (SNVs) in high complexity regions (FIG. 13A). This led to a hypothesis that the standard WGS may be sufficient for mhDel detection. Indeed, it was found that CODEC SFC can be successfully applied to standard Illumina WGS data for high accuracy. Some filters were implemented specifically for CODEC sequencing and were modified accordingly for standard NGS. Most importantly, a mutation was required to be supported by at least two unique fragments for error correction in the standard NGS data, and it was found that it detected mhDels with an error rate of 3×10⁻⁹ (FIG. 13A). The full list of parameters and command lines are given at https://github.com/broadinstitute/DirectHRD Additional filters for Indel calling include the following:
An Indel of interest is at least 8 bp from the ends of a read.
No germline SNP (Single nucleotide polymorphism) or Indel is adjacent (within 5 bp) to an Indel of interest.
Unique read depth of a matched germline at an Indel of interest is ≥10
The read which contains an Indel of interest does not have another SNV or Indel nearby (for SNV ±3 bp, for Indel ±10 bp).

To benchmark against other somatic Indel callers, including Strelka1 and Strelka2, and Mutect2, a high-complexity region filter was applied to all callers, requiring that an Indel must occur in these regions (GIAB V3.0 easy regions, total 2.3 B, hg19). Strelka1 was included because it is the default Indel caller by CHORD, but the next generation caller (Strelka2) was also tested for comparison.

DirectHRD Workflow: Example embodiments first benchmarked DirectHRD's ability to detect mhDels against established somatic Indel callers, including Mutect2 and Strelka1 and Strelka2 using six samples from the TNBC cohort as positive controls and four samples from healthy donors' buffy-coat DNA as negative controls. The six positive-control samples, selected for their highest matched germline WGS coverages, were downsampled from 60× to 10× to simulate low purity samples. The ground-truth mhDels were derived from the consensus results of Mutect2 and Strelka2 applied to the 60×WGS of the tumor biopsies. For the negative control data, example embodiments used the two technical replicates per healthy donor as a case-control pair, expecting no mutations would be called. On average, DirectHRD produced 7.5 false positives across the 4 negative controls, compared to 31 for Mutect2 and 20 for Strelka2. Strelka1 yielded only two false positives per sample, but failed to detect any true mhDels in the positive control samples. In contrast, DirectHRD achieved the highest recall at 40%, outperforming Strelka2 and Mutect2, both of which achieved 30% recall. In summary, DirectHRD showed superior performance for mhDel detection as compared to the state-of-the-art Indel detection methods in samples with fraction of tumor DNA (FIG. 13B-C).

Using the HRD status determined by Myriad MyChoice as the ground truth, example embodiments found DirectHRD [area under the curve (AUC): 0.99] outperformed CHORD (AUC: 0.90) and HRDetect (AUC: 0.88), two leading WGS-based classifiers (FIG. 10A). As an independent hold-out validation, example embodiments applied DirectHRD to 474 samples from the PCAWG project, focusing on the four cancer types with the highest HRD prevalence (FIG. 10B).

Example embodiments also assessed DirectHRD's performance in two additional cancer types: prostate and ovarian (FIG. 11A). The prostate cancer cohort included 25 cfDNA samples (median coverage 47×, range 33-103×; median tumor fraction 9.7%, range 0.6%-46.6%) from 20 patients with mCRPC, previously reported in published studies. No statistically significant difference in tumor fractions was observed between HRD-positive and HRD-negative cfDNA samples (FIG. 15A). Additionally, 10 cfDNA samples were analyzed (median coverage 58×, range 42-69×; median tumor fraction 3.8%, range 2.2%-12.4%) from 10 patients with stage III and IV ovarian cancer. Ground-truth HRD statuses were determined using germline and somatic BRCA status for the prostate cancer cohort (FIG. 15B) and HRD scar status derived from tumor WES for the ovarian cancer cohort (FIG. 15C-D). DirectHRD outperformed CHORD in sensitivity and NPV for prostate and ovarian cancers (FIG. 11B). In prostate cancer, DirectHRD achieved 83% sensitivity and 80% NPV, compared to CHORD's 25% sensitivity and 60% NPV. For ovarian cancer, DirectHRD demonstrated a sensitivity of 100% and an NPV of 100%, while CHORD's sensitivity was 0% and NPV was 30%.

Further, consistent with previous cohorts, DirectHRD scores derived from cfDNA demonstrated a good correlation (Spearman's correlation: 0.58) with the HRD intensities observed in patients' tumor biopsies within the ovarian cancer cohort after adjusting for tumor fractions in cfDNA (FIG. 15E).

This disclosure, for various embodiments of the disclosed approach, has: (i) demonstrated that various embodiments can reliably detect mhDels in low-purity samples, such as liquid biopsies, using moderate depths of WGS with a custom mhDel caller; (ii) proven that using mhDels alone is sufficient for HRD detection; and (iii) developed a probability model based on the HRD-associated Indel signature (Cosmic ID6), characterized by microhomology deletions, which further improved HRD detection. The disclosed approach outperforms state-of-the-art methods which use additional genomic scar features associated with HRD.

TABLE 1
Stage II-Stage III Breast Cancer Cohort Patient Information

| | | | CHORD | | | has | scarHRD | |
|---|---|---|---|---|---|---|---|---|
| Patient | HRD 33 | HRD 42 | results | Age | TD | cfDNA | score | CODEC |
| ONC138111 | NEGATIVE | NEGATIVE | | 38 | Y | Y | 36 | Y |
| ONC143854 | | NEGATIVE | | 42 | Y | Y | 34 | Y |
| ONC147579 | POSITIVE | POSITIVE | | 56 | Y | Y | 82 | N |
| ONC152713 | NEGATIVE | NEGATIVE | | 48 | Y | Y | 36 | N |
| ONC33279 | NEGATIVE | NEGATIVE | | 49 | Y | Y | 29 | N |
| ONC156134 | POSITIVE | POSITIVE | | 30 | Y | N | 73 | N |
| ONC156389 | NEGATIVE | NEGATIVE | | 38 | Y | Y | 34 | Y |
| ONC157864 | NEGATIVE | NEGATIVE | | 67 | Y | Y | 35 | Y |
| ONC159155 | POSITIVE | POSITIVE | | 38 | Y | Y | 72 | N |
| ONC159391 | POSITIVE | POSITIVE | | 52 | Y | Y | 36 | N |
| ONC161752 | POSITIVE | POSITIVE | | 33 | Y | Y | 58 | Y |
| ONC166813 | NEGATIVE | NEGATIVE | | 71 | Y | Y | 15 | Y |
| ONC167203 | POSITIVE | POSITIVE | | 41 | Y | N | 85 | N |
| ONC170292 | | NEGATIVE | | 55 | Y | Y | 44 | N |
| ONC170658 | POSITIVE | POSITIVE | | 50 | Y | Y | 73 | N |
| ONC171635 | POSITIVE | POSITIVE | | 51 | Y | N | 10 | N |
| ONC172301 | POSITIVE | POSITIVE | | 29 | Y | Y | 56 | Y |
| ONC173793 | POSITIVE | POSITIVE | | 38 | Y | Y | 65 | Y |
| ONC174333 | NEGATIVE | NEGATIVE | | 40 | Y | Y | 36 | Y |
| ONC175142 | POSITIVE | POSITIVE | | 47 | Y | Y | 83 | Y |
| ONC176222 | POSITIVE | POSITIVE | | 27 | Y | Y | 71 | N |
| ONC178033 | POSITIVE | POSITIVE | | 49 | Y | Y | 39 | N |
| ONC179846 | NEGATIVE | NEGATIVE | | 58 | Y | Y | 19 | Y |
| ONC181838 | POSITIVE | POSITIVE | | 53 | Y | Y | 65 | N |
| ONC184454 | POSITIVE | POSITIVE | | 56 | Y | Y | 65 | Y |
| ONC192824 | POSITIVE | POSITIVE | | 54 | Y | Y | 79 | N |
| ONC195700 | POSITIVE | POSITIVE | | 49 | Y | N | 89 | N |
| ONC166941 | POSITIVE | NEGATIVE | | 57 | N | Y | NA | N |
| ONC182745 | INCONCLUSIVE | INCONCLUSIVE | POSITIVE | 44 | N | Y | NA | N |
| ONC193018 | Insufficient | Insufficient | POSITIVE | 50 | N | Y | NA | N |
| | Tissue | Tissue | | | | | | |
| ONC195079 | POSITIVE | NEGATIVE | | 50 | N | Y | NA | N |
| ONC195262 | Insufficient | Insufficient | POSITIVE | 54 | N | Y | NA | N |
| | Tissue | Tissue | | | | | | |
| ONC194658 | Insufficient | Insufficient | POSITIVE | 35 | N | Y | NA | N |
| | Tissue | Tissue | | | | | | |

HRD 33 = “HRD Report Result Cutoff 33”;
HRD 42 = “HRD Report Result Cutoff 42”;
Age = “Age at Diagnosis”;
TD = “has tumor dilution”;
CODEC = “tumor biopsies sequenced by CODEC”

TABLE 2
Stage II-Stage III Breast Cancer Cohort Patient Information

| | library_{—} | tumor_{—} | Mean_{—} | Median_{—} | Duplication_{—} |
|---|---|---|---|---|---|
| out_id | type | purity | coverage | coverage | rate |
| ONC138111 | Normal | NA | 15.382672 | 15 | 0.251142 |
| ONC143854 | Normal | NA | 14.921362 | 15 | 0.111036 |
| ONC147579 | Normal | NA | 12.59911 | 12 | 0.108402 |
| ONC152713 | Normal | NA | 13.04039 | 13 | 0.103968 |
| ONC156134 | Normal | NA | 19.92791 | 20 | 0.132964 |
| ONC156389 | Normal | NA | 12.493425 | 12 | 0.093856 |
| ONC157864 | Normal | NA | 21.136053 | 21 | 0.259315 |
| ONC159155 | Normal | NA | 19.227921 | 19 | 0.238843 |
| ONC159391 | Normal | NA | 12.606444 | 13 | 0.098914 |
| ONC161752 | Normal | NA | 10.823279 | 11 | 0.083604 |
| ONC166813 | Normal | NA | 18.992497 | 19 | 0.253744 |
| ONC167203 | Normal | NA | 19.051118 | 19 | 0.244371 |
| ONC170292 | Normal | NA | 20.397992 | 20 | 0.270279 |
| ONC170658 | Normal | NA | 16.307949 | 16 | 0.253071 |
| ONC171635 | Normal | NA | 18.113292 | 18 | 0.258143 |
| ONC172301 | Normal | NA | 17.948987 | 18 | 0.229414 |
| ONC173793 | Normal | NA | 13.681386 | 14 | 0.106416 |
| ONC174333 | Normal | NA | 17.495865 | 17 | 0.238795 |
| ONC175142 | Normal | NA | 17.8194 | 18 | 0.229232 |
| ONC176222 | Normal | NA | 13.201849 | 13 | 0.103738 |
| ONC178033 | Normal | NA | 11.953952 | 12 | 0.098134 |
| ONC179846 | Normal | NA | 17.033673 | 17 | 0.234657 |
| ONC181838 | Normal | NA | 11.490406 | 11 | 0.1086 |
| ONC184454 | Normal | NA | 17.368013 | 17 | 0.242342 |
| ONC192824 | Normal | NA | 14.013803 | 14 | 0.107445 |
| ONC195700 | Normal | NA | 19.408113 | 19 | 0.231529 |
| ONC33279 | Normal | NA | 16.18562 | 16 | 0.231187 |
| ONC156134 | Normal | NA | 19.92791 | 20 | 0.132964 |
| ONC167203 | Normal | NA | 19.051118 | 19 | 0.244371 |
| ONC171635 | Normal | NA | 18.113292 | 18 | 0.258143 |
| ONC195700 | Normal | NA | 19.408113 | 19 | 0.324702 |
| ONC138111 | Tumor | 0.315789474 | 13.862526 | 13 | 0.078524 |
| ONC143854 | Tumor | 0.19047619 | 14.670238 | 14 | 0.025555 |
| ONC147579 | Tumor | 0.366197183 | 13.728138 | 13 | 0.027545 |
| ONC152713 | Tumor | 0.247191236 | 14.165738 | 14 | 0.028691 |
| ONC156134 | Tumor | 0.309677419 | 14.657555 | 14 | 0.024887 |
| ONC156389 | Tumor | 0.19047619 | 13.40265 | 13 | 0.024727 |
| ONC157864 | Tumor | 0.622222222 | 14.128662 | 14 | 0.063912 |
| ONC159155 | Tumor | 0.352941176 | 13.861312 | 13 | 0.079564 |
| ONC159391 | Tumor | 0.205882353 | 13.532559 | 13 | 0.027506 |
| ONC161752 | Tumor | 0.173913043 | 11.595709 | 11 | 0.022513 |
| ONC166813 | Tumor | 0.238095238 | 13.950626 | 14 | 0.075883 |
| ONC166941 | Tumor | NA | 16.824915 | 16 | 0.026077 |
| ONC167203 | Tumor | 0.512822821 | 14.018095 | 13 | 0.068445 |
| ONC170292 | Tumor | 0.461538462 | 13.989366 | 14 | 0.073321 |
| ONC170658 | Tumor | 0.553191489 | 14.075095 | 13 | 0.065358 |
| ONC171635 | Tumor | 0.222222222 | 13.947282 | 14 | 0.075693 |
| ONC172301 | Tumor | 0.285714286 | 13.925718 | 14 | 0.075502 |
| ONC173793 | Tumor | 0.4 | 14.588571 | 14 | 0.028887 |
| ONC174333 | Tumor | 0.242424242 | 14.194673 | 13 | 0.060821 |
| ONC175142 | Tumor | 0.64 | 13.858754 | 13 | 0.080389 |
| ONC176222 | Tumor | 0.239130435 | 14.406352 | 14 | 0.021915 |
| ONC178033 | Tumor | 0.136363636 | 12.930624 | 13 | 0.02312 |
| ONC179846 | Tumor | 0.416666667 | 14.121103 | 14 | 0.061983 |
| ONC181838 | Tumor | 0.295454545 | 12.557578 | 12 | 0.021252 |
| ONC182745 | Tumor | NA | 16.442495 | 16 | 0.07143 |
| ONC184454 | Tumor | 0.742857143 | 13.995908 | 13 | 0.07281 |
| ONC192824 | Tumor | 0.333333333 | 14.715723 | 14 | 0.02623 |
| ONC193018 | Tumor | NA | 16.435128 | 16 | 0.023043 |
| ONC194658 | Tumor | NA | 16.183846 | 16 | 0.081899 |
| ONC195079 | Tumor | NA | 16.420391 | 16 | 0.075118 |
| ONC195262 | Tumor | NA | 16.368359 | 15 | 0.030582 |
| ONC195700 | Tumor | 0.392156863 | 14.001001 | 14 | 0.070383 |
| ONC33279 | Tumor | 0.28 | 14.052981 | 14 | 0.069739 |
| ONC175142_W00 | cfDNA | 0.9 | 24.808888 | 23 | 0.210258 |
| ONC159155_W00 | cfDNA | 0.360833257 | 14.278357 | 11 | 0.268794 |
| ONC172301_W00 | cfDNA | 0.105865618 | 44.209719 | 45 | 0.259827 |
| ONC192824_W00 | cfDNA | 0.071655658 | 54.442237 | 53 | 0.22948 |
| ONC159155_W12 | cfDNA | 0.0369 | 35.209756 | 31 | 0.182391 |
| ONC161752_W00 | cfDNA | 0.03429801 | 34.235098 | 18 | 0.335872 |
| ONC147579_W00 | cfDNA | 0.029227016 | 42.076344 | 43 | 0.255077 |
| ONC178033_W00 | cfDNA | 0.023662579 | 37.275217 | 37 | 0.238891 |
| ONC184454_W00 | cfDNA | 0.018401025 | 62.030782 | 63 | 0.255572 |
| ONC170658_W00 | cfDNA | 0.013007833 | 56.71407 | 58 | 0.284971 |
| ONC173793_W00 | cfDNA | 0.011626978 | 39.030062 | 34 | 0.244116 |
| ONC178033_W12 | cfDNA | 0.0068 | 50.838492 | 50 | 0.266542 |
| ONC181838_W12 | cfDNA | 0.0043 | 40.819935 | 41 | 0.3176 |
| ONC176222_W00 | cfDNA | 0.00334 | 44.11422 | 37 | 0.286963 |
| ONC159391_W00 | cfDNA | 0.001655133 | 37.913612 | 38 | 0.314592 |
| ONC195262_W00 | cfDNA | 0.0988 | 43.82526 | 40 | 0.197616 |
| ONC195262_W12 | cfDNA | 0.0682 | 29.514335 | 28 | 0.274661 |
| ONC195262_W03 | cfDNA | 0.0536 | 29.296902 | 28 | 0.270944 |
| ONC194658_W00 | cfDNA | 0.0087 | 50.266752 | 51 | 0.284408 |
| ONC193018_W00 | cfDNA | 0.0065 | 45.842148 | 45 | 0.292107 |
| ONC182745_W00 | cfDNA | 0.003 | 62.842605 | 64 | 0.258038 |
| ONC179846_W00 | cfDNA | 0.056716743 | 44.986312 | 44 | 0.21543 |
| ONC156389_W03 | cfDNA | 0.0461 | 42.57487 | 43 | 0.236939 |
| ONC138111_W00 | cfDNA | 0.042618085 | 43.674535 | 44 | 0.299259 |
| ONC152713_W00 | cfDNA | 0.025245997 | 39.699431 | 40 | 0.295518 |
| ONC143854_W00 | cfDNA | 0.02138264 | 38.725449 | 39 | 0.27634 |
| ONC166941_W12 | cfDNA | 0.019 | 54.984752 | 45 | 0.286612 |
| ONC170292_W00 | cfDNA | 0.016919926 | 40.649149 | 35 | 0.254141 |
| ONC138111_W03 | cfDNA | 0.00981 | 54.665333 | 56 | 0.296514 |
| ONC138111_W12 | cfDNA | 0.0091 | 47.828086 | 48 | 0.270227 |
| ONC166813_W00 | cfDNA | 0.006978915 | 36.964667 | 36 | 0.284734 |
| ONC166941_W00 | cfDNA | 0.004850131 | 38.641535 | 32 | 0.25662 |
| ONC152713_W03 | cfDNA | 0.00364 | 46.331815 | 47 | 0.362898 |
| ONC33279_W00 | cfDNA | 0.003023931 | 43.116213 | 44 | 0.228794 |
| ONC157864_W00 | cfDNA | 0.001581093 | 31.770043 | 23 | 0.223251 |
| ONC195079_W00 | cfDNA | 0.00146698 | 41.425643 | 42 | 0.246772 |
| ONC174333_W00 | cfDNA | 0.001452381 | 47.310323 | 48 | 0.275539 |

TABLE 3

| Participant | HRD | scarHRD_score | Analyzed_by_directHRD |
|---|---|---|---|
| 05246_185 | Pos | 69 | Y |
| 05246_252 | Pos | 60 | Y |
| 05246_292 | Pos | 56 | Y |
| 05246_300 | Neg | 11 | N |
| 05246_303 | Pos | 52 | Y |
| 05246_306 | Neg | 21 | N |
| 05246_313 | Neg | 25 | Y |
| 05246_315 | Neg | 30 | Y |
| 05246_318 | Pos | 56 | Y |
| 05246_321 | Pos | 70 | Y |
| 05246_331 | Neg | 39 | Y |
| 05246_343 | Neg | 18 | Y |
| 05246_350 | Neg | 27 | Y |
| 05246_357 | Neg | 19 | Y |
| 05246_359 | Pos | 73 | Y |
| 05246_371 | Pos | 51 | Y |
| 05246_376 | Neg | 28 | Y |
| 05246_465 | Pos | 63 | Y |
| 05246_473 | Pos | 49 | Y |
| 05246_8 | Neg | 25 | Y |

TABLE 4

| | Library | Mn. | Md. | | | | | Wes |
|---|---|---|---|---|---|---|---|---|
| Patient | type | Cov. | Cov. | DR | ploidy | TF | 1D | informed_tfx |
| 05246_185 | cfDNA | 18.169408 | 18 | 0.382375 | 2.015 | 0.04696 | TRUE | 3.64E−04 |
| 05246_252 | cfDNA | 27.498802 | 27 | 0.377883 | 3.684 | 0.1807 | TRUE | 0.16027333 |
| 05246_292 | cfDNA | 20.861574 | 20 | 0.374865 | 1.814 | 0.2252 | TRUE | 0.07371484 |
| 05246_300 | cfDNA | 3.744799 | 1 | 0.320814 | 1.884 | 0.4026 | TRUE | 0 |
| 05246_303 | cfDNA | 19.436086 | 19 | 0.388616 | 1.765 | 0.4051 | TRUE | 0.20381953 |
| 05246_306 | cfDNA | 18.565488 | 18 | 0.381992 | 1.998 | 0 | TRUE | 0 |
| 05246_313 | cfDNA | 17.180492 | 17 | 0.432914 | 2.283 | 0.2855 | TRUE | 0.08199019 |
| 05246_315 | cfDNA | 24.309714 | 24 | 0.410192 | 1.909 | 0.2528 | TRUE | 0.11120616 |
| 05246_318 | cfDNA | 26.551289 | 24 | 0.350575 | 2.039 | 0.654 | TRUE | 0.30427555 |
| 05246_321 | cfDNA | 24.112224 | 23 | 0.349562 | 2.917 | 0.2693 | TRUE | 0.23842065 |
| 05246_331 | cfDNA | 17.232789 | 16 | 0.344039 | 1.875 | 0.3984 | TRUE | 0.2819537 |
| 05246_343 | cfDNA | 27.003259 | 27 | 0.446276 | 2.018 | 0.3751 | TRUE | 0.37287022 |
| 05246_350 | cfDNA | 16.036608 | 16 | 0.371112 | 2.091 | 0.03359 | TRUE | 0.01635992 |
| 05246_357 | cfDNA | 17.041674 | 17 | 0.355822 | 1.913 | 0.06843 | TRUE | 0.03830142 |
| 05246_359 | cfDNA | 16.753044 | 16 | 0.400272 | 2.632 | 0.3357 | TRUE | 0.26379847 |
| 05246_371 | cfDNA | 27.270272 | 25 | 0.337257 | 2.928 | 0.1718 | TRUE | 0.20718632 |
| 05246_376 | cfDNA | 19.118079 | 17 | 0.355866 | 1.937 | 0.027 | TRUE | 0.00449944 |
| 05246_465 | cfDNA | 26.455875 | 26 | 0.338031 | 1.817 | 0.2455 | TRUE | 0.124839 |
| 05246_473 | cfDNA | 19.964601 | 17 | 0.327866 | 2.006 | 0.1501 | TRUE | 0.09619386 |
| 05246_8 | cfDNA | 17.269691 | 17 | 0.364657 | 2.234 | 0.1939 | TRUE | 0.17846323 |
| 05246_185 | Normal | 9.351267 | 9 | 0.303522 | | | | |
| 05246_252 | Normal | 10.94164 | 11 | 0.3002 | | | | |
| 05246_292 | Normal | 11.655174 | 12 | 0.344585 | | | | |
| 05246_303 | Normal | 10.961139 | 11 | 0.307848 | | | | |
| 05246_318 | Normal | 8.435958 | 8 | 0.299512 | | | | |
| 05246_321 | Normal | 9.474694 | 9 | 0.302245 | | | | |
| 05246_357 | Normal | 10.646683 | 11 | 0.306611 | | | | |
| 05246_376 | Normal | 11.084272 | 11 | 0.291787 | | | | |
| 05246_8 | Normal | 10.94002 | 11 | 0.315588 | | | | |
| 05246_300 | Normal | 8.669524 | 9 | 0.245925 | | | | |
| 05246_359 | Normal | 9.824685 | 10 | 0.265482 | | | | |
| 05246_371 | Normal | 11.255898 | 11 | 0.264288 | | | | |
| 05246_465 | Normal | 9.473947 | 9 | 0.260779 | | | | |
| 05246_473 | Normal | 11.475988 | 11 | 0.287466 | | | | |
| 05246_306 | Normal | 6.887593 | 7 | 0.273647 | | | | |
| 05246_313 | Normal | 6.893474 | 7 | 0.271048 | | | | |
| 05246_315 | Normal | 6.293162 | 6 | 0.254265 | | | | |
| 05246_331 | Normal | 9.107408 | 9 | 0.279206 | | | | |
| 05246_343 | Normal | 6.885233 | 7 | 0.261521 | | | | |
| 05246_350 | Normal | 7.238577 | 7 | 0.265743 | | | | |

Mn. Cov. = “Mean Coverage”;
Md. Cov. = “Median Coverage”;
DR = “Duplication Rate”;
ploidy = “ulp_wgs ploidy”;
TF = “ulp_wgs tumor fraction”;
1D = “is_first_draw”

TABLE 5

| | | | | | total | | | |
|---|---|---|---|---|---|---|---|---|
| Sample | DS | HRDscore | pos_prob | neg_prob | mhDel | mh2h | mh5d | UBS |
| ONC138111 | TBF | 0.35 | 0.197566789 | 0.802433211 | 9 | 3 | 7 | N |
| ONC143854 | TBF | 0.02 | 0.051374082 | 0.948625918 | 9 | 6 | 2 | N |
| ONC147579 | TBF | 136.82 | 0.802147303 | 0.197852697 | 218 | 178 | 196 | N |
| ONC152713 | TBF | 0 | 0.000265716 | 0.999734284 | 12 | 6 | 4 | N |
| ONC156134 | TBF | 41.94 | 0.830813628 | 0.169186372 | 62 | 44 | 59 | N |
| ONC156389 | TBF | 0.34 | 0.221973566 | 0.778026434 | 7 | 3 | 3 | N |
| ONC157864 | TBF | 0.62 | 0.128269423 | 0.871730577 | 39 | 28 | 19 | N |
| ONC159155 | TBF | 139.91 | 0.896458585 | 0.103541415 | 178 | 147 | 168 | N |
| ONC159391 | TBF | 66.21 | 0.837592237 | 0.162407763 | 95 | 77 | 88 | N |
| ONC161752 | TBF | 18.52 | 0.736381549 | 0.263618451 | 35 | 26 | 31 | N |
| ONC166813 | TBF | 0.01 | 0.037087596 | 0.962912404 | 8 | 1 | 5 | N |
| ONC167203 | TBF | 34.22 | 0.772060435 | 0.227939565 | 58 | 40 | 55 | N |
| ONC170292 | TBF | 1.05 | 0.296873466 | 0.703126534 | 12 | 6 | 9 | N |
| ONC170658 | TBF | 78.62 | 0.811569471 | 0.188430529 | 124 | 99 | 112 | N |
| ONC171635 | TBF | 29 | 0.999927597 | 7.24E−05 | 29 | 23 | 29 | Y |
| ONC172301 | TBF | 30.22 | 0.701043224 | 0.298956776 | 62 | 42 | 57 | N |
| ONC173793 | TBF | 58.23 | 0.763416338 | 0.236583662 | 103 | 81 | 92 | N |
| ONC174333 | TBF | 4.11 | 0.514022876 | 0.485977124 | 16 | 11 | 13 | N |
| ONC175142 | TBF | 81.74 | 0.716840353 | 0.283159647 | 163 | 120 | 144 | N |
| ONC176222 | TBF | 131.99 | 0.99994833 | 5.17E−05 | 134 | 112 | 132 | N |
| ONC178033 | TBF | 1.25 | 0.569004148 | 0.430995852 | 4 | 4 | 3 | N |
| ONC179846 | TBF | 0.01 | 0.04079994 | 0.95920006 | 8 | 4 | 3 | N |
| ONC181838 | TBF | 66.09 | 0.793916158 | 0.206083842 | 107 | 75 | 101 | N |
| ONC184454 | TBF | 108.12 | 0.875790956 | 0.124209044 | 144 | 115 | 135 | N |
| ONC192824 | TBF | 285.08 | 0.815265982 | 0.184734018 | 440 | 348 | 401 | N |
| ONC195700 | TBF | 142.67 | 0.768770142 | 0.231229858 | 247 | 183 | 225 | N |
| ONC33279 | TBF | 0.59 | 0.30559672 | 0.69440328 | 7 | 3 | 4 | N |
| ONC138111 | TB5 | 0.05 | 0.114450483 | 0.885549517 | 4 | 1 | 2 | N |
| ONC143854 | TB5 | 0 | 0.042276633 | 0.957723367 | 3 | 2 | 0 | N |
| ONC147579 | TB5 | 26.62 | 0.898767114 | 0.101232886 | 33 | 26 | 32 | N |
| ONC152713 | TB5 | 0.02 | 0.067382433 | 0.932617567 | 5 | 3 | 2 | N |
| ONC156134 | TB5 | 5.83 | 0.556986789 | 0.443013211 | 19 | 11 | 17 | N |
| ONC156389 | TB5 | 1.00E−05 | 0.5 | 0.5 | 0 | 0 | 0 | N |
| ONC157864 | TB5 | 0.47 | 0.244664942 | 0.755335058 | 8 | 3 | 6 | N |
| ONC159155 | TB5 | 14.78 | 0.787002587 | 0.212997413 | 24 | 19 | 22 | N |
| ONC159391 | TB5 | 9.85 | 0.992544366 | 0.007455634 | 10 | 9 | 10 | N |
| ONC161752 | TB5 | 0.51 | 0.301850371 | 0.698149629 | 6 | 5 | 3 | N |
| ONC166813 | TB5 | 0.1 | 0.316661414 | 0.683338586 | 1 | 0 | 1 | N |
| ONC167203 | TB5 | 5.39 | 0.820613931 | 0.179386069 | 8 | 6 | 8 | N |
| ONC170292 | TB5 | 1.00E−05 | 0.5 | 0.5 | 0 | 0 | 0 | N |
| ONC170658 | TB5 | 16.48 | 0.872003866 | 0.127996134 | 22 | 18 | 21 | N |
| ONC171635 | TB5 | 4.42 | 0.939864041 | 0.060135959 | 5 | 5 | 5 | N |
| ONC172301 | TB5 | 5.86 | 0.987913585 | 0.012086415 | 6 | 6 | 6 | N |
| ONC173793 | TB5 | 11.47 | 0.803777701 | 0.196222299 | 18 | 14 | 17 | N |
| ONC174333 | TB5 | 0.76 | 0.281529831 | 0.718470169 | 10 | 7 | 6 | N |
| ONC175142 | TB5 | 8.28 | 0.463273963 | 0.536726037 | 39 | 21 | 35 | N |
| ONC176222 | TB5 | 28.41 | 0.84295488 | 0.15704512 | 41 | 33 | 38 | N |
| ONC178033 | TB5 | 0.47 | 0.68604651 | 0.31395349 | 1 | 1 | 1 | N |
| ONC179846 | TB5 | 0 | 0.023788826 | 0.976211174 | 4 | 1 | 1 | N |
| ONC181838 | TB5 | 2.19 | 0.461447671 | 0.538552329 | 11 | 9 | 8 | N |
| ONC184454 | TB5 | 17 | 0.999874102 | 1.26E−04 | 17 | 16 | 17 | N |
| ONC192824 | TB5 | 89.49 | 0.915538164 | 0.084461836 | 110 | 95 | 103 | N |
| ONC195700 | TB5 | 21.82 | 0.744700104 | 0.255299896 | 40 | 32 | 36 | N |
| ONC33279 | TB5 | 0.22 | 0.348945119 | 0.651054881 | 2 | 2 | 1 | N |
| ONC138111 | TB2 | 0 | 0.005434094 | 0.994565906 | 1 | 0 | 0 | N |
| ONC143854 | TB2 | 1.00E−05 | 0.5 | 0.5 | 0 | 0 | 0 | N |
| ONC147579 | TB2 | 1.46 | 0.614937586 | 0.385062414 | 4 | 4 | 3 | N |
| ONC152713 | TB2 | 0.1 | 0.316661414 | 0.683338586 | 1 | 0 | 1 | N |
| ONC156134 | TB2 | 0.04 | 0.159240824 | 0.840759176 | 2 | 0 | 1 | N |
| ONC156389 | TB2 | 1.00E−05 | 0.5 | 0.5 | 0 | 0 | 0 | N |
| ONC157864 | TB2 | 0.47 | 0.68604651 | 0.31395349 | 1 | 1 | 1 | N |
| ONC159155 | TB2 | 3.19 | 0.799193617 | 0.200806383 | 5 | 4 | 5 | N |
| ONC159391 | TB2 | 0.56 | 0.530653778 | 0.469346222 | 2 | 1 | 2 | N |
| ONC161752 | TB2 | 0.17 | 0.208624058 | 0.791375942 | 4 | 0 | 4 | N |
| ONC166813 | TB2 | 1.00E−05 | 0.5 | 0.5 | 0 | 0 | 0 | N |
| ONC167203 | TB2 | 1.00E−05 | 0.5 | 0.5 | 0 | 0 | 0 | N |
| ONC170292 | TB2 | 1.00E−05 | 0.5 | 0.5 | 0 | 0 | 0 | N |
| ONC170658 | TB2 | 0.73 | 0.502317832 | 0.497682168 | 3 | 3 | 2 | N |
| ONC171635 | TB2 | 1.00E−05 | 0.5 | 0.5 | 0 | 0 | 0 | N |
| ONC172301 | TB2 | 0.1 | 0.316661414 | 0.683338586 | 1 | 0 | 1 | N |
| ONC173793 | TB2 | 0.47 | 0.68604651 | 0.31395349 | 1 | 1 | 1 | N |
| ONC174333 | TB2 | 0 | 0.051049235 | 0.948950765 | 2 | 2 | 0 | N |
| ONC175142 | TB2 | 1.46 | 0.613518259 | 0.386481741 | 4 | 4 | 3 | N |
| ONC176222 | TB2 | 3.26 | 0.902766219 | 0.097233781 | 4 | 4 | 4 | N |
| ONC178033 | TB2 | 1.00E−05 | 0.5 | 0.5 | 0 | 0 | 0 | N |
| ONC179846 | TB2 | 0.1 | 0.316661414 | 0.683338586 | 1 | 0 | 1 | N |
| ONC181838 | TB2 | 0.1 | 0.161475467 | 0.838524533 | 4 | 2 | 2 | N |
| ONC184454 | TB2 | 0.66 | 0.469330909 | 0.530669091 | 3 | 1 | 3 | N |
| ONC192824 | TB2 | 11.44 | 0.976228069 | 0.023771931 | 12 | 10 | 12 | N |
| ONC195700 | TB2 | 0.65 | 0.404212927 | 0.595787073 | 4 | 2 | 3 | N |
| ONC33279 | TB2 | 1.00E−05 | 0.5 | 0.5 | 0 | 0 | 0 | N |
| 05246_185 | McfDNA | 1.00E−05 | NA | NA | 0 | 0 | 0 | N |
| 05246_252 | McfDNA | 460.72 | 0.890403116 | 0.109596884 | 599 | 500 | 557 | N |
| 05246_292 | McfDNA | 57.73 | 0.698269317 | 0.301730683 | 120 | 80 | 109 | N |
| 05246_300 | McfDNA | 1.00E−05 | NA | NA | 0 | 0 | 0 | N |
| 05246_303 | McfDNA | 286.84 | 0.906016035 | 0.093983965 | 359 | 305 | 336 | N |
| 05246_306 | McfDNA | 0.24 | 0.355896653 | 0.644103347 | 2 | 1 | 1 | N |
| 05246_313 | McfDNA | 0.52 | 0.23156484 | 0.76843516 | 10 | 6 | 6 | N |
| 05246_315 | McfDNA | 0.57 | 0.186948382 | 0.813051618 | 17 | 11 | 10 | N |
| 05246_318 | McfDNA | 187.84 | 0.892877281 | 0.107122719 | 243 | 214 | 224 | N |
| 05246_321 | McfDNA | 268.66 | 0.83818553 | 0.16181447 | 390 | 321 | 357 | N |
| 05246_331 | McfDNA | 2.73 | 0.434745138 | 0.565254862 | 15 | 10 | 10 | N |
| 05246_343 | McfDNA | 0 | 9.92E−07 | 0.999999008 | 992 | 224 | 42 | N |
| 05246_350 | McfDNA | 1.00E−05 | NA | NA | 0 | 0 | 0 | N |
| 05246_357 | McfDNA | 0.32 | 0.398523773 | 0.601476227 | 2 | 1 | 1 | N |
| 05246_359 | McfDNA | 126.92 | 0.869130739 | 0.130869261 | 172 | 144 | 159 | N |
| 05246_371 | McfDNA | 2.93 | 0.170932729 | 0.829067271 | 106 | 36 | 78 | Y |
| 05246_376 | McfDNA | 0.64 | 0.80171158 | 0.19828842 | 1 | 1 | 1 | N |
| 05246_465 | McfDNA | 204.37 | 0.801546389 | 0.198453611 | 327 | 260 | 294 | N |
| 05246_473 | McfDNA | 1.11 | 0.276665369 | 0.723334631 | 15 | 10 | 7 | N |
| 05246_8 | McfDNA | 0.02 | 0.03446613 | 0.96553387 | 20 | 11 | 10 | N |
| ONC138111_W00 | EScfDNA | 0 | 0.003041565 | 0.996958435 | 10 | 6 | 3 | N |
| ONC138111_W03 | EScfDNA | 0 | 0.00284395 | 0.99715605 | 12 | 4 | 2 | N |
| ONC138111_W12 | EScfDNA | 0 | 0.048037901 | 0.951962099 | 3 | 3 | 0 | N |
| ONC143854_W00 | EScfDNA | 0 | 0.005434094 | 0.994565906 | 1 | 0 | 0 | N |
| ONC147579_W00 | EScfDNA | 8.14 | 0.527228391 | 0.472771609 | 30 | 20 | 24 | N |
| ONC152713_W00 | EScfDNA | 0.23 | 0.343348855 | 0.656651145 | 2 | 1 | 1 | N |
| ONC152713_W03 | EScfDNA | 0 | 0.005434094 | 0.994565906 | 1 | 0 | 0 | N |
| ONC156389_W03 | EScfDNA | 0 | 0.014095705 | 0.985904295 | 2 | 0 | 0 | N |
| ONC157864_W00 | EScfDNA | 0.64 | 0.80171158 | 0.19828842 | 1 | 1 | 1 | N |
| ONC159155_W00 | EScfDNA | 102.81 | 0.847431616 | 0.152568384 | 146 | 116 | 136 | N |
| ONC159155_W12 | EScfDNA | 12.94 | 0.684777394 | 0.315222606 | 28 | 22 | 24 | N |
| ONC159391_W00 | EScfDNA | 1.00E−05 | NA | NA | 0 | 0 | 0 | N |
| ONC161752_W00 | EScfDNA | 4.22 | 0.569671133 | 0.430328867 | 13 | 7 | 13 | N |
| ONC166813_W00 | EScfDNA | 0 | 0.005434094 | 0.994565906 | 1 | 0 | 0 | N |
| ONC166941_W00 | EScfDNA | 0.24 | 0.355896653 | 0.644103347 | 2 | 1 | 1 | N |
| ONC166941_W12 | EScfDNA | 0.48 | 0.35042735 | 0.64957265 | 4 | 2 | 2 | N |
| ONC170292_W00 | EScfDNA | 0.47 | 0.68604651 | 0.31395349 | 1 | 1 | 1 | N |
| ONC170658_W00 | EScfDNA | 1.02 | 0.27316884 | 0.72683116 | 14 | 7 | 7 | N |
| ONC172301_W00 | EScfDNA | 41.84 | 0.652701375 | 0.347298625 | 99 | 62 | 91 | N |
| ONC173793_W00 | EScfDNA | 5.86 | 0.987913585 | 0.012086415 | 6 | 6 | 6 | N |
| ONC174333_W00 | EScfDNA | 0.01 | 0.045035129 | 0.954964871 | 6 | 2 | 1 | N |
| ONC175142_W00 | EScfDNA | 104.39 | 0.754157593 | 0.245842407 | 187 | 143 | 167 | N |
| ONC176222_W00 | EScfDNA | 2.05 | 0.370858858 | 0.629141142 | 15 | 7 | 12 | N |
| ONC178033_W00 | EScfDNA | 0 | 0.064938703 | 0.935061297 | 2 | 1 | 0 | N |
| ONC178033_W12 | EScfDNA | 0 | 0.064938703 | 0.935061297 | 2 | 1 | 0 | N |
| ONC179846_W00 | EScfDNA | 0 | 7.29E−05 | 0.999927097 | 13 | 8 | 2 | N |
| ONC181838_W12 | EScfDNA | 0.1 | 0.18510401 | 0.81489599 | 3 | 1 | 1 | N |
| ONC182745_W00 | EScfDNA | 0.26 | 0.230427038 | 0.769572962 | 5 | 3 | 2 | N |
| ONC184454_W00 | EScfDNA | 2.73 | 0.363237161 | 0.636762839 | 21 | 11 | 13 | N |
| ONC192824_W00 | EScfDNA | 153.38 | 0.824108455 | 0.175891545 | 229 | 187 | 210 | N |
| ONC193018_W00 | EScfDNA | 0.55 | 0.524930155 | 0.475069845 | 2 | 1 | 2 | N |
| ONC194658_W00 | EScfDNA | 2.98 | 0.411752073 | 0.588247927 | 18 | 13 | 11 | N |
| ONC195079_W00 | EScfDNA | 0 | 0.063134962 | 0.936865038 | 1 | 1 | 0 | N |
| ONC195262_W00 | EScfDNA | 9.5 | 0.458016934 | 0.541983066 | 47 | 36 | 32 | N |
| ONC195262_W03 | EScfDNA | 1 | 0.262847669 | 0.737152331 | 15 | 11 | 9 | N |
| ONC195262_W12 | EScfDNA | 8.38 | 0.812138844 | 0.187861156 | 13 | 10 | 12 | N |
| ONC33279_W00 | EScfDNA | 0.18 | 0.180438813 | 0.819561187 | 6 | 4 | 2 | N |

TBF = “tumor_biopsy_fullpurity”;
TB5 = “tumor_biopsy_5%”;
TB2 = “tumor_biopsy_2%”;
McfDNA = “metastatic_cfdna”;
EScfDNA = “early-stage_cfdna”;
mh2h = “mhDel >=2 bp homology”;
mh5d = “mhDel >=5 bp del”;
UBS = “use backup signature”

TABLE 5B
Direct HRD Results

| | | HRD | | | | use backup |
|---|---|---|---|---|---|---|
| sample | dataset | pred | HRDscore | ID6_prob | total_mhDel | signature |
| ONC138111 | tumor_biopsy_fullpurity | HRD− | 0.35 | 0.19756679 | 9 | N |
| ONC143854 | tumor_biopsy_fullpurity | HRD− | 0.02 | 0.05137408 | 9 | N |
| ONC147579 | tumor_biopsy_fullpurity | HRD+ | 136.82 | 0.8021473 | 218 | N |
| ONC152713 | tumor_biopsy_fullpurity | HRD− | 0 | 0.00026572 | 12 | N |
| ONC156134 | tumor_biopsy_fullpurity | HRD+ | 41.94 | 0.83081363 | 62 | N |
| ONC156389 | tumor_biopsy_fullpurity | HRD− | 0.34 | 0.22197357 | 7 | N |
| ONC157864 | tumor_biopsy_fullpurity | HRD− | 0.62 | 0.12826942 | 39 | N |
| ONC159155 | tumor_biopsy_fullpurity | HRD+ | 139.91 | 0.89645858 | 178 | N |
| ONC159391 | tumor_biopsy_fullpurity | HRD+ | 66.21 | 0.83759224 | 95 | N |
| ONC161752 | tumor_biopsy_fullpurity | HRD+ | 18.52 | 0.73638155 | 35 | N |
| ONC166813 | tumor_biopsy_fullpurity | HRD− | 0.01 | 0.0370876 | 8 | N |
| ONC167203 | tumor_biopsy_fullpurity | HRD+ | 34.22 | 0.77206044 | 58 | N |
| ONC170292 | tumor_biopsy_fullpurity | HRD+ | 1.05 | 0.29687347 | 12 | N |
| ONC170658 | tumor_biopsy_fullpurity | HRD+ | 78.62 | 0.81156947 | 124 | N |
| ONC171635 | tumor_biopsy_fullpurity | HRD+ | 29 | 0.9999276 | 29 | Y |
| ONC172301 | tumor_biopsy_fullpurity | HRD+ | 30.22 | 0.70104322 | 62 | N |
| ONC173793 | tumor_biopsy_fullpurity | HRD+ | 58.23 | 0.76341634 | 103 | N |
| ONC174333 | tumor_biopsy_fullpurity | HRD+ | 4.11 | 0.51402288 | 16 | N |
| ONC175142 | tumor_biopsy_fullpurity | HRD+ | 81.74 | 0.71684035 | 163 | N |
| ONC176222 | tumor_biopsy_fullpurity | HRD+ | 131.99 | 0.99994833 | 134 | N |
| ONC178033 | tumor_biopsy_fullpurity | HRD+ | 1.25 | 0.56900415 | 4 | N |
| ONC179846 | tumor_biopsy_fullpurity | HRD− | 0.01 | 0.04079994 | 8 | N |
| ONC181838 | tumor_biopsy_fullpurity | HRD+ | 66.09 | 0.79391616 | 107 | N |
| ONC184454 | tumor_biopsy_fullpurity | HRD+ | 108.12 | 0.87579096 | 144 | N |
| ONC192824 | tumor_biopsy_fullpurity | HRD+ | 285.08 | 0.81526598 | 440 | N |
| ONC195700 | tumor_biopsy_fullpurity | HRD+ | 142.67 | 0.76877014 | 247 | N |
| ONC33279 | tumor_biopsy_fullpurity | HRD− | 0.59 | 0.30559672 | 7 | N |
| ONC138111 | tumor_biopsy_5% | HRD− | 0.05 | 0.11445048 | 4 | N |
| ONC143854 | tumor_biopsy_5% | HRD− | 0 | 0.04227663 | 3 | N |
| ONC147579 | tumor_biopsy_5% | HRD+ | 26.62 | 0.89876711 | 33 | N |
| ONC152713 | tumor_biopsy_5% | HRD− | 0.02 | 0.06738243 | 5 | N |
| ONC156134 | tumor_biopsy_5% | HRD+ | 5.83 | 0.55698679 | 19 | N |
| ONC156389 | tumor_biopsy_5% | HRD− | 1.00E−05 | 0.5 | 0 | N |
| ONC157864 | tumor_biopsy_5% | HRD− | 0.47 | 0.24466494 | 8 | N |
| ONC159155 | tumor_biopsy_5% | HRD+ | 14.78 | 0.78700259 | 24 | N |
| ONC159391 | tumor_biopsy_5% | HRD+ | 9.85 | 0.99254437 | 10 | N |
| ONC161752 | tumor_biopsy_5% | HRD− | 0.51 | 0.30185037 | 6 | N |
| ONC166813 | tumor_biopsy_5% | HRD− | 0.1 | 0.31666141 | 1 | N |
| ONC167203 | tumor_biopsy_5% | HRD+ | 5.39 | 0.82061393 | 8 | N |
| ONC170292 | tumor_biopsy_5% | HRD− | 1.00E−05 | 0.5 | 0 | N |
| ONC170658 | tumor_biopsy_5% | HRD+ | 16.48 | 0.87200387 | 22 | N |
| ONC171635 | tumor_biopsy_5% | HRD+ | 4.42 | 0.93986404 | 5 | N |
| ONC172301 | tumor_biopsy_5% | HRD+ | 5.86 | 0.98791359 | 6 | N |
| ONC173793 | tumor_biopsy_5% | HRD+ | 11.47 | 0.8037777 | 18 | N |
| ONC174333 | tumor_biopsy_5% | HRD− | 0.76 | 0.28152983 | 10 | N |
| ONC175142 | tumor_biopsy_5% | HRD+ | 8.28 | 0.46327396 | 39 | N |
| ONC176222 | tumor_biopsy_5% | HRD+ | 28.41 | 0.84295488 | 41 | N |
| ONC178033 | tumor_biopsy_5% | HRD− | 0.47 | 0.68604651 | 1 | N |
| ONC179846 | tumor_biopsy_5% | HRD− | 0 | 0.02378883 | 4 | N |
| ONC181838 | tumor_biopsy_5% | HRD+ | 2.19 | 0.46144767 | 11 | N |
| ONC184454 | tumor_biopsy_5% | HRD+ | 17 | 0.9998741 | 17 | N |
| ONC192824 | tumor_biopsy_5% | HRD+ | 89.49 | 0.91553816 | 110 | N |
| ONC195700 | tumor_biopsy_5% | HRD+ | 21.82 | 0.7447001 | 40 | N |
| ONC33279 | tumor_biopsy_5% | HRD− | 0.22 | 0.34894512 | 2 | N |
| ONC138111 | tumor_biopsy_2% | HRD− | 0 | 0.00543409 | 1 | N |
| ONC143854 | tumor_biopsy_2% | HRD− | 1.00E−05 | 0.5 | 0 | N |
| ONC147579 | tumor_biopsy_2% | HRD+ | 1.46 | 0.61493759 | 4 | N |
| ONC152713 | tumor_biopsy_2% | HRD− | 0.1 | 0.31666141 | 1 | N |
| ONC156134 | tumor_biopsy_2% | HRD− | 0.04 | 0.15924082 | 2 | N |
| ONC156389 | tumor_biopsy_2% | HRD− | 1.00E−05 | 0.5 | 0 | N |
| ONC157864 | tumor_biopsy_2% | HRD− | 0.47 | 0.68604651 | 1 | N |
| ONC159155 | tumor_biopsy_2% | HRD+ | 3.19 | 0.79919362 | 5 | N |
| ONC159391 | tumor_biopsy_2% | HRD− | 0.56 | 0.53065378 | 2 | N |
| ONC161752 | tumor_biopsy_2% | HRD− | 0.17 | 0.20862406 | 4 | N |
| ONC166813 | tumor_biopsy_2% | HRD− | 1.00E−05 | 0.5 | 0 | N |
| ONC167203 | tumor_biopsy_2% | HRD− | 1.00E−05 | 0.5 | 0 | N |
| ONC170292 | tumor_biopsy_2% | HRD− | 1.00E−05 | 0.5 | 0 | N |
| ONC170658 | tumor_biopsy_2% | HRD− | 0.73 | 0.50231783 | 3 | N |
| ONC171635 | tumor_biopsy_2% | HRD− | 1.00E−05 | 0.5 | 0 | N |
| ONC172301 | tumor_biopsy_2% | HRD− | 0.1 | 0.31666141 | 1 | N |
| ONC173793 | tumor_biopsy_2% | HRD− | 0.47 | 0.68604651 | 1 | N |
| ONC174333 | tumor_biopsy_2% | HRD− | 0 | 0.05104924 | 2 | N |
| ONC175142 | tumor_biopsy_2% | HRD+ | 1.46 | 0.61351826 | 4 | N |
| ONC176222 | tumor_biopsy_2% | HRD+ | 3.26 | 0.90276622 | 4 | N |
| ONC178033 | tumor_biopsy_2% | HRD− | 1.00E−05 | 0.5 | 0 | N |
| ONC179846 | tumor_biopsy_2% | HRD− | 0.1 | 0.31666141 | 1 | N |
| ONC181838 | tumor_biopsy_2% | HRD− | 0.1 | 0.16147547 | 4 | N |
| ONC184454 | tumor_biopsy_2% | HRD− | 0.66 | 0.46933091 | 3 | N |
| ONC192824 | tumor_biopsy_2% | HRD+ | 11.44 | 0.97622807 | 12 | N |
| ONC195700 | tumor_biopsy_2% | HRD− | 0.65 | 0.40421293 | 4 | N |
| ONC33279 | tumor_biopsy_2% | HRD− | 1.00E−05 | 0.5 | 0 | N |
| 05246_185 | metastatic_cfdna | HRD− | 1.00E−05 | NA | 0 | N |
| 05246_252 | metastatic_cfdna | HRD+ | 460.72 | 0.89040312 | 599 | N |
| 05246_292 | metastatic_cfdna | HRD+ | 57.73 | 0.69826932 | 120 | N |
| 05246_300 | metastatic_cfdna | HRD− | 1.00E−05 | NA | 0 | N |
| 05246_303 | metastatic_cfdna | HRD+ | 286.84 | 0.90601604 | 359 | N |
| 05246_306 | metastatic_cfdna | HRD− | 0.24 | 0.35589665 | 2 | N |
| 05246_313 | metastatic_cfdna | HRD− | 0.52 | 0.23156484 | 10 | N |
| 05246_315 | metastatic_cfdna | HRD− | 0.57 | 0.18694838 | 17 | N |
| 05246_318 | metastatic_cfdna | HRD+ | 187.84 | 0.89287728 | 243 | N |
| 05246_321 | metastatic_cfdna | HRD+ | 268.66 | 0.83818553 | 390 | N |
| 05246_331 | metastatic_cfdna | HRD+ | 2.73 | 0.43474514 | 15 | N |
| 05246_343 | metastatic_cfdna | HRD− | 0 | 9.92E−07 | 992 | N |
| 05246_350 | metastatic_cfdna | HRD− | 1.00E−05 | NA | 0 | N |
| 05246_357 | metastatic_cfdna | HRD− | 0.32 | 0.39852377 | 2 | N |
| 05246_359 | metastatic_cfdna | HRD+ | 126.92 | 0.86913074 | 172 | N |
| 05246_371 | metastatic_cfdna | HRD+ | 2.93 | 0.17093273 | 106 | Y |
| 05246_376 | metastatic_cfdna | HRD− | 0.64 | 0.80171158 | 1 | N |
| 05246_465 | metastatic_cfdna | HRD+ | 204.37 | 0.80154639 | 327 | N |
| 05246_473 | metastatic_cfdna | HRD+ | 1.11 | 0.27666537 | 15 | N |
| 05246_8 | metastatic_cfdna | HRD− | 0.02 | 0.03446613 | 20 | N |
| ONC138111_W00 | early-stage_cfdna | HRD− | 0 | 0.00304157 | 10 | N |
| ONC138111_W03 | early-stage_cfdna | HRD− | 0 | 0.00284395 | 12 | N |
| ONC138111_W12 | early-stage_cfdna | HRD− | 0 | 0.0480379 | 3 | N |
| ONC143854_W00 | early-stage_cfdna | HRD− | 0 | 0.00543409 | 1 | N |
| ONC147579_W00 | early-stage_cfdna | HRD+ | 8.14 | 0.52722839 | 30 | N |
| ONC152713_W00 | early-stage_cfdna | HRD− | 0.23 | 0.34334886 | 2 | N |
| ONC152713_W03 | early-stage_cfdna | HRD− | 0 | 0.00543409 | 1 | N |
| ONC156389_W03 | early-stage_cfdna | HRD− | 0 | 0.01409571 | 2 | N |
| ONC157864_W00 | early-stage_cfdna | HRD− | 0.64 | 0.80171158 | 1 | N |
| ONC159155_W00 | early-stage_cfdna | HRD+ | 102.81 | 0.84743162 | 146 | N |
| ONC159155_W12 | early-stage_cfdna | HRD+ | 12.94 | 0.68477739 | 28 | N |
| ONC159391_W00 | early-stage_cfdna | HRD− | 1.00E−05 | NA | 0 | N |
| ONC161752_W00 | early-stage_cfdna | HRD+ | 4.22 | 0.56967113 | 13 | N |
| ONC166813_W00 | early-stage_cfdna | HRD− | 0 | 0.00543409 | 1 | N |
| ONC166941_W00 | early-stage_cfdna | HRD− | 0.24 | 0.35589665 | 2 | N |
| ONC166941_W12 | early-stage_cfdna | HRD− | 0.48 | 0.35042735 | 4 | N |
| ONC170292_W00 | early-stage_cfdna | HRD− | 0.47 | 0.68604651 | 1 | N |
| ONC170658_W00 | early-stage_cfdna | HRD+ | 1.02 | 0.27316884 | 14 | N |
| ONC172301_W00 | early-stage_cfdna | HRD+ | 41.84 | 0.65270138 | 99 | N |
| ONC173793_W00 | early-stage_cfdna | HRD+ | 5.86 | 0.98791359 | 6 | N |
| ONC174333_W00 | early-stage_cfdna | HRD− | 0.01 | 0.04503513 | 6 | N |
| ONC175142_W00 | early-stage_cfdna | HRD+ | 104.39 | 0.75415759 | 187 | N |
| ONC176222_W00 | early-stage_cfdna | HRD+ | 2.05 | 0.37085886 | 15 | N |
| ONC178033_W00 | early-stage_cfdna | HRD− | 0 | 0.0649387 | 2 | N |
| ONC178033_W12 | early-stage_cfdna | HRD− | 0 | 0.0649387 | 2 | N |
| ONC179846_W00 | early-stage_cfdna | HRD− | 0 | 7.29E−05 | 13 | N |
| ONC181838_W12 | early-stage_cfdna | HRD− | 0.1 | 0.18510401 | 3 | N |
| ONC182745_W00 | early-stage_cfdna | HRD− | 0.26 | 0.23042704 | 5 | N |
| ONC184454_W00 | early-stage_cfdna | HRD+ | 2.73 | 0.36323716 | 21 | N |
| ONC192824_W00 | early-stage_cfdna | HRD+ | 153.38 | 0.82410846 | 229 | N |
| ONC193018_W00 | early-stage_cfdna | HRD− | 0.55 | 0.52493016 | 2 | N |
| ONC194658_W00 | early-stage_cfdna | HRD+ | 2.98 | 0.41175207 | 18 | N |
| ONC195079_W00 | early-stage_cfdna | HRD− | 0 | 0.06313496 | 1 | N |
| ONC195262_W00 | early-stage_cfdna | HRD+ | 9.5 | 0.45801693 | 47 | N |
| ONC195262_W03 | early-stage_cfdna | HRD+ | 1 | 0.26284767 | 15 | N |
| ONC195262_W12 | early-stage_cfdna | HRD+ | 8.38 | 0.81213884 | 13 | N |
| ONC33279_W00 | early-stage_cfdna | HRD− | 0.18 | 0.18043881 | 6 | N |
| 108200_posttx138d | prostate | HRD− | 0 | 0.02643505 | 4 | N |
| 108200_pretx530d | prostate | HRD− | 0.06 | 0.15508498 | 4 | N |
| 128311_posttx127d | prostate | HRD+ | 2.67 | 0.45931247 | 14 | N |
| 128311_pretx1333d | prostate | HRD+ | 3.92 | 0.78212854 | 6 | N |
| 384102_pretx78d | prostate | HRD+ | 2.11 | 0.39619588 | 11 | N |
| 476703_pretx57d | prostate | HRD− | 0.02 | 0.02262107 | 33 | N |
| 718458_pretx0d | prostate | HRD− | 0.72 | 0.33478048 | 9 | N |
| 566691_posttx26d | prostate | HRD− | 0.4 | 0.14232529 | 22 | N |
| 566691_pretx0d | prostate | HRD+ | 2.01 | 0.37340144 | 17 | N |
| 653096_pretx0d | prostate | HRD+ | 74.3 | 0.54590262 | 244 | N |
| 653096_posttx31d | prostate | HRD+ | 344.73 | 0.67216444 | 745 | N |
| 646059_pretx6d | prostate | HRD+ | 144.42 | 0.71366537 | 291 | N |
| 500560_pretx11d | prostate | HRD− | 0 | 0.02128651 | 7 | N |
| 407120_pretx683d | prostate | HRD+ | 2.53 | 0.76982168 | 5 | N |
| 407120_posttx489d | prostate | HRD+ | 5.72 | 0.54009574 | 18 | N |
| 566240_pretx437d | prostate | HRD− | 0.85 | 0.77810979 | 2 | N |
| 752770_pretx0d | prostate | HRD+ | 241.88 | 0.75390777 | 418 | N |
| GENP2455-2 | prostate | HRD+ | 80.24 | 0.85022692 | 127 | N |
| GENP2560-2 | prostate | HRD+ | 94.72 | 0.74238646 | 174 | N |
| GENP3534-1 | prostate | HRD+ | 3.24 | 0.36278924 | 24 | N |
| GENP4005-1 | prostate | HRD− | 0 | 0.04393946 | 0 | N |
| RGENP1401_P2 | prostate | HRD− | 0.03 | 0.03706524 | 26 | N |
| RGENP462_2 | prostate | HRD+ | 239.93 | 0.78516014 | 410 | N |
| RGENP490_1 | prostate | HRD+ | 347.23 | 0.73522203 | 641 | N |
| RGENP544_1 | prostate | HRD− | 0 | 2.62E−04 | 23 | N |
| OC_NACT_A | ovary | HRD+ | 3.18 | 0.66441508 | 6 | N |
| OC_NACT_B | ovary | HRD+ | 19.65 | 0.46959388 | 69 | N |
| OC_NACT_D | ovary | HRD+ | 2.92 | 0.5269625 | 11 | N |
| OC_NACT_F | ovary | HRD− | 0.16 | 0.1970491 | 5 | N |
| OC_NACT_M | ovary | HRD+ | 65.26 | 0.73600722 | 117 | N |
| OC_NACT_S | ovary | HRD+ | 72.64 | 0.5367661 | 212 | N |
| OC_NACT_U | ovary | HRD+ | 8.25 | 0.54311764 | 28 | N |
| OC_NACT_V | ovary | HRD− | 0.45 | 0.1667422 | 19 | N |
| OC_NACT_W | ovary | HRD+ | 4.5 | 0.75981943 | 7 | N |
| OC_NACT_X | ovary | HRD+ | 5.59 | 0.44083286 | 18 | N |

TABLE 6

| | | | | | | | remarks |
|---|---|---|---|---|---|---|---|
| sample | dataset | p_BRCA1 | p_BRCA2 | p_hrd | hr status | hrd type | hr status |
| ONC138111 | TBFP | 0 | 0 | 0 | HRP | none | |
| ONC143854 | TBFP | 0 | 0 | 0 | HRP | none | |
| ONC147579 | TBFP | 0.214 | 0.522 | 0.736 | HRP | BRCA2_type | |
| ONC152713 | TBFP | 0 | 0 | 0 | HRP | none | |
| ONC156134 | TBFP | 0.106 | 0.564 | 0.67 | HRP | BRCA2_type | |
| ONC156389 | TBFP | 0 | 0 | 0 | HRP | none | |
| ONC157864 | TBFP | 0 | 0.004 | 0.004 | HRP | none | |
| ONC159155 | TBFP | 0.13 | 0.498 | 0.628 | HRP | BRCA2_type | |
| ONC159391 | TBFP | 0 | 0 | 0 | HRP | none | |
| ONC161752 | TBFP | 0.172 | 0.464 | 0.636 | HRP | BRCA2_type | |
| ONC166813 | TBFP | 0 | 0 | 0 | HRP | none | |
| ONC167203 | TBFP | 0.168 | 0.468 | 0.636 | HRP | BRCA2_type | |
| ONC170292 | TBFP | 0.004 | 0.01 | 0.014 | HRP | none | |
| ONC170658 | TBFP | 0.208 | 0.544 | 0.752 | HRP | BRCA2_type | |
| ONC171635 | TBFP | 0 | 0 | 0 | HRP | none | |
| ONC172301 | TBFP | 0 | 0 | 0 | HRP | none | |
| ONC173793 | TBFP | 0.016 | 0.014 | 0.03 | HRP | none | |
| ONC174333 | TBFP | 0.032 | 0.018 | 0.05 | HRP | none | |
| ONC175142 | TBFP | 0.272 | 0.494 | 0.766 | HRP | BRCA2_type | |
| ONC176222 | TBFP | 0 | 0 | 0 | HRP | none | |
| ONC178033 | TBFP | 0 | 0.002 | 0.002 | HRP | none | |
| ONC179846 | TBFP | 0 | 0 | 0 | HRP | none | |
| ONC181838 | TBFP | 0.014 | 0.014 | 0.028 | HRP | none | |
| ONC184454 | TBFP | 0.118 | 0.578 | 0.696 | HRDef | BRCA2_type | |
| ONC192824 | TBFP | 0.162 | 0.55 | 0.712 | HRDef | BRCA2_type | |
| ONC195700 | TBFP | 0.236 | 0.49 | 0.726 | HRDef | BRCA2_type | |
| ONC33279 | TBFP | 0 | 0 | 0 | HRP | none | |
| ONC138111 | TB5 | 0 | 0 | 0 | CBD | none | <1 indels |
| ONC143854 | TB5 | 0 | 0.002 | 0.002 | CBD | none | <1 indels |
| ONC147579 | TB5 | 0.086 | 0.594 | 0.68 | HRDef | BRCA2_type | |
| ONC152713 | TB5 | 0 | 0 | 0 | CBD | none | <1 indels |
| ONC156134 | TB5 | 0 | 0 | 0 | CBD | none | <1 indels |
| ONC156389 | TB5 | 0 | 0 | 0 | HRP | none | |
| ONC157864 | TB5 | 0.016 | 0.012 | 0.028 | CBD | none | <1 indels |
| ONC159155 | TB5 | 0 | 0 | 0 | HRP | none | |
| ONC159391 | TB5 | 0.098 | 0.578 | 0.676 | HRDef | BRCA2_type | |
| ONC161752 | TB5 | 0.126 | 0.554 | 0.68 | HRDef | BRCA2_type | |
| ONC166813 | TB5 | 0 | 0 | 0 | CBD | none | <1 indels |
| ONC167203 | TB5 | 0 | 0 | 0 | CBD | none | <1 indels |
| ONC170292 | TB5 | 0 | 0 | 0 | CBD | none | <1 indels |
| ONC170658 | TB5 | 0 | 0 | 0 | CBD | none | <1 indels |
| ONC171635 | TB5 | 0 | 0 | 0 | CBD | none | <1 indels |
| ONC172301 | TB5 | 0 | 0 | 0 | CBD | none | <1 indels |
| ONC173793 | TB5 | 0.01 | 0.012 | 0.022 | CBD | none | <1 indels |
| ONC174333 | TB5 | 0 | 0 | 0 | CBD | none | <1 indels |
| ONC175142 | TB5 | 0 | 0 | 0 | CBD | none | <1 indels |
| ONC176222 | TB5 | 0 | 0 | 0 | HRP | none | |
| ONC178033 | TB5 | 0 | 0 | 0 | CBD | none | <1 indels |
| ONC179846 | TB5 | 0 | 0.002 | 0.002 | CBD | none | <1 indels |
| ONC181838 | TB5 | 0 | 0.012 | 0.012 | CBD | none | <1 indels |
| ONC184454 | TB5 | 0.006 | 0 | 0.006 | CBD | none | <1 indels |
| ONC192824 | TB5 | 0.004 | 0.02 | 0.024 | CBD | none | <1 indels |
| ONC195700 | TB5 | 0 | 0.002 | 0.002 | CBD | none | <1 indels |
| ONC33279 | TB5 | 0 | 0 | 0 | CBD | none | <1 indels |
| ONC138111 | TB2 | 0.006 | 0 | 0.006 | CBD | none | <1 indels |
| ONC143854 | TB2 | 0 | 0 | 0 | CBD | none | <1 indels |
| ONC147579 | TB2 | 0.008 | 0 | 0.008 | CBD | none | <1 indels |
| ONC152713 | TB2 | 0 | 0 | 0 | CBD | none | <1 indels |
| ONC156134 | TB2 | 0 | 0 | 0 | CBD | none | <1 indels |
| ONC156389 | TB2 | 0 | 0 | 0 | CBD | none | <1 indels |
| ONC157864 | TB2 | 0 | 0.002 | 0.002 | CBD | none | <1 indels |
| ONC159155 | TB2 | 0 | 0 | 0 | CBD | none | <1 indels |
| ONC159391 | TB2 | 0.098 | 0.558 | 0.656 | HRDef | BRCA2_type | |
| ONC161752 | TB2 | 0.078 | 0.576 | 0.654 | HRDef | BRCA2_type | |
| ONC166813 | TB2 | 0 | 0 | 0 | CBD | none | <1 indels |
| ONC167203 | TB2 | 0.018 | 0.022 | 0.04 | CBD | none | <1 indels |
| ONC170292 | TB2 | 0 | 0 | 0 | CBD | none | <1 indels |
| ONC170658 | TB2 | 0 | 0.002 | 0.002 | CBD | none | <1 indels |
| ONC171635 | TB2 | 0 | 0 | 0 | CBD | none | <1 indels |
| ONC172301 | TB2 | 0 | 0 | 0 | CBD | none | <1 indels |
| ONC173793 | TB2 | 0 | 0 | 0 | CBD | none | <1 indels |
| ONC174333 | TB2 | 0 | 0 | 0 | CBD | none | <1 indels |
| ONC175142 | TB2 | 0.002 | 0.016 | 0.018 | CBD | none | <1 indels |
| ONC176222 | TB2 | 0 | 0 | 0 | HRP | none | |
| ONC178033 | TB2 | 0 | 0 | 0 | CBD | none | <1 indels |
| ONC179846 | TB2 | 0 | 0 | 0 | CBD | none | <1 indels |
| ONC181838 | TB2 | 0 | 0 | 0 | CBD | none | <1 indels |
| ONC184454 | TB2 | 0.008 | 0 | 0.008 | CBD | none | <1 indels |
| ONC192824 | TB2 | 0.008 | 0 | 0.008 | CBD | none | <1 indels |
| ONC195700 | TB2 | 0 | 0 | 0 | CBD | none | <1 indels |
| ONC33279 | TB2 | 0 | 0 | 0 | CBD | none | <1 indels |
| 05246_185 | S4 | 0 | 0 | 0 | HRP | none | |
| 05246_252 | S4 | 0 | 0.004 | 0.004 | HRP | none | |
| 05246_292 | S4 | 0.002 | 0.01 | 0.012 | HRP | none | |
| 05246_300 | S4 | 0 | 0 | 0 | HRP | none | |
| 05246_303 | S4 | 0.224 | 0.44 | 0.664 | HRP | BRCA2_type | |
| 05246_306 | S4 | 0 | 0 | 0 | HRP | none | |
| 05246_313 | S4 | 0 | 0 | 0 | HRP | none | |
| 05246_315 | S4 | 0 | 0 | 0 | HRP | none | |
| 05246_318 | S4 | 0.01 | 0.016 | 0.026 | HRP | none | |
| 05246_321 | S4 | 0 | 0.004 | 0.004 | HRP | none | |
| 05246_331 | S4 | 0 | 0.002 | 0.002 | HRP | none | |
| 05246_343 | S4 | 0 | 0 | 0 | CBD | none | Has MSI |
| | | | | | | | (>14000 |
| | | | | | | | indel. rep) |
| 05246_350 | S4 | 0 | 0 | 0 | HRP | none | |
| 05246_357 | S4 | 0 | 0 | 0 | HRP | none | |
| 05246_359 | S4 | 0.142 | 0.516 | 0.658 | HRDef | BRCA2_type | |
| 05246_371 | S4 | 0 | 0 | 0 | CBD | none | |
| 05246_376 | S4 | 0 | 0 | 0 | CBD | none | |
| 05246_465 | S4 | 0 | 0 | 0 | CBD | none | |
| 05246_473 | S4 | 0 | 0 | 0 | CBD | none | |
| 05246_8 | S4 | 0 | 0 | 0 | CBD | none | |
| ONC138111_W00 | ES | 0 | 0 | 0 | CBD | none | |
| ONC138111_W03 | ES | 0 | 0 | 0 | CBD | none | |
| ONC138111_W12 | ES | 0 | 0 | 0 | CBD | none | |
| ONC143854_W00 | ES | 0 | 0 | 0 | CBD | none | |
| ONC147579_W00 | ES | 0 | 0 | 0 | CBD | none | |
| ONC152713_W00 | ES | 0 | 0 | 0 | CBD | none | |
| ONC152713_W03 | ES | 0 | 0 | 0 | CBD | none | |
| ONC156389_W03 | ES | 0 | 0 | 0 | CBD | none | |
| ONC157864_W00 | ES | 0 | 0 | 0 | CBD | none | |
| ONC159155_W00 | ES | 0 | 0 | 0 | CBD | none | |
| ONC159155_W12 | ES | 0 | 0 | 0 | CBD | none | |
| ONC159391_W00 | ES | 0 | 0 | 0 | CBD | none | |
| ONC161752_W00 | ES | 0 | 0 | 0 | CBD | none | |
| ONC166813_W00 | ES | 0 | 0 | 0 | CBD | none | |
| ONC166941_W00 | ES | 0 | 0 | 0 | CBD | none | |
| ONC166941_W12 | ES | 0 | 0 | 0 | CBD | none | |
| ONC170292_W00 | ES | 0 | 0 | 0 | CBD | none | |
| ONC170658_W00 | ES | 0 | 0 | 0 | CBD | none | |
| ONC172301_W00 | ES | 0 | 0 | 0 | CBD | none | |
| ONC173793_W00 | ES | 0 | 0 | 0 | CBD | none | |
| ONC174333_W00 | ES | 0 | 0 | 0 | CBD | none | |
| ONC175142_W00 | ES | 0.27 | 0.412 | 0.682 | HRDef | BRCA2_type | |
| ONC176222_W00 | ES | 0 | 0 | 0 | CBD | none | |
| ONC178033_W00 | ES | 0 | 0 | 0 | CBD | none | |
| ONC178033_W12 | ES | 0 | 0 | 0 | CBD | none | |
| ONC179846_W00 | ES | 0 | 0 | 0 | CBD | none | |
| ONC181838_W12 | ES | 0.018 | 0.004 | 0.022 | CBD | none | |
| ONC182745_W00 | ES | 0 | 0 | 0 | CBD | none | |
| ONC184454_W00 | ES | 0 | 0 | 0 | CBD | none | |
| ONC192824_W00 | ES | 0 | 0 | 0 | CBD | none | |
| ONC193018_W00 | ES | 0 | 0 | 0 | CBD | none | |
| ONC194658_W00 | ES | 0 | 0 | 0 | CBD | none | |
| ONC195079_W00 | ES | 0 | 0 | 0 | CBD | none | |
| ONC195262_W00 | ES | 0 | 0 | 0 | CBD | none | |
| ONC195262_W03 | ES | 0.002 | 0.008 | 0.01 | CBD | none | |
| ONC195262_W12 | ES | 0 | 0 | 0 | CBD | none | |
| ONC33279_W00 | ES | 0 | 0 | 0 | HRP | none | |

TBFP = “tumor_biopsy_fullpurity”;
TB5 = “tumor_biopsy_5%”;
TB2 = “tumor_biopsy_2%”;
S4 = “stage IV”;
ES = “early-stage”;
HRP = “HR_proficient”;
HRDef = “HR_deficient”;
CBD = “cannot be determined”

| sample | pB1.5 | pB1.50 | pB1.95 | pB2.5 | p_B2.50 | pB2.95 | phrd5 | phrd5 0 | phrd95 |
|---|---|---|---|---|---|---|---|---|---|
| ONC138111 | 0 | 0 | 0 | 0 | 0 | 1.00E−04 | 0 | 0 | 1.00E−04 |
| ONC143854 | 0 | 0 | 1.00E−04 | 0 | 0 | 9.00E−04 | 0 | 0 | 0.001 |
| ONC147579 | 0.1417 | 0.199 | 0.2608 | 0.4574 | 0.523 | 0.6205 | 0.6998 | 0.726 | 0.7622 |
| ONC152713 | 0 | 0 | 1.00E−04 | 0 | 0 | 1.00E−04 | 0 | 0 | 0.002 |
| ONC156134 | 0 | 0.106 | 0.1061 | 0.002 | 0.566 | 0.5664 | 0.002 | 0.672 | 0.672 |
| ONC156389 | 0 | 0 | 0.0165 | 0 | 0 | 0.0325 | 0 | 0 | 0.0471 |
| ONC157864 | 0 | 0 | 0.0328 | 0 | 0.002 | 0.0297 | 0 | 0.002 | 0.0625 |
| ONC159155 | 0 | 0.091 | 0.14 | 0.002 | 0.502 | 0.5942 | 0.002 | 0.633 | 0.6769 |
| ONC159391 | 0 | 0 | 0.0046 | 0 | 0 | 0.0067 | 0 | 0 | 0.0094 |
| ONC161752 | 0 | 0.13 | 0.1646 | 0 | 0.5 | 0.5233 | 0 | 0.638 | 0.6632 |
| ONC166813 | 0 | 0 | 0 | 0 | 0 | 0.002 | 0 | 0 | 0.002 |
| ONC167203 | 0.0114 | 0.106 | 0.1703 | 0.0172 | 0.469 | 0.5684 | 0.0286 | 0.642 | 0.6781 |
| ONC170292 | 0 | 0.008 | 0.1927 | 0 | 0.009 | 0.485 | 0 | 0.022 | 0.6643 |
| ONC170658 | 0.018 | 0.079 | 0.2521 | 0.0099 | 0.311 | 0.6183 | 0.028 | 0.475 | 0.7746 |
| ONC171635 | 0 | 0 | 0 | 0 | 0 | 0.002 | 0 | 0 | 0.002 |
| ONC172301 | 0 | 0 | 0 | 0 | 0 | 0.0021 | 0 | 0 | 0.0021 |
| ONC173793 | 0 | 0.016 | 0.1605 | 0 | 0.018 | 0.4635 | 0 | 0.034 | 0.6225 |
| ONC174333 | 0 | 0.03 | 0.2301 | 0.002 | 0.016 | 0.5025 | 0.002 | 0.045 | 0.7005 |
| ONC175142 | 0.208 | 0.258 | 0.2841 | 0.417 | 0.512 | 0.591 | 0.6878 | 0.767 | 0.7985 |
| ONC176222 | 0 | 0.003 | 0.0321 | 0 | 0.013 | 0.0917 | 0 | 0.017 | 0.1238 |
| ONC178033 | 0 | 0 | 0 | 0 | 0.002 | 0.002 | 0 | 0.002 | 0.002 |
| ONC179846 | 0 | 0 | 0 | 0 | 0 | 0 | 0 | 0 | 0 |
| ONC181838 | 0 | 0.019 | 0.2573 | 0 | 0.012 | 0.4844 | 0 | 0.029 | 0.689 |
| ONC184454 | 0.0735 | 0.117 | 0.1305 | 0.536 | 0.572 | 0.6264 | 0.6755 | 0.689 | 0.6981 |
| ONC192824 | 0.1118 | 0.185 | 0.2705 | 0.444 | 0.524 | 0.5765 | 0.6618 | 0.703 | 0.756 |
| ONC195700 | 0.1021 | 0.224 | 0.2743 | 0.0643 | 0.485 | 0.5269 | 0.1664 | 0.716 | 0.744 |
| ONC33279 | 0 | 0 | 0 | 0 | 0 | 0 | 0 | 0 | 0 |
| ONC138111 | 0 | 0 | 0.0141 | 0 | 0.004 | 0.0181 | 0 | 0.004 | 0.0282 |
| ONC143854 | 0 | 0 | 0 | 0 | 0 | 0.002 | 0 | 0 | 0.002 |
| ONC147579 | 0.066 | 0.083 | 0.0984 | 0.5758 | 0.61 | 0.6264 | 0.6696 | 0.69 | 0.7025 |
| ONC152713 | 0 | 0 | 0 | 0 | 0 | 0.002 | 0 | 0 | 0.002 |
| ONC156134 | 0 | 0 | 0 | 0 | 0 | 0.0042 | 0 | 0 | 0.0042 |
| ONC156389 | 0 | 0 | 0 | 0 | 0 | 0 | 0 | 0 | 0 |
| ONC157864 | 0 | 0.01 | 0.02 | 0 | 0.012 | 0.0181 | 0 | 0.022 | 0.038 |
| ONC159155 | 0 | 0 | 0 | 0 | 0 | 0.004 | 0 | 0 | 0.004 |
| ONC159391 | 0.0759 | 0.095 | 0.1082 | 0.5657 | 0.58 | 0.6026 | 0.6656 | 0.674 | 0.69 |
| ONC161752 | 0 | 0.106 | 0.1422 | 0 | 0.548 | 0.5841 | 0 | 0.67 | 0.6861 |
| ONC166813 | 0 | 0 | 0.0061 | 0 | 0 | 0 | 0 | 0 | 0.0061 |
| ONC167203 | 0 | 0 | 0.0121 | 0 | 0.002 | 0.0161 | 0 | 0.002 | 0.0242 |
| ONC170292 | 0 | 0 | 0.0046 | 0 | 0.001 | 0.018 | 0 | 0.001 | 0.0226 |
| ONC170658 | 0 | 0 | 0.0083 | 0 | 0 | 0.018 | 0 | 0 | 0.0206 |
| ONC171635 | 0 | 0 | 0 | 0 | 0 | 1.00E−04 | 0 | 0 | 1.00E−04 |
| ONC172301 | 0 | 0 | 0 | 0 | 0 | 0 | 0 | 0 | 0 |
| ONC173793 | 0 | 0.008 | 0.016 | 0 | 0 | 0.018 | 0 | 0.008 | 0.034 |
| ONC174333 | 0 | 0 | 0 | 0 | 0 | 0.002 | 0 | 0 | 0.002 |
| ONC175142 | 0 | 0 | 0 | 0 | 0 | 1.00E−04 | 0 | 0 | 1.00E−04 |
| ONC176222 | 0 | 0 | 0 | 0 | 0.001 | 0.0081 | 0 | 0.001 | 0.0081 |
| ONC178033 | 0 | 0 | 0 | 0 | 0 | 1.00E−04 | 0 | 0 | 1.00E−04 |
| ONC179846 | 0 | 0 | 0 | 0 | 0 | 0.0021 | 0 | 0 | 0.0021 |
| ONC181838 | 0 | 0 | 0.0065 | 0 | 0.002 | 0.018 | 0 | 0.002 | 0.0245 |
| ONC184454 | 0 | 0.003 | 0.0061 | 0 | 0 | 0 | 0 | 0.003 | 0.0061 |
| ONC192824 | 0 | 0.002 | 0.0061 | 0.0058 | 0.014 | 0.0201 | 0.0058 | 0.015 | 0.0241 |
| ONC195700 | 0 | 0 | 0 | 0 | 0.002 | 0.004 | 0 | 0.002 | 0.004 |
| ONC33279 | 0 | 0 | 0 | 0 | 0 | 0.0021 | 0 | 0 | 0.0021 |
| ONC138111 | 0 | 0.006 | 0.008 | 0 | 0 | 0 | 0 | 0.006 | 0.008 |
| ONC143854 | 0 | 0 | 0 | 0 | 0 | 0 | 0 | 0 | 0 |
| ONC147579 | 0 | 0.006 | 0.008 | 0 | 0 | 5.00E−04 | 0 | 0.006 | 0.0081 |
| ONC152713 | 0 | 0 | 0.006 | 0 | 0 | 0.0162 | 0 | 0 | 0.0184 |
| ONC156134 | 0 | 0 | 2.00E−04 | 0 | 0 | 0.0027 | 0 | 0 | 0.0029 |
| ONC156389 | 0 | 0 | 0.014 | 0 | 0 | 0.0161 | 0 | 0.002 | 0.03 |
| ONC157864 | 0 | 0 | 0 | 0 | 0 | 0.0021 | 0 | 0 | 0.0021 |
| ONC159155 | 0 | 0 | 3.00E−04 | 0 | 0 | 0.002 | 0 | 0 | 0.0022 |
| ONC159391 | 0.0659 | 0.098 | 0.1082 | 0.544 | 0.565 | 0.587 | 0.6498 | 0.656 | 0.6728 |
| ONC161752 | 0.0735 | 0.078 | 0.108 | 0.544 | 0.576 | 0.58 | 0.6419 | 0.654 | 0.656 |
| ONC166813 | 0 | 0 | 0.006 | 0 | 0 | 0.0044 | 0 | 0 | 0.0063 |
| ONC167203 | 0 | 0.004 | 0.018 | 0 | 0.002 | 0.022 | 0 | 0.008 | 0.0381 |
| ONC170292 | 0 | 0 | 0.006 | 0 | 0 | 0 | 0 | 0 | 0.006 |
| ONC170658 | 0 | 0 | 0 | 0.0019 | 0.002 | 0.002 | 0.0019 | 0.002 | 0.002 |
| ONC171635 | 0 | 0 | 0 | 0 | 0 | 0.002 | 0 | 0 | 0.002 |
| ONC172301 | 0 | 0 | 0.008 | 0 | 0 | 0.0241 | 0 | 0 | 0.0261 |
| ONC173793 | 0 | 0 | 3.00E−04 | 0 | 0 | 0 | 0 | 0 | 3.00E−04 |
| ONC174333 | 0 | 0 | 0 | 0 | 0 | 0.002 | 0 | 0 | 0.002 |
| ONC175142 | 0 | 0 | 0.018 | 0 | 0 | 0.022 | 0 | 0.004 | 0.04 |
| ONC176222 | 0 | 0 | 0 | 0 | 0 | 0.0041 | 0 | 0 | 0.0041 |
| ONC178033 | 0 | 0 | 6.00E−04 | 0 | 0 | 0 | 0 | 0 | 6.00E−04 |
| ONC179846 | 0 | 0 | 3.00E−04 | 0 | 0 | 0 | 0 | 0 | 3.00E−04 |
| ONC181838 | 0 | 0 | 0 | 0 | 0 | 0 | 0 | 0 | 0 |
| ONC184454 | 0 | 0.007 | 0.008 | 0 | 0 | 0 | 0 | 0.007 | 0.008 |
| ONC192824 | 0 | 0.001 | 0.008 | 0 | 0 | 6.00E−04 | 0 | 0.003 | 0.0083 |
| ONC195700 | 0 | 0 | 0.006 | 0 | 0 | 0.0101 | 0 | 0 | 0.0122 |
| ONC33279 | 0 | 0 | 0.006 | 0 | 0 | 0 | 0 | 0 | 0.006 |
| 05246_185 | 0 | 0 | 0 | 0 | 0 | 0 | 0 | 0 | 0 |
| 05246_252 | 0 | 0.006 | 0.1439 | 0 | 0.008 | 0.4571 | 0 | 0.014 | 0.6208 |
| 05246_292 | 0 | 0.002 | 0.01 | 0 | 0.005 | 0.014 | 0 | 0.01 | 0.0221 |
| 05246_300 | 0 | 0 | 0 | 0 | 0 | 0 | 0 | 0 | 0 |
| 05246_303 | 0.0417 | 0.178 | 0.2485 | 0.0627 | 0.43 | 0.5431 | 0.1044 | 0.665 | 0.7106 |
| 05246_306 | 0 | 0 | 0.0416 | 0 | 0 | 0.0933 | 0 | 0 | 0.1349 |
| 05246_313 | 0 | 0 | 0 | 0 | 0 | 0 | 0 | 0 | 0 |
| 05246_315 | 0 | 0 | 0.1101 | 0 | 0 | 0.177 | 0 | 0 | 0.2861 |
| 05246_318 | 0 | 0.015 | 0.1403 | 0 | 0.027 | 0.4926 | 0 | 0.041 | 0.6383 |
| 05246_321 | 0 | 0 | 0.0537 | 0 | 0 | 0.1105 | 0 | 0 | 0.1642 |
| 05246_331 | 0 | 0 | 1.00E−04 | 0.002 | 0.002 | 0.0022 | 0.002 | 0.002 | 0.0023 |
| 05246_343 | 0 | 0 | 0 | 0 | 0 | 0 | 0 | 0 | 0 |
| 05246_350 | 0 | 0 | 0.176 | 0 | 0 | 0.4406 | 0 | 0.001 | 0.5878 |
| 05246_357 | 0 | 0 | 0 | 0 | 0 | 0 | 0 | 0 | 0 |
| 05246_359 | 0.1278 | 0.142 | 0.1676 | 0.3507 | 0.513 | 0.5323 | 0.4915 | 0.655 | 0.6661 |
| 05246_371 | 0 | 0 | 1.00E−04 | 0 | 0 | 0.002 | 0 | 0 | 0.002 |
| 05246_376 | 0 | 0 | 7.00E−04 | 0 | 0 | 0.0023 | 0 | 0 | 0.003 |
| 05246_465 | 0 | 0 | 0.0468 | 0 | 0 | 0.1166 | 0 | 0 | 0.1634 |
| 05246_473 | 0 | 0 | 0.0021 | 0 | 0 | 0.002 | 0 | 0 | 0.0041 |
| 05246_8 | 0 | 0 | 0.1401 | 0 | 0 | 0.448 | 0 | 0.001 | 0.5881 |
| ONC138111_W00 | 0 | 0 | 0 | 0 | 0 | 0 | 0 | 0 | 0 |
| ONC138111_W03 | 0 | 0 | 0.0275 | 0 | 0.003 | 0.0274 | 0 | 0.003 | 0.0549 |
| ONC138111_W12 | 0 | 0 | 1.00E−04 | 0 | 0 | 0.002 | 0 | 0 | 0.0021 |
| ONC143854_W00 | 0 | 0 | 0 | 0 | 0 | 0 | 0 | 0 | 0 |
| ONC147579_W00 | 0 | 0 | 0.004 | 0 | 0 | 0 | 0 | 0 | 0.004 |
| ONC152713_W00 | 0 | 0 | 0 | 0 | 0 | 2.00E−04 | 0 | 0 | 2.00E−04 |
| ONC152713_W03 | 0 | 0 | 0 | 0 | 0 | 0 | 0 | 0 | 0 |
| ONC156389_W03 | 0 | 0 | 7.00E−04 | 0 | 0 | 0.0081 | 0 | 0 | 0.0106 |
| ONC157864_W00 | 0 | 0 | 0.0126 | 0 | 0 | 0.0112 | 0 | 0 | 0.0238 |
| ONC159155_W00 | 0 | 0 | 2.00E−04 | 0 | 0 | 1.00E−04 | 0 | 0 | 3.00E−04 |
| ONC159155_W12 | 0 | 0 | 0 | 0 | 0 | 0 | 0 | 0 | 0 |
| ONC159391_W00 | 0 | 0 | 1.00E−04 | 0 | 0 | 0 | 0 | 0 | 1.00E−04 |
| ONC161752_W00 | 0 | 0 | 0.0619 | 0 | 0 | 0.1255 | 0 | 0 | 0.1817 |
| ONC166813_W00 | 0 | 0 | 0 | 0 | 0 | 0.002 | 0 | 0 | 0.002 |
| ONC166941_W00 | 0 | 0 | 0 | 0 | 0 | 0 | 0 | 0 | 0 |
| ONC166941_W12 | 0 | 0 | 2.00E−04 | 0 | 0 | 4.00E−04 | 0 | 0 | 6.00E−04 |
| ONC170292_W00 | 0 | 0 | 0 | 0 | 0 | 0 | 0 | 0 | 0 |
| ONC170658_W00 | 0 | 0 | 0 | 0 | 0 | 0 | 0 | 0 | 0 |
| ONC172301_W00 | 0 | 0 | 0.0013 | 0 | 0 | 0.0057 | 0 | 0 | 0.007 |
| ONC173793_W00 | 0 | 0 | 0.0162 | 0 | 0 | 0.0243 | 0 | 0 | 0.0405 |
| ONC174333_W00 | 0 | 0 | 0.0064 | 0 | 0 | 0.006 | 0 | 0 | 0.0104 |
| ONC175142_W00 | 0.2313 | 0.259 | 0.2766 | 0.3869 | 0.416 | 0.4843 | 0.6432 | 0.685 | 0.7221 |
| ONC176222_W00 | 0 | 0 | 0 | 0 | 0 | 4.00E−04 | 0 | 0 | 4.00E−04 |
| ONC178033_W00 | 0 | 0 | 0.0191 | 0 | 0 | 0.0313 | 0 | 0 | 0.0466 |
| ONC178033_W12 | 0 | 0 | 0.0103 | 0 | 0 | 0.0022 | 0 | 0 | 0.0125 |
| ONC179846_W00 | 0 | 0 | 8.00E−04 | 0 | 0 | 0.0024 | 0 | 0 | 0.0032 |
| ONC181838_W12 | 0 | 0.017 | 0.0261 | 0 | 0.002 | 0.004 | 0 | 0.019 | 0.0281 |
| ONC182745_W00 | 0 | 0 | 0 | 0 | 0 | 0.004 | 0 | 0 | 0.004 |
| ONC184454_W00 | 0 | 0 | 0 | 0 | 0 | 0 | 0 | 0 | 0 |
| ONC192824_W00 | 0 | 0 | 0 | 0 | 0 | 1.00E−04 | 0 | 0 | 1.00E−04 |
| ONC193018_W00 | 0 | 0 | 0.0023 | 0 | 0 | 0.0043 | 0 | 0 | 0.0066 |
| ONC194658_W00 | 0 | 0 | 0.0282 | 0 | 0 | 0.034 | 0 | 0 | 0.0622 |
| ONC195079_W00 | 0 | 0 | 0.0195 | 0 | 0 | 0.0165 | 0 | 0 | 0.0341 |
| ONC195262_W00 | 0 | 0 | 0 | 0 | 0 | 0 | 0 | 0 | 0 |
| ONC195262_W03 | 0 | 0.009 | 0.0192 | 0 | 0.006 | 0.0196 | 0 | 0.016 | 0.035 |
| ONC195262_W12 | 0 | 0 | 0.0041 | 0 | 0 | 0.0063 | 0 | 0 | 0.0103 |
| ONC33279_W00 | 0 | 0 | 0.002 | 0 | 0 | 0.006 | 0 | 0.002 | 0.006 |

pB1.5 = “p_BRCA1.5%”;
pB1.50 = “p_BRCA1.50%”;
pB1.95 = “p_BRCA1.95%”;
pB2.5 = “p_BRCA2.5%”;
pB2.50 = “p_BRCA2.50%”;
pB2.95 = “p_BRCA2.95%”;
phrd5 = “p_hrd.5%”;
phrd.50 = “p_hrd.50%”;
phrd.95 = “p_hrd.95%”

TABLE 6B

| sample | dataset | p_BRCA1 | p_BRCA2 | p_hrd | hr_status | hrd_type |
|---|---|---|---|---|---|---|
| ONC138111 | tumor_biopsy_fullpurity | 0.002 | 0.01 | 0.012 | HR_proficient | none |
| ONC143854 | tumor_biopsy_fullpurity | 0.02 | 0.118 | 0.138 | HR_proficient | none |
| ONC147579 | tumor_biopsy_fullpurity | 0.082 | 0.856 | 0.938 | HR_deficient | BRCA2_type |
| ONC152713 | tumor_biopsy_fullpurity | 0.002 | 0.058 | 0.06 | HR_proficient | none |
| ONC156134 | tumor_biopsy_fullpurity | 0.022 | 0.786 | 0.808 | HR_deficient | BRCA2_type |
| ONC156389 | tumor_biopsy_fullpurity | 0.012 | 0.076 | 0.088 | HR_proficient | none |
| ONC157864 | tumor_biopsy_fullpurity | 0.048 | 0.154 | 0.202 | HR_proficient | none |
| ONC159155 | tumor_biopsy_fullpurity | 0.078 | 0.68 | 0.758 | HR_deficient | BRCA2_type |
| ONC159391 | tumor_biopsy_fullpurity | 0.022 | 0.12 | 0.142 | HR_proficient | none |
| ONC161752 | tumor_biopsy_fullpurity | 0.084 | 0.588 | 0.672 | HR_deficient | BRCA2_type |
| ONC166813 | tumor_biopsy_fullpurity | 0.002 | 0.012 | 0.014 | HR_proficient | none |
| ONC167203 | tumor_biopsy_fullpurity | 0.104 | 0.648 | 0.752 | HR_deficient | BRCA2_type |
| ONC170292 | tumor_biopsy_fullpurity | 0.058 | 0.15 | 0.208 | HR_proficient | none |
| ONC170658 | tumor_biopsy_fullpurity | 0.118 | 0.838 | 0.956 | HR_deficient | BRCA2_type |
| ONC171635 | tumor_biopsy_fullpurity | 0.026 | 0.11 | 0.136 | HR_proficient | none |
| ONC172301 | tumor_biopsy_fullpurity | 0.032 | 0.144 | 0.176 | HR_proficient | none |
| ONC173793 | tumor_biopsy_fullpurity | 0.07 | 0.176 | 0.246 | HR_proficient | none |
| ONC174333 | tumor_biopsy_fullpurity | 0.08 | 0.17 | 0.25 | HR_proficient | none |
| ONC175142 | tumor_biopsy_fullpurity | 0.21 | 0.748 | 0.958 | HR_deficient | BRCA2_type |
| ONC176222 | tumor_biopsy_fullpurity | 0.022 | 0.142 | 0.164 | HR_proficient | none |
| ONC178033 | tumor_biopsy_fullpurity | 0.026 | 0.132 | 0.158 | HR_proficient | none |
| ONC179846 | tumor_biopsy_fullpurity | 0.004 | 0.068 | 0.072 | HR_proficient | none |
| ONC181838 | tumor_biopsy_fullpurity | 0.08 | 0.172 | 0.252 | HR_proficient | none |
| ONC184454 | tumor_biopsy_fullpurity | 0.064 | 0.798 | 0.862 | HR_deficient | BRCA2_type |
| ONC192824 | tumor_biopsy_fullpurity | 0.1 | 0.796 | 0.896 | HR_deficient | BRCA2_type |
| ONC195700 | tumor_biopsy_fullpurity | 0.11 | 0.808 | 0.918 | HR_deficient | BRCA2_type |
| ONC33279 | tumor_biopsy_fullpurity | 0.004 | 0.03 | 0.034 | HR_proficient | none |
| ONC138111 | tumor_biopsy_5% | 0 | 0 | 0 | cannot_be_determined | none |
| ONC143854 | tumor_biopsy_5% | 0 | 0.002 | 0.002 | cannot_be_determined | none |
| ONC147579 | tumor_biopsy_5% | 0.086 | 0.594 | 0.68 | HR_deficient | BRCA2_type |
| ONC152713 | tumor_biopsy_5% | 0 | 0 | 0 | cannot_be_determined | none |
| ONC156134 | tumor_biopsy_5% | 0 | 0 | 0 | cannot_be_determined | none |
| ONC156389 | tumor_biopsy_5% | 0 | 0 | 0 | HR_proficient | none |
| ONC157864 | tumor_biopsy_5% | 0.016 | 0.012 | 0.028 | cannot_be_determined | none |
| ONC159155 | tumor_biopsy_5% | 0 | 0 | 0 | HR_proficient | none |
| ONC159391 | tumor_biopsy_5% | 0.098 | 0.578 | 0.676 | HR_deficient | BRCA2_type |
| ONC161752 | tumor_biopsy_5% | 0.126 | 0.554 | 0.68 | HR_deficient | BRCA2_type |
| ONC166813 | tumor_biopsy_5% | 0 | 0 | 0 | cannot_be_determined | none |
| ONC167203 | tumor_biopsy_5% | 0 | 0 | 0 | cannot_be_determined | none |
| ONC170292 | tumor_biopsy_5% | 0 | 0 | 0 | cannot_be_determined | none |
| ONC170658 | tumor_biopsy_5% | 0 | 0 | 0 | cannot_be_determined | none |
| ONC171635 | tumor_biopsy_5% | 0 | 0 | 0 | cannot_be_determined | none |
| ONC172301 | tumor_biopsy_5% | 0 | 0 | 0 | cannot_be_determined | none |
| ONC173793 | tumor_biopsy_5% | 0.01 | 0.012 | 0.022 | cannot_be_determined | none |
| ONC174333 | tumor_biopsy_5% | 0 | 0 | 0 | cannot_be_determined | none |
| ONC175142 | tumor_biopsy_5% | 0 | 0 | 0 | cannot_be_determined | none |
| ONC176222 | tumor_biopsy_5% | 0 | 0 | 0 | HR_proficient | none |
| ONC178033 | tumor_biopsy_5% | 0 | 0 | 0 | cannot_be_determined | none |
| ONC179846 | tumor_biopsy_5% | 0 | 0.002 | 0.002 | cannot_be_determined | none |
| ONC181838 | tumor_biopsy_5% | 0 | 0.012 | 0.012 | cannot_be_determined | none |
| ONC184454 | tumor_biopsy_5% | 0.006 | 0 | 0.006 | cannot_be_determined | none |
| ONC192824 | tumor_biopsy_5% | 0.004 | 0.02 | 0.024 | cannot_be_determined | none |
| ONC195700 | tumor_biopsy_5% | 0 | 0.002 | 0.002 | cannot_be_determined | none |
| ONC33279 | tumor_biopsy_5% | 0 | 0 | 0 | cannot_be_determined | none |
| ONC138111 | tumor_biopsy_2% | 0.006 | 0 | 0.006 | cannot_be_determined | none |
| ONC143854 | tumor_biopsy_2% | 0 | 0 | 0 | cannot_be_determined | none |
| ONC147579 | tumor_biopsy_2% | 0.008 | 0 | 0.008 | cannot_be_determined | none |
| ONC152713 | tumor_biopsy_2% | 0 | 0 | 0 | cannot_be_determined | none |
| ONC156134 | tumor_biopsy_2% | 0 | 0 | 0 | cannot_be_determined | none |
| ONC156389 | tumor_biopsy_2% | 0 | 0 | 0 | cannot_be_determined | none |
| ONC157864 | tumor_biopsy_2% | 0 | 0.002 | 0.002 | cannot_be_determined | none |
| ONC159155 | tumor_biopsy_2% | 0 | 0 | 0 | cannot_be_determined | none |
| ONC159391 | tumor_biopsy_2% | 0.098 | 0.558 | 0.656 | HR_deficient | BRCA2_type |
| ONC161752 | tumor_biopsy_2% | 0.078 | 0.576 | 0.654 | HR_deficient | BRCA2_type |
| ONC166813 | tumor_biopsy_2% | 0 | 0 | 0 | cannot_be_determined | none |
| ONC167203 | tumor_biopsy_2% | 0.018 | 0.022 | 0.04 | cannot_be_determined | none |
| ONC170292 | tumor_biopsy_2% | 0 | 0 | 0 | cannot_be_determined | none |
| ONC170658 | tumor_biopsy_2% | 0 | 0.002 | 0.002 | cannot_be_determined | none |
| ONC171635 | tumor_biopsy_2% | 0 | 0 | 0 | cannot_be_determined | none |
| ONC172301 | tumor_biopsy_2% | 0 | 0 | 0 | cannot_be_determined | none |
| ONC173793 | tumor_biopsy_2% | 0 | 0 | 0 | cannot_be_determined | none |
| ONC174333 | tumor_biopsy_2% | 0 | 0 | 0 | cannot_be_determined | none |
| ONC175142 | tumor_biopsy_2% | 0.002 | 0.016 | 0.018 | cannot_be_determined | none |
| ONC176222 | tumor_biopsy_2% | 0 | 0 | 0 | HR_proficient | none |
| ONC178033 | tumor_biopsy_2% | 0 | 0 | 0 | cannot_be_determined | none |
| ONC179846 | tumor_biopsy_2% | 0 | 0 | 0 | cannot_be_determined | none |
| ONC181838 | tumor_biopsy_2% | 0 | 0 | 0 | cannot_be_determined | none |
| ONC184454 | tumor_biopsy_2% | 0.008 | 0 | 0.008 | cannot_be_determined | none |
| ONC192824 | tumor_biopsy_2% | 0.008 | 0 | 0.008 | cannot_be_determined | none |
| ONC195700 | tumor_biopsy_2% | 0 | 0 | 0 | cannot_be_determined | none |
| ONC33279 | tumor_biopsy_2% | 0 | 0 | 0 | cannot_be_determined | none |
| 05246_185 | stage IV | 0 | 0 | 0 | HR_proficient | none |
| 05246_252 | stage IV | 0 | 0.004 | 0.004 | HR_proficient | none |
| 05246_292 | stage IV | 0.002 | 0.01 | 0.012 | HR_proficient | none |
| 05246_300 | stage IV | 0 | 0 | 0 | HR_proficient | none |
| 05246_303 | stage IV | 0.224 | 0.44 | 0.664 | HR_deficient | BRCA2_type |
| 05246_306 | stage IV | 0 | 0 | 0 | HR_proficient | none |
| 05246_313 | stage IV | 0 | 0 | 0 | HR_proficient | none |
| 05246_315 | stage IV | 0 | 0 | 0 | HR_proficient | none |
| 05246_318 | stage IV | 0.01 | 0.016 | 0.026 | HR_proficient | none |
| 05246_321 | stage IV | 0 | 0.004 | 0.004 | HR_proficient | none |
| 05246_331 | stage IV | 0 | 0.002 | 0.002 | HR_proficient | none |
| 05246_343 | stage IV | 0 | 0 | 0 | cannot_be_determined | none |
| 05246_350 | stage IV | 0 | 0 | 0 | HR_proficient | none |
| 05246_357 | stage IV | 0 | 0 | 0 | HR_proficient | none |
| 05246_359 | stage IV | 0.142 | 0.516 | 0.658 | HR_deficient | BRCA2_type |
| 05246_371 | stage IV | 0 | 0 | 0 | HR_proficient | none |
| 05246_376 | stage IV | 0 | 0 | 0 | HR_proficient | none |
| 05246_465 | stage IV | 0 | 0 | 0 | HR_proficient | none |
| 05246_473 | stage IV | 0 | 0 | 0 | HR_proficient | none |
| 05246_8 | stage IV | 0 | 0 | 0 | HR_proficient | none |
| ONC138111_W00 | early-stage | 0.004 | 0.046 | 0.05 | HR_proficient | none |
| | breast | | | | | |
| ONC138111_W03 | early-stage | 0.004 | 0.054 | 0.058 | HR_proficient | none |
| | breast | | | | | |
| ONC138111_W12 | early-stage | 0.01 | 0.074 | 0.084 | HR_proficient | none |
| | breast | | | | | |
| ONC143854_W00 | early-stage | 0.002 | 0.028 | 0.03 | HR_proficient | none |
| | breast | | | | | |
| ONC147579_W00 | early-stage | 0.002 | 0.034 | 0.036 | HR_proficient | none |
| | breast | | | | | |
| ONC152713_W00 | early-stage | 0.004 | 0.046 | 0.05 | HR_proficient | none |
| | breast | | | | | |
| ONC152713_W03 | early-stage | 0.002 | 0.046 | 0.048 | HR_proficient | none |
| | breast | | | | | |
| ONC156389_W03 | early-stage | 0.002 | 0.038 | 0.04 | HR_proficient | none |
| | breast | | | | | |
| ONC157864_W00 | early-stage | 0.002 | 0.03 | 0.032 | HR_proficient | none |
| | breast | | | | | |
| ONC159155_W00 | early-stage | 0.01 | 0.074 | 0.084 | HR_proficient | none |
| | breast | | | | | |
| ONC159155_W12 | early-stage | 0.004 | 0.046 | 0.05 | HR_proficient | none |
| | breast | | | | | |
| ONC159391_W00 | early-stage | 0 | 0.026 | 0.026 | HR_proficient | none |
| | breast | | | | | |
| ONC161752_W00 | early-stage | 0.002 | 0.03 | 0.032 | HR_proficient | none |
| | breast | | | | | |
| ONC166813_W00 | early-stage | 0.006 | 0.054 | 0.06 | HR_proficient | none |
| | breast | | | | | |
| ONC166941_W00 | early-stage | 0.002 | 0.036 | 0.038 | HR_proficient | none |
| | breast | | | | | |
| ONC166941_W12 | early-stage | 0.002 | 0.028 | 0.03 | HR_proficient | none |
| | breast | | | | | |
| ONC170292_W00 | early-stage | 0.004 | 0.04 | 0.044 | HR_proficient | none |
| | breast | | | | | |
| ONC170658_W00 | early-stage | 0.004 | 0.036 | 0.04 | HR_proficient | none |
| | breast | | | | | |
| ONC172301_W00 | early-stage | 0.01 | 0.072 | 0.082 | HR_proficient | none |
| | breast | | | | | |
| ONC173793_W00 | early-stage | 0.002 | 0.034 | 0.036 | HR_proficient | none |
| | breast | | | | | |
| ONC174333_W00 | early-stage | 0.016 | 0.082 | 0.098 | HR_proficient | none |
| | breast | | | | | |
| ONC175142_W00 | early-stage | 0.174 | 0.652 | 0.826 | HR_deficient | BRCA2_type |
| | breast | | | | | |
| ONC176222_W00 | early-stage | 0.004 | 0.044 | 0.048 | HR_proficient | none |
| | breast | | | | | |
| ONC178033_W00 | early-stage | 0.006 | 0.044 | 0.05 | HR_proficient | none |
| | breast | | | | | |
| ONC178033_W12 | early-stage | 0.004 | 0.058 | 0.062 | HR_proficient | none |
| | breast | | | | | |
| ONC179846_W00 | early-stage | 0.01 | 0.064 | 0.074 | HR_proficient | none |
| | breast | | | | | |
| ONC181838_W12 | early-stage | 0.028 | 0.048 | 0.076 | HR_proficient | none |
| | breast | | | | | |
| ONC182745_W00 | early-stage | 0.008 | 0.066 | 0.074 | HR_proficient | none |
| | breast | | | | | |
| ONC184454_W00 | early-stage | 0.01 | 0.062 | 0.072 | HR_proficient | none |
| | breast | | | | | |
| ONC192824_W00 | early-stage | 0.008 | 0.078 | 0.086 | HR_proficient | none |
| | breast | | | | | |
| ONC193018_W00 | early-stage | 0.002 | 0.042 | 0.044 | HR_proficient | none |
| | breast | | | | | |
| ONC194658_W00 | early-stage | 0.006 | 0.044 | 0.05 | HR_proficient | none |
| | breast | | | | | |
| ONC195079_W00 | early-stage | 0.012 | 0.076 | 0.088 | HR_proficient | none |
| | breast | | | | | |
| ONC195262_W00 | early-stage | 0.008 | 0.08 | 0.088 | HR_proficient | none |
| | breast | | | | | |
| ONC195262_W03 | early-stage | 0.014 | 0.106 | 0.12 | HR_proficient | none |
| | breast | | | | | |
| ONC195262_W12 | early-stage | 0.004 | 0.086 | 0.09 | HR_proficient | none |
| | breast | | | | | |
| ONC33279_W00 | early-stage | 0.012 | 0.072 | 0.084 | HR_proficient | none |
| | breast | | | | | |
| 108200_posttx138d | prostate | 0.004 | 0.034 | 0.038 | HR_proficient | none |
| 108200_pretx530d | prostate | 0.004 | 0.034 | 0.038 | HR_proficient | none |
| 128311_posttx127d | prostate | 0.002 | 0.036 | 0.038 | HR_proficient | none |
| 128311_pretx1333d | prostate | 0.002 | 0.032 | 0.034 | HR_proficient | none |
| 384102_pretx78d | prostate | 0 | 0.024 | 0.024 | HR_proficient | none |
| 407120_posttx489d | prostate | 0.002 | 0.036 | 0.038 | HR_proficient | none |
| 407120_pretx683d | prostate | 0 | 0.034 | 0.034 | HR_proficient | none |
| 476703_pretx57d | prostate | 0.006 | 0.056 | 0.062 | HR_proficient | none |
| 500560_pretx11d | prostate | 0.002 | 0.036 | 0.038 | HR_proficient | none |
| 566240_pretx437d | prostate | 0.004 | 0.038 | 0.042 | HR_proficient | none |
| 566691_posttx26d | prostate | 0.004 | 0.042 | 0.046 | HR_proficient | none |
| 566691_pretx0d | prostate | 0 | 0.028 | 0.028 | HR_proficient | none |
| 646059_pretx6d | prostate | 0.166 | 0.574 | 0.74 | HR_deficient | BRCA2_type |
| 653096_posttx31d | prostate | 0.104 | 0.762 | 0.866 | HR_deficient | BRCA2_type |
| 653096_pretx0d | prostate | 0.054 | 0.088 | 0.142 | HR_proficient | none |
| 718458_pretx0d | prostate | 0 | 0.022 | 0.022 | HR_proficient | none |
| 752770_pretx0d | prostate | 0.022 | 0.062 | 0.084 | HR_proficient | none |
| GENP2455-2 | prostate | 0.012 | 0.06 | 0.072 | HR_proficient | none |
| GENP2560-2 | prostate | 0.01 | 0.056 | 0.066 | HR_proficient | none |
| GENP3534-1 | prostate | 0 | 0.03 | 0.03 | HR_proficient | none |
| GENP4005-1 | prostate | 0.004 | 0.04 | 0.044 | HR_proficient | none |
| RGENP1401_P2 | prostate | 0 | 0.02 | 0.02 | HR_proficient | none |
| RGENP462_2 | prostate | 0.168 | 0.636 | 0.804 | HR_deficient | BRCA2_type |
| RGENP490_1 | prostate | 0.1 | 0.33 | 0.43 | HR_proficient | none |
| RGENP544_1 | prostate | 0.004 | 0.044 | 0.048 | HR_proficient | none |
| OC_NACT_A_PL_T1_vs_WB | ovary | 0.004 | 0.038 | 0.042 | HR_proficient | none |
| RP- | ovary | 0.01 | 0.058 | 0.068 | HR_proficient | none |
| 3265_OC_NACT_B_PL_T1_vs_WB | | | | | | |
| RP- | ovary | 0.008 | 0.054 | 0.062 | HR_proficient | none |
| 3265_OC_NACT_D_PL_T1_vs_WB | | | | | | |
| RP- | ovary | 0.008 | 0.048 | 0.056 | HR_proficient | none |
| 3265_OC_NACT_F_PL_T1_vs_WB | | | | | | |
| RP- | ovary | 0.008 | 0.056 | 0.064 | HR_proficient | none |
| 3265_OC_NACT_M_PL_T1_vs_WB | | | | | | |
| RP- | ovary | 0.012 | 0.062 | 0.074 | HR_proficient | none |
| 3265_OC_NACT_S_PL_T1_vs_WB | | | | | | |
| RP- | ovary | 0.008 | 0.054 | 0.062 | HR_proficient | none |
| 3265_OC_NACT_U_PL_T1_vs_WB | | | | | | |
| RP- | ovary | 0.008 | 0.056 | 0.064 | HR_proficient | none |
| 3265_OC_NACT_V_PL_T1_vs_WB | | | | | | |
| RP- | ovary | 0.002 | 0.034 | 0.036 | HR_proficient | none |
| 3265_OC_NACT_W_PL_T1_vs_WB | | | | | | |
| RP- | ovary | 0.008 | 0.048 | 0.056 | HR_proficient | none |
| 3265_OC_NACT_X_PL_T1_vs_WB | | | | | | |

| sample | p_BRCA1.5% | p_BRCA1.50% | p_BRCA1.95% | p_BRCA2.5% | p_BRCA2.50% |
|---|---|---|---|---|---|
| ONC138111 | 0 | 0.002 | 0.0066 | 0.0099 | 0.013 |
| ONC143854 | 0.016 | 0.02 | 0.0241 | 0.1078 | 0.12 |
| ONC147579 | 0.0535 | 0.084 | 0.2073 | 0.6677 | 0.824 |
| ONC152713 | 0 | 0.003 | 0.012 | 0.0359 | 0.068 |
| ONC156134 | 0.0159 | 0.021 | 0.0261 | 0.1339 | 0.773 |
| ONC156389 | 0.0039 | 0.018 | 0.053 | 0.0629 | 0.101 |
| ONC157864 | 0.0119 | 0.047 | 0.0722 | 0.0835 | 0.131 |
| ONC159155 | 0.0079 | 0.032 | 0.0801 | 0.144 | 0.684 |
| ONC159391 | 0.0159 | 0.025 | 0.0598 | 0.1116 | 0.124 |
| ONC161752 | 0.0116 | 0.062 | 0.0882 | 0.0675 | 0.638 |
| ONC166813 | 0 | 0 | 0.0106 | 0.0098 | 0.019 |
| ONC167203 | 0.014 | 0.065 | 0.11 | 0.1278 | 0.689 |
| ONC170292 | 0.0059 | 0.032 | 0.1024 | 0.0394 | 0.117 |
| ONC170658 | 0.0038 | 0.068 | 0.1882 | 0.1705 | 0.703 |
| ONC171635 | 0.0099 | 0.025 | 0.0322 | 0.0844 | 0.115 |
| ONC172301 | 0.0177 | 0.03 | 0.034 | 0.1297 | 0.137 |
| ONC173793 | 0.0219 | 0.072 | 0.1089 | 0.1354 | 0.25 |
| ONC174333 | 0.0178 | 0.074 | 0.0872 | 0.116 | 0.166 |
| ONC175142 | 0.0819 | 0.206 | 0.233 | 0.6178 | 0.749 |
| ONC176222 | 0.0199 | 0.065 | 0.0947 | 0.1295 | 0.19 |
| ONC178033 | 0.026 | 0.028 | 0.032 | 0.1059 | 0.123 |
| ONC179846 | 0 | 0.006 | 0.0102 | 0.012 | 0.08 |
| ONC181838 | 0.0236 | 0.076 | 0.211 | 0.1238 | 0.169 |
| ONC184454 | 0.006 | 0.063 | 0.1062 | 0.7049 | 0.797 |
| ONC192824 | 0.0217 | 0.104 | 0.1761 | 0.699 | 0.808 |
| ONC195700 | 0.0855 | 0.104 | 0.1874 | 0.2838 | 0.803 |
| ONC33279 | 0 | 0.006 | 0.0162 | 0.0099 | 0.047 |
| ONC138111 | 0.0141 | 0 | 0.004 | 0.0181 | 0 |
| ONC143854 | 0 | 0 | 0 | 0.002 | 0 |
| ONC147579 | 0.066 | 0.083 | 0.0984 | 0.5758 | 0.61 |
| ONC152713 | 0 | 0 | 0 | 0.002 | 0 |
| ONC156134 | 0 | 0 | 0 | 0.0042 | 0 |
| ONC156389 | 0 | 0 | 0 | 0 | 0 |
| ONC157864 | 0.02 | 0 | 0.012 | 0.0181 | 0 |
| ONC159155 | 0 | 0 | 0 | 0 | 0 |
| ONC159391 | 0.0759 | 0.095 | 0.1082 | 0.5657 | 0.58 |
| ONC161752 | 0 | 0.106 | 0.1422 | 0 | 0.548 |
| ONC166813 | 0.0061 | 0 | 0 | 0 | 0 |
| ONC167203 | 0.0121 | 0 | 0.002 | 0.0161 | 0 |
| ONC170292 | 0.0046 | 0 | 0.001 | 0.018 | 0 |
| ONC170658 | 0.0083 | 0 | 0 | 0.018 | 0 |
| ONC171635 | 0 | 0 | 0 | 1.00E−04 | 0 |
| ONC172301 | 0 | 0 | 0 | 0 | 0 |
| ONC173793 | 0.016 | 0 | 0 | 0.018 | 0 |
| ONC174333 | 0 | 0 | 0 | 0.002 | 0 |
| ONC175142 | 0 | 0 | 0 | 1.00E−04 | 0 |
| ONC176222 | 0 | 0 | 0 | 0 | 0.001 |
| ONC178033 | 0 | 0 | 0 | 1.00E−04 | 0 |
| ONC179846 | 0 | 0 | 0 | 0.0021 | 0 |
| ONC181838 | 0.0065 | 0 | 0.002 | 0.018 | 0 |
| ONC184454 | 0.0061 | 0 | 0 | 0 | 0 |
| ONC192824 | 0.0061 | 0.0058 | 0.014 | 0.0201 | 0.006 |
| ONC195700 | 0 | 0 | 0.002 | 0.004 | 0 |
| ONC33279 | 0 | 0 | 0 | 0.0021 | 0 |
| ONC138111 | 0.008 | 0 | 0 | 0 | 0 |
| ONC143854 | 0 | 0 | 0 | 0 | 0 |
| ONC147579 | 0.008 | 0 | 0 | 5.00E−04 | 0 |
| ONC152713 | 0.006 | 0 | 0 | 0.0162 | 0 |
| ONC156134 | 2.00E−04 | 0 | 0 | 0.0027 | 0 |
| ONC156389 | 0.014 | 0 | 0 | 0.0161 | 0 |
| ONC157864 | 0 | 0 | 0 | 0.0021 | 0 |
| ONC159155 | 3.00E−04 | 0 | 0 | 0.002 | 0 |
| ONC159391 | 0.0659 | 0.098 | 0.1082 | 0.544 | 0.565 |
| ONC161752 | 0.0735 | 0.078 | 0.108 | 0.544 | 0.576 |
| ONC166813 | 0.006 | 0 | 0 | 0.0044 | 0 |
| ONC167203 | 0.018 | 0 | 0.002 | 0.022 | 0 |
| ONC170292 | 0.006 | 0 | 0 | 0 | 0 |
| ONC170658 | 0 | 0.0019 | 0.002 | 0.002 | 0.002 |
| ONC171635 | 0 | 0 | 0 | 0.002 | 0 |
| ONC172301 | 0.008 | 0 | 0 | 0.0241 | 0 |
| ONC173793 | 3.00E−04 | 0 | 0 | 0 | 0 |
| ONC174333 | 0 | 0 | 0 | 0.002 | 0 |
| ONC175142 | 0.018 | 0 | 0 | 0.022 | 0 |
| ONC176222 | 0 | 0 | 0 | 0 | 0 |
| ONC178033 | 6.00E−04 | 0 | 0 | 0 | 0 |
| ONC179846 | 3.00E−04 | 0 | 0 | 0 | 0 |
| ONC181838 | 0 | 0 | 0 | 0 | 0 |
| ONC184454 | 0.008 | 0 | 0 | 0 | 0 |
| ONC192824 | 0.008 | 0 | 0 | 6.00E−04 | 0 |
| ONC195700 | 0.006 | 0 | 0 | 0.0101 | 0 |
| ONC33279 | 0.006 | 0 | 0 | 0 | 0 |
| 05246_185 | 0 | 0 | 0 | 0 | 0 |
| 05246_252 | 0 | 0.006 | 0.1439 | 0 | 0.008 |
| 05246_292 | 0 | 0.002 | 0.01 | 0 | 0.005 |
| 05246_300 | 0 | 0 | 0 | 0 | 0 |
| 05246_303 | 0.0417 | 0.178 | 0.2485 | 0.0627 | 0.43 |
| 05246_306 | 0 | 0 | 0.0416 | 0 | 0 |
| 05246_313 | 0 | 0 | 0 | 0 | 0 |
| 05246_315 | 0 | 0 | 0.1101 | 0 | 0 |
| 05246_318 | 0 | 0.015 | 0.1403 | 0 | 0.027 |
| 05246_321 | 0 | 0 | 0.0537 | 0 | 0 |
| 05246_331 | 0 | 0 | 1.00E−04 | 0.002 | 0.002 |
| 05246_343 | 0 | 0 | 0 | 0 | 0 |
| 05246_350 | 0 | 0 | 0.176 | 0 | 0 |
| 05246_357 | 0 | 0 | 0 | 0 | 0 |
| 05246_359 | 0.1278 | 0.142 | 0.1676 | 0.3507 | 0.513 |
| 05246_371 | 0 | 0 | 1.00E−04 | 0 | 0 |
| 05246_376 | 0 | 0 | 7.00E−04 | 0 | 0 |
| 05246_465 | 0 | 0 | 0.0468 | 0 | 0 |
| 05246_473 | 0 | 0 | 0.0021 | 0 | 0 |
| 05246_8 | 0 | 0 | 0.1401 | 0 | 0 |
| ONC138111_W00 | 0.0019 | 0.004 | 0.0201 | 0.042 | 0.054 |
| ONC138111_W03 | 0.004 | 0.011 | 0.0344 | 0.04 | 0.058 |
| ONC138111_W12 | 0.004 | 0.01 | 3.62E−02 | 0.042 | 0.07 |
| ONC143854_W00 | 0 | 0.001 | 0.002 | 0.018 | 0.029 |
| ONC147579_W00 | 0 | 0.002 | 0.021 | 0.0179 | 0.036 |
| ONC152713_W00 | 0.002 | 0.004 | 0.008 | 0.0338 | 0.04 |
| ONC152713_W03 | 0 | 0.004 | 0.0101 | 0.0333 | 0.043 |
| ONC156389_W03 | 0 | 0.002 | 1.89E−02 | 0.032 | 0.041 |
| ONC157864_W00 | 0.0019 | 0.002 | 0.0432 | 0.0199 | 0.03 |
| ONC159155_W00 | 0.008 | 0.011 | 1.94E−02 | 0.0618 | 0.076 |
| ONC159155_W12 | 0 | 0.004 | 0.02 | 0.0239 | 0.045 |
| ONC159391_W00 | 0 | 0.002 | 2.25E−02 | 0.016 | 0.034 |
| ONC161752_W00 | 0 | 0.002 | 0.0848 | 0.0236 | 0.036 |
| ONC166813_W00 | 0.002 | 0.007 | 0.01 | 0.04 | 0.056 |
| ONC166941_W00 | 0 | 0.002 | 0.004 | 0.0159 | 0.03 |
| ONC166941_W12 | 0 | 0.002 | 2.91E−02 | 0.0139 | 0.024 |
| ONC170292_W00 | 0.002 | 0.004 | 0.0151 | 0.0338 | 0.04 |
| ONC170658_W00 | 0 | 0.002 | 0.0101 | 0.0239 | 0.037 |
| ONC172301_W00 | 0.0039 | 0.01 | 0.014 | 0.0518 | 0.07 |
| ONC173793_W00 | 0 | 0.002 | 0.0422 | 0.022 | 0.04 |
| ONC174333_W00 | 0.0119 | 0.015 | 0.0426 | 0.0638 | 0.076 |
| ONC175142_W00 | 0.1051 | 0.181 | 0.2069 | 0.5899 | 0.654 |
| ONC176222_W00 | 0.0019 | 0.002 | 0.0041 | 0.0337 | 0.042 |
| ONC178033_W00 | 0.002 | 0.006 | 0.0725 | 0.028 | 0.041 |
| ONC178033_W12 | 0.0019 | 0.005 | 0.036 | 0.0377 | 0.049 |
| ONC179846_W00 | 0.0097 | 0.01 | 4.07E−02 | 0.0596 | 0.071 |
| ONC181838_W12 | 0 | 0.027 | 0.0341 | 0.0118 | 0.045 |
| ONC182745_W00 | 0.0019 | 0.006 | 0.024 | 0.0373 | 0.047 |
| ONC184454_W00 | 0.004 | 0.01 | 0.0148 | 0.0419 | 0.059 |
| ONC192824_W00 | 0.0059 | 0.006 | 0.012 | 0.0399 | 0.07 |
| ONC193018_W00 | 0 | 0.004 | 0.0084 | 0.0297 | 0.036 |
| ONC194658_W00 | 0.002 | 0.008 | 0.0122 | 0.0319 | 0.052 |
| ONC195079_W00 | 0.008 | 0.016 | 0.0402 | 0.062 | 0.074 |
| ONC195262_W00 | 0.0039 | 0.008 | 0.0141 | 0.0494 | 0.079 |
| ONC195262_W03 | 0.002 | 0.016 | 0.0418 | 0.0459 | 0.083 |
| ONC195262_W12 | 0.002 | 0.006 | 0.0206 | 0.0497 | 0.085 |
| ONC33279_W00 | 0.002 | 0.015 | 0.0383 | 0.038 | 0.066 |
| 108200_posttx138d | 0.0019 | 0.004 | 0.008 | 0.0296 | 0.038 |
| 108200_pretx530d | 0.002 | 0.007 | 0.0101 | 0.028 | 0.047 |
| 128311_posttx127d | 0 | 0.003 | 0.0061 | 0.028 | 0.033 |
| 128311_pretx1333d | 0 | 0.004 | 0.008 | 0.032 | 0.036 |
| 384102_pretx78d | 0 | 0 | 0.002 | 0.0199 | 0.024 |
| 407120_posttx489d | 0 | 0.002 | 0.008 | 0.0279 | 0.032 |
| 407120_pretx683d | 0 | 0 | 0.002 | 0.0177 | 0.03 |
| 476703_pretx57d | 0.0058 | 0.01 | 0.0366 | 0.0478 | 0.074 |
| 500560_pretx11d | 0.0019 | 0.002 | 0.0062 | 0.0298 | 0.035 |
| 566240_pretx437d | 0.002 | 0.004 | 0.01 | 0.0339 | 0.038 |
| 566691_posttx26d | 0.002 | 0.003 | 0.006 | 0.0338 | 0.042 |
| 566691_pretx0d | 0 | 0.002 | 0.0021 | 0.0258 | 0.033 |
| 646059_pretx6d | 0.0793 | 0.126 | 0.181 | 0.1846 | 0.554 |
| 653096_posttx31d | 0.0845 | 0.098 | 0.1142 | 0.7431 | 0.766 |
| 653096_pretx0d | 0.0099 | 0.066 | 0.1523 | 0.0398 | 0.174 |
| 718458_pretx0d | 0 | 0 | 0.002 | 0.0179 | 0.024 |
| 752770_pretx0d | 0.004 | 0.014 | 0.068 | 0.0439 | 0.062 |
| GENP2455-2 | 0.004 | 0.01 | 0.0121 | 0.0398 | 0.059 |
| GENP2560-2 | 0.002 | 0.008 | 0.0102 | 0.0377 | 0.054 |
| GENP3534-1 | 0 | 0 | 0.0022 | 0.0258 | 0.032 |
| GENP4005-1 | 0.002 | 0.004 | 0.0081 | 0.0318 | 0.04 |
| RGENP1401_P2 | 0 | 0 | 0 | 0.0159 | 0.021 |
| RGENP462_2 | 0.1134 | 0.151 | 0.1901 | 0.5978 | 0.624 |
| RGENP490_1 | 0.0524 | 0.082 | 0.1282 | 0.1091 | 0.232 |
| RGENP544_1 | 0.002 | 0.004 | 0.0062 | 0.0359 | 0.044 |
| OC_NACT_A_PL_T1_vs_WB | 0.0019 | 0.004 | 0.0081 | 0.0317 | 0.04 |
| RP- | 0.01 | 0.012 | 0.0142 | 0.0598 | 0.065 |
| 3265_OC_NACT_B_PL_T1_vs_WB | | | | | |
| RP- | 0.0058 | 0.01 | 0.014 | 0.0413 | 0.056 |
| 3265_OC_NACT_D_PL_T1_vs_WB | | | | | |
| RP- | 0.002 | 0.008 | 0.008 | 0.028 | 0.046 |
| 3265_OC_NACT_F_PL_T1_vs_WB | | | | | |
| RP- | 0.002 | 0.008 | 0.0121 | 0.0338 | 0.054 |
| 3265_OC_NACT_M_PL_T1_vs_WB | | | | | |
| RP- | 0.0077 | 0.012 | 0.0143 | 0.0559 | 0.062 |
| 3265_OC_NACT_S_PL_T1_vs_WB | | | | | |
| RP- | 0.002 | 0.008 | 0.026 | 0.05 | 0.057 |
| 3265_OC_NACT_U_PL_T1_vs_WB | | | | | |
| RP- | 0.004 | 0.008 | 0.012 | 0.036 | 0.054 |
| 3265_OC_NACT_V_PL_T1_vs_WB | | | | | |
| RP- | 0 | 0.002 | 0.008 | 0.026 | 0.034 |
| 3265_OC_NACT_W_PL_T1_vs_WB | | | | | |
| RP- | 0.0039 | 0.008 | 0.0101 | 0.0356 | 0.048 |
| 3265_OC_NACT_X_PL_T1_vs_WB | | | | | |

| sample | p_BRCA2.95% | p_hrd.5% | p_hrd.50% | p_hrd.95% |
|---|---|---|---|---|
| ONC138111 | 0.0819 | 0.01 | 0.01 | 0.0885 |
| ONC143854 | 0.128 | 0.128 | 0.14 | 0.1442 |
| ONC147579 | 0.896 | 0.872 | 0.91 | 0.9666 |
| ONC152713 | 0.1123 | 0.036 | 0.07 | 0.1243 |
| ONC156134 | 0.8021 | 0.158 | 0.8 | 0.8181 |
| ONC156389 | 0.1788 | 0.067 | 0.12 | 0.2318 |
| ONC157864 | 0.1728 | 0.095 | 0.18 | 0.2412 |
| ONC159155 | 0.8263 | 0.178 | 0.76 | 0.8361 |
| ONC159391 | 0.1858 | 0.128 | 0.14 | 0.2323 |
| ONC161752 | 0.6972 | 0.079 | 0.69 | 0.7385 |
| ONC166813 | 0.1001 | 0.012 | 0.02 | 0.1107 |
| ONC167203 | 0.7947 | 0.162 | 0.76 | 0.8127 |
| ONC170292 | 0.7132 | 0.044 | 0.15 | 0.7969 |
| ONC170658 | 0.844 | 0.189 | 0.79 | 0.939 |
| ONC171635 | 0.1322 | 0.094 | 0.14 | 0.1602 |
| ONC172301 | 0.152 | 0.155 | 0.17 | 0.1822 |
| ONC173793 | 0.6824 | 0.159 | 0.33 | 0.7635 |
| ONC174333 | 0.775 | 0.138 | 0.24 | 0.851 |
| ONC175142 | 0.9022 | 0.849 | 0.96 | 0.9841 |
| ONC176222 | 0.6322 | 0.148 | 0.25 | 0.7155 |
| ONC178033 | 0.1341 | 0.136 | 0.15 | 0.1602 |
| ONC179846 | 0.1089 | 0.012 | 0.09 | 0.1191 |
| ONC181838 | 0.7433 | 0.148 | 0.25 | 0.8384 |
| ONC184454 | 0.8661 | 0.815 | 0.86 | 0.8778 |
| ONC192824 | 0.9603 | 0.809 | 0.91 | 0.9843 |
| ONC195700 | 0.8444 | 0.442 | 0.9 | 0.9401 |
| ONC33279 | 0.1124 | 0.01 | 0.05 | 0.1286 |
| ONC138111 | 0 | | | 0.0282 |
| ONC143854 | 0 | | | 0.002 |
| ONC147579 | 0.6264 | 0.67 | 0.69 | 0.7025 |
| ONC152713 | 0 | | | 0.002 |
| ONC156134 | 0 | | | 0.0042 |
| ONC156389 | 0 | 0 | 0 | 0 |
| ONC157864 | 0.02 | | | 0.038 |
| ONC159155 | 0.004 | 0 | 0 | 0.004 |
| ONC159391 | 0.6026 | 0.666 | 0.67 | 0.69 |
| ONC161752 | 0.5841 | 0 | 0.67 | 0.6861 |
| ONC166813 | 0 | | | 0.0061 |
| ONC167203 | 0 | | | 0.0242 |
| ONC170292 | 0 | | | 0.0226 |
| ONC170658 | 0 | | | 0.0206 |
| ONC171635 | 0 | | | 1.00E−04 |
| ONC172301 | 0 | | | 0 |
| ONC173793 | 0.01 | | | 0.034 |
| ONC174333 | 0 | | | 0.002 |
| ONC175142 | 0 | | | 1.00E−04 |
| ONC176222 | 0.0081 | 0 | 0 | 0.0081 |
| ONC178033 | 0 | | | 1.00E−04 |
| ONC179846 | 0 | | | 0.0021 |
| ONC181838 | 0 | | | 0.0245 |
| ONC184454 | 0 | | | 0.0061 |
| ONC192824 | 0.02 | | | 0.0241 |
| ONC195700 | 0 | | | 0.004 |
| ONC33279 | 0 | | | 0.0021 |
| ONC138111 | 0.01 | | | 0.008 |
| ONC143854 | 0 | | | 0 |
| ONC147579 | 0.01 | | | 0.0081 |
| ONC152713 | 0 | | | 0.0184 |
| ONC156134 | 0 | | | 0.0029 |
| ONC156389 | 0 | | | 0.03 |
| ONC157864 | 0 | | | 0.0021 |
| ONC159155 | 0 | | | 0.0022 |
| ONC159391 | 0.587 | 0.65 | 0.66 | 0.6728 |
| ONC161752 | 0.58 | 0.642 | 0.65 | 0.656 |
| ONC166813 | 0 | | | 0.0063 |
| ONC167203 | 0.01 | | | 0.0381 |
| ONC170292 | 0 | | | 0.006 |
| ONC170658 | 0 | | | 0.002 |
| ONC171635 | 0 | | | 0.002 |
| ONC172301 | 0 | | | 0.0261 |
| ONC173793 | 0 | | | 3.00E−04 |
| ONC174333 | 0 | | | 0.002 |
| ONC175142 | 0 | | | 0.04 |
| ONC176222 | 0.0041 | 0 | 0 | 0.0041 |
| ONC178033 | 0 | | | 6.00E−04 |
| ONC179846 | 0 | | | 3.00E−04 |
| ONC181838 | 0 | | | 0 |
| ONC184454 | 0.01 | | | 0.008 |
| ONC192824 | 0 | | | 0.0083 |
| ONC195700 | 0 | | | 0.0122 |
| ONC33279 | 0 | | | 0.006 |
| 05246_185 | 0 | 0 | 0 | 0 |
| 05246_252 | 0.4571 | 0 | 0.01 | 0.6208 |
| 05246_292 | 0.014 | 0 | 0.01 | 0.0221 |
| 05246_300 | 0 | 0 | 0 | 0 |
| 05246_303 | 0.5431 | 0.104 | 0.67 | 0.7106 |
| 05246_306 | 0.0933 | 0 | 0 | 0.1349 |
| 05246_313 | 0 | 0 | 0 | 0 |
| 05246_315 | 0.177 | 0 | 0 | 0.2861 |
| 05246_318 | 0.4926 | 0 | 0.04 | 0.6383 |
| 05246_321 | 0.1105 | 0 | 0 | 0.1642 |
| 05246_331 | 0.0022 | 0.002 | 0 | 0.0023 |
| 05246_343 | 0 | | | 0 |
| 05246_350 | 0.4406 | 0 | 0 | 0.5878 |
| 05246_357 | 0 | 0 | 0 | 0 |
| 05246_359 | 0.5323 | 0.492 | 0.66 | 0.6661 |
| 05246_371 | 0.002 | 0 | 0 | 0.002 |
| 05246_376 | 0.0023 | 0 | 0 | 0.003 |
| 05246_465 | 0.1166 | 0 | 0 | 0.1634 |
| 05246_473 | 0.002 | 0 | 0 | 0.0041 |
| 05246_8 | 0.448 | 0 | 0 | 0.5881 |
| ONC138111_W00 | 0.0881 | 0.044 | 0.06 | 0.0964 |
| ONC138111_W03 | 0.0862 | 0.044 | 0.08 | 0.1278 |
| ONC138111_W12 | 0.0921 | 0.046 | 0.08 | 0.1225 |
| ONC143854_W00 | 0.0423 | 0.02 | 0.03 | 0.0424 |
| ONC147579_W00 | 0.0712 | 0.018 | 0.04 | 0.0903 |
| ONC152713_W00 | 6.85E−02 | 0.036 | 0.04 | 7.46E−02 |
| ONC152713_W03 | 0.0785 | 0.037 | 0.05 | 0.0848 |
| ONC156389_W03 | 0.0685 | 0.034 | 0.04 | 0.0874 |
| ONC157864_W00 | 0.0748 | 0.02 | 0.03 | 0.118 |
| ONC159155_W00 | 1.09E−01 | 0.07 | 0.09 | 1.29E−01 |
| ONC159155_W12 | 0.0683 | 0.026 | 0.05 | 0.088 |
| ONC159391_W00 | 0.046 | 0.016 | 0.04 | 6.47E−02 |
| ONC161752_W00 | 0.4513 | 0.024 | 0.04 | 0.5505 |
| ONC166813_W00 | 0.0721 | 0.044 | 0.06 | 0.0821 |
| ONC166941_W00 | 0.0481 | 0.018 | 0.03 | 0.0501 |
| ONC166941_W12 | 6.07E−02 | 0.014 | 0.03 | 8.98E−02 |
| ONC170292_W00 | 0.0708 | 0.036 | 0.04 | 0.084 |
| ONC170658_W00 | 0.0543 | 0.024 | 0.04 | 0.0643 |
| ONC172301_W00 | 0.0821 | 0.054 | 0.08 | 0.0901 |
| ONC173793_W00 | 0.1508 | 0.022 | 0.04 | 0.1928 |
| ONC174333_W00 | 0.0923 | 0.074 | 0.09 | 0.1349 |
| ONC175142_W00 | 0.7775 | 0.778 | 0.84 | 0.8826 |
| ONC176222_W00 | 7.28E−02 | 0.036 | 0.04 | 7.48E−02 |
| ONC178033_W00 | 0.5597 | 0.032 | 0.05 | 0.6322 |
| ONC178033_W12 | 0.0843 | 0.04 | 0.05 | 0.1203 |
| ONC179846_W00 | 0.0952 | 0.069 | 0.08 | 0.1359 |
| ONC181838_W12 | 0.056 | 0.014 | 0.07 | 0.084 |
| ONC182745_W00 | 0.0656 | 0.039 | 0.05 | 0.0872 |
| ONC184454_W00 | 0.0806 | 0.046 | 0.07 | 0.0954 |
| ONC192824_W00 | 8.61E−02 | 0.044 | 0.08 | 9.62E−02 |
| ONC193018_W00 | 0.0704 | 0.032 | 0.04 | 0.0731 |
| ONC194658_W00 | 0.0621 | 0.034 | 0.06 | 0.0722 |
| ONC195079_W00 | 0.1003 | 0.076 | 0.09 | 0.1441 |
| ONC195262_W00 | 0.1003 | 0.052 | 0.09 | 0.1162 |
| ONC195262_W03 | 0.1201 | 0.048 | 0.1 | 0.1581 |
| ONC195262_W12 | 0.0991 | 0.052 | 0.09 | 0.1267 |
| ONC33279_W00 | 0.1041 | 0.042 | 0.09 | 0.1325 |
| 108200_posttx138d | 0.0503 | 0.032 | 0.04 | 0.0583 |
| 108200_pretx530d | 0.0588 | 0.03 | 0.06 | 0.0685 |
| 128311_posttx127d | 0.0482 | 0.028 | 0.03 | 0.0542 |
| 128311_pretx1333d | 0.0541 | 0.032 | 0.04 | 0.062 |
| 384102_pretx78d | 0.0321 | 0.02 | 0.02 | 0.0341 |
| 407120_posttx489d | 0.052 | 0.028 | 0.04 | 0.0581 |
| 407120_pretx683d | 0.0341 | 0.02 | 0.03 | 0.036 |
| 476703_pretx57d | 0.1105 | 0.05 | 0.09 | 0.1414 |
| 500560_pretx11d | 0.0446 | 0.032 | 0.04 | 0.0508 |
| 566240_pretx437d | 0.0544 | 0.036 | 0.04 | 0.0644 |
| 566691_posttx26d | 0.0481 | 0.036 | 0.05 | 0.052 |
| 566691_pretx0d | 0.0362 | 0.026 | 0.03 | 0.0382 |
| 646059_pretx6d | 0.6919 | 0.304 | 0.7 | 0.8085 |
| 653096_posttx31d | 0.8075 | 0.83 | 0.87 | 0.8964 |
| 653096_pretx0d | 0.461 | 0.055 | 0.23 | 0.6187 |
| 718458_pretx0d | 0.032 | 0.018 | 0.02 | 0.0321 |
| 752770_pretx0d | 0.1565 | 0.05 | 0.08 | 0.2245 |
| GENP2455-2 | 0.0682 | 0.044 | 0.07 | 0.0802 |
| GENP2560-2 | 0.0689 | 0.04 | 0.06 | 0.0791 |
| GENP3534-1 | 0.0382 | 0.026 | 0.03 | 0.0404 |
| GENP4005-1 | 0.0505 | 0.034 | 0.04 | 0.0586 |
| RGENP1401_P2 | 0.0301 | 0.016 | 0.02 | 0.0301 |
| RGENP462_2 | 0.705 | 0.72 | 0.79 | 0.8261 |
| RGENP490_1 | 0.5368 | 0.162 | 0.32 | 0.6631 |
| RGENP544_1 | 0.06 | 0.038 | 0.05 | 0.0662 |
| OC_NACT_A_PL_T1_vs_WB | 0.0586 | 0.034 | 0.05 | 0.0667 |
| RP- | 0.0784 | 0.07 | 0.08 | 0.0926 |
| 3265_OC_NACT_B_PL_T1_vs_WB | | | | |
| RP- | 0.0661 | 0.047 | 0.07 | 0.0801 |
| 3265_OC_NACT_D_PL_T1_vs_WB | | | | |
| RP- | 0.052 | 0.03 | 0.05 | 0.06 |
| 3265_OC_NACT_F_PL_T1_vs_WB | | | | |
| RP- | 0.0721 | 0.038 | 0.06 | 0.0842 |
| 3265_OC_NACT_M_PL_T1_vs_WB | | | | |
| RP- | 0.0721 | 0.064 | 0.07 | 0.0845 |
| 3265_OC_NACT_S_PL_T1_vs_WB | | | | |
| RP- | 0.0743 | 0.058 | 0.06 | 0.0941 |
| 3265_OC_NACT_U_PL_T1_vs_WB | | | | |
| RP- | 0.0601 | 0.04 | 0.06 | 0.0721 |
| 3265_OC_NACT_V_PL_T1_vs_WB | | | | |
| RP- | 0.0483 | 0.026 | 0.04 | 0.0563 |
| 3265_OC_NACT_W_PL_T1_vs_WB | | | | |
| RP- | 0.0602 | 0.039 | 0.06 | 0.0703 |
| 3265_OC_NACT_X_PL_T1_vs_WB | | | | |

TABLE 7

| | | All plasma | >=1% TFx |
|---|---|---|---|
| | HRD+ | 13 | 10 |
| | HRD− | 12 | 6 |
| DirectHRD | PPV | 10/10 (100%) | 9/9 (100%) |
| | NPV | 12/15 (80%) | 6/7 (86%) |
| | Sens. | 10/13 (77%) | 9/10 (90%) |
| | Spec. | 12/12 (100%) | 6/6 (100%) |
| CHORD | PPV | 1/1 (100%) | 1/1 (100%) |
| | NPV | 12/24 (50%) | 6/15 (40%) |
| | Sens. | 1/13 (8%) | 1/10 (10%) |
| | Spec. | 12/12 (100%) | 6/6 (100%) |

TABLE 8

| | | All plasma | >=1% TFx |
|---|---|---|---|
| | HRD+ | 16 | 9 |
| | HRD− | 7 | 3 |
| DirectHRD | PPV | 10/10 (100%) | 8/8 (100%) |
| | NPV | 7/13 (54%) | 3/4 (75%) |
| | Sens. | 10/16 (63%) | 8/9 (88%) |
| | Spec. | 7/7 (100%) | 3/3 (100%) |
| CHORD | PPV | 0/0 (NA) | 0/0 (NA) |
| | NPV | 7/23 (30%) | 3/12 (25%) |
| | Sens. | 0/16 (0%) | 0/9 (0%) |
| | Spec. | 7/7 (100%) | 3/3 (100%) |

TABLE 9
List of Indel Signatures − HRD positive = “HRD
positive from 27 PCAWG breast cancers”

| Indel type | ID6 | ID8 | HRD negative | HRD positive |
|---|---|---|---|---|
| 1:Del:C:0 | 0.0305068 | 0.03982756 | 0.05716624 | 0.04927042 |
| 1:Del:C:1 | 0.0054919 | 0.02391594 | 0.04407295 | 0.0234982 |
| 1:Del:C:2 | 0.00803399 | 0.00333055 | 0.02314707 | 0.01497063 |
| 1:Del:C:3 | 0.00110238 | 0.00540315 | 0.01087211 | 0.00505338 |
| 1:Del:C:4 | 0.00247817 | 0.00127571 | 0.00619593 | 0.00372687 |
| 1:Del:C:5 | 0.00132207 | 0.00093373 | 0.00455927 | 0.00183185 |
| 1:Del:T:0 | 0.00428416 | 0.03424369 | 0.04138415 | 0.02545638 |
| 1:Del:T:1 | 0.00067769 | 0.01045208 | 0.03261632 | 0.01294928 |
| 1:Del:T:2 | 0.01165967 | 0.00899203 | 0.03507131 | 0.0231192 |
| 1:Del:T:3 | 0.02224239 | 0.00982505 | 0.02595277 | 0.02476154 |
| 1:Del:T:4 | 0.01769714 | 0.00127378 | 0.02829086 | 0.02071884 |
| 1:Del:T:5 | 0.00795435 | 0.01062034 | 0.02291326 | 0.01061209 |
| 1:Ins:C:0 | 0.00208689 | 0.00276427 | 0.00385784 | 0.00309519 |
| 1:Ins:C:1 | 0.00177343 | 0.00216912 | 0.00748188 | 0.00277936 |
| 1:Ins:C:2 | 0.00024617 | 0.001527 | 0.00385784 | 0.00183185 |
| 1:Ins:C:3 | 0.00023217 | 0.00011652 | 0.00245499 | 0.00094751 |
| 1:Ins:C:4 | 0.00037659 | 0.00055418 | 0.00303951 | 0.00101068 |
| 1:Ins:C:5 | 0.000248 | 0.00048949 | 0.00981997 | 0.00107384 |
| 1:Ins:T:0 | 0.00639625 | 0.00450798 | 0.00561141 | 0.00562188 |
| 1:Ins:T:1 | 0.00104119 | 0.0048687 | 0.02782324 | 0.00871707 |
| 1:Ins:T:2 | 0.00073885 | 0.00142964 | 0.01379472 | 0.0040427 |
| 1:Ins:T:3 | 0.00093064 | 0.00108729 | 0.01227496 | 0.0040427 |
| 1:Ins:T:4 | 0.00134985 | 0.00082262 | 0.01192425 | 0.00334786 |
| 1:Ins:T:5 | 0.01439004 | 0.02016125 | 0.11187748 | 0.01825532 |
| 2:Del:R:0 | 0.00178365 | 0.00876922 | 0.02197802 | 0.00821174 |
| 2:Del:R:1 | 0.00245435 | 0.01030715 | 0.03378536 | 0.00972775 |
| 2:Del:R:2 | 0.0020886 | 0.00053203 | 0.00981997 | 0.00322153 |
| 2:Del:R:3 | 0.00052575 | 2.71E−05 | 0.00245499 | 0.00075801 |
| 2:Del:R:4 | 0.00023253 | 0.00027403 | 0 | 0 |
| 2:Del:R:5 | 0.00021569 | 0.00083308 | 0 | 0 |
| 3:Del:R:0 | 0.00105105 | 0.0109641 | 0.01040449 | 0.00429537 |
| 3:Del:R:1 | 0.00541365 | 0.00134887 | 0.02642039 | 0.01086476 |
| 3:Del:R:2 | 0.00053605 | 0.00037657 | 0.00642974 | 0.00113701 |
| 3:Del:R:3 | 4.70E−05 | 0.00025351 | 0.00093524 | 0.00025267 |
| 3:Del:R:4 | 7.31E−05 | 2.68E−06 | 0 | 0 |
| 3:Del:R:5 | 0.00015062 | 9.10E−05 | 0 | 0 |
| 4:Del:R:0 | 0.0023151 | 0.00669131 | 0.00818331 | 0.00480071 |
| 4:Del:R:1 | 0.00280673 | 0.00162296 | 0.02186112 | 0.0078959 |
| 4:Del:R:2 | 0.00047449 | 0.00027262 | 0.00303951 | 0.00101068 |
| 4:Del:R:3 | 0.00029135 | 2.88E−05 | 0.00058452 | 0.00031584 |
| 4:Del:R:4 | 0.00011058 | 9.73E−05 | 0 | 0 |
| 4:Del:R:5 | 5.68E−05 | 6.34E−06 | 0 | 0 |
| 5:Del:R:0 | 0.01933329 | 0.23797886 | 0.0508534 | 0.07194745 |
| 5:Del:R:1 | 0.00750183 | 0.00057226 | 0.00783259 | 0.00429537 |
| 5:Del:R:2 | 0.00037052 | 0.00051684 | 0.0001169 | 6.32E−05 |
| 5:Del:R:3 | 0.00016818 | 8.16E−06 | 0 | 0 |
| 5:Del:R:4 | 5.32E−05 | 0.0001129 | 0 | 0 |
| 5:Del:R:5 | 1.15E−05 | 4.43E−05 | 0 | 0 |
| 2:Ins:R:0 | 0.00199839 | 0.00191671 | 0.00303951 | 0.00208452 |
| 2:Ins:R:1 | 0.00020853 | 0.00166182 | 0.00549451 | 0.00164235 |
| 2:Ins:R:2 | 0.00079041 | 0.00030459 | 0.00292261 | 0.00138968 |
| 2:Ins:R:3 | 0.00049907 | 0.00017758 | 0.00093524 | 0.0001895 |
| 2:Ins:R:4 | 0.00018947 | 1.66E−05 | 0.00140285 | 0.000379 |
| 2:Ins:R:5 | 0.00074864 | 0.00176438 | 0 | 0 |
| 3:Ins:R:0 | 0.00121919 | 0.00066786 | 0.00151976 | 0.00113701 |
| 3:Ins:R:1 | 0.00028862 | 0.00067172 | 0.00666355 | 0.00145285 |
| 3:Ins:R:2 | 0.00074653 | 0.00013577 | 0.00081833 | 0.00044217 |
| 3:Ins:R:3 | 0.00024974 | 3.79E−05 | 0.0001169 | 0.00025267 |
| 3:Ins:R:4 | 0.00015595 | 0.00012204 | 0.0001169 | 0 |
| 3:Ins:R:5 | 1.98E−05 | 3.66E−05 | 0 | 0 |
| 4:Ins:R:0 | 0.00146792 | 0.00077776 | 0.00128595 | 0.00132651 |
| 4:Ins:R:1 | 0.00047628 | 0.0011039 | 0.00549451 | 0.00132651 |
| 4:Ins:R:2 | 0.00052866 | 7.99E−05 | 0.00046762 | 0.00031584 |
| 4:Ins:R:3 | 0.00033482 | 8.58E−06 | 0.0001169 | 0.00025267 |
| 4:Ins:R:4 | 5.60E−05 | 0.00020552 | 0.0001169 | 0 |
| 4:Ins:R:5 | 0.00010686 | 0.00088523 | 0 | 0 |
| 5:Ins:R:0 | 0.00871678 | 0.00031833 | 0.0026888 | 0.00637989 |
| 5:Ins:R:1 | 0.00540564 | 0.00976111 | 0.01823708 | 0.01301244 |
| 5:Ins:R:2 | 0.00132184 | 0.00010115 | 0.00023381 | 0.00101068 |
| 5:Ins:R:3 | 2.88E−05 | 7.09E−05 | 0 | 6.32E−05 |
| 5:Ins:R:4 | 4.83E−05 | 6.74E−06 | 0 | 0 |
| 5:Ins:R:5 | 6.17E−05 | 0.00019055 | 0 | 0 |
| 2:Del:M:1 | 0.00063287 | 0.00491367 | 0.02922609 | 0.0078959 |
| 3:Del:M:1 | 0.0009901 | 0.00929034 | 0.00865092 | 0.00511654 |
| 3:Del:M:2 | 0.00443363 | 0.00430269 | 0.01660042 | 0.0077064 |
| 4:Del:M:1 | 0.0027454 | 0.00580955 | 0.00678045 | 0.00492704 |
| 4:Del:M:2 | 0.00868271 | 0.00159159 | 0.01262567 | 0.00783273 |
| 4:Del:M:3 | 0.00477309 | 0.00109834 | 0.01730185 | 0.00625355 |
| 5:Del:M:1 | 0.07106658 | 0.28072426 | 0.03869535 | 0.10252037 |
| 5:Del:M:2 | 0.25310835 | 0.12361016 | 0.02922609 | 0.16947761 |
| 5:Del:M:3 | 0.21680603 | 0.04613041 | 0.02115969 | 0.13050344 |
| 5:Del:M:4 | 0.12048394 | 0.01636726 | 0.01110592 | 0.07257912 |
| 5:Del:M:5 | 0.099283 | 0.01281488 | 0.00619593 | 0.05299728 |

In Table 9, indel types are coded as follows: the first number indicates the deletion or insertion length, the following three letters indicate whether there is an insertion or deletion, the single letter indicates the context of the insertion or deletion, where C=a region containing C repeats, T=a region containing T repeats, R=a region not proximal to microhomology or repeats, and M=a region having microhomology, and the final number indicates the length of the repeat or homology (e.g., 5:Del:M:4 indicates a 5 bp deletion in a region proximal to a 4 bp section of microhomology).

TABLE 10

| | | DirectHRD_HRDre- | | | | CHORD_BRCA_sta- | |
|---|---|---|---|---|---|---|---|
| sample | DirectHRD_HRDscore | sult | ID6_prob | mhdels | group | tus | CHORD_p_BRCA1 |
| f221cbb5-eefa- | 54.45 | HRD+ | 0.5704139 | 136 | PCAWG | BRCA1 | 0.812 |
| 187f-e040- | | | | | | | |
| 11ac0c481708 | | | | | | | |
| f393ba16-9361- | 0 | HRD− | 3.14E−11 | 17 | PCAWG | none | 0 |
| 5df4-e040- | | | | | | | |
| 11ac0d4844e8 | | | | | | | |
| f393baf9-2710- | 55.31 | HRD+ | 0.4908542 | 190 | PCAWG | BRCA1 | 0.876 |
| 9203-e040- | | | | | | | |
| 11ac0d484504 | | | | | | | |
| f393bafd-1baa- | 213.68 | HRD+ | 0.8105175 | 355 | PCAWG | BRCA2 | 0.162 |
| e5f4-e040- | | | | | | | |
| 11ac0d48450b | | | | | | | |
| f393bafe-7503- | 371.29 | HRD+ | 0.8002512 | 591 | PCAWG | BRCA2 | 0.156 |
| 5c45-e040- | | | | | | | |
| 11ac0d484511 | | | | | | | |
| f393bb01-6ed7- | 35.67 | HRD+ | 0.4334909 | 154 | PCAWG | BRCA1 | 0.774 |
| 9533-e040- | | | | | | | |
| 11ac0d484521 | | | | | | | |
| f393bb05-53c2- | 0 | HRD− | 0.0044433 | 24 | PCAWG | none | 0 |
| f80a-e040- | | | | | | | |
| 11ac0d484528 | | | | | | | |
| f393bb05-c737- | 0.01 | HRD− | 0.0347355 | 9 | PCAWG | none | 0 |
| 4cc3-e040- | | | | | | | |
| 11ac0d48452a | | | | | | | |
| f393bb05-ec1c- | 0.21 | HRD− | 0.0593968 | 63 | PCAWG | none | 0 |
| 17be-e040- | | | | | | | |
| 11ac0d48452f | | | | | | | |
| f393bb07-270c- | 235.53 | HRD+ | 0.8304964 | 371 | PCAWG | BRCA2 | 0.06 |
| 2c93-e040- | | | | | | | |
| 11ac0d484533 | | | | | | | |
| f393bb08-4121- | 317.88 | HRD+ | 0.7893509 | 541 | PCAWG | BRCA2 | 0.112 |
| cad8-e040- | | | | | | | |
| 11ac0d484535 | | | | | | | |
| f7b847e5-0f5d- | 0 | HRD− | 0.0069498 | 20 | PCAWG | none | 0 |
| 69b4-e040- | | | | | | | |
| 11ac0c4845fe | | | | | | | |
| f7b84c09-15d4- | 0.01 | HRD− | 0.0290735 | 18 | PCAWG | none | 0 |
| 3046-e040- | | | | | | | |
| 11ac0c4847ff | | | | | | | |
| f7c6486a-bb08- | 0.45 | HRD− | 0.1567816 | 23 | PCAWG | none | 0.132 |
| f77a-e040- | | | | | | | |
| 11ac0c482953 | | | | | | | |
| f7c6a336-3126- | 0.58 | HRD− | 0.1556679 | 21 | PCAWG | none | 0 |
| 7580-e040- | | | | | | | |
| 11ac0c485971 | | | | | | | |
| f7d77a03-28a0- | 0 | HRD− | 0.0139893 | 23 | PCAWG | none | 0 |
| f446-e040- | | | | | | | |
| 11ac0c4866fb | | | | | | | |
| f7d7b3db-02af- | 0 | HRD− | 1.02E−08 | 25 | PCAWG | none | 0.008 |
| 61cb-e040- | | | | | | | |
| 11ac0c480c03 | | | | | | | |
| f7e4a2a8-093d- | 129.52 | HRD+ | 0.7319971 | 245 | PCAWG | BRCA2 | 0.13 |
| 9e36-e040- | | | | | | | |
| 11ac0d480c7d | | | | | | | |
| f7e7d61f-e2dc- | 0 | HRD− | 1.13E−05 | 120 | PCAWG | none | 0 |
| b523-e040- | | | | | | | |
| 11ac0c482000 | | | | | | | |
| f7ed28a6-8f5f- | 0 | HRD− | 6.35E−04 | 15 | PCAWG | none | 0 |
| e87b-e040- | | | | | | | |
| 11ac0c48120e | | | | | | | |
| f7f66114-e321- | 0 | HRD− | 0.0113674 | 17 | PCAWG | none | 0 |
| 1d4c-e040- | | | | | | | |
| 11ac0d48541a | | | | | | | |
| f7f6619b-fae8- | 0 | HRD− | 0.0076058 | 16 | PCAWG | none | 0 |
| 3381-e040- | | | | | | | |
| 11ac0c483f78 | | | | | | | |
| f80293b8-ddb7- | 0.11 | HRD− | 0.0748822 | 19 | PCAWG | none | 0 |
| f575-e040- | | | | | | | |
| 11ac0d48309d | | | | | | | |
| fc447d51-cdc2- | 0.76 | HRD− | 0.1046561 | 56 | PCAWG | none | 0 |
| a180-e040- | | | | | | | |
| 11ac0c4846a4 | | | | | | | |
| fc8130df-18fe- | 0.47 | HRD− | 0.1596304 | 20 | PCAWG | none | 0 |
| c74d-e040- | | | | | | | |
| 11ac0d485df2 | | | | | | | |
| fc8130df-1cdd- | 0.11 | HRD− | 0.0704225 | 22 | PCAWG | none | 0.042 |
| cade-e040- | | | | | | | |
| 11ac0d485dec | | | | | | | |
| fc8130df-1f21- | 0 | HRD− | 2.54E−06 | 32 | PCAWG | none | 0.014 |
| c8f9-e040- | | | | | | | |
| 11ac0d485dfc | | | | | | | |
| fc8130df-2211- | 147.41 | HRD+ | 0.73547 | 263 | PCAWG | BRCA2 | 0.246 |
| cfcc-e040- | | | | | | | |
| 11ac0d485dde | | | | | | | |
| fc8130df-2628- | 1.74 | HRD+ | 0.2583596 | 25 | PCAWG | none | 0 |
| 304a-e040- | | | | | | | |
| 11ac0d485dfa | | | | | | | |
| fc8130df-2ca4- | 0.08 | HRD− | 0.0727255 | 15 | PCAWG | none | 0 |
| 39d2-e040- | | | | | | | |
| 11ac0d485e00 | | | | | | | |
| fc8130df-2e39- | 0 | HRD− | 1.76E−11 | 25 | PCAWG | none | 0 |
| 3814-e040- | | | | | | | |
| 11ac0d485de0 | | | | | | | |
| fc8130df-30a5- | 214.69 | HRD+ | 0.7530784 | 397 | PCAWG | BRCA2 | 0.186 |
| 3e6a-e040- | | | | | | | |
| 11ac0d485de2 | | | | | | | |
| fc8130df-3147- | 0.04 | HRD− | 0.0452784 | 23 | PCAWG | none | 0 |
| 3e94-e040- | | | | | | | |
| 11ac0d485df8 | | | | | | | |
| fc8130df-3361- | 0.17 | HRD− | 0.0753533 | 33 | PCAWG | none | 0.008 |
| 3c8a-e040- | | | | | | | |
| 11ac0d485dea | | | | | | | |
| fc8130df-34f8- | 0.13 | HRD− | 0.0704357 | 15 | PCAWG | none | 0 |
| 22c6-e040- | | | | | | | |
| 11ac0d485de8 | | | | | | | |
| fc8130df-35ac- | 10.09 | HRD+ | 0.3479363 | 73 | PCAWG | none | 0.056 |
| 2304-e040- | | | | | | | |
| 11ac0d485dda | | | | | | | |
| fc8130df-6860- | 223.41 | HRD+ | 0.708577 | 464 | PCAWG | BRCA2 | 0.128 |
| 7677-e040- | | | | | | | |
| 11ac0d485ddc | | | | | | | |
| fc8130df-6977- | 0.67 | HRD− | 0.1735713 | 24 | PCAWG | none | 0 |
| 7704-e040- | | | | | | | |
| 11ac0d485de4 | | | | | | | |
| fc8130df-8e4e- | 0 | HRD− | 0.006233 | 32 | PCAWG | none | 0 |
| 586b-e040- | | | | | | | |
| 11ac0d485e08 | | | | | | | |
| fc8130df-90ba- | 343.04 | HRD+ | 0.7993676 | 546 | PCAWG | BRCA2 | 0.164 |
| 5d94-e040- | | | | | | | |
| 11ac0d485e0c | | | | | | | |
| fc8130df-e399- | 0.01 | HRD− | 0.0189083 | 21 | PCAWG | none | 0 |
| e34d-e040- | | | | | | | |
| 11ac0c483279 | | | | | | | |
| fc8130e0-08dd- | 0.47 | HRD− | 0.1305951 | 26 | PCAWG | none | 0 |
| b996-e040- | | | | | | | |
| 11ac0c48327b | | | | | | | |
| fc8130e0-095f- | 0 | HRD− | 0.0042375 | 21 | PCAWG | none | 0 |
| b953-e040- | | | | | | | |
| 11ac0c483287 | | | | | | | |
| fc8130e0-096a- | 0.25 | HRD− | 0.0958898 | 22 | PCAWG | none | 0 |
| b991-e040- | | | | | | | |
| 11ac0c48327d | | | | | | | |
| fc8130e0-09f1- | 0.02 | HRD− | 0.0348102 | 18 | PCAWG | none | 0 |
| b964-e040- | | | | | | | |
| 11ac0c48326e | | | | | | | |
| fc8130e0-0bc1- | 0.01 | HRD− | 0.0123074 | 31 | PCAWG | none | 0 |
| bba7-e040- | | | | | | | |
| 11ac0c483289 | | | | | | | |
| fc8130e0-0bfa- | 0.27 | HRD− | 0.0975575 | 24 | PCAWG | none | 0.002 |
| bba4-e040- | | | | | | | |
| 11ac0c48328d | | | | | | | |
| fc8130e0-0c53- | 0.07 | HRD− | 0.0642481 | 19 | PCAWG | none | 0 |
| b432-e040- | | | | | | | |
| 11ac0c483283 | | | | | | | |
| fc8130e0-0d25- | 0 | HRD− | 0.0020279 | 15 | PCAWG | none | 0.05 |
| b475-e040- | | | | | | | |
| 11ac0c483277 | | | | | | | |
| fc8130e0-0db6- | 0 | HRD− | 0.0055876 | 20 | PCAWG | none | 0 |
| b568-e040- | | | | | | | |
| 11ac0c483281 | | | | | | | |
| fc8130e0-0f1a- | 112.52 | HRD+ | 0.6588418 | 254 | PCAWG | BRCA2 | 0.278 |
| b6eb-e040- | | | | | | | |
| 11ac0c48328f | | | | | | | |
| fc8130e0-a38a- | 220.41 | HRD+ | 0.7356181 | 413 | PCAWG | BRCA2 | 0.13 |
| 23b9-e040- | | | | | | | |
| 11ac0c48327f | | | | | | | |
| fc8130e0-a774- | 0 | HRD− | 4.61E−09 | 40 | PCAWG | none | 0 |
| d834-e040- | | | | | | | |
| 11ac0c483268 | | | | | | | |
| fc8130e0-ad63- | 0 | HRD− | 2.04E−04 | 25 | PCAWG | none | 0 |
| b804-e040- | | | | | | | |
| 11ac0d485e10 | | | | | | | |
| fc8130e0-ad8a- | 0 | HRD− | 4.20E−09 | 31 | PCAWG | none | 0 |
| b832-e040- | | | | | | | |
| 11ac0d485e14 | | | | | | | |
| fc8130e3-01ba- | 306.16 | HRD+ | 0.7950127 | 498 | PCAWG | BRCA2 | 0.134 |
| c740-e040- | | | | | | | |
| 11ac0c483291 | | | | | | | |
| fc8130e5-18b8- | 0 | HRD− | 2.12E−07 | 31 | PCAWG | none | 0 |
| ef38-e040- | | | | | | | |
| 11ac0c483297 | | | | | | | |
| fc81312a-d451- | 0.02 | HRD− | 0.0312673 | 20 | PCAWG | none | 0.002 |
| 38fb-e040- | | | | | | | |
| 11ac0c4832a4 | | | | | | | |
| 2b40a733-7a63- | 31.68 | HRD+ | 0.3938275 | 152 | PCAWG | BRCA1 | 0.868 |
| 4bb8-a953- | | | | | | | |
| 95a4ee28f962 | | | | | | | |
| 42465bbd-289b- | 28.09 | HRD+ | 0.4303881 | 125 | PCAWG | BRCA1 | 0.798 |
| 4e96-98fe- | | | | | | | |
| 76809c5e1520 | | | | | | | |
| 51b25b37-f75c- | 85.21 | HRD+ | 0.635781 | 211 | PCAWG | BRCA1 | 0.872 |
| 4380-a0f6- | | | | | | | |
| 5273e0b7ee33 | | | | | | | |
| 6f981023-4269- | 74.41 | HRD+ | 0.5066232 | 230 | PCAWG | BRCA1 | 0.836 |
| 4e8e-a4ab- | | | | | | | |
| 2c92bb27273c | | | | | | | |
| 8888e808-594b- | 86.77 | HRD+ | 0.4403354 | 320 | PCAWG | BRCA1 | 0.888 |
| 4c76-b2e4- | | | | | | | |
| 62aa56736f7c | | | | | | | |
| 9e6806c4-d331- | 515.26 | HRD+ | 0.7915649 | 849 | PCAWG | BRCA2 | 0.166 |
| 4025-abee- | | | | | | | |
| 2f576e3bf182 | | | | | | | |
| bc9b66f5-fcb8- | 73.92 | HRD+ | 0.4871393 | 242 | PCAWG | BRCA1 | 0.866 |
| 4545-ab2d- | | | | | | | |
| 438bb810edc0 | | | | | | | |
| c2ec7f57-8510- | 294.42 | HRD+ | 0.8080389 | 457 | PCAWG | BRCA2 | 0.242 |
| 4bbf-a2e9- | | | | | | | |
| dbd9ce8dcad1 | | | | | | | |
| d4bf6034-aeae- | 80.24 | HRD+ | 0.4458975 | 315 | PCAWG | BRCA1 | 0.926 |
| 48a6-907b- | | | | | | | |
| 10e2cc904015 | | | | | | | |
| d8c2b4b2-e12b- | 322.42 | HRD+ | 0.5604134 | 828 | PCAWG | BRCA1 | 0.872 |
| 43d2-bafc- | | | | | | | |
| 87b29f027797 | | | | | | | |
| da43386c-47f8- | 170.7 | HRD+ | 0.5387 | 523 | PCAWG | BRCA1 | 0.888 |
| 4e03-b6ca- | | | | | | | |
| 8b94e13792e7 | | | | | | | |
| dbbd54db- | 239.63 | HRD+ | 0.7391147 | 414 | PCAWG | BRCA2 | 0.352 |
| 4470-4df6- | | | | | | | |
| b5be- | | | | | | | |
| 3e175c7133b0 | | | | | | | |
| e84debc4-b47d- | 51.99 | HRD+ | 0.5772328 | 152 | PCAWG | BRCA1 | 0.782 |
| 48ed-a0d0- | | | | | | | |
| 2859f0ebf987 | | | | | | | |
| efec3225-de07- | 95.39 | HRD+ | 0.5959183 | 233 | PCAWG | BRCA1 | 0.798 |
| 4559-9a90- | | | | | | | |
| 95223495cc61 | | | | | | | |
| f1504811-8363- | 0.27 | HRD− | 0.0731946 | 46 | PCAWG | none | 0.02 |
| 41e6-b43c- | | | | | | | |
| 62452b1262d3 | | | | | | | |
| f6189828-eeaa- | 89.38 | HRD+ | 0.6839363 | 177 | PCAWG | BRCA1 | 0.838 |
| 4d21-b163- | | | | | | | |
| 53bf3d47a640 | | | | | | | |
| f988e698-9b34- | 73.53 | HRD+ | 0.4785048 | 236 | PCAWG | BRCA1 | 0.892 |
| 45ce-ba4c- | | | | | | | |
| 74e06e9cae4a | | | | | | | |
| 00154198-023b- | 1.53 | HRD+ | 0.210005 | 30 | PCAWG | none | 0 |
| 5137-ba0c- | | | | | | | |
| c7a612883ccf | | | | | | | |
| 011fb67d-2b38- | 69.12 | HRD+ | 0.7041661 | 147 | PCAWG | BRCA2 | 0.132 |
| 5b36-b967- | | | | | | | |
| 1f8454cbb2aa | | | | | | | |
| 0285dfea-ec3a- | 0 | HRD− | 1.78E−07 | 110 | PCAWG | none | 0 |
| 5900-95d9- | | | | | | | |
| b712f070745a | | | | | | | |
| 044bc566-299a- | 0 | HRD− | 2.47E−08 | 29 | PCAWG | none | 0 |
| 53b6-9919- | | | | | | | |
| 979a7feb4afa | | | | | | | |
| 06f5711b-8457- | 0 | HRD− | 4.97E−04 | 101 | PCAWG | none | 0 |
| 5948-8747- | | | | | | | |
| 4fe6a0e8ad89 | | | | | | | |
| 0708fca7-ec5b- | 0 | HRD− | 1.19E−07 | 81 | PCAWG | none | 0 |
| 5a46-a743- | | | | | | | |
| 701a04106db6 | | | | | | | |
| 07c64c28-5b9d- | 0 | HRD− | 5.04E−14 | 45 | PCAWG | none | 0 |
| 5e96-9de7- | | | | | | | |
| dcfd748b9ecd | | | | | | | |
| 0cb8ce81-8235- | 0 | HRD− | 2.55E−04 | 42 | PCAWG | none | 0 |
| 52f8-a698- | | | | | | | |
| fa4e0c96b39a | | | | | | | |
| 0f58a603-4bfc- | 0 | HRD− | 6.69E−06 | 42 | PCAWG | none | 0 |
| 59bb-b5ff- | | | | | | | |
| a83d02981ba0 | | | | | | | |
| 11068ec5-cab4- | 0.28 | HRD− | 0.0703276 | 50 | PCAWG | none | 0 |
| 541d-93df- | | | | | | | |
| 452ac28c5c6c | | | | | | | |
| 1128ed36-1fd5- | 0 | HRD− | 3.49E−05 | 34 | PCAWG | none | 0 |
| 590a-b615- | | | | | | | |
| 7dd76f7d9e02 | | | | | | | |
| 13cffb6c-f7bc- | 0 | HRD− | 1.00E−05 | 27 | PCAWG | none | 0 |
| 5adc-8179- | | | | | | | |
| 31cc2848bcdb | | | | | | | |
| 144edc49-56d0- | 0 | HRD− | 5.36E−12 | 73 | PCAWG | none | 0 |
| 53ae-a4a0- | | | | | | | |
| aa3d82682ab7 | | | | | | | |
| 167ffc5a-e5f2- | 28.14 | HRD+ | 0.5798242 | 83 | PCAWG | none | 0.306 |
| 50eb-b239- | | | | | | | |
| ae05e04741c7 | | | | | | | |
| 169b915f-747b- | 1.57 | HRD+ | 0.1818 | 51 | PCAWG | none | 0 |
| 5107-bdf0- | | | | | | | |
| dcb2175c69c9 | | | | | | | |
| 16c48c28-e744- | 0.49 | HRD− | 0.1678275 | 20 | PCAWG | none | 0 |
| 5034-9b8d- | | | | | | | |
| af424c2dc1cc | | | | | | | |
| 177fa3ac-eec3- | 0 | HRD− | 2.51E−07 | 55 | PCAWG | none | 0 |
| 5f38-8204- | | | | | | | |
| 3d97b85af187 | | | | | | | |
| 18738c38-a99e- | 0.15 | HRD− | 0.093403 | 14 | PCAWG | none | 0 |
| 53c0-b2e3- | | | | | | | |
| baede8ad5e14 | | | | | | | |
| 19fc59e1-ace5- | 0 | HRD− | 2.97E−16 | 77 | PCAWG | none | 0 |
| 58c5-a825- | | | | | | | |
| 64178a1c065d | | | | | | | |
| 1af15027-de25- | 0 | HRD− | 3.20E−10 | 65 | PCAWG | none | 0 |
| 597a-b45c- | | | | | | | |
| dbb840337b31 | | | | | | | |
| 1b02bdd0- | 0.04 | HRD− | 0.0304114 | 34 | PCAWG | none | 0 |
| 9d26-584a- | | | | | | | |
| 9d4f- | | | | | | | |
| 9a8c3a130686 | | | | | | | |
| 1db88eec-789e- | 0 | HRD− | 2.42E−07 | 95 | PCAWG | none | 0 |
| 5ccc-9e0b- | | | | | | | |
| 0b7897a7a4be | | | | | | | |
| 1e8aeaf2-04c3- | 0 | HRD− | 9.04E−07 | 14 | PCAWG | none | 0 |
| 53a5-8420- | | | | | | | |
| 6c12dbdb6b11 | | | | | | | |
| 1efa395b-642e- | 0 | HRD− | 4.03E−10 | 86 | PCAWG | none | 0 |
| 58a4-8dad- | | | | | | | |
| 647bb54d3f8c | | | | | | | |
| 204daa0f-aa70- | 0 | HRD− | 3.73E−04 | 31 | PCAWG | none | 0 |
| 5173-b974- | | | | | | | |
| c13886ff1e86 | | | | | | | |
| 20a74c97-747a- | 0 | HRD− | 1.75E−08 | 342 | PCAWG | none | 0 |
| 5a48-b217- | | | | | | | |
| f2883996d57d | | | | | | | |
| 20ac8d6c-9b38- | 0 | HRD− | 4.05E−09 | 48 | PCAWG | none | 0 |
| 5bc7-a7f3- | | | | | | | |
| 6c597e764456 | | | | | | | |
| 2644b145-e218- | 0 | HRD− | 1.96E−08 | 75 | PCAWG | none | 0 |
| 5ca6-80ca- | | | | | | | |
| da03350eb140 | | | | | | | |
| 265febb1-200b- | 0.1 | HRD− | 0.040036 | 56 | PCAWG | none | 0 |
| 5417-8424- | | | | | | | |
| 767cea751935 | | | | | | | |
| 27c5beb6-38a8- | 0.17 | HRD− | 0.1004457 | 21 | PCAWG | none | 0 |
| 550c-a500- | | | | | | | |
| 6f3cf4aab9f1 | | | | | | | |
| 2a040e2e-ab16- | 0.08 | HRD− | 0.0405077 | 43 | PCAWG | none | 0 |
| 504c-aae7- | | | | | | | |
| 2a3caf6ea053 | | | | | | | |
| 2a138e2a-293d- | 0.05 | HRD− | 0.0517249 | 21 | PCAWG | none | 0 |
| 502b-8fa5- | | | | | | | |
| b3da1dccfadd | | | | | | | |
| 2d84abcd-0a7e- | 0 | HRD− | 6.43E−08 | 117 | PCAWG | none | 0 |
| 5dc1-84d8- | | | | | | | |
| 0e5184ea3d0d | | | | | | | |
| 2da68bcd-11ef- | 0 | HRD− | 4.39E−06 | 44 | PCAWG | none | 0 |
| 562d-9332- | | | | | | | |
| 77ac387e2d91 | | | | | | | |
| 2e1b33e2-54de- | 568.22 | HRD+ | 0.748316 | 1016 | PCAWG | BRCA2 | 0.102 |
| 5693-ac6c- | | | | | | | |
| d18d5285cf23 | | | | | | | |
| 301db1d1-307e- | 1.69 | HRD+ | 0.1783568 | 49 | PCAWG | none | 0 |
| 5652-9faa- | | | | | | | |
| 4849ccbad997 | | | | | | | |
| 314cecfe-92fe- | 0 | HRD− | 5.05E−08 | 158 | PCAWG | none | 0 |
| 596c-aabd- | | | | | | | |
| 2216b3692b6e | | | | | | | |
| 319995f4-5632- | 117.25 | HRD+ | 0.6758653 | 243 | PCAWG | BRCA2 | 0.092 |
| 5189-83d8- | | | | | | | |
| ba63c797ef28 | | | | | | | |
| 31cada43-4c06- | 0 | HRD− | 5.47E−10 | 62 | PCAWG | none | 0 |
| 5a78-b607- | | | | | | | |
| 87d02a4d886c | | | | | | | |
| 32f4b3db-f057- | 25.09 | HRD+ | 0.5841293 | 74 | PCAWG | none | 0.332 |
| 52c9-9e98- | | | | | | | |
| aac05a825b7f | | | | | | | |
| 32fa55db-71bf- | 137.43 | HRD+ | 0.4663976 | 494 | PCAWG | BRCA1 | 0.882 |
| 5bc9-9bfc- | | | | | | | |
| e00709b16f63 | | | | | | | |
| 33623629-be74- | 0 | HRD− | 1.40E−06 | 52 | PCAWG | none | 0 |
| 5373-8457- | | | | | | | |
| 0e43f805b935 | | | | | | | |
| 349cd0b4-0ab9- | 0 | HRD− | 1.94E−04 | 64 | PCAWG | none | 0 |
| 5786-bac4- | | | | | | | |
| cb95ea6052b1 | | | | | | | |
| 3826a48c-9632- | 0 | HRD− | 0.0022131 | 21 | PCAWG | none | 0 |
| 52ee-8fc7- | | | | | | | |
| befdf0ffdc2a | | | | | | | |
| 388feaa6-bc87- | 0 | HRD− | 5.47E−13 | 96 | PCAWG | none | 0 |
| 5d2e-8acb- | | | | | | | |
| 4fa8cd8adf2b | | | | | | | |
| 3b46813a-e351- | 0.24 | HRD− | 0.0756219 | 38 | PCAWG | none | 0 |
| 5f8d-aa4c- | | | | | | | |
| d08fc4713710 | | | | | | | |
| 3b5ee9fb-cf66- | 0 | HRD− | 5.26E−05 | 71 | PCAWG | none | 0 |
| 5e9d-a4d7- | | | | | | | |
| 0ec7606bc182 | | | | | | | |
| 3cbf1093-8f79- | 0 | HRD− | 1.19E−07 | 84 | PCAWG | none | 0 |
| 59ca-babd- | | | | | | | |
| 325b1e3beb44 | | | | | | | |
| 3ffb6b77-73ec- | 0 | HRD− | 7.22E−05 | 87 | PCAWG | none | 0 |
| 5398-9002- | | | | | | | |
| d28c72dad54c | | | | | | | |
| 4033e0a8-d52c- | 0 | HRD− | 3.37E−11 | 87 | PCAWG | none | 0 |
| 5ad8-b576- | | | | | | | |
| 268068bee8a9 | | | | | | | |
| 4222a30d-e190- | 0 | HRD− | 1.46E−10 | 35 | PCAWG | none | 0 |
| 59b4-93ef- | | | | | | | |
| 0d1b8b68827d | | | | | | | |
| 42c75c6c-eeb1- | 0 | HRD− | 2.23E−08 | 59 | PCAWG | none | 0 |
| 5dc2-8239- | | | | | | | |
| fe5718cea7f0 | | | | | | | |
| 42dad23b-8b78- | 0 | HRD− | 2.50E−06 | 57 | PCAWG | none | 0 |
| 58f8-8027- | | | | | | | |
| 9389a71f05e5 | | | | | | | |
| 43ba96f4-5e7f- | 0 | HRD− | 7.48E−07 | 95 | PCAWG | none | 0 |
| 55b5-bd3c- | | | | | | | |
| a303d625a3f8 | | | | | | | |
| 474668a4-6f29- | 0 | HRD− | 3.20E−11 | 18 | PCAWG | none | 0 |
| 5851-a063- | | | | | | | |
| 2ad4964a6f90 | | | | | | | |
| 47b69a2b-faab- | 0 | HRD− | 0.00714 | 15 | PCAWG | none | 0 |
| 55db-9436- | | | | | | | |
| b4600d5d52ae | | | | | | | |
| 47c173cf-3630- | 0 | HRD− | 1.60E−06 | 86 | PCAWG | none | 0 |
| 5223-b7f6- | | | | | | | |
| 960e8b54f39f | | | | | | | |
| 481e94d1-03f5- | 0 | HRD− | 8.40E−09 | 76 | PCAWG | none | 0 |
| 5c5c-add6- | | | | | | | |
| 7603dfb4da73 | | | | | | | |
| 4d9df4c3-89fb- | 0 | HRD− | 3.32E−05 | 68 | PCAWG | none | 0 |
| 5d80-b5b3- | | | | | | | |
| deae8088abf6 | | | | | | | |
| 501ceb06-d90e- | 0 | HRD− | 6.91E−07 | 55 | PCAWG | none | 0 |
| 5907-8f31- | | | | | | | |
| c28605e90b1a | | | | | | | |
| 52d8403e-7872- | 0 | HRD− | 3.14E−08 | 59 | PCAWG | none | 0 |
| 5e34-8b6f- | | | | | | | |
| 8f767c1457d1 | | | | | | | |
| 5342d185-c30d- | 22.65 | HRD+ | 0.3931374 | 159 | PCAWG | none | 0.21 |
| 5b45-b3dc- | | | | | | | |
| ffd064947bf1 | | | | | | | |
| 55f32d60-b1e0- | 0 | HRD− | 3.52E−12 | 20 | PCAWG | none | 0 |
| 55c6-b5a0- | | | | | | | |
| 263912972c48 | | | | | | | |
| 57be9fdb-fc38- | 0 | HRD− | 1.23E−05 | 169 | PCAWG | none | 0.002 |
| 529d-8aaf- | | | | | | | |
| 46d0b7c75704 | | | | | | | |
| 59dcef8d-513a- | 0 | HRD− | 8.48E−07 | 66 | PCAWG | none | 0 |
| 53a0-b56f- | | | | | | | |
| 5bac2f400ff7 | | | | | | | |
| 5e73036f-d33f- | 0 | HRD− | 1.60E−04 | 31 | PCAWG | none | 0 |
| 58ed-92a4- | | | | | | | |
| abc536e4ee52 | | | | | | | |
| 5f157436-48b7- | 0 | HRD− | 3.35E−04 | 97 | PCAWG | none | 0 |
| 5f3c-8f34- | | | | | | | |
| c17058bff221 | | | | | | | |
| 5fa51650-7759- | 0 | HRD− | 0.0167139 | 11 | PCAWG | none | 0 |
| 50fa-be54- | | | | | | | |
| 11930947454e | | | | | | | |
| 60644314-5e6c- | 0 | HRD− | 9.82E−07 | 77 | PCAWG | none | 0 |
| 57f9-8574- | | | | | | | |
| 8c0c532fda3e | | | | | | | |
| 610efdc3-690f- | 0 | HRD− | 0.0050228 | 12 | PCAWG | none | 0 |
| 5e97-ae9f- | | | | | | | |
| 3fcb9124afa8 | | | | | | | |
| 6278f7b4-2858- | 0 | HRD− | 1.91E−08 | 233 | PCAWG | none | 0 |
| 5e1f-9bce- | | | | | | | |
| 661fafab14af | | | | | | | |
| 62918f7c-470f- | 0.25 | HRD− | 0.2458824 | 11 | PCAWG | none | 0 |
| 5527-bf52- | | | | | | | |
| fa3a6fac9da9 | | | | | | | |
| 63db6f5a-316a- | 0.02 | HRD− | 0.0248391 | 40 | PCAWG | none | 0 |
| 5273-a0f5- | | | | | | | |
| 9c86c8554c2a | | | | | | | |
| 648f8223-ac91- | 0 | HRD− | 3.49E−08 | 150 | PCAWG | none | 0 |
| 5119-bad3- | | | | | | | |
| ab4f28e0ff15 | | | | | | | |
| 652f80ca-72e6- | 0 | HRD− | 1.42E−06 | 1050 | PCAWG | none | 0 |
| 5521-b6ad- | | | | | | | |
| 32aa7e5920e0 | | | | | | | |
| 67a4d799-fe2b- | 0 | HRD− | 1.35E−05 | 29 | PCAWG | none | 0 |
| 570e-b75a- | | | | | | | |
| cd1220947bb9 | | | | | | | |
| 6ab3463c-bf14- | 0 | HRD− | 2.78E−06 | 67 | PCAWG | none | 0 |
| 5c5d-8cd0- | | | | | | | |
| 5926a52d4b31 | | | | | | | |
| 6bec564e-3701- | 0 | HRD− | 1.45E−06 | 73 | PCAWG | none | 0 |
| 58e9-bf1f- | | | | | | | |
| 6b75f870823f | | | | | | | |
| 6db0a33a-a450- | 0 | HRD− | 1.74E−06 | 40 | PCAWG | none | 0 |
| 5da7-81c8- | | | | | | | |
| 43b2a5e73462 | | | | | | | |
| 6e4ed651-a190- | 613.3 | HRD+ | 0.7922489 | 1023 | PCAWG | BRCA2 | 0.11 |
| 5b1a-ade7- | | | | | | | |
| b4a649b72f73 | | | | | | | |
| 6fd1df41-1cca- | 0 | HRD− | 8.57E−06 | 100 | PCAWG | none | 0 |
| 51db-a150- | | | | | | | |
| a146c81482e1 | | | | | | | |
| 70a6e645-1d59- | 0.43 | HRD− | 0.108851 | 37 | PCAWG | none | 0 |
| 535b-b997- | | | | | | | |
| 5018c3543964 | | | | | | | |
| 728fb047-981b- | 0.04 | HRD− | 0.0511705 | 12 | PCAWG | none | 0 |
| 5f9f-ae60- | | | | | | | |
| 4054d70d1aae | | | | | | | |
| 7439ad9d-4560- | 0 | HRD− | 5.73E−08 | 78 | PCAWG | none | 0 |
| 5486-be61- | | | | | | | |
| 36bdbf52c686 | | | | | | | |
| 76e741a7-3812- | 0 | HRD− | 1.86E−05 | 15 | PCAWG | none | 0 |
| 53b1-8351- | | | | | | | |
| a3025378ad58 | | | | | | | |
| 782f9ff6-0fca- | 0.08 | HRD− | 0.0421859 | 41 | PCAWG | none | 0 |
| 5c39-abb1- | | | | | | | |
| f37a32692f4e | | | | | | | |
| 795a44b6-13ab- | 0 | HRD− | 4.00E−10 | 67 | PCAWG | none | 0 |
| 587c-bceb- | | | | | | | |
| e80acbbe6669 | | | | | | | |
| 7bb3a679-d6fa- | 0 | HRD− | 6.58E−04 | 17 | PCAWG | none | 0 |
| 5414-811d- | | | | | | | |
| 9807275ce1bf | | | | | | | |
| 7d9065e3-718b- | 0.01 | HRD− | 0.0063975 | 147 | PCAWG | none | 0 |
| 5b66-b1ac- | | | | | | | |
| 03a11687649e | | | | | | | |
| 7fed2782-c1bc- | 0.07 | HRD− | 0.0359047 | 38 | PCAWG | none | 0 |
| 5990-aab8- | | | | | | | |
| 4b8fb45bb5f4 | | | | | | | |
| 80cbcf40-4e92- | 0 | HRD− | 1.54E−09 | 71 | PCAWG | none | 0 |
| 5b50-a461- | | | | | | | |
| 79babec067c7 | | | | | | | |
| 81c28bfc-36a8- | 0 | HRD− | 5.30E−07 | 79 | PCAWG | none | 0 |
| 550f-a167- | | | | | | | |
| 7ba9c3a16555 | | | | | | | |
| 8241a7ef-ba71- | 0 | HRD− | 1.29E−04 | 25 | PCAWG | none | 0 |
| 5ae8-828d- | | | | | | | |
| c03bf77570ee | | | | | | | |
| 83383700-b21e- | 0 | HRD− | 4.39E−09 | 66 | PCAWG | none | 0 |
| 5f4c-b1b5- | | | | | | | |
| 6ab215cc0707 | | | | | | | |
| 8621c8eb-5c34- | 0 | HRD− | 1.10E−10 | 56 | PCAWG | none | 0 |
| 580e-9bc3- | | | | | | | |
| d88aac77c4d7 | | | | | | | |
| 865eca7a-3dc9- | 0 | HRD− | 4.75E−11 | 56 | PCAWG | none | 0 |
| 5cfd-9b11- | | | | | | | |
| 930afcc4fed7 | | | | | | | |
| 888c800e-d85f- | 0 | HRD− | 0.0042775 | 12 | PCAWG | none | 0 |
| 53e9-9ef3- | | | | | | | |
| 5db944ecf26b | | | | | | | |
| 88c140d5-8880- | 0 | HRD− | 6.89E−06 | 169 | PCAWG | none | 0 |
| 55d1-a322- | | | | | | | |
| 5ef66431a36e | | | | | | | |
| 8b8645ea-0010- | 0 | HRD− | 0.00617 | 37 | PCAWG | none | 0 |
| 5815-8c98- | | | | | | | |
| 95fe322fdcb7 | | | | | | | |
| 8d6de6a5-5bd2- | 0 | HRD− | 6.20E−09 | 123 | PCAWG | none | 0 |
| 5c43-b357- | | | | | | | |
| dca27947818c | | | | | | | |
| 8fb77a13-45d6- | 0 | HRD− | 2.55E−06 | 74 | PCAWG | none | 0 |
| 54b7-ad1a- | | | | | | | |
| e8539ca32022 | | | | | | | |
| 91db5a58-5f0e- | 3.93 | HRD+ | 0.2428072 | 62 | PCAWG | none | 0 |
| 5b80-81a7- | | | | | | | |
| 96408ddbcfcc | | | | | | | |
| 91e7e8ac-8e86- | 0 | HRD− | 3.66E−07 | 83 | PCAWG | none | 0 |
| 5a68-a7bc- | | | | | | | |
| 898f2fba03b7 | | | | | | | |
| 961e114d-5b6a- | 0 | HRD− | 2.44E−05 | 44 | PCAWG | none | 0 |
| 5c51-b069- | | | | | | | |
| 85d3a742d7a7 | | | | | | | |
| 9698d459-01d5- | 0 | HRD− | 2.42E−08 | 141 | PCAWG | none | 0 |
| 5fe6-827c- | | | | | | | |
| d75dfcff5669 | | | | | | | |
| 975669c5-fc96- | 1.02 | HRD+ | 0.1992339 | 25 | PCAWG | none | 0 |
| 56f8-b0d1- | | | | | | | |
| eadc1ff7f0c4 | | | | | | | |
| 9b12ed50-6da8- | 0 | HRD− | 4.43E−12 | 87 | PCAWG | none | 0 |
| 54b7-9dfb- | | | | | | | |
| 621720408ec9 | | | | | | | |
| 9ba249b8-0856- | 0 | HRD− | 4.32E−09 | 43 | PCAWG | none | 0 |
| 520b-a630- | | | | | | | |
| 3fe835c77a30 | | | | | | | |
| 9d70bc61-e795- | 0 | HRD− | 2.77E−06 | 68 | PCAWG | none | 0 |
| 5592-91b8- | | | | | | | |
| 118ea4a8a9c3 | | | | | | | |
| 9f65bfd1-0846- | 0.34 | HRD− | 0.1333911 | 18 | PCAWG | none | 0 |
| 55ef-9043- | | | | | | | |
| 4bfa0bc3fdef | | | | | | | |
| 9f8c2b6f-fd32- | 0.62 | HRD− | 0.1934562 | 16 | PCAWG | none | 0 |
| 5ea3-b11b- | | | | | | | |
| 53df56016e5e | | | | | | | |
| a0659c6a-2682- | 0 | HRD− | 4.89E−06 | 42 | PCAWG | none | 0 |
| 529e-aa70- | | | | | | | |
| f4bc064eebfa | | | | | | | |
| a196e49f-9a45- | 0 | HRD− | 3.82E−08 | 99 | PCAWG | none | 0 |
| 5c77-b706- | | | | | | | |
| 9add0246b114 | | | | | | | |
| a5322930-562f- | 0 | HRD− | 1.12E−07 | 91 | PCAWG | none | 0 |
| 5ed1-b40f- | | | | | | | |
| 1f19f19cd152 | | | | | | | |
| a60c4297-856f- | 0 | HRD− | 6.17E−09 | 1296 | PCAWG | none | 0 |
| 5f54-85fd- | | | | | | | |
| d11293f67b49 | | | | | | | |
| a8f075c0-ba1d- | 0.45 | HRD− | 0.1565583 | 21 | PCAWG | none | 0 |
| 5e19-bb54- | | | | | | | |
| 26e5d2b27a91 | | | | | | | |
| aa16ed58-a498- | 0 | HRD− | 1.50E−06 | 89 | PCAWG | none | 0 |
| 5c83-a337- | | | | | | | |
| 39d082940e0a | | | | | | | |
| aba5a504-6840- | 0 | HRD− | 7.21E−06 | 171 | PCAWG | none | 0 |
| 5238-b628- | | | | | | | |
| 9b9ee4e96a14 | | | | | | | |
| ad2092d2-a673- | 0 | HRD− | 0.0014703 | 117 | PCAWG | none | 0 |
| 5382-8af4- | | | | | | | |
| d7fbe48e1678 | | | | | | | |
| ad7b63f1-7a45- | 8.87 | HRD+ | 0.3696733 | 64 | PCAWG | none | 0 |
| 5f71-9a42- | | | | | | | |
| 9bb426cb36bb | | | | | | | |
| b0d8663e-5726- | 0 | HRD− | 0.0015998 | 19 | PCAWG | none | 0 |
| 5f3b-9222- | | | | | | | |
| 3735b61fb463 | | | | | | | |
| b2101e56-fde5- | 0 | HRD− | 1.20E−06 | 99 | PCAWG | none | 0 |
| 5066-a95b- | | | | | | | |
| 848cbf926953 | | | | | | | |
| b2d2601e-e73d- | 0 | HRD− | 2.52E−08 | 72 | PCAWG | none | 0 |
| 586b-8ef1- | | | | | | | |
| cba156697c36 | | | | | | | |
| b44cfc59-cce5- | 0 | HRD− | 0.0120585 | 22 | PCAWG | none | 0 |
| 5b5b-aa26- | | | | | | | |
| 3348ff4a072f | | | | | | | |
| b74cebec-2639- | 0 | HRD− | 1.35E−06 | 89 | PCAWG | none | 0 |
| 55eb-9555- | | | | | | | |
| 03b758fbb352 | | | | | | | |
| ba225dda-f0e3- | 0 | HRD− | 6.30E−08 | 42 | PCAWG | none | 0 |
| 57ea-9d24- | | | | | | | |
| eb407e0f39ef | | | | | | | |
| bb889c67-42c7- | 0 | HRD− | 4.09E−08 | 77 | PCAWG | none | 0 |
| 5d52-8c7c- | | | | | | | |
| b46cbb2b7f50 | | | | | | | |
| bbd548b0-9da5- | 0.07 | HRD− | 0.0156956 | 157 | PCAWG | none | 0 |
| 58fc-ae51- | | | | | | | |
| aa3387ce4223 | | | | | | | |
| bc3f7d54-152e- | 0 | HRD− | 2.22E−09 | 96 | PCAWG | none | 0 |
| 545a-b32d- | | | | | | | |
| 169e744e4a90 | | | | | | | |
| c0878092-ee77- | 0 | HRD− | 4.35E−06 | 27 | PCAWG | none | 0 |
| 54ee-ba80- | | | | | | | |
| 07035536ebfa | | | | | | | |
| c0f83a8b-0027- | 0 | HRD− | 3.02E−04 | 170 | PCAWG | none | 0 |
| 5130-ad6f- | | | | | | | |
| 52e1b296176b | | | | | | | |
| c1b0f8bd-b2ad- | 0 | HRD− | 4.08E−08 | 145 | PCAWG | none | 0 |
| 5d03-84cd- | | | | | | | |
| 942971bf193a | | | | | | | |
| c24a35b4-f507- | 0 | HRD− | 1.33E−05 | 52 | PCAWG | none | 0 |
| 5aa3-a4e3- | | | | | | | |
| dffb6a4a75f2 | | | | | | | |
| c2ec5d59-5ead- | 0 | HRD− | 1.66E−13 | 56 | PCAWG | none | 0 |
| 5792-aacb- | | | | | | | |
| 80078ceff799 | | | | | | | |
| c4002b91-2b56- | 106.59 | HRD+ | 0.418349 | 481 | PCAWG | BRCA1 | 0.882 |
| 5368-aed2- | | | | | | | |
| 2b792f4dbee7 | | | | | | | |
| c4f6cca8-a8eb- | 0 | HRD− | 7.53E−10 | 90 | PCAWG | none | 0 |
| 5e9d-a9e7- | | | | | | | |
| 99d26e6ae67a | | | | | | | |
| c64376d2-bdfc- | 0.1 | HRD− | 0.0644932 | 25 | PCAWG | none | 0 |
| 540b-930a- | | | | | | | |
| 8b7d510997bf | | | | | | | |
| c67c5149-2753- | 0 | HRD− | 1.14E−06 | 43 | PCAWG | none | 0 |
| 5175-9e2d- | | | | | | | |
| 13d1290323ac | | | | | | | |
| c781dd37-aba7- | 0 | HRD− | 5.78E−06 | 24 | PCAWG | none | 0 |
| 5427-b267- | | | | | | | |
| decc93da4df8 | | | | | | | |
| c9476a45-6c52- | 0 | HRD− | 2.50E−12 | 44 | PCAWG | none | 0 |
| 5abe-a959- | | | | | | | |
| 7713f1f1126b | | | | | | | |
| cad23c3f-f06a- | 0 | HRD− | 5.05E−06 | 18 | PCAWG | none | 0 |
| 5d55-9dcf- | | | | | | | |
| bbeec8764372 | | | | | | | |
| cb80f145-46f5- | 0.84 | HRD− | 0.1872936 | 25 | PCAWG | none | 0 |
| 5c3b-9842- | | | | | | | |
| 8686daf34306 | | | | | | | |
| cbb4e48c-4bd0- | 1.11 | HRD+ | 0.1349387 | 54 | PCAWG | none | 0 |
| 58e9-b132- | | | | | | | |
| cc6c2b9cb9c5 | | | | | | | |
| cecea6c7-ec8c- | 0 | HRD− | 1.39E−12 | 40 | PCAWG | none | 0 |
| 5d66-965c- | | | | | | | |
| 82a5df8d6a95 | | | | | | | |
| cefd90ea-eda5- | 0 | HRD− | 0.0033948 | 45 | PCAWG | none | 0 |
| 55a8-83eb- | | | | | | | |
| 0a66aa5c65a7 | | | | | | | |
| cf046015-3f38- | 0.02 | HRD− | 0.0458506 | 12 | PCAWG | none | 0 |
| 58ce-811b- | | | | | | | |
| 1044fc0c5f2c | | | | | | | |
| d0f782c5-3615- | 0 | HRD− | 7.83E−08 | 75 | PCAWG | none | 0 |
| 5cc4-9ac4- | | | | | | | |
| 6bafc93d5caf | | | | | | | |
| d287c6a6-b6e1- | 0 | HRD− | 7.40E−10 | 37 | PCAWG | none | 0 |
| 5c30-b2cf- | | | | | | | |
| 0bd7c993af0c | | | | | | | |
| d413b834-09c8- | 0 | HRD− | 1.61E−10 | 79 | PCAWG | none | 0 |
| 5c76-a0fe- | | | | | | | |
| d55b67fdfe00 | | | | | | | |
| d440c1f6-264a- | 0 | HRD− | 1.87E−13 | 45 | PCAWG | none | 0 |
| 51fa-be0e- | | | | | | | |
| 0145bbae6710 | | | | | | | |
| d6002f62-30b6- | 0 | HRD− | 8.48E−07 | 104 | PCAWG | none | 0 |
| 576a-89b9- | | | | | | | |
| 78e4fd50c7b4 | | | | | | | |
| d744a1ee-a212- | 0 | HRD− | 4.17E−06 | 81 | PCAWG | none | 0 |
| 5229-9d50- | | | | | | | |
| bb01c4256f76 | | | | | | | |
| d873e58c-2c42- | 24.49 | HRD+ | 0.5546172 | 82 | PCAWG | none | 0.338 |
| 5bfa-aea2- | | | | | | | |
| 79ab6790914d | | | | | | | |
| daec4402-3b95- | 0 | HRD− | 1.30E−12 | 98 | PCAWG | none | 0 |
| 5229-82bf- | | | | | | | |
| 2274155f6d7c | | | | | | | |
| dc2b7124-5b28- | 640.69 | HRD+ | 0.7645879 | 1113 | PCAWG | BRCA2 | 0.122 |
| 547f-b37a- | | | | | | | |
| d7ae37082cec | | | | | | | |
| dc334f20-ce24- | 0 | HRD− | 3.77E−05 | 18 | PCAWG | none | 0 |
| 5c08-b189- | | | | | | | |
| b03fcd5361ea | | | | | | | |
| dfb3ea4b-2ac7- | 0 | HRD− | 6.89E−07 | 29 | PCAWG | none | 0 |
| 52a3-b5a5- | | | | | | | |
| 5910b0a1a6e1 | | | | | | | |
| dfe3a3b4-9c46- | 0 | HRD− | 1.06E−09 | 1219 | PCAWG | none | 0.054 |
| 5a7a-95a6- | | | | | | | |
| 648362aa75ba | | | | | | | |
| e16dab0b-dc33- | 0 | HRD− | 2.92E−07 | 58 | PCAWG | none | 0 |
| 5df4-8752- | | | | | | | |
| dd3a1b70a5b3 | | | | | | | |
| e3e8cf24-e8be- | 0 | HRD− | 2.58E−09 | 18 | PCAWG | none | 0 |
| 5540-924e- | | | | | | | |
| a38d2fa80704 | | | | | | | |
| e468b4a3-9cc0- | 0 | HRD− | 7.87E−06 | 164 | PCAWG | none | 0 |
| 5339-a178- | | | | | | | |
| ece248a3d047 | | | | | | | |
| e5bf367d-d70c- | 25.71 | HRD+ | 0.6205903 | 69 | PCAWG | none | 0.31 |
| 5dd4-8b7f- | | | | | | | |
| 17f5bb27e4f6 | | | | | | | |
| e62a1fc5-1f58- | 0 | HRD− | 2.25E−06 | 54 | PCAWG | none | 0 |
| 5d80-a5aa- | | | | | | | |
| ef80a9d2200c | | | | | | | |
| e76d10a0-fa72- | 0 | HRD− | 2.04E−07 | 58 | PCAWG | none | 0 |
| 5f0a-9fa3- | | | | | | | |
| 8691ce475510 | | | | | | | |
| e8593980-a873- | 0 | HRD− | 6.29E−06 | 16 | PCAWG | none | 0 |
| 5c72-b5ae- | | | | | | | |
| 8deae82d4fe9 | | | | | | | |
| e89c60a0-a344- | 0 | HRD− | 6.40E−19 | 48 | PCAWG | none | 0 |
| 55f1-82f4- | | | | | | | |
| ce61a6611c76 | | | | | | | |
| e8a6cc14-b310- | 0.02 | HRD− | 0.0375346 | 19 | PCAWG | none | 0 |
| 5398-9cb9- | | | | | | | |
| b5b6d3ec45c5 | | | | | | | |
| e9dc71bf-577b- | 0 | HRD− | 5.73E−10 | 89 | PCAWG | none | 0 |
| 57f1-96c2- | | | | | | | |
| 43ffcf3c2444 | | | | | | | |
| ea320e35-11be- | 0.07 | HRD− | 0.0702631 | 18 | PCAWG | none | 0 |
| 5231-881b- | | | | | | | |
| b4e283789d30 | | | | | | | |
| eb233756-a262- | 0 | HRD− | 7.22E−13 | 41 | PCAWG | none | 0 |
| 5fde-bd00- | | | | | | | |
| 83f3aed1fb25 | | | | | | | |
| ed01c279-2d23- | 0 | HRD− | 3.16E−06 | 22 | PCAWG | none | 0 |
| 5d92-9680- | | | | | | | |
| d61a4ceb0ff3 | | | | | | | |
| ed969e61-0ff7- | 0 | HRD− | 1.39E−04 | 95 | PCAWG | none | 0 |
| 59a6-9e14- | | | | | | | |
| 4f0dfaa10be5 | | | | | | | |
| eeee9982-61f2- | 0 | HRD− | 0.0144855 | 24 | PCAWG | none | 0.01 |
| 515c-be99- | | | | | | | |
| 85680830d48c | | | | | | | |
| f3d3ca98-020a- | 0 | HRD− | 3.01E−12 | 29 | PCAWG | none | 0 |
| 5115-922c- | | | | | | | |
| edcad9fcf143 | | | | | | | |
| f476071d-ca26- | 0 | HRD− | 1.67E−06 | 81 | PCAWG | none | 0 |
| 51dc-87f7- | | | | | | | |
| d5962bb3f66d | | | | | | | |
| f63896f2-21bb- | 0 | HRD− | 4.47E−05 | 70 | PCAWG | none | 0 |
| 5259-bb3b- | | | | | | | |
| a38963b3d2a9 | | | | | | | |
| f712af1a-496f- | 0 | HRD− | 7.71E−07 | 91 | PCAWG | none | 0 |
| 5264-b787- | | | | | | | |
| 65eb441855b9 | | | | | | | |
| fb4faccb-cbcd- | 0 | HRD− | 3.24E−07 | 130 | PCAWG | none | 0 |
| 5706-bb5a- | | | | | | | |
| a8180c7f22bd | | | | | | | |
| fc677c26-ac56- | 0 | HRD− | 3.63E−08 | 42 | PCAWG | none | 0 |
| 5522-ba62- | | | | | | | |
| 7b86072c8703 | | | | | | | |
| fd22f0c2-d50f- | 0 | HRD− | 5.34E−08 | 100 | PCAWG | none | 0 |
| 551c-9b11- | | | | | | | |
| 5496df8a56ef | | | | | | | |
| fd246758-136b- | 97.63 | HRD+ | 0.4142586 | 435 | PCAWG | BRCA1 | 0.888 |
| 5783-8377- | | | | | | | |
| e8760dff9a6a | | | | | | | |
| fed6c599-7e8a- | 0.05 | HRD− | 0.0401749 | 32 | PCAWG | none | 0 |
| 52b4-a9b0- | | | | | | | |
| 12aa1044559f | | | | | | | |
| ffd69c4f-7134- | 1.55 | HRD+ | 0.1613814 | 54 | PCAWG | none | 0 |
| 55cd-95a2- | | | | | | | |
| 30d5e6d5d84e | | | | | | | |
| 00213c2e-10b7- | 0 | HRD− | 3.52E−04 | 18 | PCAWG | none | 0 |
| 508a-a4cf- | | | | | | | |
| 98868df04abb | | | | | | | |
| 004e8577-7340- | 0 | HRD− | 0.0045705 | 23 | PCAWG | none | 0 |
| 5c55-8381- | | | | | | | |
| 41d90a40fed2 | | | | | | | |
| 00b81c0d-198c- | 0 | HRD− | 2.96E−10 | 46 | PCAWG | none | 0 |
| 59f7-b492- | | | | | | | |
| 64825c916dad | | | | | | | |
| 01083a3c-2715- | 0 | HRD− | 1.23E−06 | 153 | PCAWG | none | 0 |
| 54f0-99d6- | | | | | | | |
| 7385f0824189 | | | | | | | |
| 02025ab4-4962- | 0 | HRD− | 1.51E−06 | 308 | PCAWG | none | 0 |
| 5284-8d06- | | | | | | | |
| cf913d035734 | | | | | | | |
| 021639d8-e5de- | 0 | HRD− | 3.11E−06 | 19 | PCAWG | none | 0 |
| 596e-82f0- | | | | | | | |
| fe89dfb09747 | | | | | | | |
| 026f6cfc-12a2- | 0.01 | HRD− | 0.009105 | 127 | PCAWG | none | 0 |
| 545a-a4e0- | | | | | | | |
| ec865462a296 | | | | | | | |
| 02f891ae-efaf- | 0.01 | HRD− | 0.0187671 | 29 | PCAWG | none | 0 |
| 5e5e-8259- | | | | | | | |
| d2cfd96ed72d | | | | | | | |
| 040c742c-b160- | 0 | HRD− | 1.73E−07 | 23 | PCAWG | none | 0 |
| 5661-ae1c- | | | | | | | |
| c6a1cb2075ae | | | | | | | |
| 084aeeb0-e733- | 0 | HRD− | 1.88E−08 | 148 | PCAWG | none | 0 |
| 5925-a469- | | | | | | | |
| f92bd69a7bb5 | | | | | | | |
| 08d85f2d-6019- | 0 | HRD− | 0.0099434 | 52 | PCAWG | none | 0 |
| 50e7-b122- | | | | | | | |
| c62827b36570 | | | | | | | |
| 093a92ae-fb05- | 0 | HRD− | 6.19E−07 | 479 | PCAWG | none | 0 |
| 5164-aad3- | | | | | | | |
| 76f47dd301dc | | | | | | | |
| 09df28c2-a195- | 0 | HRD− | 5.63E−06 | 126 | PCAWG | none | 0 |
| 5bfb-8474- | | | | | | | |
| 6615433f7696 | | | | | | | |
| 0b0ee084-0bcc- | 213.45 | HRD+ | 0.8091524 | 336 | PCAWG | BRCA2 | 0.094 |
| 57d1-96f8- | | | | | | | |
| b11d0ba7e795 | | | | | | | |
| 0f880c0f-5296- | 0.14 | HRD− | 0.0704126 | 32 | PCAWG | none | 0 |
| 51d7-9a04- | | | | | | | |
| f3c935be66ea | | | | | | | |
| 1086891c-4ab2- | 0 | HRD− | 1.53E−07 | 53 | PCAWG | none | 0 |
| 5c8a-9111- | | | | | | | |
| 8d214006536b | | | | | | | |
| 11631912-4d0b- | 0 | HRD− | 2.78E−05 | 25 | PCAWG | none | 0 |
| 571f-8c75- | | | | | | | |
| 9a94ad3d5384 | | | | | | | |
| 11758875-0852- | 0.27 | HRD− | 0.0896433 | 31 | PCAWG | none | 0 |
| 5b3f-95d0- | | | | | | | |
| 1e8dd2fb425a | | | | | | | |
| 12505851-1bad- | 0 | HRD− | 1.16E−06 | 104 | PCAWG | none | 0 |
| 5829-a768- | | | | | | | |
| b54ad7cc7425 | | | | | | | |
| 16eaa5e9-491c- | 0.82 | HRD− | 0.1354456 | 54 | PCAWG | none | 0 |
| 5427-a7ae- | | | | | | | |
| 3719c3989181 | | | | | | | |
| 1730836c-eecf- | 0 | HRD− | 1.04E−06 | 51 | PCAWG | none | 0 |
| 5c04-9497- | | | | | | | |
| 82997cbb29e9 | | | | | | | |
| 18381ad9-3fa0- | 0 | HRD− | 1.23E−07 | 60 | PCAWG | none | 0 |
| 56b8-8809- | | | | | | | |
| 03c56c2c90d0 | | | | | | | |
| 1918201b-3a2f- | 0 | HRD− | 4.97E−07 | 98 | PCAWG | none | 0 |
| 5445-b612- | | | | | | | |
| 411d6168cbe0 | | | | | | | |
| 194ee644-c3ff- | 0 | HRD− | 1.30E−04 | 33 | PCAWG | none | 0 |
| 56b3-82ae- | | | | | | | |
| 4d39bb3bfb38 | | | | | | | |
| 1f500fb1-84a1- | 0 | HRD− | 4.55E−09 | 216 | PCAWG | none | 0 |
| 5743-b285- | | | | | | | |
| d9c2a6116a0b | | | | | | | |
| 21a45669-3836- | 0 | HRD− | 1.78E−06 | 65 | PCAWG | none | 0 |
| 52e1-8be7- | | | | | | | |
| af73c5a03e8c | | | | | | | |
| 21ad4e02-eaf3- | 0 | HRD− | 1.12E−05 | 68 | PCAWG | none | 0 |
| 598b-a8d7- | | | | | | | |
| 8479b250d702 | | | | | | | |
| 246b1c1e-29e4- | 0 | HRD− | 5.09E−11 | 139 | PCAWG | none | 0 |
| 5984-aefc- | | | | | | | |
| f5644eccb02e | | | | | | | |
| 24b20aad-44fd- | 254.28 | HRD+ | 0.7593845 | 455 | PCAWG | BRCA2 | 0.056 |
| 5143-8f20- | | | | | | | |
| f36107aaa00a | | | | | | | |
| 27423c64-c787- | 0 | HRD− | 8.03E−10 | 19 | PCAWG | none | 0 |
| 5fe1-8f3f- | | | | | | | |
| 847d1f6b7df2 | | | | | | | |
| 284e9cf4-0314- | 0 | HRD− | 6.73E−07 | 23 | PCAWG | none | 0 |
| 5a62-b509- | | | | | | | |
| bdfbd142b2e1 | | | | | | | |
| 29b544db-04da- | 0 | HRD− | 4.59E−07 | 28 | PCAWG | none | 0 |
| 521a-9749- | | | | | | | |
| 958c50b1887d | | | | | | | |
| 2a8e0b23-6a95- | 0.16 | HRD− | 0.0565645 | 57 | PCAWG | none | 0 |
| 5761-937a- | | | | | | | |
| 2ae5c7a78403 | | | | | | | |
| 2abdf944-e08d- | 0.35 | HRD− | 0.3583547 | 4 | PCAWG | none | 0 |
| 5a3e-9e25- | | | | | | | |
| c05c4824cf6c | | | | | | | |
| 2bd8c64e-c5b4- | 0 | HRD− | 1.05E−04 | 35 | PCAWG | none | 0 |
| 53c9-9079- | | | | | | | |
| c938b8c5406b | | | | | | | |
| 2caeff1e-379c- | 0 | HRD− | 0.0011086 | 22 | PCAWG | none | 0 |
| 5853-a3d8- | | | | | | | |
| 579aa99eba9d | | | | | | | |
| 2d1ebf8c-b5b8- | 30.18 | HRD+ | 0.5949133 | 92 | PCAWG | none | 0 |
| 5bef-b816- | | | | | | | |
| 53d050d7456c | | | | | | | |
| 2e885294-1461- | 179.78 | HRD+ | 0.7747511 | 298 | PCAWG | none | 0.122 |
| 5a27-a3c0- | | | | | | | |
| fee7be206947 | | | | | | | |
| 2e8a4934-ce3f- | 0 | HRD− | 1.64E−06 | 70 | PCAWG | none | 0 |
| 5c0c-bb84- | | | | | | | |
| 82e463e3f88d | | | | | | | |
| 2eea14ec-8c3f- | 534.07 | HRD+ | 0.8026275 | 869 | PCAWG | BRCA2 | 0.158 |
| 54d1-a589- | | | | | | | |
| 8897a1e033e1 | | | | | | | |
| 2f75ea2e-ac46- | 0.06 | HRD− | 0.0477069 | 25 | PCAWG | none | 0 |
| 5266-8b28- | | | | | | | |
| 1632d9764fab | | | | | | | |
| 30704065-1b62- | 1.81 | HRD+ | 0.204169 | 34 | PCAWG | none | 0.02 |
| 5a7f-b4b3- | | | | | | | |
| 6a2cecbe88e8 | | | | | | | |
| 33c0c336-e79d- | 0 | HRD− | 1.90E−06 | 94 | PCAWG | none | 0 |
| 53e7-8d48- | | | | | | | |
| f8755e536d3e | | | | | | | |
| 33d37b9f-4104- | 0 | HRD− | 6.58E−04 | 68 | PCAWG | none | 0.008 |
| 5a19-8848- | | | | | | | |
| dc64b12b80db | | | | | | | |
| 34bb9fa3-85c0- | 0 | HRD− | 3.72E−10 | 48 | PCAWG | none | 0 |
| 5c18-a82d- | | | | | | | |
| 0556f10cc607 | | | | | | | |
| 37474afb-38a7- | 0 | HRD− | 4.52E−13 | 86 | PCAWG | none | 0 |
| 5c20-b385- | | | | | | | |
| d7571b687901 | | | | | | | |
| 374ff8bf-a2e2- | 0.05 | HRD− | 0.0507451 | 17 | PCAWG | none | 0 |
| 5d3f-a706- | | | | | | | |
| a2ec12227afd | | | | | | | |
| 38ba9f2c-bcd8- | 0 | HRD− | 0.0153213 | 21 | PCAWG | none | 0 |
| 594c-8d44- | | | | | | | |
| 99d3f434f95a | | | | | | | |
| 3990a431-ad52- | 0 | HRD− | 9.78E−10 | 71 | PCAWG | none | 0 |
| 50d7-8d60- | | | | | | | |
| ff294b4739b6 | | | | | | | |
| 3a82c2a0-e2c8- | 0 | HRD− | 4.70E−06 | 87 | PCAWG | none | 0 |
| 559e-8eae- | | | | | | | |
| 55bad0a6fdd1 | | | | | | | |
| 3b649306-5d50- | 417.22 | HRD+ | 0.8461915 | 610 | PCAWG | BRCA2 | 0.082 |
| 5ffd-a508- | | | | | | | |
| 0a42f49f65f9 | | | | | | | |
| 3c6ab0f4-1170- | 0 | HRD− | 0.0192732 | 14 | PCAWG | none | 0 |
| 55fe-a092- | | | | | | | |
| 12a100d1d65c | | | | | | | |
| 3cb282a8-e7c8- | 1.52 | HRD+ | 0.150713 | 60 | PCAWG | none | 0 |
| 58f2-bebb- | | | | | | | |
| 7e355da96cc0 | | | | | | | |
| 3ce8bd0d-dc8e- | 0 | HRD− | 8.41E−11 | 177 | PCAWG | none | 0.012 |
| 5bee-a351- | | | | | | | |
| ab79418a8755 | | | | | | | |
| 3f2f425e-00fa- | 0 | HRD− | 1.13E−04 | 21 | PCAWG | none | 0 |
| 5100-84cc- | | | | | | | |
| 335bcbb8b604 | | | | | | | |
| 3fab3482-0253- | 0 | HRD− | 8.45E−07 | 346 | PCAWG | none | 0 |
| 5f38-8b63- | | | | | | | |
| 4ddac2c7933e | | | | | | | |
| 4115ccd5-4e6f- | 0 | HRD− | 1.25E−07 | 49 | PCAWG | none | 0 |
| 584a-9c3f- | | | | | | | |
| 67d367f3a3f2 | | | | | | | |
| 418c2c49-c470- | 0.15 | HRD− | 0.0617822 | 40 | PCAWG | none | 0.002 |
| 52a3-9895- | | | | | | | |
| 29d33a557404 | | | | | | | |
| 432629d7-4f6f- | 0 | HRD− | 7.19E−05 | 105 | PCAWG | none | 0 |
| 5739-abda- | | | | | | | |
| 24da355f1d52 | | | | | | | |
| 43b1206f-7129- | 0.04 | HRD− | 0.0323432 | 36 | PCAWG | none | 0 |
| 5493-8472- | | | | | | | |
| f14886ae9785 | | | | | | | |
| 47344f5f-467f- | 0 | HRD− | 4.53E−06 | 88 | PCAWG | none | 0 |
| 5f91-8695- | | | | | | | |
| 59d1164e7755 | | | | | | | |
| 4790fb9e-04fd- | 0.22 | HRD− | 0.08744 | 24 | PCAWG | none | 0.006 |
| 51fd-8a8b- | | | | | | | |
| 0df0e8372b3c | | | | | | | |
| 47c573a7-b108- | 0 | HRD− | 1.11E−05 | 60 | PCAWG | none | 0 |
| 5c73-a221- | | | | | | | |
| c0c6103a7330 | | | | | | | |
| 48849c94-a5f0- | 0 | HRD− | 1.36E−06 | 79 | PCAWG | none | 0 |
| 516e-89ac- | | | | | | | |
| 2db95b570480 | | | | | | | |
| 48bc879e-2cbb- | 0 | HRD− | 0.0088774 | 15 | PCAWG | none | 0.002 |
| 54cb-82c3- | | | | | | | |
| 7be3ebf971d2 | | | | | | | |
| 4c913ea0-f24c- | 0.15 | HRD− | 0.1056748 | 15 | PCAWG | none | 0.018 |
| 5be4-a0c7- | | | | | | | |
| df56c0fa2ca4 | | | | | | | |
| 4df913af-9ce5- | 0.09 | HRD− | 0.0528381 | 34 | PCAWG | none | 0 |
| 59f3-ba8e- | | | | | | | |
| b52ccc267718 | | | | | | | |
| 4e13d88e-fef2- | 0 | HRD− | 6.20E−05 | 24 | PCAWG | none | 0 |
| 568d-982f- | | | | | | | |
| bf6158e23262 | | | | | | | |
| 4e6c5a6d-024c- | 0.02 | HRD− | 0.0290822 | 32 | PCAWG | none | 0 |
| 5e3a-a515- | | | | | | | |
| 7de51265ce96 | | | | | | | |
| 4fecdafc-ee08- | 0.01 | HRD− | 0.0190393 | 20 | PCAWG | none | 0 |
| 5caa-9225- | | | | | | | |
| 9335f0b2fa63 | | | | | | | |
| 5096f0b3-5a43- | 0 | HRD− | 9.97E−09 | 37 | PCAWG | none | 0 |
| 5cee-9dd3- | | | | | | | |
| 20d5c4f9c423 | | | | | | | |
| 5189306c-7195- | 0.02 | HRD− | 0.0251597 | 30 | PCAWG | none | 0 |
| 55cf-9aa6- | | | | | | | |
| 70b93456b011 | | | | | | | |
| 538ccbff-bf39- | 0 | HRD− | 8.36E−06 | 22 | PCAWG | none | 0 |
| 5c45-acf8- | | | | | | | |
| 09938858b4c2 | | | | | | | |
| 560e9919-d34d- | 0 | HRD− | 1.57E−04 | 49 | PCAWG | none | 0 |
| 53bd-acea- | | | | | | | |
| e248f89b756e | | | | | | | |
| 569a3222-c189- | 0 | HRD− | 0.0045766 | 10 | PCAWG | none | 0.006 |
| 5b0e-8ced- | | | | | | | |
| 01d75b5108f2 | | | | | | | |
| 56a634de-b0ed- | 0 | HRD− | 8.88E−06 | 68 | PCAWG | none | 0.002 |
| 5bdc-ae07- | | | | | | | |
| 8d22aa762b61 | | | | | | | |
| 57cb4f59-796a- | 0 | HRD− | 7.81E−07 | 181 | PCAWG | none | 0 |
| 56e9-8b86- | | | | | | | |
| cf0239040336 | | | | | | | |
| 57d038bb-2b7f- | 0 | HRD− | 4.84E−04 | 66 | PCAWG | none | 0 |
| 5e26-b10e- | | | | | | | |
| e15e60a879ec | | | | | | | |
| 599463f7-b9aa- | 0 | HRD− | 1.25E−07 | 47 | PCAWG | none | 0 |
| 583b-80dd- | | | | | | | |
| 2556bf385eae | | | | | | | |
| 59a98d91-2b50- | 0 | HRD− | 2.89E−06 | 118 | PCAWG | none | 0 |
| 54cd-8162- | | | | | | | |
| 08d3b7e132cd | | | | | | | |
| 5c36bbb2-015a- | 0 | HRD− | 3.45E−09 | 88 | PCAWG | none | 0 |
| 5587-8f14- | | | | | | | |
| 63f7e4c5183c | | | | | | | |
| 60fdfca2-8a8a- | 0 | HRD− | 9.75E−12 | 47 | PCAWG | none | 0 |
| 595a-b11b- | | | | | | | |
| b40d95b353ff | | | | | | | |
| 62211e76-5da0- | 94.66 | HRD+ | 0.6400022 | 223 | PCAWG | none | 0.122 |
| 57aa-a8ae- | | | | | | | |
| 1b0cbc14b41b | | | | | | | |
| 62b4e4d7-67db- | 1.15 | HRD+ | 0.1781586 | 40 | PCAWG | none | 0.004 |
| 5f21-902d- | | | | | | | |
| b5d01677505e | | | | | | | |
| 64eb7730-c9af- | 0 | HRD− | 0.0064917 | 50 | PCAWG | none | 0 |
| 5eb1-939e- | | | | | | | |
| 519d93ca878e | | | | | | | |
| 690b056d-3a55- | 0.45 | HRD− | 0.1311351 | 29 | PCAWG | none | 0.048 |
| 546d-a239- | | | | | | | |
| 5da99d2166b3 | | | | | | | |
| 69dda2c3-1f62- | 0 | HRD− | 2.89E−10 | 64 | PCAWG | none | 0.006 |
| 5d58-a482- | | | | | | | |
| 991d959c10e5 | | | | | | | |
| 6c178066-4e9d- | 0 | HRD− | 3.50E−06 | 52 | PCAWG | none | 0 |
| 5f87-88a4- | | | | | | | |
| bbcca334b5a2 | | | | | | | |
| 6c6a2f4c-aad4- | 0 | HRD− | 1.13E−06 | 50 | PCAWG | none | 0 |
| 5944-abef- | | | | | | | |
| 3d7cd0144429 | | | | | | | |
| 6d7dab97-8dac- | 0 | HRD− | 1.12E−06 | 49 | PCAWG | none | 0 |
| 58bc-974e- | | | | | | | |
| 7dba2df938f8 | | | | | | | |
| 6d91daa8-f8a5- | 0.02 | HRD− | 0.037602 | 12 | PCAWG | none | 0 |
| 5065-a40c- | | | | | | | |
| 03cf6484f612 | | | | | | | |
| 6e0789d0-e8bc- | 0 | HRD− | 0.0089532 | 13 | PCAWG | none | 0 |
| 5e6d-a83a- | | | | | | | |
| b8563d61ec73 | | | | | | | |
| 6f8aec20-dbb0- | 0 | HRD− | 2.91E−06 | 55 | PCAWG | none | 0 |
| 5232-a2b6- | | | | | | | |
| fb8ed3fe9c4f | | | | | | | |
| 73a2e451-5e53- | 0.22 | HRD− | 0.0908638 | 24 | PCAWG | none | 0 |
| 5ff6-8348- | | | | | | | |
| 69292c2b3ef3 | | | | | | | |
| 75f0ebd2-d791- | 1.13 | HRD+ | 0.1608325 | 44 | PCAWG | none | 0.002 |
| 520c-980b- | | | | | | | |
| 1264ecf7dc5f | | | | | | | |
| 76ebb33a-7405- | 0 | HRD− | 0.0068299 | 35 | PCAWG | none | 0 |
| 5871-be7d- | | | | | | | |
| 6520ee988899 | | | | | | | |
| 789bf8d1-ac6e- | 0 | HRD− | 4.60E−06 | 92 | PCAWG | none | 0 |
| 57da-9f34- | | | | | | | |
| 1545d40155eb | | | | | | | |
| 78b1787a-6191- | 0.03 | HRD− | 0.0417104 | 20 | PCAWG | none | 0.016 |
| 50f6-9c20- | | | | | | | |
| 0dfacb87bfa4 | | | | | | | |
| 78d4c74a-11d8- | 494.69 | HRD+ | 0.7634681 | 901 | PCAWG | BRCA2 | 0.402 |
| 5fe2-9be3- | | | | | | | |
| ffa09d30c4ea | | | | | | | |
| 79fce701-1b11- | 0 | HRD− | 2.73E−08 | 49 | PCAWG | none | 0 |
| 5849-8b7d- | | | | | | | |
| 677e49ecff49 | | | | | | | |
| 7a447ffc-f4a2- | 0.64 | HRD− | 0.1057814 | 63 | PCAWG | none | 0 |
| 5d15-82f9- | | | | | | | |
| e0b624b5632f | | | | | | | |
| 7c24476b-c721- | 0 | HRD− | 6.78E−05 | 38 | PCAWG | none | 0 |
| 559e-b945- | | | | | | | |
| ad21682ebed7 | | | | | | | |
| 7cee07d4-d459- | 0.92 | HRD− | 0.2273896 | 19 | PCAWG | none | 0 |
| 5e98-894b- | | | | | | | |
| ba74575e8de2 | | | | | | | |
| 7db45081-5eeb- | 0 | HRD− | 7.14E−07 | 19 | PCAWG | none | 0.056 |
| 5496-a51f- | | | | | | | |
| a209b81d9c12 | | | | | | | |
| 7f3b6b4f-4876- | 0 | HRD− | 6.71E−06 | 36 | PCAWG | none | 0 |
| 56e4-beab- | | | | | | | |
| 082d1c975710 | | | | | | | |
| 7f4312bc-fe88- | 0.05 | HRD− | 0.0316305 | 56 | PCAWG | none | 0 |
| 5b9a-a292- | | | | | | | |
| 2f87cf248ccd | | | | | | | |
| 7fd9535f-3512- | 0 | HRD− | 1.81E−06 | 114 | PCAWG | none | 0 |
| 5765-a369- | | | | | | | |
| 82d139705567 | | | | | | | |
| 801dcc66-92e6- | 0 | HRD− | 0.0029729 | 24 | PCAWG | none | 0 |
| 5575-a2a5- | | | | | | | |
| d478e5bf6773 | | | | | | | |
| 80c539c4-ad9e- | 0 | HRD− | 1.26E−06 | 69 | PCAWG | none | 0.066 |
| 5ac0-83a7- | | | | | | | |
| 8277de850443 | | | | | | | |
| 82dc9348-35d3- | 0.01 | HRD− | 0.0137492 | 38 | PCAWG | none | 0 |
| 5818-9d53- | | | | | | | |
| 83cb5405c323 | | | | | | | |
| 84e48eaf-fa44- | 0.01 | HRD− | 0.018903 | 17 | PCAWG | none | 0 |
| 5a3e-99e1- | | | | | | | |
| 59123d2b1bee | | | | | | | |
| 8656f03e-ae65- | 0.09 | HRD− | 0.0498357 | 34 | PCAWG | none | 0 |
| 5568-aa1c- | | | | | | | |
| 54fd964d4305 | | | | | | | |
| 871fc242-e557- | 0.8 | HRD− | 0.1375537 | 45 | PCAWG | none | 0 |
| 52c6-a996- | | | | | | | |
| c535669a855f | | | | | | | |
| 880574c0-99b7- | 0 | HRD− | 2.36E−06 | 73 | PCAWG | none | 0 |
| 5494-b3d8- | | | | | | | |
| 994692b11d95 | | | | | | | |
| 8ac7e33f-0e0d- | 0 | HRD− | 4.38E−07 | 125 | PCAWG | none | 0 |
| 529f-a653- | | | | | | | |
| 0ceeeafb0f34 | | | | | | | |
| 8c1e49f9-ed1d- | 0 | HRD− | 1.61E−11 | 126 | PCAWG | none | 0 |
| 5e1a-9913- | | | | | | | |
| 509003f6cebc | | | | | | | |
| 8c4fa1eb-6426- | 0 | HRD− | 1.04E−07 | 100 | PCAWG | none | 0 |
| 5733-acdc- | | | | | | | |
| 81b881c31c69 | | | | | | | |
| 8e3a5956-23da- | 0.37 | HRD− | 0.1300599 | 14 | PCAWG | none | 0 |
| 585c-99d3- | | | | | | | |
| b8f905d3cdf8 | | | | | | | |
| 915b1720-17e3- | 0 | HRD− | 2.05E−07 | 59 | PCAWG | none | 0 |
| 54bf-924e- | | | | | | | |
| 365b49647aa4 | | | | | | | |
| 926e8cd0-ffcd- | 9.69 | HRD+ | 0.2198416 | 155 | PCAWG | BRCA1 | 0.514 |
| 5cdc-9f63- | | | | | | | |
| 95a53a23c443 | | | | | | | |
| 933061cd-7229- | 625.15 | HRD+ | 0.7165775 | 1232 | PCAWG | BRCA2 | 0.15 |
| 533c-92a5- | | | | | | | |
| 0f01be6498c7 | | | | | | | |
| 938ef410-e08e- | 0.76 | HRD− | 0.1371136 | 38 | PCAWG | none | 0 |
| 59eb-918d- | | | | | | | |
| 1b87c296ca96 | | | | | | | |
| 94669dbe-8d87- | 0 | HRD− | 0.0118502 | 19 | PCAWG | none | 0 |
| 55d8-80ae- | | | | | | | |
| 678c6b2be998 | | | | | | | |
| 99afadf2-07f7- | 230.64 | HRD+ | 0.7431862 | 428 | PCAWG | BRCA2 | 0.146 |
| 5105-8b1b- | | | | | | | |
| 5328be8f7c69 | | | | | | | |
| 9a36a27e-4037- | 0 | HRD− | 1.71E−07 | 28 | PCAWG | none | 0 |
| 53d9-9652- | | | | | | | |
| b898c5f8b088 | | | | | | | |
| 9a5b350a-2387- | 0 | HRD− | 5.78E−07 | 77 | PCAWG | none | 0 |
| 54fb-a4de- | | | | | | | |
| c4c09b0194f4 | | | | | | | |
| 9c5f0679-4745- | 0 | HRD− | 4.98E−10 | 45 | PCAWG | none | 0 |
| 50b9-ae5c- | | | | | | | |
| 8279c14d84ed | | | | | | | |
| 9d3f34b2-6497- | 1.32 | HRD+ | 0.167428 | 43 | PCAWG | none | 0.02 |
| 5886-89ca- | | | | | | | |
| 924673e28abc | | | | | | | |
| 9d5907d3-180f- | 0.1 | HRD− | 0.0599548 | 26 | PCAWG | none | 0 |
| 5364-b75f- | | | | | | | |
| 281459b60aa2 | | | | | | | |
| 9dfe717d-acb3- | 0.01 | HRD− | 0.0306428 | 11 | PCAWG | none | 0 |
| 5e86-8d14- | | | | | | | |
| 14f56a1472fe | | | | | | | |
| 9efcb184-a6b4- | 0 | HRD− | 9.22E−05 | 24 | PCAWG | none | 0 |
| 52e7-b37d- | | | | | | | |
| 4bfa12ef57f5 | | | | | | | |
| a1713a31-68bf- | 0 | HRD− | 1.32E−08 | 28 | PCAWG | none | 0 |
| 5e0a-a27d- | | | | | | | |
| bc266aff2dc0 | | | | | | | |
| a1b6eac7-71f1- | 0.13 | HRD− | 0.0793322 | 22 | PCAWG | none | 0 |
| 59c7-b6fd- | | | | | | | |
| 03be54cda35c | | | | | | | |
| a5c8b2b5-ef5a- | 0.15 | HRD− | 0.0706961 | 26 | PCAWG | none | 0 |
| 5475-acbf- | | | | | | | |
| 87cd0072d3a2 | | | | | | | |
| a6c0456e-f76f- | 0 | HRD− | 2.89E−06 | 55 | PCAWG | none | 0 |
| 59ee-892e- | | | | | | | |
| 1d3fe03da47f | | | | | | | |
| a9c962ff-5a4f- | 0 | HRD− | 3.71E−06 | 29 | PCAWG | none | 0.004 |
| 5464-8c53- | | | | | | | |
| e43adcd26e36 | | | | | | | |
| aa05ac1c-b123- | 120.78 | HRD+ | 0.4020316 | 606 | PCAWG | BRCA1 | 0.77 |
| 5d97-9c32- | | | | | | | |
| 24bc40831b76 | | | | | | | |
| ac398ef4-7baf- | 0 | HRD− | 1.96E−06 | 29 | PCAWG | none | 0 |
| 573c-82c4- | | | | | | | |
| e78f3a86bc33 | | | | | | | |
| ac6a5c70-e9c7- | 0 | HRD− | 6.23E−08 | 131 | PCAWG | none | 0 |
| 5f9a-b1ed- | | | | | | | |
| 09ca6aa65e0a | | | | | | | |
| ae3d51c8-0a2d- | 0 | HRD− | 1.24E−08 | 82 | PCAWG | none | 0 |
| 55ac-9e51- | | | | | | | |
| 3d8d275ca715 | | | | | | | |
| ae515006-2778- | 0 | HRD− | 1.84E−06 | 33 | PCAWG | none | 0.002 |
| 591f-ae50- | | | | | | | |
| 8858b5af1a0a | | | | | | | |
| ae9722c4-3f1d- | 0.01 | HRD− | 0.0267014 | 10 | PCAWG | none | 0 |
| 5424-9999- | | | | | | | |
| 354ecd6d6619 | | | | | | | |
| b25c51ec-b5e3- | 0 | HRD− | 6.25E−05 | 46 | PCAWG | none | 0 |
| 5c6a-8904- | | | | | | | |
| 94319ba2e1e0 | | | | | | | |
| b43d709a-657d- | 0 | HRD− | 9.61E−08 | 31 | PCAWG | none | 0.002 |
| 5c49-9500- | | | | | | | |
| a98c44930fa7 | | | | | | | |
| b4f221e9-2c0b- | 0 | HRD− | 2.23E−07 | 121 | PCAWG | none | 0 |
| 5ad3-aac2- | | | | | | | |
| 4fefcfa1aea6 | | | | | | | |
| b5237253-7214- | 0 | HRD− | 1.24E−06 | 72 | PCAWG | none | 0 |
| 5dcd-8dff- | | | | | | | |
| 9c1a429a7c57 | | | | | | | |
| b54de531-e7da- | 0 | HRD− | 8.77E−08 | 127 | PCAWG | none | 0 |
| 521f-a292- | | | | | | | |
| 68740cc967a9 | | | | | | | |
| b7b176c9-0e5d- | 0 | HRD− | 9.41E−04 | 48 | PCAWG | none | 0 |
| 5de9-a679- | | | | | | | |
| 4555f441d914 | | | | | | | |
| b8269bc3-8c0f- | 0 | HRD− | 3.13E−07 | 113 | PCAWG | none | 0 |
| 5af2-af9b- | | | | | | | |
| ce2cd9319848 | | | | | | | |
| b9d3cf7f-80df- | 1.82 | HRD+ | 0.1294388 | 118 | PCAWG | none | 0.414 |
| 5107-ba85- | | | | | | | |
| f287ac250aa7 | | | | | | | |
| bb3ecb9d-3b53- | 0 | HRD− | 1.25E−07 | 81 | PCAWG | none | 0 |
| 5e0e-88ea- | | | | | | | |
| 69081a11c993 | | | | | | | |
| bc4d5c8f-277b- | 0 | HRD− | 0.0045352 | 42 | PCAWG | none | 0 |
| 5946-8ce0- | | | | | | | |
| ea58f5e2fa4c | | | | | | | |
| bcd38401-cb6c- | 2.71 | HRD+ | 0.2442707 | 50 | PCAWG | none | 0 |
| 5cd9-914a- | | | | | | | |
| 3501fab6e771 | | | | | | | |
| bda9e129-780e- | 625.3 | HRD+ | 0.8094094 | 1041 | PCAWG | none | 0.222 |
| 5182-a056- | | | | | | | |
| d614617da60b | | | | | | | |
| bdf175be-a89a- | 1.25 | HRD+ | 0.2665956 | 19 | PCAWG | none | 0 |
| 5a16-af67- | | | | | | | |
| 8135ee914b4e | | | | | | | |
| be92e51a-6d14- | 374.14 | HRD+ | 0.7799098 | 632 | PCAWG | BRCA2 | 0.436 |
| 505d-b997- | | | | | | | |
| e1695c765ef0 | | | | | | | |
| bf6c08fa-bf28- | 0.52 | HRD− | 0.0883683 | 70 | PCAWG | none | 0 |
| 5971-912f- | | | | | | | |
| 340e5bf73a0e | | | | | | | |
| c01807ea-b622- | 0.48 | HRD− | 0.0878232 | 58 | PCAWG | none | 0.002 |
| 5197-b88d- | | | | | | | |
| bbededc15025 | | | | | | | |
| c28296f4-c609- | 0 | HRD− | 1.11E−05 | 49 | PCAWG | none | 0 |
| 56d9-9af7- | | | | | | | |
| 5b7977eb0c67 | | | | | | | |
| c4f4f1c9-ddd7- | 0 | HRD− | 7.60E−04 | 16 | PCAWG | none | 0 |
| 500f-8a9a- | | | | | | | |
| 1bd3fba46e53 | | | | | | | |
| c678c547-b34d- | 0 | HRD− | 7.10E−07 | 58 | PCAWG | none | 0 |
| 5ec5-b052- | | | | | | | |
| 2d946b1ea6a1 | | | | | | | |
| c8f8440c-da1a- | 2.41 | HRD+ | 0.2680887 | 30 | PCAWG | none | 0 |
| 5a85-9c63- | | | | | | | |
| 95f6615279f4 | | | | | | | |
| ca1b6fbb-b794- | 0 | HRD− | 0.0045063 | 32 | PCAWG | none | 0 |
| 5f30-a727- | | | | | | | |
| 7ed7d7a061d4 | | | | | | | |
| ca8a0cc6-6dd3- | 0 | HRD− | 0.0010279 | 37 | PCAWG | none | 0 |
| 5d91-8de3- | | | | | | | |
| b0324728bfb4 | | | | | | | |
| cb1d1bfd-5588- | 0 | HRD− | 1.15E−06 | 95 | PCAWG | none | 0 |
| 57a7-a962- | | | | | | | |
| b354a6740dbe | | | | | | | |
| cb30608b-1362- | 0.26 | HRD− | 0.0518102 | 77 | PCAWG | none | 0 |
| 5330-ba65- | | | | | | | |
| e55176b295cf | | | | | | | |
| cbce448e-62ee- | 99.06 | HRD+ | 0.4541084 | 521 | PCAWG | BRCA2 | 0.172 |
| 5fcf-87b8- | | | | | | | |
| 4fd0abeed013 | | | | | | | |
| cc605155-d350- | 0 | HRD− | 1.36E−08 | 142 | PCAWG | none | 0 |
| 5da6-881b- | | | | | | | |
| 85ba7414e273 | | | | | | | |
| cd323cd5-7b72- | 0 | HRD− | 1.17E−09 | 101 | PCAWG | none | 0.002 |
| 59b0-bcf9- | | | | | | | |
| e89712635f3b | | | | | | | |
| cf3db688-8f76- | 0 | HRD− | 1.22E−05 | 25 | PCAWG | none | 0.014 |
| 5e3f-b4c3- | | | | | | | |
| 8ea6f1dbfb6a | | | | | | | |
| cfd68b84-43a9- | 0 | HRD− | 0.0020043 | 41 | PCAWG | none | 0 |
| 5458-8822- | | | | | | | |
| cb30fcbcfc05 | | | | | | | |
| d1beb958-66fb- | 0 | HRD− | 3.87E−06 | 61 | PCAWG | none | 0 |
| 5cf0-86ec- | | | | | | | |
| f569177e8e02 | | | | | | | |
| d214b014-9a5a- | 0 | HRD− | 5.32E−10 | 320 | PCAWG | none | 0 |
| 5450-92dc- | | | | | | | |
| 029ec14b563f | | | | | | | |
| d2732e94-a759- | 0.03 | HRD− | 0.0356601 | 19 | PCAWG | none | 0 |
| 59d4-9f35- | | | | | | | |
| d09d25a3fda3 | | | | | | | |
| d34c6934-d0c8- | 0 | HRD− | 3.55E−07 | 89 | PCAWG | none | 0 |
| 58b5-9a34- | | | | | | | |
| 9e1f2a54fea7 | | | | | | | |
| d4a4bd38-5f52- | 0 | HRD− | 1.62E−06 | 136 | PCAWG | none | 0 |
| 5cda-b442- | | | | | | | |
| f9f380e11421 | | | | | | | |
| d64b3fe1-5b2d- | 0.01 | HRD− | 0.0150004 | 37 | PCAWG | none | 0 |
| 5845-85bc- | | | | | | | |
| ed5e5c2071a4 | | | | | | | |
| d68a2d08-a9d5- | 0 | HRD− | 1.37E−06 | 106 | PCAWG | none | 0 |
| 52c3-82ba- | | | | | | | |
| 07514970f061 | | | | | | | |
| d6eb18f4-c295- | 0 | HRD− | 9.55E−07 | 29 | PCAWG | none | 0 |
| 5700-9a69- | | | | | | | |
| e41efba30457 | | | | | | | |
| d6fece5a-3a0b- | 0 | HRD− | 7.02E−04 | 45 | PCAWG | none | 0.024 |
| 54f9-a074- | | | | | | | |
| 3c2b44713584 | | | | | | | |
| d7af903d-3e21- | 0 | HRD− | 2.83E−09 | 29 | PCAWG | none | 0.004 |
| 5b92-ac3d- | | | | | | | |
| 85a25fe3c99b | | | | | | | |
| d8a76e59-a22c- | 0 | HRD− | 1.99E−06 | 66 | PCAWG | none | 0.028 |
| 55d6-98f4- | | | | | | | |
| 0f5e627f13e3 | | | | | | | |
| d997c058-ad11- | 0 | HRD− | 0.004612 | 32 | PCAWG | none | 0 |
| 545d-9257- | | | | | | | |
| 927c93385a2f | | | | | | | |
| db04b66e-f1e7- | 0 | HRD− | 5.04E−07 | 77 | PCAWG | none | 0 |
| 5753-a289- | | | | | | | |
| 546d36cbb904 | | | | | | | |
| df20f849-202a- | 0 | HRD− | 1.07E−04 | 19 | PCAWG | none | 0 |
| 5018-8343- | | | | | | | |
| f966daf012ac | | | | | | | |
| df8a5ed8-acff- | 0 | HRD− | 5.96E−15 | 133 | PCAWG | none | 0 |
| 53fc-80ee- | | | | | | | |
| efde0acdceed | | | | | | | |
| e059a9fe-8842- | 0 | HRD− | 0.0013814 | 26 | PCAWG | none | 0 |
| 525d-85f5- | | | | | | | |
| fe4e47baa9df | | | | | | | |
| e2d9369c-761d- | 0.8 | HRD− | 0.1339382 | 46 | PCAWG | none | 0 |
| 56d6-9365- | | | | | | | |
| 9e8eb6c85552 | | | | | | | |
| e3e12acb-66bf- | 0 | HRD− | 0.0079267 | 41 | PCAWG | none | 0 |
| 5b50-b052- | | | | | | | |
| 9351ade4bd9d | | | | | | | |
| e43bf8db-9d73- | 0 | HRD− | 4.24E−05 | 36 | PCAWG | none | 0 |
| 5555-8648- | | | | | | | |
| da1d3b78bdc3 | | | | | | | |
| e44cded3-7144- | 0.06 | HRD− | 0.0434029 | 27 | PCAWG | none | 0 |
| 5dac-b95c- | | | | | | | |
| 727a2995f15c | | | | | | | |
| e491ad53-9222- | 0 | HRD− | 6.88E−07 | 4112 | PCAWG | none | 0 |
| 592d-93a1- | | | | | | | |
| 79d34ccf4e76 | | | | | | | |
| e4b3fa16-98b2- | 0 | HRD− | 5.32E−06 | 23 | PCAWG | none | 0.058 |
| 5b02-9b8f- | | | | | | | |
| b76d5c6526f3 | | | | | | | |
| e5d84bc0-9013- | 0.01 | HRD− | 0.0147327 | 24 | PCAWG | none | 0 |
| 53bf-91c0- | | | | | | | |
| 676f305c5102 | | | | | | | |
| e6efbe00-7125- | 0 | HRD− | 2.61E−04 | 19 | PCAWG | none | 0 |
| 5a98-a695- | | | | | | | |
| a34be6f7ddb4 | | | | | | | |
| e6f965d2-f2fc- | 0 | HRD− | 7.13E−11 | 75 | PCAWG | none | 0 |
| 56e6-ae12- | | | | | | | |
| cb2671e228e0 | | | | | | | |
| e78f4f84-3a46- | 0 | HRD− | 1.68E−06 | 11 | PCAWG | none | 0 |
| 5ab5-b5c3- | | | | | | | |
| 4b59b61c378a | | | | | | | |
| e7bd6083-1cb8- | 0 | HRD− | 3.49E−06 | 33 | PCAWG | none | 0 |
| 5da3-b701- | | | | | | | |
| fd32791d5ce9 | | | | | | | |
| ebdcf8f1-fcd5- | 0.08 | HRD− | 0.0407286 | 60 | PCAWG | none | 0 |
| 5269-ab53- | | | | | | | |
| 15ff9635e797 | | | | | | | |
| f077e1c8-5729- | 0 | HRD− | 1.91E−07 | 46 | PCAWG | none | 0 |
| 57e8-9029- | | | | | | | |
| eb1d2238e0c0 | | | | | | | |
| f28cb778-14f1- | 156.85 | HRD+ | 0.7323078 | 311 | PCAWG | none | 0.182 |
| 5064-8641- | | | | | | | |
| df9a341d56b6 | | | | | | | |
| f368e8a1-a788- | 0.02 | HRD− | 0.0226402 | 37 | PCAWG | none | 0 |
| 546f-8a7a- | | | | | | | |
| ac42588a5a5a | | | | | | | |
| f3c0e89a-0a00- | 0 | HRD− | 9.23E−07 | 8874 | PCAWG | none | 0 |
| 521d-bc30- | | | | | | | |
| c4893de5fb98 | | | | | | | |
| f42009f2-dba1- | 258.27 | HRD+ | 0.7087948 | 547 | PCAWG | BRCA2 | 0.094 |
| 52b0-b52b- | | | | | | | |
| 44c7c8148df0 | | | | | | | |
| f65fc85d-fb73- | 0 | HRD− | 1.17E−04 | 93 | PCAWG | none | 0.008 |
| 5e2e-bd81- | | | | | | | |
| c54baccaa436 | | | | | | | |
| f68c50fd-a3bb- | 0 | HRD− | 1.29E−06 | 134 | PCAWG | none | 0 |
| 5761-a194- | | | | | | | |
| 0bc201da44f6 | | | | | | | |
| f6b383ef-b07e- | 0 | HRD− | 0.0035069 | 15 | PCAWG | none | 0 |
| 5b38-b2ea- | | | | | | | |
| 19690ea97423 | | | | | | | |
| f7ba5d0c-70d3- | 0 | HRD− | 1.11E−06 | 110 | PCAWG | none | 0 |
| 5130-92d7- | | | | | | | |
| 5bcfed0eed5f | | | | | | | |
| f8a54ba5-236d- | 0 | HRD− | 6.48E−05 | 41 | PCAWG | none | 0 |
| 5b27-a292- | | | | | | | |
| e68e2fc3b8ac | | | | | | | |
| f8ae0285-c759- | 275.2 | HRD+ | 0.7182012 | 566 | PCAWG | BRCA2 | 0.046 |
| 52f6-a4ed- | | | | | | | |
| 7f25b8f686a5 | | | | | | | |
| f927eb70-4a26- | 0.03 | HRD− | 0.0326486 | 27 | PCAWG | none | 0 |
| 5e11-91ed- | | | | | | | |
| ae138ce666dc | | | | | | | |
| fabc6e53-cb9e- | 177.62 | HRD+ | 0.7487964 | 319 | PCAWG | BRCA2 | 0.054 |
| 5053-82ba- | | | | | | | |
| f557aa277a3c | | | | | | | |
| fb506451-7ed9- | 0 | HRD− | 4.94E−06 | 37 | PCAWG | none | 0 |
| 5609-aea6- | | | | | | | |
| de565f96e967 | | | | | | | |
| fb732684-1372- | 0 | HRD− | 2.06E−06 | 69 | PCAWG | none | 0 |
| 5230-9dc7- | | | | | | | |
| 209814d8a7f7 | | | | | | | |
| fda90ec7-e3ef- | 0 | HRD− | 2.63E−06 | 82 | PCAWG | none | 0 |
| 5089-b784- | | | | | | | |
| 97d0fb742f15 | | | | | | | |
| fff213d8-3d8c- | 0 | HRD− | 2.03E−06 | 79 | PCAWG | none | 0 |
| 583d-8f89- | | | | | | | |
| 65f2c33572cd | | | | | | | |

| | | | | | | CHORD_used_{—} |
|---|---|---|---|---|---|---|
| sample | CHORD_p_BRCA2 | CHORD_p_hrd | CHORD_hr_status | CHORD_hrd_type | cancer_type | for_perf_eval |
| f221cbb5-eefa-187f- | 0.08 | 0.892 | HR_deficient | BRCA1_type | Breast | TRUE |
| e040-11ac0c481708 | | | | | | |
| f393ba16-9361-5df4- | 0 | 0 | HR_proficient | none | Breast | TRUE |
| e040-11ac0d4844e8 | | | | | | |
| f393baf9-2710-9203- | 0.054 | 0.93 | HR_deficient | BRCA1_type | Breast | TRUE |
| e040-11ac0d484504 | | | | | | |
| f393bafd-1baa-e5f4- | 0.66 | 0.822 | HR_deficient | BRCA2_type | Breast | TRUE |
| e040-11ac0d48450b | | | | | | |
| f393bafe-7503-5c45- | 0.702 | 0.858 | HR_deficient | BRCA2_type | Breast | TRUE |
| e040-11ac0d484511 | | | | | | |
| f393bb01-6ed7-9533- | 0.052 | 0.826 | HR_deficient | BRCA1_type | Breast | TRUE |
| e040-11ac0d484521 | | | | | | |
| f393bb05-53c2-f80a- | 0 | 0 | HR_proficient | none | Breast | TRUE |
| e040-11ac0d484528 | | | | | | |
| f393bb05-c737-4cc3- | 0 | 0 | HR_proficient | none | Breast | TRUE |
| e040-11ac0d48452a | | | | | | |
| f393bb05-ec1c-17be- | 0 | 0 | HR_proficient | none | Breast | TRUE |
| e040-11ac0d48452f | | | | | | |
| f393bb07-270c-2c93- | 0.732 | 0.792 | HR_deficient | BRCA2_type | Breast | TRUE |
| e040-11ac0d484533 | | | | | | |
| f393bb08-4121-cad8- | 0.748 | 0.86 | HR_deficient | BRCA2_type | Breast | TRUE |
| e040-11ac0d484535 | | | | | | |
| f7b847e5-0f5d-69b4- | 0 | 0 | HR_proficient | none | Breast | TRUE |
| e040-11ac0c4845fe | | | | | | |
| f7b84c09-15d4-3046- | 0 | 0 | HR_proficient | none | Breast | TRUE |
| e040-11ac0c4847ff | | | | | | |
| f7c6486a-bb08-f77a- | 0.348 | 0.48 | HR_proficient | none | Breast | TRUE |
| e040-11ac0c482953 | | | | | | |
| f7c6a336-3126-7580- | 0 | 0 | HR_proficient | none | Breast | TRUE |
| e040-11ac0c485971 | | | | | | |
| f7d77a03-28a0-f446- | 0 | 0 | HR_proficient | none | Breast | TRUE |
| e040-11ac0c4866fb | | | | | | |
| f7d7b3db-02af-61cb- | 0 | 0.008 | HR_proficient | none | Breast | TRUE |
| e040-11ac0c480c03 | | | | | | |
| f7e4a2a8-093d-9e36- | 0.714 | 0.844 | HR_deficient | BRCA2_type | Breast | TRUE |
| e040-11ac0d480c7d | | | | | | |
| f7e7d61f-e2dc-b523- | 0 | 0 | HR_proficient | none | Breast | TRUE |
| e040-11ac0c482000 | | | | | | |
| f7ed28a6-8f5f-e87b- | 0.002 | 0.002 | HR_proficient | none | Breast | TRUE |
| e040-11ac0c48120e | | | | | | |
| f7f66114-e321-1d4c- | 0 | 0 | HR_proficient | none | Breast | TRUE |
| e040-11ac0d48541a | | | | | | |
| f7f6619b-fae8-3381- | 0 | 0 | HR_proficient | none | Breast | TRUE |
| e040-11ac0c483f78 | | | | | | |
| f80293b8-ddb7-f575- | 0 | 0 | HR_proficient | none | Breast | TRUE |
| e040-11ac0d48309d | | | | | | |
| fc447d51-cdc2-a180- | 0 | 0 | HR_proficient | none | Breast | TRUE |
| e040-11ac0c4846a4 | | | | | | |
| fc8130df-18fe-c74d- | 0 | 0 | HR_proficient | none | Breast | TRUE |
| e040-11ac0d485df2 | | | | | | |
| fc8130df-1cdd-cade- | 0 | 0.042 | HR_proficient | none | Breast | TRUE |
| e040-11ac0d485dec | | | | | | |
| fc8130df-1f21-c8f9- | 0.002 | 0.016 | HR_proficient | none | Breast | TRUE |
| e040-11ac0d485dfc | | | | | | |
| fc8130df-2211-cfcc- | 0.574 | 0.82 | HR_deficient | BRCA2_type | Breast | TRUE |
| e040-11ac0d485dde | | | | | | |
| fc8130df-2628-304a- | 0 | 0 | HR_proficient | none | Breast | TRUE |
| e040-11ac0d485dfa | | | | | | |
| fc8130df-2ca4-39d2- | 0 | 0 | HR_proficient | none | Breast | TRUE |
| e040-11ac0d485e00 | | | | | | |
| fc8130df-2e39-3814- | 0 | 0 | HR_proficient | none | Breast | TRUE |
| e040-11ac0d485de0 | | | | | | |
| fc8130df-30a5-3e6a- | 0.644 | 0.83 | HR_deficient | BRCA2_type | Breast | TRUE |
| e040-11ac0d485de2 | | | | | | |
| fc8130df-3147-3e94- | 0 | 0 | HR_proficient | none | Breast | TRUE |
| e040-11ac0d485df8 | | | | | | |
| fc8130df-3361-3c8a- | 0.006 | 0.014 | HR_proficient | none | Breast | TRUE |
| e040-11ac0d485dea | | | | | | |
| fc8130df-34f8-22c6- | 0 | 0 | HR_proficient | none | Breast | TRUE |
| e040-11ac0d485de8 | | | | | | |
| fc8130df-35ac-2304- | 0.014 | 0.07 | HR_proficient | none | Breast | TRUE |
| e040-11ac0d485dda | | | | | | |
| fc8130df-6860-7677- | 0.718 | 0.846 | HR_deficient | BRCA2_type | Breast | TRUE |
| e040-11ac0d485ddc | | | | | | |
| fc8130df-6977-7704- | 0 | 0 | HR_proficient | none | Breast | TRUE |
| e040-11ac0d485de4 | | | | | | |
| fc8130df-8e4e-586b- | 0 | 0 | HR_proficient | none | Breast | TRUE |
| e040-11ac0d485e08 | | | | | | |
| fc8130df-90ba-5d94- | 0.652 | 0.816 | HR_deficient | BRCA2_type | Breast | TRUE |
| e040-11ac0d485e0c | | | | | | |
| fc8130df-e399-e34d- | 0.002 | 0.002 | HR_proficient | none | Breast | TRUE |
| e040-11ac0c483279 | | | | | | |
| fc8130e0-08dd-b996- | 0 | 0 | HR_proficient | none | Breast | TRUE |
| e040-11ac0c48327b | | | | | | |
| fc8130e0-095f-b953- | 0 | 0 | HR_proficient | none | Breast | TRUE |
| e040-11ac0c483287 | | | | | | |
| fc8130e0-096a-b991- | 0 | 0 | HR_proficient | none | Breast | TRUE |
| e040-11ac0c48327d | | | | | | |
| fc8130e0-09f1-b964- | 0 | 0 | HR_proficient | none | Breast | TRUE |
| e040-11ac0c48326e | | | | | | |
| fc8130e0-0bc1-bba7- | 0 | 0 | HR_proficient | none | Breast | TRUE |
| e040-11ac0c483289 | | | | | | |
| fc8130e0-0bfa-bba4- | 0.002 | 0.004 | HR_proficient | none | Breast | TRUE |
| e040-11ac0c48328d | | | | | | |
| fc8130e0-0c53-b432- | 0 | 0 | HR_proficient | none | Breast | TRUE |
| e040-11ac0c483283 | | | | | | |
| fc8130e0-0d25-b475- | 0 | 0.05 | HR_proficient | none | Breast | TRUE |
| e040-11ac0c483277 | | | | | | |
| fc8130e0-0db6-b568- | 0 | 0 | HR_proficient | none | Breast | TRUE |
| e040-11ac0c483281 | | | | | | |
| fc8130e0-0f1a-b6eb- | 0.478 | 0.756 | HR_deficient | BRCA2_type | Breast | TRUE |
| e040-11ac0c48328f | | | | | | |
| fc8130e0-a38a-23b9- | 0.732 | 0.862 | HR_deficient | BRCA2_type | Breast | TRUE |
| e040-11ac0c48327f | | | | | | |
| fc8130e0-a774-d834- | 0.002 | 0.002 | HR_proficient | none | Breast | TRUE |
| e040-11ac0c483268 | | | | | | |
| fc8130e0-ad63-b804- | 0 | 0 | HR_proficient | none | Breast | TRUE |
| e040-11ac0d485e10 | | | | | | |
| fc8130e0-ad8a-b832- | 0 | 0 | HR_proficient | none | Breast | TRUE |
| e040-11ac0d485e14 | | | | | | |
| fc8130e3-01ba-c740- | 0.708 | 0.842 | HR_deficient | BRCA2_type | Breast | TRUE |
| e040-11ac0c483291 | | | | | | |
| fc8130e5-18b8-ef38- | 0 | 0 | HR_proficient | none | Breast | TRUE |
| e040-11ac0c483297 | | | | | | |
| fc81312a-d451-38fb- | 0.002 | 0.004 | HR_proficient | none | Breast | TRUE |
| e040-11ac0c4832a4 | | | | | | |
| 2b40a733-7a63-4bb8- | 0.072 | 0.94 | HR_deficient | BRCA1_type | Ovary | TRUE |
| a953-95a4ee28f962 | | | | | | |
| 42465bbd-289b-4e96- | 0.066 | 0.864 | HR_deficient | BRCA1_type | Ovary | TRUE |
| 98fe-76809c5e1520 | | | | | | |
| 51b25b37-f75c-4380- | 0.064 | 0.936 | HR_deficient | BRCA1_type | Ovary | TRUE |
| a0f6-5273e0b7ee33 | | | | | | |
| 6f981023-4269-4e8e- | 0.078 | 0.914 | HR_deficient | BRCA1_type | Ovary | TRUE |
| a4ab-2c92bb27273c | | | | | | |
| 8888e808-594b-4c76- | 0.056 | 0.944 | HR_deficient | BRCA1_type | Ovary | TRUE |
| b2e4-62aa56736f7c | | | | | | |
| 9e6806c4-d331-4025- | 0.676 | 0.842 | HR_deficient | BRCA2_type | Ovary | TRUE |
| abee-2f576e3bf182 | | | | | | |
| bc9b66f5-fcb8-4545- | 0.066 | 0.932 | HR_deficient | BRCA1_type | Ovary | TRUE |
| ab2d-438bb810edc0 | | | | | | |
| c2ec7f57-8510-4bbf- | 0.61 | 0.852 | HR_deficient | BRCA2_type | Ovary | TRUE |
| a2e9-dbd9ce8dcad1 | | | | | | |
| d4bf6034-aeae-48a6- | 0.016 | 0.942 | HR_deficient | BRCA1_type | Ovary | TRUE |
| 907b-10e2cc904015 | | | | | | |
| d8c2b4b2-e12b-43d2- | 0.072 | 0.944 | HR_deficient | BRCA1_type | Ovary | TRUE |
| bafc-87b29f027797 | | | | | | |
| da43386c-47f8-4e03- | 0.064 | 0.952 | HR_deficient | BRCA1_type | Ovary | TRUE |
| b6ca-8b94e13792e7 | | | | | | |
| dbbd54db-4470-4df6- | 0.5 | 0.852 | HR_deficient | BRCA2_type | Ovary | TRUE |
| b5be-3e175c7133b0 | | | | | | |
| e84debc4-b47d-48ed- | 0.124 | 0.906 | HR_deficient | BRCA1_type | Ovary | TRUE |
| a0d0-2859f0ebf987 | | | | | | |
| efec3225-de07-4559- | 0.126 | 0.924 | HR_deficient | BRCA1_type | Ovary | TRUE |
| 9a90-95223495cc61 | | | | | | |
| f1504811-8363-41e6- | 0.002 | 0.022 | HR_proficient | none | Ovary | TRUE |
| b43c-62452b1262d3 | | | | | | |
| f6189828-eeaa-4d21- | 0.09 | 0.928 | HR_deficient | BRCA1_type | Ovary | TRUE |
| b163-53bf3d47a640 | | | | | | |
| f988e698-9b34-45ce- | 0.044 | 0.936 | HR_deficient | BRCA1_type | Ovary | TRUE |
| ba4c-74e06e9cae4a | | | | | | |
| 00154198-023b-5137- | 0 | 0 | HR_proficient | none | Prostate | TRUE |
| ba0c-c7a612883ccf | | | | | | |
| 011fb67d-2b38-5b36- | 0.326 | 0.458 | HR_proficient | none | Prostate | TRUE |
| b967-1f8454cbb2aa | | | | | | |
| 0285dfea-ec3a-5900- | 0 | 0 | HR_proficient | none | Prostate | TRUE |
| 95d9-b712f070745a | | | | | | |
| 044bc566-299a-53b6- | 0 | 0 | HR_proficient | none | Prostate | TRUE |
| 9919-979a7feb4afa | | | | | | |
| 06f5711b-8457-5948- | 0 | 0 | HR_proficient | none | Prostate | TRUE |
| 8747-4fe6a0e8ad89 | | | | | | |
| 0708fca7-ec5b-5a46- | 0 | 0 | HR_proficient | none | Prostate | TRUE |
| a743-701a04106db6 | | | | | | |
| 07c64c28-5b9d-5e96- | 0 | 0 | HR_proficient | none | Prostate | TRUE |
| 9de7-dcfd748b9ecd | | | | | | |
| 0cb8ce81-8235-52f8- | 0 | 0 | HR_proficient | none | Prostate | TRUE |
| a698-fa4e0c96b39a | | | | | | |
| 0f58a603-4bfc-59bb- | 0 | 0 | HR_proficient | none | Prostate | TRUE |
| b5ff-a83d02981ba0 | | | | | | |
| 11068ec5-cab4-541d- | 0 | 0 | HR_proficient | none | Prostate | TRUE |
| 93df-452ac28c5c6c | | | | | | |
| 1128ed36-1fd5-590a- | 0 | 0 | HR_proficient | none | Prostate | TRUE |
| b615-7dd76f7d9e02 | | | | | | |
| 13cffb6c-f7bc-5adc- | 0 | 0 | HR_proficient | none | Prostate | TRUE |
| 8179-31cc2848bcdb | | | | | | |
| 144edc49-56d0-53ae- | 0 | 0 | HR_proficient | none | Prostate | TRUE |
| a4a0-aa3d82682ab7 | | | | | | |
| 167ffc5a-e5f2-50eb- | 0.176 | 0.482 | HR_proficient | none | Prostate | TRUE |
| b239-ae05e04741c7 | | | | | | |
| 169b915f-747b-5107- | 0 | 0 | HR_proficient | none | Prostate | TRUE |
| bdf0-dcb2175c69c9 | | | | | | |
| 16c48c28-e744-5034- | 0 | 0 | HR_proficient | none | Prostate | TRUE |
| 9b8d-af424c2dc1cc | | | | | | |
| 177fa3ac-eec3-5f38- | 0.002 | 0.002 | HR_proficient | none | Prostate | TRUE |
| 8204-3d97b85af187 | | | | | | |
| 18738c38-a99e-53c0- | 0 | 0 | HR_proficient | none | Prostate | TRUE |
| b2e3-baede8ad5e14 | | | | | | |
| 19fc59e1-ace5-58c5- | 0 | 0 | HR_proficient | none | Prostate | TRUE |
| a825-64178a1c065d | | | | | | |
| 1af15027-de25-597a- | 0 | 0 | HR_proficient | none | Prostate | TRUE |
| b45c-dbb840337b31 | | | | | | |
| 1b02bdd0-9d26-584a- | 0 | 0 | HR_proficient | none | Prostate | TRUE |
| 9d4f-9a8c3a130686 | | | | | | |
| 1db88eec-789e-5ccc- | 0 | 0 | HR_proficient | none | Prostate | TRUE |
| 9e0b-0b7897a7a4be | | | | | | |
| 1e8aeaf2-04c3-53a5- | 0 | 0 | HR_proficient | none | Prostate | TRUE |
| 8420-6c12dbdb6b11 | | | | | | |
| 1efa395b-642e-58a4- | 0.002 | 0.002 | HR_proficient | none | Prostate | TRUE |
| 8dad-647bb54d3f8c | | | | | | |
| 204daa0f-aa70-5173- | 0 | 0 | HR_proficient | none | Prostate | TRUE |
| b974-c13886ff1e86 | | | | | | |
| 20a74c97-747a-5a48- | 0 | 0 | HR_proficient | none | Prostate | TRUE |
| b217-f2883996d57d | | | | | | |
| 20ac8d6c-9b38-5bc7- | 0 | 0 | HR_proficient | none | Prostate | TRUE |
| a7f3-6c597e764456 | | | | | | |
| 2644b145-e218-5ca6- | 0 | 0 | HR_proficient | none | Prostate | TRUE |
| 80ca-da03350eb140 | | | | | | |
| 265febb1-200b-5417- | 0 | 0 | HR_proficient | none | Prostate | TRUE |
| 8424-767cea751935 | | | | | | |
| 27c5beb6-38a8-550c- | 0 | 0 | HR_proficient | none | Prostate | TRUE |
| a500-6f3cf4aab9f1 | | | | | | |
| 2a040e2e-ab16-504c- | 0 | 0 | HR_proficient | none | Prostate | TRUE |
| aae7-2a3caf6ea053 | | | | | | |
| 2a138e2a-293d-502b- | 0 | 0 | HR_proficient | none | Prostate | TRUE |
| 8fa5-b3da1dccfadd | | | | | | |
| 2d84abcd-0a7e-5dc1- | 0 | 0 | HR_proficient | none | Prostate | TRUE |
| 84d8-0e5184ea3d0d | | | | | | |
| 2da68bcd-11ef-562d- | 0 | 0 | HR_proficient | none | Prostate | TRUE |
| 9332-77ac387e2d91 | | | | | | |
| 2e1b33e2-54de-5693- | 0.748 | 0.85 | HR_deficient | BRCA2_type | Prostate | TRUE |
| ac6c-d18d5285cf23 | | | | | | |
| 301db1d1-307e-5652- | 0 | 0 | HR_proficient | none | Prostate | TRUE |
| 9faa-4849ccbad997 | | | | | | |
| 314cecfe-92fe-596c- | 0 | 0 | HR_proficient | none | Prostate | TRUE |
| aabd-2216b3692b6e | | | | | | |
| 319995f4-5632-5189- | 0.62 | 0.712 | HR_deficient | BRCA2_type | Prostate | TRUE |
| 83d8-ba63c797ef28 | | | | | | |
| 31cada43-4c06-5a78- | 0 | 0 | HR_proficient | none | Prostate | TRUE |
| b607-87d02a4d886c | | | | | | |
| 32f4b3db-f057-52c9- | 0.296 | 0.628 | HR_deficient | BRCA1_type | Prostate | TRUE |
| 9e98-aac05a825b7f | | | | | | |
| 32fa55db-71bf-5bc9- | 0.022 | 0.904 | HR_deficient | BRCA1_type | Prostate | TRUE |
| 9bfc-e00709b16f63 | | | | | | |
| 33623629-be74-5373- | 0 | 0 | HR_proficient | none | Prostate | TRUE |
| 8457-0e43f805b935 | | | | | | |
| 349cd0b4-0ab9-5786- | 0 | 0 | HR_proficient | none | Prostate | TRUE |
| bac4-cb95ea6052b1 | | | | | | |
| 3826a48c-9632-52ee- | 0 | 0 | HR_proficient | none | Prostate | TRUE |
| 8fc7-befdf0ffdc2a | | | | | | |
| 388feaa6-bc87-5d2e- | 0 | 0 | HR_proficient | none | Prostate | TRUE |
| 8acb-4fa8cd8adf2b | | | | | | |
| 3b46813a-e351-5f8d- | 0 | 0 | HR_proficient | none | Prostate | TRUE |
| aa4c-d08fc4713710 | | | | | | |
| 3b5ee9fb-cf66-5e9d- | 0 | 0 | HR_proficient | none | Prostate | TRUE |
| a4d7-0ec7606bc182 | | | | | | |
| 3cbf1093-8f79-59ca- | 0 | 0 | HR_proficient | none | Prostate | TRUE |
| babd-325b1e3beb44 | | | | | | |
| 3ffb6b77-73ec-5398- | 0 | 0 | HR_proficient | none | Prostate | TRUE |
| 9002-d28c72dad54c | | | | | | |
| 4033e0a8-d52c-5ad8- | 0 | 0 | HR_proficient | none | Prostate | TRUE |
| b576-268068bee8a9 | | | | | | |
| 4222a30d-e190-59b4- | 0 | 0 | HR_proficient | none | Prostate | TRUE |
| 93ef-0d1b8b68827d | | | | | | |
| 42c75c6c-eeb1-5dc2- | 0 | 0 | HR_proficient | none | Prostate | TRUE |
| 8239-fe5718cea7f0 | | | | | | |
| 42dad23b-8b78-58f8- | 0 | 0 | HR_proficient | none | Prostate | TRUE |
| 8027-9389a71f05e5 | | | | | | |
| 43ba96f4-5e7f-55b5- | 0 | 0 | HR_proficient | none | Prostate | TRUE |
| bd3c-a303d625a3f8 | | | | | | |
| 474668a4-6f29-5851- | 0 | 0 | HR_proficient | none | Prostate | TRUE |
| a063-2ad4964a6f90 | | | | | | |
| 47b69a2b-faab-55db- | 0 | 0 | HR_proficient | none | Prostate | TRUE |
| 9436-b4600d5d52ae | | | | | | |
| 47c173cf-3630-5223- | 0 | 0 | HR_proficient | none | Prostate | TRUE |
| b7f6-960e8b54f39f | | | | | | |
| 481e94d1-03f5-5c5c- | 0 | 0 | HR_proficient | none | Prostate | TRUE |
| add6-7603dfb4da73 | | | | | | |
| 4d9df4c3-89fb-5d80- | 0 | 0 | HR_proficient | none | Prostate | TRUE |
| b5b3-deae8088abf6 | | | | | | |
| 501ceb06-d90e-5907- | 0 | 0 | HR_proficient | none | Prostate | TRUE |
| 8f31-c28605e90b1a | | | | | | |
| 52d8403e-7872-5e34- | 0 | 0 | HR_proficient | none | Prostate | TRUE |
| 8b6f-8f767c1457d1 | | | | | | |
| 5342d185-c30d-5b45- | 0.074 | 0.284 | HR_proficient | none | Prostate | TRUE |
| b3dc-ffd064947bf1 | | | | | | |
| 55f32d60-b1e0-55c6- | 0 | 0 | HR_proficient | none | Prostate | TRUE |
| b5a0-263912972c48 | | | | | | |
| 57be9fdb-fc38-529d- | 0 | 0.002 | HR_proficient | none | Prostate | TRUE |
| 8aaf-46d0b7c75704 | | | | | | |
| 59dcef8d-513a-53a0- | 0 | 0 | HR_proficient | none | Prostate | TRUE |
| b56f-5bac2f400ff7 | | | | | | |
| 5e73036f-d33f-58ed- | 0 | 0 | HR_proficient | none | Prostate | TRUE |
| 92a4-abc536e4ee52 | | | | | | |
| 5f157436-48b7-5f3c- | 0 | 0 | HR_proficient | none | Prostate | TRUE |
| 8f34-c17058bff221 | | | | | | |
| 5fa51650-7759-50fa- | 0 | 0 | HR_proficient | none | Prostate | TRUE |
| be54-11930947454e | | | | | | |
| 60644314-5e6c-57f9- | 0 | 0 | HR_proficient | none | Prostate | TRUE |
| 8574-8c0c532fda3e | | | | | | |
| 610efdc3-690f-5e97- | 0 | 0 | HR_proficient | none | Prostate | TRUE |
| ae9f-3fcb9124afa8 | | | | | | |
| 6278f7b4-2858-5e1f- | 0 | 0 | HR_proficient | none | Prostate | TRUE |
| 9bce-661fafab14af | | | | | | |
| 62918f7c-470f-5527- | 0 | 0 | HR_proficient | none | Prostate | TRUE |
| bf52-fa3a6fac9da9 | | | | | | |
| 63db6f5a-316a-5273- | 0 | 0 | HR_proficient | none | Prostate | TRUE |
| a0f5-9c86c8554c2a | | | | | | |
| 648f8223-ac91-5119- | 0 | 0 | HR_proficient | none | Prostate | TRUE |
| bad3-ab4f28e0ff15 | | | | | | |
| 652f80ca-72e6-5521- | 0 | 0 | HR_proficient | none | Prostate | TRUE |
| b6ad-32aa7e5920e0 | | | | | | |
| 67a4d799-fe2b-570e- | 0 | 0 | HR_proficient | none | Prostate | TRUE |
| b75a-cd1220947bb9 | | | | | | |
| 6ab3463c-bf14-5c5d- | 0.006 | 0.006 | HR_proficient | none | Prostate | TRUE |
| 8cd0-5926a52d4b31 | | | | | | |
| 6bec564e-3701-58e9- | 0 | 0 | HR_proficient | none | Prostate | TRUE |
| bf1f-6b75f870823f | | | | | | |
| 6db0a33a-a450-5da7- | 0 | 0 | HR_proficient | none | Prostate | TRUE |
| 81c8-43b2a5e73462 | | | | | | |
| 6e4ed651-a190-5b1a- | 0.736 | 0.846 | HR_deficient | BRCA2_type | Prostate | TRUE |
| ade7-b4a649b72f73 | | | | | | |
| 6fd1df41-1cca-51db- | 0 | 0 | HR_proficient | none | Prostate | TRUE |
| a150-a146c81482e1 | | | | | | |
| 70a6e645-1d59-535b- | 0 | 0 | HR_proficient | none | Prostate | TRUE |
| b997-5018c3543964 | | | | | | |
| 728fb047-981b-5f9f- | 0 | 0 | HR_proficient | none | Prostate | TRUE |
| ae60-4054d70d1aae | | | | | | |
| 7439ad9d-4560-5486- | 0 | 0 | HR_proficient | none | Prostate | TRUE |
| be61-36bdbf52c686 | | | | | | |
| 76e741a7-3812-53b1- | 0 | 0 | HR_proficient | none | Prostate | TRUE |
| 8351-a3025378ad58 | | | | | | |
| 782f9ff6-0fca-5c39- | 0 | 0 | HR_proficient | none | Prostate | TRUE |
| abb1-f37a32692f4e | | | | | | |
| 795a44b6-13ab-587c- | 0 | 0 | HR_proficient | none | Prostate | TRUE |
| bceb-e80acbbe6669 | | | | | | |
| 7bb3a679-d6fa-5414- | 0 | 0 | HR_proficient | none | Prostate | TRUE |
| 811d-9807275ce1bf | | | | | | |
| 7d9065e3-718b-5b66- | 0 | 0 | HR_proficient | none | Prostate | TRUE |
| b1ac-03a11687649e | | | | | | |
| 7fed2782-c1bc-5990- | 0 | 0 | HR_proficient | none | Prostate | TRUE |
| aab8-4b8fb45bb5f4 | | | | | | |
| 80cbcf40-4e92-5b50- | 0 | 0 | HR_proficient | none | Prostate | TRUE |
| a461-79babec067c7 | | | | | | |
| 81c28bfc-36a8-550f- | 0 | 0 | HR_proficient | none | Prostate | TRUE |
| a167-7ba9c3a16555 | | | | | | |
| 8241a7ef-ba71-5ae8- | 0 | 0 | HR_proficient | none | Prostate | TRUE |
| 828d-c03bf77570ee | | | | | | |
| 83383700-b21e-5f4c- | 0 | 0 | HR_proficient | none | Prostate | TRUE |
| b1b5-6ab215cc0707 | | | | | | |
| 8621c8eb-5c34-580e- | 0 | 0 | HR_proficient | none | Prostate | TRUE |
| 9bc3-d88aac77c4d7 | | | | | | |
| 865eca7a-3dc9-5cfd- | 0.002 | 0.002 | HR_proficient | none | Prostate | TRUE |
| 9b11-930afcc4fed7 | | | | | | |
| 888c800e-d85f-53e9- | 0 | 0 | HR_proficient | none | Prostate | TRUE |
| 9ef3-5db944ecf26b | | | | | | |
| 88c140d5-8880-55d1- | 0 | 0 | HR_proficient | none | Prostate | TRUE |
| a322-5ef66431a36e | | | | | | |
| 8b8645ea-0010-5815- | 0.002 | 0.002 | HR_proficient | none | Prostate | TRUE |
| 8c98-95fe322fdcb7 | | | | | | |
| 8d6de6a5-5bd2-5c43- | 0 | 0 | HR_proficient | none | Prostate | TRUE |
| b357-dca27947818c | | | | | | |
| 8fb77a13-45d6-54b7- | 0 | 0 | HR_proficient | none | Prostate | TRUE |
| ad1a-e8539ca32022 | | | | | | |
| 91db5a58-5f0e-5b80- | 0 | 0 | HR_proficient | none | Prostate | TRUE |
| 81a7-96408ddbcfcc | | | | | | |
| 91e7e8ac-8e86-5a68- | 0 | 0 | HR_proficient | none | Prostate | TRUE |
| a7bc-898f2fba03b7 | | | | | | |
| 961e114d-5b6a-5c51- | 0 | 0 | HR_proficient | none | Prostate | TRUE |
| b069-85d3a742d7a7 | | | | | | |
| 9698d459-01d5-5fe6- | 0 | 0 | HR_proficient | none | Prostate | TRUE |
| 827c-d75dfcff5669 | | | | | | |
| 975669c5-fc96-56f8- | 0 | 0 | HR_proficient | none | Prostate | TRUE |
| b0d1-eadc1ff7f0c4 | | | | | | |
| 9b12ed50-6da8-54b7- | 0 | 0 | HR_proficient | none | Prostate | TRUE |
| 9dfb-621720408ec9 | | | | | | |
| 9ba249b8-0856-520b- | 0 | 0 | HR_proficient | none | Prostate | TRUE |
| a630-3fe835c77a30 | | | | | | |
| 9d70bc61-e795-5592- | 0 | 0 | HR_proficient | none | Prostate | TRUE |
| 91b8-118ea4a8a9c3 | | | | | | |
| 9f65bfd1-0846-55ef- | 0 | 0 | HR_proficient | none | Prostate | TRUE |
| 9043-4bfa0bc3fdef | | | | | | |
| 9f8c2b6f-fd32-5ea3- | 0 | 0 | HR_proficient | none | Prostate | TRUE |
| b11b-53df56016e5e | | | | | | |
| a0659c6a-2682-529e- | 0 | 0 | HR_proficient | none | Prostate | TRUE |
| aa70-f4bc064eebfa | | | | | | |
| a196e49f-9a45-5c77- | 0 | 0 | HR_proficient | none | Prostate | TRUE |
| b706-9add0246b114 | | | | | | |
| a5322930-562f-5ed1- | 0 | 0 | HR_proficient | none | Prostate | TRUE |
| b40f-1f19f19cd152 | | | | | | |
| a60c4297-856f-5f54- | 0.002 | 0.002 | HR_proficient | none | Prostate | TRUE |
| 85fd-d11293f67b49 | | | | | | |
| a8f075c0-ba1d-5e19- | 0 | 0 | HR_proficient | none | Prostate | TRUE |
| bb54-26e5d2b27a91 | | | | | | |
| aa16ed58-a498-5c83- | 0 | 0 | HR_proficient | none | Prostate | TRUE |
| a337-39d082940e0a | | | | | | |
| aba5a504-6840-5238- | 0.002 | 0.002 | HR_proficient | none | Prostate | TRUE |
| b628-9b9ee4e96a14 | | | | | | |
| ad2092d2-a673-5382- | 0 | 0 | HR_proficient | none | Prostate | TRUE |
| 8af4-d7fbe48e1678 | | | | | | |
| ad7b63f1-7a45-5f71- | 0 | 0 | HR_proficient | none | Prostate | TRUE |
| 9a42-9bb426cb36bb | | | | | | |
| b0d8663e-5726-5f3b- | 0 | 0 | HR_proficient | none | Prostate | TRUE |
| 9222-3735b61fb463 | | | | | | |
| b2101e56-fde5-5066- | 0.004 | 0.004 | HR_proficient | none | Prostate | TRUE |
| a95b-848cbf926953 | | | | | | |
| b2d2601e-e73d-586b- | 0 | 0 | HR_proficient | none | Prostate | TRUE |
| 8ef1-cba156697c36 | | | | | | |
| b44cfc59-cce5-5b5b- | 0 | 0 | HR_proficient | none | Prostate | TRUE |
| aa26-3348ff4a072f | | | | | | |
| b74cebec-2639-55eb- | 0 | 0 | HR_proficient | none | Prostate | TRUE |
| 9555-03b758fbb352 | | | | | | |
| ba225dda-f0e3-57ea- | 0 | 0 | HR_proficient | none | Prostate | TRUE |
| 9d24-eb407e0f39ef | | | | | | |
| bb889c67-42c7-5d52- | 0 | 0 | HR_proficient | none | Prostate | TRUE |
| 8c7c-b46cbb2b7f50 | | | | | | |
| bbd548b0-9da5-58fc- | 0 | 0 | HR_proficient | none | Prostate | TRUE |
| ae51-aa3387ce4223 | | | | | | |
| bc3f7d54-152e-545a- | 0.002 | 0.002 | HR_proficient | none | Prostate | TRUE |
| b32d-169e744e4a90 | | | | | | |
| c0878092-ee77-54ee- | 0 | 0 | HR_proficient | none | Prostate | TRUE |
| ba80-07035536ebfa | | | | | | |
| c0f83a8b-0027-5130- | 0 | 0 | HR_proficient | none | Prostate | TRUE |
| ad6f-52e1b296176b | | | | | | |
| c1b0f8bd-b2ad-5d03- | 0 | 0 | HR_proficient | none | Prostate | TRUE |
| 84cd-942971bf193a | | | | | | |
| c24a35b4-f507-5aa3- | 0 | 0 | HR_proficient | none | Prostate | TRUE |
| a4e3-dffb6a4a75f2 | | | | | | |
| c2ec5d59-5ead-5792- | 0 | 0 | HR_proficient | none | Prostate | TRUE |
| aacb-80078ceff799 | | | | | | |
| c4002b91-2b56-5368- | 0.022 | 0.904 | HR_deficient | BRCA1_type | Prostate | TRUE |
| aed2-2b792f4dbee7 | | | | | | |
| c4f6cca8-a8eb-5e9d- | 0 | 0 | HR_proficient | none | Prostate | TRUE |
| a9e7-99d26e6ae67a | | | | | | |
| c64376d2-bdfc-540b- | 0 | 0 | HR_proficient | none | Prostate | TRUE |
| 930a-8b7d510997bf | | | | | | |
| c67c5149-2753-5175- | 0 | 0 | HR_proficient | none | Prostate | TRUE |
| 9e2d-13d1290323ac | | | | | | |
| c781dd37-aba7-5427- | 0 | 0 | HR_proficient | none | Prostate | TRUE |
| b267-decc93da4df8 | | | | | | |
| c9476a45-6c52-5abe- | 0 | 0 | HR_proficient | none | Prostate | TRUE |
| a959-7713f1f1126b | | | | | | |
| cad23c3f-f06a-5d55- | 0 | 0 | HR_proficient | none | Prostate | TRUE |
| 9dcf-bbeec8764372 | | | | | | |
| cb80f145-46f5-5c3b- | 0 | 0 | HR_proficient | none | Prostate | TRUE |
| 9842-8686daf34306 | | | | | | |
| cbb4e48c-4bd0-58e9- | 0 | 0 | HR_proficient | none | Prostate | TRUE |
| b132-cc6c2b9cb9c5 | | | | | | |
| cecea6c7-ec8c-5d66- | 0 | 0 | HR_proficient | none | Prostate | TRUE |
| 965c-82a5df8d6a95 | | | | | | |
| cefd90ea-eda5-55a8- | 0 | 0 | HR_proficient | none | Prostate | TRUE |
| 83eb-0a66aa5c65a7 | | | | | | |
| cf046015-3f38-58ce- | 0 | 0 | HR_proficient | none | Prostate | TRUE |
| 811b-1044fc0c5f2c | | | | | | |
| dof782c5-3615-5cc4- | 0 | 0 | HR_proficient | none | Prostate | TRUE |
| 9ac4-6bafc93d5caf | | | | | | |
| d287c6a6-b6e1-5c30- | 0 | 0 | HR_proficient | none | Prostate | TRUE |
| b2cf-0bd7c993af0c | | | | | | |
| d413b834-09c8-5c76- | 0 | 0 | HR_proficient | none | Prostate | TRUE |
| a0fe-d55b67fdfe00 | | | | | | |
| d440c1f6-264a-51fa- | 0 | 0 | HR_proficient | none | Prostate | TRUE |
| be0e-0145bbae6710 | | | | | | |
| d6002f62-30b6-576a- | 0 | 0 | HR_proficient | none | Prostate | TRUE |
| 89b9-78e4fd50c7b4 | | | | | | |
| d744a1ee-a212-5229- | 0 | 0 | HR_proficient | none | Prostate | TRUE |
| 9d50-bb01c4256f76 | | | | | | |
| d873e58c-2c42-5bfa- | 0.264 | 0.602 | HR_deficient | BRCA1_type | Prostate | TRUE |
| aea2-79ab6790914d | | | | | | |
| daec4402-3b95-5229- | 0 | 0 | HR_proficient | none | Prostate | TRUE |
| 82bf-2274155f6d7c | | | | | | |
| dc2b7124-5b28-547f- | 0.718 | 0.84 | HR_deficient | BRCA2_type | Prostate | TRUE |
| b37a-d7ae37082cec | | | | | | |
| dc334f20-ce24-5c08- | 0 | 0 | HR_proficient | none | Prostate | TRUE |
| b189-b03fcd5361ea | | | | | | |
| dfb3ea4b-2ac7-52a3- | 0 | 0 | HR_proficient | none | Prostate | TRUE |
| b5a5-5910b0a1a6e1 | | | | | | |
| dfe3a3b4-9c46-5a7a- | 0 | 0.054 | HR_proficient | none | Prostate | TRUE |
| 95a6-648362aa75ba | | | | | | |
| e16dab0b-dc33-5df4- | 0 | 0 | HR_proficient | none | Prostate | TRUE |
| 8752-dd3a1b70a5b3 | | | | | | |
| e3e8cf24-e8be-5540- | 0 | 0 | HR_proficient | none | Prostate | TRUE |
| 924e-a38d2fa80704 | | | | | | |
| e468b4a3-9cc0-5339- | 0 | 0 | HR_proficient | none | Prostate | TRUE |
| a178-ece248a3d047 | | | | | | |
| e5bf367d-d70c-5dd4- | 0.308 | 0.618 | HR_deficient | BRCA1_type | Prostate | TRUE |
| 8b7f-17f5bb27e4f6 | | | | | | |
| e62a1fc5-1f58-5d80- | 0 | 0 | HR_proficient | none | Prostate | TRUE |
| a5aa-ef80a9d2200c | | | | | | |
| e76d10a0-fa72-5f0a- | 0 | 0 | HR_proficient | none | Prostate | TRUE |
| 9fa3-8691ce475510 | | | | | | |
| e8593980-a873-5c72- | 0 | 0 | HR_proficient | none | Prostate | TRUE |
| b5ae-8deae82d4fe9 | | | | | | |
| e89c60a0-a344-55f1- | 0.004 | 0.004 | HR_proficient | none | Prostate | TRUE |
| 82f4-ce61a6611c76 | | | | | | |
| e8a6cc14-b310-5398- | 0 | 0 | HR_proficient | none | Prostate | TRUE |
| 9cb9-b5b6d3ec45c5 | | | | | | |
| e9dc71bf-577b-57f1- | 0 | 0 | HR_proficient | none | Prostate | TRUE |
| 96c2-43ffcf3c2444 | | | | | | |
| ea320e35-11be-5231- | 0 | 0 | HR_proficient | none | Prostate | TRUE |
| 881b-b4e283789d30 | | | | | | |
| eb233756-a262-5fde- | 0 | 0 | HR_proficient | none | Prostate | TRUE |
| bd00-83f3aed1fb25 | | | | | | |
| ed01c279-2d23-5d92- | 0 | 0 | HR_proficient | none | Prostate | TRUE |
| 9680-d61a4ceb0ff3 | | | | | | |
| ed969e61-0ff7-59a6- | 0 | 0 | HR_proficient | none | Prostate | TRUE |
| 9e14-4f0dfaa10be5 | | | | | | |
| eeee9982-61f2-515c- | 0.006 | 0.016 | HR_proficient | none | Prostate | TRUE |
| be99-85680830d48c | | | | | | |
| f3d3ca98-020a-5115- | 0 | 0 | HR_proficient | none | Prostate | TRUE |
| 922c-edcad9fcf143 | | | | | | |
| f476071d-ca26-51dc- | 0 | 0 | HR_proficient | none | Prostate | TRUE |
| 87f7-d5962bb3f66d | | | | | | |
| f63896f2-21bb-5259- | 0 | 0 | HR_proficient | none | Prostate | TRUE |
| bb3b-a38963b3d2a9 | | | | | | |
| f712af1a-496f-5264- | 0 | 0 | HR_proficient | none | Prostate | TRUE |
| b787-65eb441855b9 | | | | | | |
| fb4faccb-cbcd-5706- | 0 | 0 | HR_proficient | none | Prostate | TRUE |
| bb5a-a8180c7f22bd | | | | | | |
| fc677c26-ac56-5522- | 0 | 0 | HR_proficient | none | Prostate | TRUE |
| ba62-7b86072c8703 | | | | | | |
| fd22f0c2-d50f-551c- | 0 | 0 | HR_proficient | none | Prostate | TRUE |
| 9b11-5496df8a56ef | | | | | | |
| fd246758-136b-5783- | 0.022 | 0.91 | HR_deficient | BRCA1_type | Prostate | TRUE |
| 8377-e8760dff9a6a | | | | | | |
| fed6c599-7e8a-52b4- | 0 | 0 | HR_proficient | none | Prostate | TRUE |
| a9b0-12aa1044559f | | | | | | |
| ffd69c4f-7134-55cd- | 0 | 0 | HR_proficient | none | Prostate | TRUE |
| 95a2-30d5e6d5d84e | | | | | | |
| 00213c2e-10b7-508a- | 0 | 0 | HR_proficient | none | Pancreas | TRUE |
| a4cf-98868df04abb | | | | | | |
| 004e8577-7340-5c55- | 0 | 0 | HR_proficient | none | Pancreas | TRUE |
| 8381-41d90a40fed2 | | | | | | |
| 00b81c0d-198c-59f7- | 0 | 0 | HR_proficient | none | Pancreas | TRUE |
| b492-64825c916dad | | | | | | |
| 01083a3c-2715-54f0- | 0 | 0 | HR_proficient | none | Pancreas | TRUE |
| 99d6-7385f0824189 | | | | | | |
| 02025ab4-4962-5284- | 0 | 0 | HR_proficient | none | Pancreas | TRUE |
| 8d06-cf913d035734 | | | | | | |
| 021639d8-e5de-596e- | 0.002 | 0.002 | HR_proficient | none | Pancreas | TRUE |
| 82f0-fe89dfb09747 | | | | | | |
| 026f6cfc-12a2-545a- | 0 | 0 | HR_proficient | none | Pancreas | TRUE |
| a4e0-ec865462a296 | | | | | | |
| 02f891ae-efaf-5e5e- | 0 | 0 | HR_proficient | none | Pancreas | TRUE |
| 8259-d2cfd96ed72d | | | | | | |
| 040c742c-b160-5661- | 0 | 0 | HR_proficient | none | Pancreas | TRUE |
| ae1c-c6a1cb2075ae | | | | | | |
| 084aeeb0-e733-5925- | 0 | 0 | HR_proficient | none | Pancreas | TRUE |
| a469-f92bd69a7bb5 | | | | | | |
| 08d85f2d-6019-50e7- | 0 | 0 | HR_proficient | none | Pancreas | TRUE |
| b122-c62827b36570 | | | | | | |
| 093a92ae-fb05-5164- | 0 | 0 | HR_proficient | none | Pancreas | TRUE |
| aad3-76f47dd301dc | | | | | | |
| 09df28c2-a195-5bfb- | 0 | 0 | HR_proficient | none | Pancreas | TRUE |
| 8474-6615433f7696 | | | | | | |
| 0b0ee084-0bcc-57d1- | 0.68 | 0.774 | HR_deficient | BRCA2_type | Pancreas | TRUE |
| 96f8-b11d0ba7e795 | | | | | | |
| 0f880c0f-5296-51d7- | 0 | 0 | HR_proficient | none | Pancreas | TRUE |
| 9a04-f3c935be66ea | | | | | | |
| 1086891c-4ab2-5c8a- | 0 | 0 | HR_proficient | none | Pancreas | TRUE |
| 9111-8d214006536b | | | | | | |
| 11631912-4d0b-571f- | 0 | 0 | HR_proficient | none | Pancreas | TRUE |
| 8c75-9a94ad3d5384 | | | | | | |
| 11758875-0852-5b3f- | 0 | 0 | HR_proficient | none | Pancreas | TRUE |
| 95d0-1e8dd2fb425a | | | | | | |
| 12505851-1bad-5829- | 0 | 0 | HR_proficient | none | Pancreas | TRUE |
| a768-b54ad7cc7425 | | | | | | |
| 16eaa5e9-491c-5427- | 0.002 | 0.002 | HR_proficient | none | Pancreas | TRUE |
| a7ae-3719c3989181 | | | | | | |
| 1730836c-eecf-5c04- | 0.002 | 0.002 | HR_proficient | none | Pancreas | TRUE |
| 9497-82997cbb29e9 | | | | | | |
| 18381ad9-3fa0-56b8- | 0 | 0 | HR_proficient | none | Pancreas | TRUE |
| 8809-03c56c2c90d0 | | | | | | |
| 1918201b-3a2f-5445- | 0 | 0 | HR_proficient | none | Pancreas | TRUE |
| b612-411d6168cbe0 | | | | | | |
| 194ee644-c3ff-56b3- | 0 | 0 | HR_proficient | none | Pancreas | TRUE |
| 82ae-4d39bb3bfb38 | | | | | | |
| 1f500fb1-84a1-5743- | 0 | 0 | \|HR_proficient | none | Pancreas | TRUE |
| b285-d9c2a6116a0b | | | | | | |
| 21a45669-3836-52e1- | 0 | 0 | HR_proficient | none | Pancreas | TRUE |
| 8be7-af73c5a03e8c | | | | | | |
| 21ad4e02-eaf3-598b- | 0 | 0 | HR_proficient | none | Pancreas | TRUE |
| a8d7-8479b250d702 | | | | | | |
| 246b1c1e-29e4-5984- | 0 | 0 | HR_proficient | none | Pancreas | TRUE |
| aefc-f5644eccb02e | | | | | | |
| 24b20aad-44fd-5143- | 0.742 | 0.798 | HR_deficient | BRCA2_type | Pancreas | TRUE |
| 8f20-f36107aaa00a | | | | | | |
| 27423c64-c787-5fe1- | 0 | 0 | HR_proficient | none | Pancreas | TRUE |
| 8f3f-847d1f6b7df2 | | | | | | |
| 284e9cf4-0314-5a62- | 0 | 0 | HR_proficient | none | Pancreas | TRUE |
| b509-bdfbd142b2e1 | | | | | | |
| 29b544db-04da-521a- | 0.002 | 0.002 | HR_proficient | none | Pancreas | TRUE |
| 9749-958c50b1887d | | | | | | |
| 2a8e0b23-6a95-5761- | 0 | 0 | HR_proficient | none | Pancreas | TRUE |
| 937a-2ae5c7a78403 | | | | | | |
| 2abdf944-e08d-5a3e- | 0 | 0 | HR_proficient | none | Pancreas | TRUE |
| 9e25-c05c4824cf6c | | | | | | |
| 2bd8c64e-c5b4-53c9- | 0 | 0 | HR_proficient | none | Pancreas | TRUE |
| 9079-c938b8c5406b | | | | | | |
| 2caeff1e-379c-5853- | 0 | 0 | HR_proficient | none | Pancreas | TRUE |
| a3d8-579aa99eba9d | | | | | | |
| 2d1ebf8c-b5b8-5bef- | 0 | 0 | HR_proficient | none | Pancreas | TRUE |
| b816-53d050d7456c | | | | | | |
| 2e885294-1461-5a27- | 0.596 | 0.718 | HR_deficient | BRCA2_type | Pancreas | TRUE |
| a3c0-fee7be206947 | | | | | | |
| 2e8a4934-ce3f-5c0c- | 0 | 0 | HR_proficient | none | Pancreas | TRUE |
| bb84-82e463e3f88d | | | | | | |
| 2eea14ec-8c3f-54d1- | 0.632 | 0.79 | HR_deficient | BRCA2_type | Pancreas | TRUE |
| a589-8897a1e033e1 | | | | | | |
| 2f75ea2e-ac46-5266- | 0 | 0 | HR_proficient | none | Pancreas | TRUE |
| 8b28-1632d9764fab | | | | | | |
| 30704065-1b62-5a7f- | 0 | 0.02 | HR_proficient | none | Pancreas | TRUE |
| b4b3-6a2cecbe88e8 | | | | | | |
| 33c0c336-e79d-53e7- | 0 | 0 | HR_proficient | none | Pancreas | TRUE |
| 8d48-f8755e536d3e | | | | | | |
| 33d37b9f-4104-5a19- | 0.004 | 0.012 | HR_proficient | none | Pancreas | TRUE |
| 8848-dc64b12b80db | | | | | | |
| 34bb9fa3-85c0-5c18- | 0 | 0 | HR_proficient | none | Pancreas | TRUE |
| a82d-0556f10cc607 | | | | | | |
| 37474afb-38a7-5c20- | 0 | 0 | HR_proficient | none | Pancreas | TRUE |
| b385-d7571b687901 | | | | | | |
| 374ff8bf-a2e2-5d3f- | 0 | 0 | HR_proficient | none | Pancreas | TRUE |
| a706-a2ec12227afd | | | | | | |
| 38ba9f2c-bcd8-594c- | 0.002 | 0.002 | HR_proficient | none | Pancreas | TRUE |
| 8d44-99d3f434f95a | | | | | | |
| 3990a431-ad52-50d7- | 0 | 0 | HR_proficient | none | Pancreas | TRUE |
| 8d60-ff294b4739b6 | | | | | | |
| 3a82c2a0-e2c8-559e- | 0 | 0 | HR_proficient | none | Pancreas | TRUE |
| 8eae-55bad0a6fdd1 | | | | | | |
| 3b649306-5d50-5ffd- | 0.624 | 0.706 | HR_deficient | BRCA2_type | Pancreas | TRUE |
| a508-0a42f49f65f9 | | | | | | |
| 3c6ab0f4-1170-55fe- | 0 | 0 | HR_proficient | none | Pancreas | TRUE |
| a092-12a100d1d65c | | | | | | |
| 3cb282a8-e7c8-58f2- | 0 | 0 | HR_proficient | none | Pancreas | TRUE |
| bebb-7e355da96cc0 | | | | | | |
| 3ce8bd0d-dc8e-5bee- | 0 | 0.012 | HR_proficient | none | Pancreas | TRUE |
| a351-ab79418a8755 | | | | | | |
| 3f2f425e-00fa-5100- | 0 | 0 | HR_proficient | none | Pancreas | TRUE |
| 84cc-335bcbb8b604 | | | | | | |
| 3fab3482-0253-5f38- | 0 | 0 | HR_proficient | none | Pancreas | TRUE |
| 8b63-4ddac2c7933e | | | | | | |
| 4115ccd5-4e6f-584a- | 0 | 0 | HR_proficient | none | Pancreas | TRUE |
| 9c3f-67d367f3a3f2 | | | | | | |
| 418c2c49-c470-52a3- | 0 | 0.002 | HR_proficient | none | Pancreas | TRUE |
| 9895-29d33a557404 | | | | | | |
| 432629d7-4f6f-5739- | 0 | 0 | HR_proficient | none | Pancreas | TRUE |
| abda-24da355f1d52 | | | | | | |
| 43b1206f-7129-5493- | 0 | 0 | HR_proficient | none | Pancreas | TRUE |
| 8472-f14886ae9785 | | | | | | |
| 47344f5f-467f-5f91- | 0 | 0 | HR_proficient | none | Pancreas | TRUE |
| 8695-59d1164e7755 | | | | | | |
| 4790fb9e-04fd-51fd- | 0.002 | 0.008 | HR_proficient | none | Pancreas | TRUE |
| 8a8b-0df0e8372b3c | | | | | | |
| 47c573a7-b108-5c73- | 0 | 0 | HR_proficient | none | Pancreas | TRUE |
| a221-c0c6103a7330 | | | | | | |
| 48849c94-a5f0-516e- | 0 | 0 | HR_proficient | none | Pancreas | TRUE |
| 89ac-2db95b570480 | | | | | | |
| 48bc879e-2cbb-54cb- | 0 | 0.002 | HR_proficient | none | Pancreas | TRUE |
| 82c3-7be3ebf971d2 | | | | | | |
| 4c913ea0-f24c-5be4- | 0.012 | 0.03 | HR_proficient | none | Pancreas | TRUE |
| a0c7-df56c0fa2ca4 | | | | | | |
| 4df913af-9ce5-59f3- | 0 | 0 | HR_proficient | none | Pancreas | TRUE |
| ba8e-b52ccc267718 | | | | | | |
| 4e13d88e-fef2-568d- | 0 | 0 | HR_proficient | none | Pancreas | TRUE |
| 982f-bf6158e23262 | | | | | | |
| 4e6c5a6d-024c-5e3a- | 0 | 0 | HR_proficient | none | Pancreas | TRUE |
| a515-7de51265ce96 | | | | | | |
| 4fecdafc-ee08-5caa- | 0.002 | 0.002 | HR_proficient | none | Pancreas | TRUE |
| 9225-9335f0b2fa63 | | | | | | |
| 5096f0b3-5a43-5cee- | 0 | 0 | HR_proficient | none | Pancreas | TRUE |
| 9dd3-20d5c4f9c423 | | | | | | |
| 5189306c-7195-55cf- | 0 | 0 | HR_proficient | none | Pancreas | TRUE |
| 9aa6-70b93456b011 | | | | | | |
| 538ccbff-bf39-5c45- | 0 | 0 | HR_proficient | none | Pancreas | TRUE |
| acf8-09938858b4c2 | | | | | | |
| 560e9919-d34d-53bd- | 0 | 0 | HR_proficient | none | Pancreas | TRUE |
| acea-e248f89b756e | | | | | | |
| 569a3222-c189-5b0e- | 0 | 0.006 | HR_proficient | none | Pancreas | TRUE |
| 8ced-01d75b5108f2 | | | | | | |
| 56a634de-b0ed-5bdc- | 0 | 0.002 | HR_proficient | none | Pancreas | TRUE |
| ae07-8d22aa762b61 | | | | | | |
| 57cb4f59-796a-56e9- | 0 | 0 | HR_proficient | none | Pancreas | TRUE |
| 8b86-cf0239040336 | | | | | | |
| 57d038bb-2b7f-5e26- | 0 | 0 | HR_proficient | none | Pancreas | TRUE |
| b10e-e15e60a879ec | | | | | | |
| 599463f7-b9aa-583b- | 0.002 | 0.002 | HR_proficient | none | Pancreas | TRUE |
| 80dd-2556bf385eae | | | | | | |
| 59a98d91-2b50-54cd- | 0 | 0 | HR_proficient | none | Pancreas | TRUE |
| 8162-08d3b7e132cd | | | | | | |
| 5c36bbb2-015a-5587- | 0 | 0 | HR_proficient | none | Pancreas | TRUE |
| 8f14-63f7e4c5183c | | | | | | |
| 60fdfca2-8a8a-595a- | 0 | 0 | HR_proficient | none | Pancreas | TRUE |
| b11b-b40d95b353ff | | | | | | |
| 62211e76-5da0-57aa- | 0.582 | 0.704 | HR_deficient | BRCA2_type | Pancreas | TRUE |
| a8ae-1b0cbc14b41b | | | | | | |
| 62b4e4d7-67db-5f21- | 0 | 0.004 | HR_proficient | none | Pancreas | TRUE |
| 902d-b5d01677505e | | | | | | |
| 64eb7730-c9af-5eb1- | 0 | 0 | HR_proficient | none | Pancreas | TRUE |
| 939e-519d93ca878e | | | | | | |
| 690b056d-3a55-546d- | 0 | 0.048 | HR_proficient | none | Pancreas | TRUE |
| a239-5da99d2166b3 | | | | | | |
| 69dda2c3-1f62-5d58- | 0.002 | 0.008 | HR_proficient | none | Pancreas | TRUE |
| a482-991d959c10e5 | | | | | | |
| 6c178066-4e9d-5f87- | 0 | 0 | HR_proficient | none | Pancreas | TRUE |
| 88a4-bbcca334b5a2 | | | | | | |
| 6c6a2f4c-aad4-5944- | 0 | 0 | HR_proficient | none | Pancreas | TRUE |
| abef-3d7cd0144429 | | | | | | |
| 6d7dab97-8dac-58bc- | 0 | 0 | HR_proficient | none | Pancreas | TRUE |
| 974e-7dba2df938f8 | | | | | | |
| 6d91daa8-f8a5-5065- | 0 | 0 | HR_proficient | none | Pancreas | TRUE |
| a40c-03cf6484f612 | | | | | | |
| 6e0789d0-e8bc-5e6d- | 0 | 0 | HR_proficient | none | Pancreas | TRUE |
| a83a-b8563d61ec73 | | | | | | |
| 6f8aec20-dbb0-5232- | 0 | 0 | HR_proficient | none | Pancreas | TRUE |
| a2b6-fb8ed3fe9c4f | | | | | | |
| 73a2e451-5e53-5ff6- | 0 | 0 | HR_proficient | none | Pancreas | TRUE |
| 8348-69292c2b3ef3 | | | | | | |
| 75f0ebd2-d791-520c- | 0 | 0.002 | HR_proficient | none | Pancreas | TRUE |
| 980b-1264ecf7dc5f | | | | | | |
| 76ebb33a-7405-5871- | 0 | 0 | HR_proficient | none | Pancreas | TRUE |
| be7d-6520ee988899 | | | | | | |
| 789bf8d1-ac6e-57da- | 0 | 0 | HR_proficient | none | Pancreas | TRUE |
| 9f34-1545d40155eb | | | | | | |
| 78b1787a-6191-50f6- | 0 | 0.016 | HR_proficient | none | Pancreas | TRUE |
| 9c20-0dfacb87bfa4 | | | | | | |
| 78d4c74a-11d8-5fe2- | 0.368 | 0.77 | HR_deficient | BRCA1_type | Pancreas | TRUE |
| 9be3-ffa09d30c4ea | | | | | | |
| 79fce701-1b11-5849- | 0 | 0 | HR_proficient | none | Pancreas | TRUE |
| 8b7d-677e49ecff49 | | | | | | |
| 7a447ffc-f4a2-5d15- | 0 | 0 | HR_proficient | none | Pancreas | TRUE |
| 82f9-e0b624b5632f | | | | | | |
| 7c24476b-c721-559e- | 0 | 0 | HR_proficient | none | Pancreas | TRUE |
| b945-ad21682ebed7 | | | | | | |
| 7cee07d4-d459-5e98- | 0 | 0 | HR_proficient | none | Pancreas | TRUE |
| 894b-ba74575e8de2 | | | | | | |
| 7db45081-5eeb-5496- | 0 | 0.056 | HR_proficient | none | Pancreas | TRUE |
| a51f-a209b81d9c12 | | | | | | |
| 7f3b6b4f-4876-56e4- | 0 | 0 | HR_proficient | none | Pancreas | TRUE |
| beab-082d1c975710 | | | | | | |
| 7f4312bc-fe88-5b9a- | 0 | 0 | HR_proficient | none | Pancreas | TRUE |
| a292-2f87cf248ccd | | | | | | |
| 7fd9535f-3512-5765- | 0 | 0 | HR_proficient | none | Pancreas | TRUE |
| a369-82d139705567 | | | | | | |
| 801dcc66-92e6-5575- | 0 | 0 | HR_proficient | none | Pancreas | TRUE |
| a2a5-d478e5bf6773 | | | | | | |
| 80c539c4-ad9e-5ac0- | 0 | 0.066 | HR_proficient | none | Pancreas | TRUE |
| 83a7-8277de850443 | | | | | | |
| 82dc9348-35d3-5818- | 0 | 0 | HR_proficient | none | Pancreas | TRUE |
| 9d53-83cb5405c323 | | | | | | |
| 84e48eaf-fa44-5a3e- | 0 | 0 | HR_proficient | none | Pancreas | TRUE |
| 99e1-59123d2b1bee | | | | | | |
| 8656f03e-ae65-5568- | 0 | 0 | HR_proficient | none | Pancreas | TRUE |
| aa1c-54fd964d4305 | | | | | | |
| 871fc242-e557-52c6- | 0 | 0 | HR_proficient | none | Pancreas | TRUE |
| a996-c535669a855f | | | | | | |
| 880574c0-99b7-5494- | 0 | 0 | HR_proficient | none | Pancreas | TRUE |
| b3d8-994692b11d95 | | | | | | |
| 8ac7e33f-0e0d-529f- | 0 | 0 | HR_proficient | none | Pancreas | TRUE |
| a653-0ceeeafb0f34 | | | | | | |
| 8c1e49f9-ed1d-5e1a- | 0 | 0 | HR_proficient | none | Pancreas | TRUE |
| 9913-509003f6cebc | | | | | | |
| 8c4fa1eb-6426-5733- | 0 | 0 | HR_proficient | none | Pancreas | TRUE |
| acdc-81b881c31c69 | | | | | | |
| 8e3a5956-23da-585c- | 0 | 0 | HR_proficient | none | Pancreas | TRUE |
| 99d3-b8f905d3cdf8 | | | | | | |
| 915b1720-17e3-54bf- | 0 | 0 | HR_proficient | none | Pancreas | TRUE |
| 924e-365b49647aa4 | | | | | | |
| 926e8cd0-ffcd-5cdc- | 0.176 | 0.69 | HR_deficient | BRCA1_type | Pancreas | TRUE |
| 9f63-95a53a23c443 | | | | | | |
| 933061cd-7229-533c- | 0.668 | 0.818 | HR_deficient | BRCA2_type | Pancreas | TRUE |
| 92a5-0f01be6498c7 | | | | | | |
| 938ef410-e08e-59eb- | 0 | 0 | HR_proficient | none | Pancreas | TRUE |
| 918d-1b87c296ca96 | | | | | | |
| 94669dbe-8d87-55d8- | 0 | 0 | HR_proficient | none | Pancreas | TRUE |
| 80ae-678c6b2be998 | | | | | | |
| 99afadf2-07f7-5105- | 0.472 | 0.618 | HR_deficient | BRCA2_type | Pancreas | TRUE |
| 8b1b-5328be8f7c69 | | | | | | |
| 9a36a27e-4037-53d9- | 0 | 0 | HR_proficient | none | Pancreas | TRUE |
| 9652-b898c5f8b088 | | | | | | |
| 9a5b350a-2387-54fb- | 0 | 0 | HR_proficient | none | Pancreas | TRUE |
| a4de-c4c09b0194f4 | | | | | | |
| 9c5f0679-4745-50b9- | 0 | 0 | HR_proficient | none | Pancreas | TRUE |
| ae5c-8279c14d84ed | | | | | | |
| 9d3f34b2-6497-5886- | 0 | 0.02 | HR_proficient | none | Pancreas | TRUE |
| 89ca-924673e28abc | | | | | | |
| 9d5907d3-180f-5364- | 0 | 0 | HR_proficient | none | Pancreas | TRUE |
| b75f-281459b60aa2 | | | | | | |
| 9dfe717d-acb3-5e86- | 0 | 0 | HR_proficient | none | Pancreas | TRUE |
| 8d14-14f56a1472fe | | | | | | |
| 9efcb184-a6b4-52e7- | 0 | 0 | HR_proficient | none | Pancreas | TRUE |
| b37d-4bfa12ef57f5 | | | | | | |
| a1713a31-68bf-5e0a- | 0 | 0 | HR_proficient | none | Pancreas | TRUE |
| a27d-bc266aff2dc0 | | | | | | |
| a1b6eac7-71f1-59c7- | 0 | 0 | HR_proficient | none | Pancreas | TRUE |
| b6fd-03be54cda35c | | | | | | |
| a5c8b2b5-ef5a-5475- | 0 | 0 | HR_proficient | none | Pancreas | TRUE |
| acbf-87cd0072d3a2 | | | | | | |
| a6c0456e-f76f-59ee- | 0 | 0 | HR_proficient | none | Pancreas | TRUE |
| 892e-1d3fe03da47f | | | | | | |
| a9c962ff-5a4f-5464- | 0.002 | 0.006 | HR_proficient | none | Pancreas | TRUE |
| 8c53-e43adcd26e36 | | | | | | |
| aa05ac1c-b123-5d97- | 0.128 | 0.898 | HR_deficient | BRCA1_type | Pancreas | TRUE |
| 9c32-24bc40831b76 | | | | | | |
| ac398ef4-7baf-573c- | 0 | 0 | HR_proficient | none | Pancreas | TRUE |
| 82c4-e78f3a86bc33 | | | | | | |
| ac6a5c70-e9c7-5f9a- | 0 | 0 | HR_proficient | none | Pancreas | TRUE |
| b1ed-09ca6aa65e0a | | | | | | |
| ae3d51c8-0a2d-55ac- | 0 | 0 | HR_proficient | none | Pancreas | TRUE |
| 9e51-3d8d275ca715 | | | | | | |
| ae515006-2778-591f- | 0 | 0.002 | HR_proficient | none | Pancreas | TRUE |
| ae50-8858b5af1a0a | | | | | | |
| ae9722c4-3f1d-5424- | 0 | 0 | HR_proficient | none | Pancreas | TRUE |
| 9999-354ecd6d6619 | | | | | | |
| b25c51ec-b5e3-5c6a- | 0.002 | 0.002 | HR_proficient | none | Pancreas | TRUE |
| 8904-94319ba2e1e0 | | | | | | |
| b43d709a-657d-5c49- | 0 | 0.002 | HR_proficient | none | Pancreas | TRUE |
| 9500-a98c44930fa7 | | | | | | |
| b4f221e9-2c0b-5ad3- | 0 | 0 | HR_proficient | none | Pancreas | TRUE |
| aac2-4fefcfa1aea6 | | | | | | |
| b5237253-7214-5dcd- | 0 | 0 | HR_proficient | none | Pancreas | TRUE |
| 8dff-9c1a429a7c57 | | | | | | |
| b54de531-e7da-521f- | 0 | 0 | HR_proficient | none | Pancreas | TRUE |
| a292-68740cc967a9 | | | | | | |
| b7b176c9-0e5d-5de9- | 0 | 0 | HR_proficient | none | Pancreas | TRUE |
| a679-4555f441d914 | | | | | | |
| b8269bc3-8c0f-5af2- | 0 | 0 | HR_proficient | none | Pancreas | TRUE |
| af9b-ce2cd9319848 | | | | | | |
| b9d3cf7f-80df-5107- | 0.176 | 0.59 | HR_deficient | BRCA1_type | Pancreas | TRUE |
| ba85-f287ac250aa7 | | | | | | |
| bb3ecb9d-3b53-5e0e- | 0 | 0 | HR_proficient | none | Pancreas | TRUE |
| 88ea-69081a11c993 | | | | | | |
| bc4d5c8f-277b-5946- | 0 | 0 | HR_proficient | none | Pancreas | TRUE |
| 8ce0-ea58f5e2fa4c | | | | | | |
| bcd38401-cb6c-5cd9- | 0 | 0 | HR_proficient | none | Pancreas | TRUE |
| 914a-3501fab6e771 | | | | | | |
| bda9e129-780e-5182- | 0.508 | 0.73 | HR_deficient | BRCA2_type | Pancreas | TRUE |
| a056-d614617da60b | | | | | | |
| bdf175be-a89a-5a16- | 0 | 0 | HR_proficient | none | Pancreas | TRUE |
| af67-8135ee914b4e | | | | | | |
| be92e51a-6d14-505d- | 0.398 | 0.834 | HR_deficient | BRCA1_type | Pancreas | TRUE |
| b997-e1695c765ef0 | | | | | | |
| bf6c08fa-bf28-5971- | 0 | 0 | HR_proficient | none | Pancreas | TRUE |
| 912f-340e5bf73a0e | | | | | | |
| c01807ea-b622-5197- | 0 | 0.002 | HR_proficient | none | Pancreas | TRUE |
| b88d-bbededc15025 | | | | | | |
| c28296f4-c609-56d9- | 0 | 0 | HR_proficient | none | Pancreas | TRUE |
| 9af7-5b7977eb0c67 | | | | | | |
| c4f4f1c9-ddd7-500f- | 0 | 0 | HR_proficient | none | Pancreas | TRUE |
| 8a9a-1bd3fba46e53 | | | | | | |
| c678c547-b34d-5ec5- | 0 | 0 | HR_proficient | none | Pancreas | TRUE |
| b052-2d946b1ea6a1 | | | | | | |
| c8f8440c-da1a-5a85- | 0 | 0 | HR_proficient | none | Pancreas | TRUE |
| 9c63-95f6615279f4 | | | | | | |
| ca1b6fbb-b794-5f30- | 0 | 0 | HR_proficient | none | Pancreas | TRUE |
| a727-7ed7d7a061d4 | | | | | | |
| ca8a0cc6-6dd3-5d91- | 0 | 0 | HR_proficient | none | Pancreas | TRUE |
| 8de3-b0324728bfb4 | | | | | | |
| cb1d1bfd-5588-57a7- | 0 | 0 | HR_proficient | none | Pancreas | TRUE |
| a962-b354a6740dbe | | | | | | |
| cb30608b-1362-5330- | 0 | 0 | HR_proficient | none | Pancreas | TRUE |
| ba65-e55176b295cf | | | | | | |
| cbce448e-62ee-5fcf- | 0.676 | 0.848 | HR_deficient | BRCA2_type | Pancreas | TRUE |
| 87b8-4fd0abeed013 | | | | | | |
| cc605155-d350-5da6- | 0 | 0 | HR_proficient | none | Pancreas | TRUE |
| 881b-85ba7414e273 | | | | | | |
| cd323cd5-7b72-59b0- | 0 | 0.002 | HR_proficient | none | Pancreas | TRUE |
| bcf9-e89712635f3b | | | | | | |
| cf3db688-8f76-5e3f- | 0.004 | 0.018 | HR_proficient | none | Pancreas | TRUE |
| b4c3-8ea6f1dbfb6a | | | | | | |
| cfd68b84-43a9-5458- | 0 | 0 | HR_proficient | none | Pancreas | TRUE |
| 8822-cb30fcbcfc05 | | | | | | |
| d1beb958-66fb-5cf0- | 0 | 0 | HR_proficient | none | Pancreas | TRUE |
| 86ec-f569177e8e02 | | | | | | |
| d214b014-9a5a-5450- | 0 | 0 | HR_proficient | none | Pancreas | TRUE |
| 92dc-029ec14b563f | | | | | | |
| d2732e94-a759-59d4- | 0 | 0 | HR_proficient | none | Pancreas | TRUE |
| 9f35-d09d25a3fda3 | | | | | | |
| d34c6934-d0c8-58b5- | 0 | 0 | HR_proficient | none | Pancreas | TRUE |
| 9a34-9e1f2a54fea7 | | | | | | |
| d4a4bd38-5f52-5cda- | 0 | 0 | HR_proficient | none | Pancreas | TRUE |
| b442-f9f380e11421 | | | | | | |
| d64b3fe1-5b2d-5845- | 0 | 0 | HR_proficient | none | Pancreas | TRUE |
| 85bc-ed5e5c2071a4 | | | | | | |
| d68a2d08-a9d5-52c3- | 0 | 0 | HR_proficient | none | Pancreas | TRUE |
| 82ba-07514970f061 | | | | | | |
| d6eb18f4-c295-5700- | 0 | 0 | HR_proficient | none | Pancreas | TRUE |
| 9a69-e41efba30457 | | | | | | |
| d6fece5a-3a0b-54f9- | 0.004 | 0.028 | HR_proficient | none | Pancreas | TRUE |
| a074-3c2b44713584 | | | | | | |
| d7af903d-3e21-5b92- | 0 | 0.004 | HR_proficient | none | Pancreas | TRUE |
| ac3d-85a25fe3c99b | | | | | | |
| d8a76e59-a22c-55d6- | 0.03 | 0.058 | HR_proficient | none | Pancreas | TRUE |
| 98f4-0f5e627f13e3 | | | | | | |
| d997c058-ad11-545d- | 0 | 0 | HR_proficient | none | Pancreas | TRUE |
| 9257-927c93385a2f | | | | | | |
| db04b66e-f1e7-5753- | 0 | 0 | HR_proficient | none | Pancreas | TRUE |
| a289-546d36cbb904 | | | | | | |
| df20f849-202a-5018- | 0 | 0 | HR_proficient | none | Pancreas | TRUE |
| 8343-f966daf012ac | | | | | | |
| df8a5ed8-acff-53fc- | 0 | 0 | HR_proficient | none | Pancreas | TRUE |
| 80ee-efde0acdceed | | | | | | |
| e059a9fe-8842-525d- | 0 | 0 | HR_proficient | none | Pancreas | TRUE |
| 85f5-fe4e47baa9df | | | | | | |
| e2d9369c-761d-56d6- | 0 | 0 | HR_proficient | none | Pancreas | TRUE |
| 9365-9e8eb6c85552 | | | | | | |
| e3e12acb-66bf-5b50- | 0 | 0 | HR_proficient | none | Pancreas | TRUE |
| b052-9351ade4bd9d | | | | | | |
| e43bf8db-9d73-5555- | 0 | 0 | HR_proficient | none | Pancreas | TRUE |
| 8648-da1d3b78bdc3 | | | | | | |
| e44cded3-7144-5dac- | 0 | 0 | HR_proficient | none | Pancreas | TRUE |
| b95c-727a2995f15c | | | | | | |
| e491ad53-9222-592d- | 0 | 0 | HR_proficient | none | Pancreas | TRUE |
| 93a1-79d34ccf4e76 | | | | | | |
| e4b3fa16-98b2-5b02- | 0 | 0.058 | HR_proficient | none | Pancreas | TRUE |
| 9b8f-b76d5c6526f3 | | | | | | |
| e5d84bc0-9013-53bf- | 0 | 0 | HR_proficient | none | Pancreas | TRUE |
| 91c0-676f305c5102 | | | | | | |
| e6efbe00-7125-5a98- | 0 | 0 | HR_proficient | none | Pancreas | TRUE |
| a695-a34be6f7ddb4 | | | | | | |
| e6f965d2-f2fc-56e6- | 0 | 0 | HR_proficient | none | Pancreas | TRUE |
| ae12-cb2671e228e0 | | | | | | |
| e78f4f84-3a46-5ab5- | 0 | 0 | HR_proficient | none | Pancreas | TRUE |
| b5c3-4b59b61c378a | | | | | | |
| e7bd6083-1cb8-5da3- | 0 | 0 | HR_proficient | none | Pancreas | TRUE |
| b701-fd32791d5ce9 | | | | | | |
| ebdcf8f1-fcd5-5269- | 0 | 0 | HR_proficient | none | Pancreas | TRUE |
| ab53-15ff9635e797 | | | | | | |
| f077e1c8-5729-57e8- | 0 | 0 | HR_proficient | none | Pancreas | TRUE |
| 9029-eb1d2238e0c0 | | | | | | |
| f28cb778-14f1-5064- | 0.606 | 0.788 | HR_deficient | BRCA2_type | Pancreas | TRUE |
| 8641-df9a341d56b6 | | | | | | |
| f368e8a1-a788-546f- | 0 | 0 | HR_proficient | none | Pancreas | TRUE |
| 8a7a-ac42588a5a5a | | | | | | |
| f3c0e89a-0a00-521d- | 0 | 0 | HR_proficient | none | Pancreas | TRUE |
| bc30-c4893de5fb98 | | | | | | |
| f42009f2-dba1-52b0- | 0.754 | 0.848 | HR_deficient | BRCA2_type | Pancreas | TRUE |
| b52b-44c7c8148df0 | | | | | | |
| f65fc85d-fb73-5e2e- | 0 | 0.008 | HR_proficient | none | Pancreas | TRUE |
| bd81-c54baccaa436 | | | | | | |
| f68c50fd-a3bb-5761- | 0 | 0 | HR_proficient | none | Pancreas | TRUE |
| a194-0bc201da44f6 | | | | | | |
| f6b383ef-b07e-5b38- | 0 | 0 | HR_proficient | none | Pancreas | TRUE |
| b2ea-19690ea97423 | | | | | | |
| f7ba5d0c-70d3-5130- | 0 | 0 | HR_proficient | none | Pancreas | TRUE |
| 92d7-5bcfed0eed5f | | | | | | |
| f8a54ba5-236d-5b27- | 0 | 0 | HR_proficient | none | Pancreas | TRUE |
| a292-e68e2fc3b8ac | | | | | | |
| f8ae0285-c759-52f6- | 0.818 | 0.864 | HR_deficient | BRCA2_type | Pancreas | TRUE |
| a4ed-7f25b8f686a5 | | | | | | |
| f927eb70-4a26-5e11- | 0 | 0 | HR_proficient | none | Pancreas | TRUE |
| 91ed-ae138ce666dc | | | | | | |
| fabc6e53-cb9e-5053- | 0.7 | 0.754 | HR_deficient | BRCA2_type | Pancreas | TRUE |
| 82ba-f557aa277a3c | | | | | | |
| fb506451-7ed9-5609- | 0 | 0 | HR_proficient | none | Pancreas | TRUE |
| aea6-de565f96e967 | | | | | | |
| fb732684-1372-5230- | 0 | 0 | HR_proficient | none | Pancreas | TRUE |
| 9dc7-209814d8a7f7 | | | | | | |
| fda90ec7-e3ef-5089- | 0 | 0 | HR_proficient | none | Pancreas | TRUE |
| b784-97d0fb742f15 | | | | | | |
| fff213d8-3d8c-583d- | 0 | 0 | HR_proficient | none | Pancreas | TRUE |
| 8f89-65f2c33572cd | | | | | | |

TABLE 11
(samples)

| | library_{—} | | | ULP_tumor_m | panel_tumor_{—} |
|---|---|---|---|---|---|
| sample | type | wgs_pct_exc_dupe | Mean_coverage | fraction | fraction |
| 108200 | Normal | 0.188786 | 24.90962 | | |
| 128311 | Normal | 0.203731 | 25.691397 | | |
| 384102 | Normal | 0.149078 | 35.214779 | | |
| 407120 | Normal | 0.156925 | 34.224499 | | |
| 476703 | Normal | 0.203892 | 24.961029 | | |
| 500560 | Normal | 0.138734 | 30.40444 | | |
| 566240 | Normal | 0.175547 | 25.451119 | | |
| 566691 | Normal | 0.183536 | 24.247287 | | |
| 718458 | Normal | 0.147508 | 35.203573 | | |
| 752770 | Normal | NA | 28.89 | | |
| 646059 | Normal | 0.268933 | 21.994001 | | |
| 653096 | Normal | 0.251712 | 22.775759 | | |
| RP-2603_GENP4005 | Normal | NA | 25.095327 | | |
| RP-2603_GENP2455 | Normal | NA | 26.497502 | | |
| RP-2603_GENP2560 | Normal | NA | 26.724512 | | |
| RP-2603_GENP3534 | Normal | NA | 31.971938 | | |
| RP-2603_RGENP462 | Normal | NA | 23.970578 | | |
| RP-2603_RGENP544 | Normal | NA | 23.517434 | | |
| RP-2603_RGENP490 | Normal | NA | 32.808987 | | |
| RP-2603_RGENP1401 | Normal | NA | 32.719704 | | |
| 108200_posttx138d | cfDNA | 0.235359 | 36.162875 | 0.02715 | 0.1008 |
| 108200_pretx530d | cfDNA | 0.294852 | 43.905708 | 0.03756 | 0.0965 |
| 128311_posttx127d | cfDNA | 0.281013 | 57.08915 | 0.0251 | 0.0504228 |
| 128311_pretx1333d | cfDNA | 0.269502 | 44.706981 | 0.04665 | 0.006111 |
| 384102_pretx78d | cfDNA | 0.269321 | 67.089394 | 0.02854 | 0.097 |
| 407120_posttx489d | cfDNA | 0.229989 | 37.576113 | 0.04387 | 0.01697048 |
| 407120_pretx683d | cfDNA | 0.275717 | 39.969391 | 0.03968 | 0.027 |
| 476703_pretx57d | cfDNA | 0.233371 | 46.207721 | 0.1697 | 0.214 |
| 500560_pretx11d | cfDNA | 0.257722 | 54.542077 | 0.03338 | 0.04733333 |
| 566240_pretx437d | cfDNA | 0.269322 | 48.929916 | 0.01002 | 0.066 |
| 566691_posttx26d | cfDNA | 0.269905 | 42.891232 | 0.08552 | 0.19025 |
| 566691_pretx0d | cfDNA | 0.253287 | 48.774302 | 0.1364 | 0.23767511 |
| 646059_pretx6d | cfDNA | 0.31625 | 70.581292 | 0.2091 | 0.13717056 |
| 653096_posttx31d | cfDNA | 0.30965 | 103.134103 | 0.4032 | 0.2455025 |
| 653096_pretx0d | cfDNA | 0.276147 | 47.464978 | 0.06398 | 0.08881667 |
| 718458_pretx0d | cfDNA | 0.334724 | 60.967274 | 0.04429 | 0.04089655 |
| 752770_pretx0d | cfDNA | 0.256369 | 48.692124 | 0.2445 | 0.17309631 |
| GENP2455-2 | cfDNA | NA | 35.299119 | 0.1621 | 0.11050257 |
| GENP2560-2 | cfDNA | NA | 41.53834 | 0.1277 | 0.085625 |
| GENP3534-1 | cfDNA | NA | 48.158485 | 0.1997 | 0.08529 |
| GENP4005-1 | cfDNA | NA | 42.874261 | 0.04352 | 0.028411 |
| RGENP1401_P2 | cfDNA | NA | 50.818821 | 0.1659 | 0.4660016 |
| RGENP462_2 | cfDNA | NA | 86.882298 | 0.4817 | 0.36948991 |
| RGENP490_1 | cfDNA | NA | 47.341518 | 0.311 | 0.13783139 |
| RGENP544_1 | cfDNA | NA | 33.110691 | 0.07592 | 0.16083333 |

TABLE 12
(patients)

| | | HRD_{—} | HR_gene_{—} | BRCA_germline_{—} | |
|---|---|---|---|---|---|
| sample | study | status | mut | or_somatic | BRCA_Variant |
| 108200 | Radium-223 | neg | NA | NA | |
| 128311 | Radium-223 | neg | NA | NA | |
| 384102 | Radium-223 | neg | NA | NA | |
| 407120 | Radium-223 | pos | BRCA2 | Germline | 13:32913837:DEL:AA>− |
| 476703 | Radium-223 | neg | NA | NA | |
| 500560 | Radium-223 | pos | BRCA2 | Germline | 13:32912965:DEL:GAAA>− |
| 566240 | Radium-223 | pos | BRCA1 | Germline | 17:41276045:DEL:CT>− |
| 566691 | Radium-223 | neg | NA | NA | |
| 646059 | Radium-223 | pos | BRCA2 | Somatic | 13:32911085:SNP:G>T |
| 653096 | Radium-223 | pos | BRCA2 | Germline | 13:32914438:DEL:T>− |
| 718458 | Radium-223 | neg | NA | NA | |
| 752770 | Radium-223 | pos | BRCA2 | Somatic | 13:32910564:INS:−>A |
| GENP2455-2 | docetaxel | pos | BRCA2 | Germline | 13:32914438:DEL:T>− |
| GENP2560-2 | docetaxel | pos | BRCA2 | Germline | 13:32903605:DEL:TG>− |
| GENP3534-1 | docetaxel | neg | NA | NA | |
| GENP4005-1 | docetaxel | neg | NA | NA | |
| RGENP1401_P2 | docetaxel | neg | NA | NA | |
| RGENP462_2 | docetaxel | pos | BRCA2 | Germline | 13:32907004:DEL:AG>− |
| RGENP490_1 | docetaxel | pos | BRCA2 | Germline | 13:32906752:DEL:A>− |
| RGENP544_1 | docetaxel | neg | NA | NA | |

| | BRCA_{—} | BRCA_Variant_{—} | BRCA_Variant_{—} |
|---|---|---|---|
| sample | Variant_VAF | Classification | ClinVar_VCF_CLNSIG |
| 108200 | | | |
| 128311 | | | |
| 384102 | | | |
| 407120 | 0.46 | Frame_Shift_Del | Pathogenic |
| 476703 | | | |
| 500560 | 0.32 | Frame_Shift_Del | Pathogenic |
| 566240 | 0.49 | Frame_Shift_Del | Pathogenic |
| 566691 | | | |
| 646059 | 0.11 | Nonsense_Mutation | Pathogenic |
| 653096 | 0.47 | Frame_Shift_Del | Pathogenic |
| 718458 | | | |
| 752770 | 0.18 | Frame_Shift_Ins | Pathogenic |
| GENP2455-2 | 0.46 | Frame_Shift_Del | Pathogenic |
| GENP2560-2 | 0.47 | Frame_Shift_Del | Pathogenic |
| GENP3534-1 | | | |
| GENP4005-1 | | | |
| RGENP1401_P2 | | | |
| RGENP462_2 | 0.55 | Frame_Shift_Del | Pathogenic |
| RGENP490_1 | 0.47 | Frame_Shift_Del | Pathogenic |
| RGENP544_1 | | | |

TABLE 13
(patients)

| | sigMA_{—} | | | |
|---|---|---|---|---|
| | hrdstatus | scarHRD_{—} | scarHRD_{—} | tumor |
| patient | (sig3) | score | hrdstatus | stage |
| OC_NACT_A | TRUE | 61 | TRUE | IIIC |
| OC_NACT_B | TRUE | 93 | TRUE | IVB |
| OC_NACT_D | TRUE | 73 | TRUE | IVB |
| OC_NACT_F | FALSE | 13 | FALSE | IVA |
| OC_NACT_M | TRUE | 90 | TRUE | IIIC |
| OC_NACT_S | TRUE | 84 | TRUE | IIIC |
| OC_NACT_U | TRUE | 73 | TRUE | IIIC |
| OC_NACT_V | FALSE | 38 | FALSE | IVA |
| OC_NACT_W | FALSE | 29 | FALSE | IIIC |
| OC_NACT_X | TRUE | 49 | TRUE | IIIC |

TABLE 14
(Samples)

| | sample_{—} | wgs_mean_{—} | wgs_median_{—} | Duplication_{—} | ulp_tumor_{—} | panel_{—} |
|---|---|---|---|---|---|---|
| sample_alias | type | coverage | coverage | rate | fraction | mean_af |
| OC_NACT_A_WB_T1 | Normal | 26.709874 | 25 | 0.134584 | | |
| OC_NACT_B_WB_T1 | Normal | 28.825453 | 27 | 0.142263 | | |
| OC_NACT_D_WB_T1 | Normal | 24.421493 | 23 | 0.177018 | | |
| OC_NACT_F_WB_T5 | Normal | 28.587979 | 27 | 0.129221 | | |
| OC_NACT_M_WB_T1 | Normal | 30.135181 | 29 | 0.143177 | | |
| OC_NACT_S_WB_T1 | Normal | 23.544775 | 22 | 0.173004 | | |
| OC_NACT_U_WB_T1 | Normal | 25.764227 | 24 | 0.11907 | | |
| OC_NACT_V_WB_T1 | Normal | 26.237022 | 25 | 0.127642 | | |
| OC_NACT_W_WB_T1 | Normal | 33.931723 | 32 | 0.144899 | | |
| OC_NACT_X_WB_T1 | Normal | 30.968404 | 29 | 0.132774 | | |
| OC_NACT_A_PL_T1 | cfdna | 59.409478 | 58 | 0.150736 | 0.04245 | 0.012 |
| OC_NACT_B_PL_T1 | cfdna | 45.016973 | 42 | 0.119992 | 0.06433 | 0.03 |
| OC_NACT_D_PL_T1 | cfdna | 66.105474 | 63 | 0.1358 | 0.05381 | 0.011 |
| OC_NACT_F_PL_T1 | cfdna | 60.317104 | 56 | 0.144 | 0.02563 | 0.019 |
| OC_NACT_M_PL_T1 | cfdna | 48.349632 | 43 | 0.171696 | 0.061 | 0.034 |
| OC_NACT_S_PL_T1 | cfdna | 63.03179 | 55 | 0.13887 | 0.08478 | 0.022 |
| OC_NACT_U_PL_T1 | cfdna | 68.51843 | 63 | 0.13475 | 0.03581 | 0.012 |
| OC_NACT_V_PL_T1 | cfdna | 55.210902 | 46 | 0.153656 | 0.07147 | 0.062 |
| OC_NACT_W_PL_T1 | cfdna | 56.907273 | 50 | 0.154239 | 0.03321 | 0.019 |
| OC_NACT_X_PL_T1 | cfdna | 41.767513 | 37 | 0.150994 | 0.04685 | 0.014 |

Various Embodiments: Features described above as well as those claimed below may be combined in various ways without departing from the scope hereof. The following examples illustrate some possible, non-limiting combinations. The disclosure also provides support for a system comprising: a detector module configured to detect, in a signal series, referential events based on sequence reads in the signal series, the detector module configured to execute one or more signal processing heuristics to exclude referential event candidates based at least in part on relative positioning of the referential event candidates in the signal series; a classifier configured to assign mutational signature classes to referential events based on one or more characteristics of the referential events; a modeler configured to apply a trained model to classified referential events to obtain scores indicative of a degree to which the classified referential events are associated with a repair pathway; and an output device configured to provide, based on the scores, one or more outputs corresponding to a deficiency or a treatment for the deficiency. The disclosure moreover provides support for a method comprising: detecting, in a signal series, referential events based on sequence reads in the signal series, wherein detecting the referential events comprises executing one or more signal processing heuristics to exclude referential event candidates based at least in part on relative positioning of the referential event candidates in the signal series; assigning mutational signature classes to referential events based on one or more characteristics of the referential events; applying a trained model to classified referential events to obtain scores indicative of a degree to which the classified referential events are associated with a repair pathway; and outputting, based on the scores, one or more outputs corresponding to a deficiency or a treatment for the deficiency.

From the foregoing description, it will be apparent that variations and modifications may be made to the embodiments of the disclosure described herein to adopt it to various usages and conditions. Such embodiments are also within the scope of the following claims. The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof. All patents and publications mentioned in this specification are herein incorporated by reference to the same extent as if each independent patent and publication was specifically and individually indicated to be incorporated by reference.

Features described above as well as those claimed below may be combined in various ways without departing from the scope hereof. The following examples illustrate some possible, non-limiting combinations:

What is claimed is:

1. A computing system comprising one or more processors and a non-transitory computer-readable storage medium storing instructions executable by the one or more processors, the computing system comprising:
a detector module configured to detect, in a signal series, localized disruptions based on reads in the signal series, wherein the localized disruptions are microhomology deletions or indels less than or equal to 50 base pairs;
a classifier configured to assign signature classes to the detected localized disruptions based on one or more features of the localized disruptions to obtain classified localized disruptions, the one or more features indicative of a microhomology-based disruption type;
a modeler configured to apply a trained model to the classified localized disruptions to obtain scores indicative of a degree to which the classified localized disruptions are associated with a repair pathway, the trained model being trained based at least in part on a plurality of disruption types; and
an output module configured to provide, based on the scores, one or more outputs related to at least one of a target condition corresponding to the repair pathway or to provide a treatment for the target condition.

2. The computing system of claim 1, wherein the detector module is configured to execute one or more signal processing heuristics to exclude localized disruption candidates based at least in part on relative positioning of the localized disruption candidates in the signal series.

3. The computing system of claim 2, wherein at least one of the signal processing heuristics or the classifier increases a signal-to-noise ratio (SNR) in analysis of the signal series.

4. The computing system of claim 2, wherein the signal processing heuristics exclude candidate localized disruptions based at least in part on a distance between adjacent candidate localized disruptions or based at least in part on whether the candidate localized disruptions are situated in low-complexity regions of the signal series.

5. The computing system of claim 1, wherein the repair pathway corresponds to a homologous recombination repair pathway and/or the target condition is homologous recombination deficiency (HRD) or neoplasia.

6. The computing system of claim 1, wherein the scores are indicative of an ID6-based HRD+ signature.

7. The computing system of claim 1, wherein the modeler is configured to obtain a posterior probability of each localized disruption being associated with ID6, wherein a composite score is a sum of probabilities for deletions of ≥5 bp with ≥1 bp of homology, and wherein the composite score at least as great as a threshold indicates HRD positivity.

8. The computing system of claim 7, wherein the threshold indicating HRD positivity is ≥1.

9. The computing system of claim 1, wherein the treatment comprises at least one of a PARP inhibitor or platinum chemotherapy.

10. The computing system of claim 1, wherein the trained model comprises a multinomial mixture model.

11. The computing system of claim 1, further comprising a training module configured to train the model, the training module being configured to optimize the model based at least in part on an expectation-maximization optimization algorithm.

12. The computing system of claim 1, wherein the detector module requires localized disruption candidates to be supported by at least two unique fragments to qualify as localized disruptions.

13. A method comprising:
detecting localized structural disruptions in a signal series based on reads in the signal series;
assigning signature classes to the detected localized disruptions based on one or more features of the localized disruptions to obtain classified localized disruptions, the one or more features being indicative of a microhomologic disruption type;
applying a trained model to the classified localized disruptions to obtain scores indicative of a degree to which the classified localized disruptions are associated with a repair pathway, the trained model being trained based at least in part on a plurality of disruption types, wherein the scores correspond to microhomology deletions or indels less than or equal to 50 base pairs; and
providing one or more outputs related to at least one of a target condition corresponding to the repair pathway or a treatment for the target condition based on the scores.

14. The method of claim 13, wherein detecting the localized disruptions comprises applying one or more signal processing heuristics to exclude localized disruption candidates based at least in part on relative positioning of the localized disruption candidates in the signal series.

15. The method of claim 14, wherein the signal processing heuristics exclude candidate localized disruptions based at least in part on a distance between adjacent candidate localized disruptions or based at least in part on whether the candidate localized disruptions are situated in low-complexity regions of the signal series.

16. The method of claim 13, wherein the repair pathway corresponds to a homologous recombination repair pathway and/or the target condition is homologous recombination deficiency or neoplasia.

17. The method of claim 13, wherein the trained model comprises a multinomial mixture model.

18. The method of claim 13, further comprising training the model, wherein training the model comprises optimizing the model based at least in part on an expectation-maximization optimization algorithm.

19. The method of claim 13, wherein the scores are indicative of an ID6-based HRD+ signature.

20. The method of claim 13, wherein the modeler is configured to obtain a posterior probability of each localized disruption being associated with ID6, wherein a composite score is a sum of probabilities for deletions ≥5 bp with ≥1 bp of homology, and/or wherein the composite score at least as great as a threshold indicates HRD positivity.

21. The method of claim 13, wherein localized disruption candidates are required to be supported by at least two unique fragments to qualify as localized disruptions.

22. A non-transitory computer-readable storage medium storing instructions executable by one or more processors of a computing system to cause the computing system to:
detect localized structural disruptions in a signal series based on reads in the signal series;
assign signature classes to the detected localized disruptions based on one or more features of the localized disruptions to obtain classified localized disruptions, the one or more features indicative of a microhomology disruption type;
apply a trained model to the classified localized disruptions to obtain scores indicative of a degree to which the classified localized disruptions are associated with a repair pathway, the trained model being trained based at least in part on a plurality of disruption types; and
provide one or more outputs related to a target condition corresponding to the repair pathway and/or a treatment for the target condition based on the scores.

23. The non-transitory computer-readable medium of claim 22, wherein detecting the localized disruptions comprises applying one or more signal processing heuristics to exclude localized disruption candidates based at least in part on relative positioning of the localized disruption candidates in the signal series, wherein the localized disruptions are microhomology deletions or indels no greater than 50 base pairs.


SEQUENCE LISTING (98 sequences)

SEQ ID NO: 1 - DNA, 13 bp, synthetic construct
  source 1..13: mol_type = other DNA
gggaagggcgatc

SEQ ID NO: 2 - DNA, 12 bp, synthetic construct
  source 1..12: mol_type = other DNA
gggaagggcgat

SEQ ID NO: 3 - DNA, 13 bp, synthetic construct
  source 1..13: mol_type = other DNA
gggaagggcgatc

SEQ ID NO: 4 - DNA, 10 bp, synthetic construct
  source 1..10: mol_type = other DNA
gggaagggcg

SEQ ID NO: 5 - DNA, 12 bp, synthetic construct
  source 1..12: mol_type = other DNA
  misc_difference 6: note = T deletion at this location
acccctcgcggc

SEQ ID NO: 6 - DNA, 12 bp, synthetic construct
  source 1..12: mol_type = other DNA
  misc_difference 6: note = C deletion at this location
accaactgcggc

SEQ ID NO: 7 - DNA, 12 bp, synthetic construct
  source 1..12: mol_type = other DNA
  misc_difference 6: note = T deletion at this location
accccttgcggc

SEQ ID NO: 8 - DNA, 12 bp, synthetic construct
  source 1..12: mol_type = other DNA
  misc_difference 6: note = C deletion at this location
accaaccgcggc

SEQ ID NO: 9 - DNA, 13 bp, synthetic construct
  source 1..13: mol_type = other DNA
  misc_difference 6: note = T deletion at this location
acccctttgcggc

SEQ ID NO: 10 - DNA, 13 bp, synthetic construct
  source 1..13: mol_type = other DNA
  misc_difference 6: note = C deletion at this location
accaacccgcggc

SEQ ID NO: 11 - DNA, 14 bp, synthetic construct
  source 1..14: mol_type = other DNA
  misc_difference 6: note = T deletion at this location
accccttttgcggc

SEQ ID NO: 12 - DNA, 14 bp, synthetic construct
  source 1..14: mol_type = other DNA
  misc_difference 6: note = C deletion at this location
accaaccccgcggc

SEQ ID NO: 13 - DNA, 15 bp, synthetic construct
  source 1..15: mol_type = other DNA
  misc_difference 6: note = T deletion at this location
acccctttttgcggc

SEQ ID NO: 14 - DNA, 15 bp, synthetic construct
  source 1..15: mol_type = other DNA
  misc_difference 6: note = C deletion at this location
accaacccccgcggc

SEQ ID NO: 15 - DNA, 16 bp, synthetic construct
  source 1..16: mol_type = other DNA
  misc_difference 6: note = T deletion at this location
accccttttttgcggc

SEQ ID NO: 16 - DNA, 16 bp, synthetic construct
  source 1..16: mol_type = other DNA
  misc_difference 6: note = C deletion at this location
accaaccccccgcggc

SEQ ID NO: 17 - DNA, 12 bp, synthetic construct
  source 1..12: mol_type = other DNA
  misc_difference 6: note = T insertion at this location
acccctcgcggc

SEQ ID NO: 18 - DNA, 12 bp, synthetic construct
  source 1..12: mol_type = other DNA
  misc_difference 6: note = C insertion at this location
accaactgcggc

SEQ ID NO: 19 - DNA, 12 bp, synthetic construct
  source 1..12: mol_type = other DNA
  misc_difference 6: note = T insertion at this location
accccttgcggc

SEQ ID NO: 20 - DNA, 12 bp, synthetic construct
  source 1..12: mol_type = other DNA
  misc_difference 6: note = C insertion at this location
accaaccgcggc

SEQ ID NO: 21 - DNA, 13 bp, synthetic construct
  source 1..13: mol_type = other DNA
  misc_difference 6: note = T insertion at this location
acccctttgcggc

SEQ ID NO: 22 - DNA, 13 bp, synthetic construct
  source 1..13: mol_type = other DNA
  misc_difference 6: note = C insertion at this location
accaacccgcggc

SEQ ID NO: 23 - DNA, 14 bp, synthetic construct
  source 1..14: mol_type = other DNA
  misc_difference 6: note = T insertion at this location
accccttttgcggc

SEQ ID NO: 24 - DNA, 14 bp, synthetic construct
  source 1..14: mol_type = other DNA
  misc_difference 6: note = C insertion at this location
accaaccccgcggc

SEQ ID NO: 25 - DNA, 15 bp, synthetic construct
  source 1..15: mol_type = other DNA
  misc_difference 6: note = T insertion at this location
acccctttttgcggc

SEQ ID NO: 26 - DNA, 15 bp, synthetic construct
  source 1..15: mol_type = other DNA
  misc_difference 6: note = C insertion at this location
accaacccccgcggc

SEQ ID NO: 27 - DNA, 16 bp, synthetic construct
  source 1..16: mol_type = other DNA
  misc_difference 6: note = T insertion at this location
accccttttttgcggc

SEQ ID NO: 28 - DNA, 16 bp, synthetic construct
  source 1..16: mol_type = other DNA
  misc_difference 6: note = C insertion at this location
accaaccccccgcggc

SEQ ID NO: 29 - DNA, 14 bp, synthetic construct
  source 1..14: mol_type = other DNA
  misc_difference 6..7: note = TC deletion at this location
accaatcaagcggc

SEQ ID NO: 30 - DNA, 14 bp, synthetic construct
  source 1..14: mol_type = other DNA
  misc_difference 6..7: note = TC deletion at this location
acccctctcgcggc

SEQ ID NO: 31 - DNA, 16 bp, synthetic construct
  source 1..16: mol_type = other DNA
  misc_difference 6..7: note = TC deletion at this location
acccctctctcgcggc

SEQ ID NO: 32 - DNA, 18 bp, synthetic construct
  source 1..18: mol_type = other DNA
  misc_difference 6..7: note = TC deletion at this location
acccctctctctcgcggc

SEQ ID NO: 33 - DNA, 20 bp, synthetic construct
  source 1..20: mol_type = other DNA
  misc_difference 6..7: note = TC deletion at this location
acccctctctctctcgcggc

SEQ ID NO: 34 - DNA, 22 bp, synthetic construct
  source 1..22: mol_type = other DNA
  misc_difference 6..7: note = TC deletion at this location
acccctctctctctctcgcggc

SEQ ID NO: 35 - DNA, 17 bp, synthetic construct
  source 1..17: mol_type = other DNA
  misc_difference 7..9: note = TTC deletion at this location
accaaattcaaagcggc

SEQ ID NO: 36 - DNA, 20 bp, synthetic construct
  source 1..20: mol_type = other DNA
  misc_difference 7..9: note = TTC deletion at this location
accaaattcttcaaagcggc

SEQ ID NO: 37 - DNA, 23 bp, synthetic construct
  source 1..23: mol_type = other DNA
  misc_difference 7..9: note = TTC deletion at this location
accaaattcttcttcaaagcggc

SEQ ID NO: 38 - DNA, 26 bp, synthetic construct
  source 1..26: mol_type = other DNA
  misc_difference 7..9: note = TTC deletion at this location
accaaattcttcttcttcaaagcggc

SEQ ID NO: 39 - DNA, 29 bp, synthetic construct
  source 1..29: mol_type = other DNA
  misc_difference 7..9: note = TTC deletion at this location
accaaattcttcttcttcttcaaagcggc

SEQ ID NO: 40 - DNA, 32 bp, synthetic construct
  source 1..32: mol_type = other DNA
  misc_difference 7..9: note = TTC deletion at this location
accaaattcttcttcttcttcttcaaagcggc

SEQ ID NO: 41 - DNA, 20 bp, synthetic construct
  source 1..20: mol_type = other DNA
  misc_difference 8..11: note = TCTC deletion at this location
accaaaatctcaaaagcggc

SEQ ID NO: 42 - DNA, 24 bp, synthetic construct
  source 1..24: mol_type = other DNA
  misc_difference 8..11: note = TCTC deletion at this location
accaaaatctctctcaaaagcggc

SEQ ID NO: 43 - DNA, 28 bp, synthetic construct
  source 1..28: mol_type = other DNA
  misc_difference 8..11: note = TCTC deletion at this location
accaaaatctctctctctcaaaagcggc

SEQ ID NO: 44 - DNA, 32 bp, synthetic construct
  source 1..32: mol_type = other DNA
  misc_difference 8..11: note = TCTC deletion at this location
accaaaatctctctctctctctcaaaagcggc

SEQ ID NO: 45 - DNA, 36 bp, synthetic construct
  source 1..36: mol_type = other DNA
  misc_difference 8..11: note = TCTC deletion at this location
accaaaatctctctctctctctctctcaaaagcggc

SEQ ID NO: 46 - DNA, 40 bp, synthetic construct
  source 1..40: mol_type = other DNA
  misc_difference 8..11: note = TCTC deletion at this location
accaaaatctctctctctctctctctctctcaaaagcggc

SEQ ID NO: 47 - DNA, 23 bp, synthetic construct
  source 1..23: mol_type = other DNA
  misc_difference 9..12: note = TCATC deletion at this location
accaaaaatcatcaaaaagcggc

SEQ ID NO: 48 - DNA, 28 bp, synthetic construct
  source 1..28: mol_type = other DNA
  misc_difference 9..12: note = TCATC deletion at this location
accaaaaatcatctcatcaaaaagcggc

SEQ ID NO: 49 - DNA, 33 bp, synthetic construct
  source 1..33: mol_type = other DNA
  misc_difference 9..12: note = TCATC deletion at this location
accaaaaatcatctcatctcatcaaaaagcggc

SEQ ID NO: 50 - DNA, 38 bp, synthetic construct
  source 1..38: mol_type = other DNA
  misc_difference 9..12: note = TCATC deletion at this location
accaaaaatcatctcatctcatctcatcaaaaagcggc

SEQ ID NO: 51 - DNA, 43 bp, synthetic construct
  source 1..43: mol_type = other DNA
  misc_difference 9..12: note = TCATC deletion at this location
accaaaaatcatctcatctcatctcatctcatcaaaaagcggc

SEQ ID NO: 52 - DNA, 48 bp, synthetic construct
  source 1..48: mol_type = other DNA
  misc_difference 6: note = TCATC deletion at this location
accaaaaatcatctcatctcatctcatctcatctcatcaaaaagcggc

SEQ ID NO: 53 - DNA, 14 bp, synthetic construct
  source 1..14: mol_type = other DNA
  misc_difference 6..7: note = TC insertion at this location
accaatcaagcggc

SEQ ID NO: 54 - DNA, 14 bp, synthetic construct
  source 1..14: mol_type = other DNA
  misc_difference 6..7: note = TC insertion at this location
acccctctcgcggc

SEQ ID NO: 55 - DNA, 16 bp, synthetic construct
  source 1..16: mol_type = other DNA
  misc_difference 6..7: note = TC insertion at this location
acccctctctcgcggc

SEQ ID NO: 56 - DNA, 18 bp, synthetic construct
  source 1..18: mol_type = other DNA
  misc_difference 6..7: note = TC insertion at this location
acccctctctctcgcggc

SEQ ID NO: 57 - DNA, 20 bp, synthetic construct
  source 1..20: mol_type = other DNA
  misc_difference 6..7: note = TC insertion at this location
acccctctctctctcgcggc

SEQ ID NO: 58 - DNA, 22 bp, synthetic construct
  source 1..22: mol_type = other DNA
  misc_difference 6..7: note = TC insertion at this location
acccctctctctctctcgcggc

SEQ ID NO: 59 - DNA, 17 bp, synthetic construct
  source 1..17: mol_type = other DNA
  misc_difference 7..9: note = TTC insertion at this location
accaaattcaaagcggc

SEQ ID NO: 60 - DNA, 20 bp, synthetic construct
  source 1..20: mol_type = other DNA
  misc_difference 7..9: note = TTC insertion at this location
accaaattcttcaaagcggc

SEQ ID NO: 61 - DNA, 23 bp, synthetic construct
  source 1..23: mol_type = other DNA
  misc_difference 7..9: note = TTC insertion at this location
accaaattcttcttcaaagcggc

SEQ ID NO: 62 - DNA, 26 bp, synthetic construct
  source 1..26: mol_type = other DNA
  misc_difference 7..9: note = TTC insertion at this location
accaaattcttcttcttcaaagcggc

SEQ ID NO: 63 - DNA, 29 bp, synthetic construct
  source 1..29: mol_type = other DNA
  misc_difference 7..9: note = TTC insertion at this location
accaaattcttcttcttcttcaaagcggc

SEQ ID NO: 64 - DNA, 32 bp, synthetic construct; identical to SEQ ID NO: 40
  source 1..32: mol_type = other DNA
  misc_difference 7..9: note = TTC insertion at this location

SEQ ID NO: 65 - DNA, 20 bp, synthetic construct
  source 1..20: mol_type = other DNA
  misc_difference 8..11: note = TCTC insertion at this location
accaaaatctcaaaagcggc

SEQ ID NO: 66 - DNA, 24 bp, synthetic construct
  source 1..24: mol_type = other DNA
  misc_difference 8..11: note = TCTC insertion at this location
accaaaatctctctcaaaagcggc

SEQ ID NO: 67 - DNA, 28 bp, synthetic construct
  source 1..28: mol_type = other DNA
  misc_difference 8..11: note = TCTC insertion at this location
accaaaatctctctctctcaaaagcggc

SEQ ID NO: 68 - DNA, 32 bp, synthetic construct; identical to SEQ ID NO: 44
  source 1..32: mol_type = other DNA
  misc_difference 8..11: note = TCTC insertion at this location

SEQ ID NO: 69 - DNA, 36 bp, synthetic construct; identical to SEQ ID NO: 45
  source 1..36: mol_type = other DNA
  misc_difference 8..11: note = TCTC insertion at this location

SEQ ID NO: 70 - DNA, 40 bp, synthetic construct; identical to SEQ ID NO: 46
  source 1..40: mol_type = other DNA
  misc_difference 8..11: note = TCTC insertion at this location

SEQ ID NO: 71 - DNA, 23 bp, synthetic construct
  source 1..23: mol_type = other DNA
  misc_difference 9..12: note = TCATC insertion at this location
accaaaaatcatcaaaaagcggc

SEQ ID NO: 72 - DNA, 28 bp, synthetic construct
  source 1..28: mol_type = other DNA
  misc_difference 9..12: note = TCATC insertion at this location
accaaaaatcatctcatcaaaaagcggc

SEQ ID NO: 73 - DNA, 33 bp, synthetic construct; identical to SEQ ID NO: 49
  source 1..33: mol_type = other DNA
  misc_difference 9..12: note = TCATC insertion at this location

SEQ ID NO: 74 - DNA, 38 bp, synthetic construct; identical to SEQ ID NO: 50
  source 1..38: mol_type = other DNA
  misc_difference 9..12: note = TCATC insertion at this location

SEQ ID NO: 75 - DNA, 43 bp, synthetic construct; identical to SEQ ID NO: 51
  source 1..43: mol_type = other DNA
  misc_difference 9..12: note = TCATC insertion at this location

SEQ ID NO: 76 - DNA, 48 bp, synthetic construct; identical to SEQ ID NO: 52
  source 1..48: mol_type = other DNA
  misc_difference 9..12: note = TCATC insertion at this location

SEQ ID NO: 77 - DNA, 14 bp, synthetic construct
  source 1..14: mol_type = other DNA
  misc_difference 6..7: note = TC deletion from this location
accaatctagcggc

SEQ ID NO: 78 - DNA, 14 bp, synthetic construct
  source 1..14: mol_type = other DNA
  misc_difference 6..7: note = TC deletion at this location
acaactcaagcggc

SEQ ID NO: 79 - DNA, 15 bp, synthetic construct
  source 1..15: mol_type = other DNA
  misc_difference 6..8: note = TTC deletion at this location
acccattctagcggc

SEQ ID NO: 80 - DNA, 15 bp, synthetic construct
  source 1..15: mol_type = other DNA
  misc_difference 6..8: note = TTC deletion at this location
accccttcaagcggc

SEQ ID NO: 81 - DNA, 16 bp, synthetic construct
  source 1..16: mol_type = other DNA
  misc_difference 6..8: note = TTC deletion at this location
acccattcttagcggc

SEQ ID NO: 82 - DNA, 16 bp, synthetic construct
  source 1..16: mol_type = other DNA
  misc_difference 6..8: note = TTC deletion at this location
accctcttcaagcggc

SEQ ID NO: 83 - DNA, 17 bp, synthetic construct
  source 1..17: mol_type = other DNA
  misc_difference 7..10: note = TATC deletion at this location
acccactatcaagcggc

SEQ ID NO: 84 - DNA, 17 bp, synthetic construct
  source 1..17: mol_type = other DNA
  misc_difference 6..9: note = TATC deletion at this location
acccatatctaagcggc

SEQ ID NO: 85 - DNA, 18 bp, synthetic construct
  source 1..18: mol_type = other DNA
  misc_difference 8..11: note = TATC deletion at this location
acccgtctatcaagcggc

SEQ ID NO: 86 - DNA, 18 bp, synthetic construct
  source 1..18: mol_type = other DNA
  misc_difference 6..9: note = TATC deletion at this location
acccatatctatagcggc

SEQ ID NO: 87 - DNA, 18 bp, synthetic construct
  source 1..18: mol_type = other DNA
  misc_difference 8..11: note = TATC deletion at this location
acccatctatcaagcggc

SEQ ID NO: 88 - DNA, 18 bp, synthetic construct
  source 1..18: mol_type = other DNA
  misc_difference 6..10: note = TAGTC deletion at this location
acccatagtcttagcggc

SEQ ID NO: 89 - DNA, 18 bp, synthetic construct
  source 1..18: mol_type = other DNA
  misc_difference 7..11: note = TAGTC deletion at this location
acccactagtcaagcggc

SEQ ID NO: 90 - DNA, 18 bp, synthetic construct
  source 1..18: mol_type = other DNA
  misc_difference 6..10: note = TAGTC deletion at this location
acccatagtctaagcggc

SEQ ID NO: 91 - DNA, 19 bp, synthetic construct
  source 1..19: mol_type = other DNA
  misc_difference 8..12: note = TAGTC deletion at this location
acccctctagtcaagcggc

SEQ ID NO: 92 - DNA, 19 bp, synthetic construct
  source 1..19: mol_type = other DNA
  misc_difference 6..10: note = TAGTC deletion at this location
acccatagtctagagcggc

SEQ ID NO: 93 - DNA, 19 bp, synthetic construct
  source 1..19: mol_type = other DNA
  misc_difference 8..12: note = TAGTC deletion at this location
acccgtctagtcaagcggc

SEQ ID NO: 94 - DNA, 20 bp, synthetic construct
  source 1..20: mol_type = other DNA
  misc_difference 6..10: note = TAGTC deletion at this location
acccatagtctagtagcggc

SEQ ID NO: 95 - DNA, 20 bp, synthetic construct
  source 1..20: mol_type = other DNA
  misc_difference 9..13: note = TAGTC deletion at this location
acccagtctagtcaagcggc

SEQ ID NO: 96 - DNA, 24 bp, synthetic construct
  source 1..24: mol_type = other DNA
  misc_difference 6..12: note = TAGCCTC deletion at this location
acccatagcctctagcctagcggc

SEQ ID NO: 97 - DNA, 24 bp, synthetic construct
  source 1..24: mol_type = other DNA
  misc_difference 11..17: note = TAGCCTC deletion at this location
acccagcctctagcctcaagcggc

SEQ ID NO: 98 - DNA, 12 bp, synthetic construct
  source 1..12: mol_type = other DNA
atgcgacatgtc